(19) United States
(12) Patent Application Publication
(10) Pub. No.: US 2009/0092614 A1
(43) Pub. Date: Apr. 9, 2009

(54) Anti-IGF-1R Antibodies and Uses Thereof

(75) Inventors: Stephen Demarest, San Diego, CA (US); Kandasamy Hariharan, San Diego, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc., Cambridge, MA (US)

(21) Appl. No.: 12/200,766

(22) Filed: Aug. 28, 2008

Related U.S. Application Data
(60) Provisional application No. 60/968,540, filed on Aug. 28, 2007.

(51) Int. Cl. A61K 39/395

(52) U.S. Cl. 424/139.1; 424/172.1; 424/174.1

(57) ABSTRACT

The invention relates to antibodies which bind to insulin like growth factor receptor-1 (IGF-1R) and uses thereof, in particular in the diagnosis and treatment of cancer. Specific human and murine monoclonal antibodies which inhibit IGF-1R-mediated pro-survival and tumor proliferation pathways, and variants, fragments, and derivatives thereof are provided. Also provided are specific human and murine monoclonal antibodies capable of synergistically inhibiting the ability of the ligands, insulin like growth factor 1 (IGF-1) and insulin like growth factor 2 (IGF-2), to bind to IGF-1R; as well as fragments, variants and derivatives of such antibodies. Antibodies of the invention produce such synergistic effects via allosteric and/or competitive inhibition of IGF-1R ligand binding.

### REFERENCE TO ELECTRONICALLY FILED SEQUENCE LISTING

This application incorporates by reference a “Sequence Listing” (identified below) which is submitted concurrently herewith in text file format via the U.S. Patent Office's Electronic Filing System (EFS). The text file copy of the Sequence Listing submitted herewith is labeled “2159-1000006_Seq_Listing.txt”, is a file of 136,380 bytes in size, and was created on Aug. 28, 2008; this Sequence Listing is incorporated by reference in its entirety herein.

### CROSS REFERENCE TO RELATED APPLICATIONS

This application claims benefit under 35 U.S.C. § 119(e) of U.S. Provisional Application No. 60/968,540 filed Aug. 28, 2007. This application is also related to U.S. patent application Ser. No. 11/727,887, filed on Mar. 28, 2007, which claims benefit under 35 U.S.C. § 119(e) of U.S. Provisional Application No. 60/786,347, filed on Mar. 28, 2006 and of U.S. Provisional Application No. 60/876,554 filed on Dec. 22, 2006. Each of the above-referenced patent applications is hereby incorporated by reference in its entirety.

### BACKGROUND OF THE INVENTION

A number of epidemiological studies have shown that higher than normal circulating levels of IGF-1 are associated with increased risk for several common cancers, including breast (Hankinson et al., Lancet 1998. 351:1393-6), prostate (Chan et al., Science. 1998. 279:563-6), lung (Yu et al., J. Natl. Cancer Inst. 1999. 91:151-6) and colorectal cancers (Ma et al., J. Natl. Cancer Inst. 1999. 91:620-5). Elevated circulating levels of IGF-2 also have been shown to be associated with increases risk for endometrial cancer (Oh, J. C., et al., Cancer Epidemiol. Biomarkers. Prev. 2004. 13:748-752). On the contrary, inverse correlation was observed with elevated levels of one of the IGF binding proteins, IGF-BP3, and cancer risk. Furthermore, elevated levels of IGFs have also been found in cancer patients (Peyrat et al. Eur. J. Cancer. 1993. 351:1393-6; Oh, J. C., et al., Cancer Epidemiol. Biomarkers. Prev. 2004. 13:748-752).

IGF system plays an important role in regulating cell proliferation, differentiation, apoptosis and transformation (Jones et al., Endocrinology Rev. 1995. 16:3-34). The IGF system comprises two types of unrelated receptors, the insulin like growth factor receptor 1 (IGF-1R; CD221) and insulin like growth factor receptor 2 (IGF-2R; CD222); two ligands, insulin like growth factor 1 (IGF-1 and IGF-2); several IGF binding proteins (IGFBP-1 to IGFBP-6). In addition, a large group of IGFBP proteases (e.g.: caspases, metalloproteinases, prostate-specific antigen) hydrolyze IGF bound IGFBP to release free IGFs, which then interact with IGF-1R and IGF-2R. The IGF system is also intimately connected to insulin and insulin receptor (InsR) (Moschos et al. Oncology 2002. 63:317-32; Baserga et al., Int J. Cancer. 2003. 107:873-77; Pollak et al., Nature Reviews Cancer. 2004. 4:505-516).

In a cancer cell, receptor tyrosine kinases (TK) play important role in connecting the extra-cellular tumor microenvironment to the intracellular signaling pathways that control diverse cellular functions, such as, cell division cycle, survival, apoptosis, gene expression, cytoskeletal architecture, cell adhesion, and cell migration. As the mechanisms controlling cell signaling are better understood, therapeutic strategies of disrupting one or more of these cellular functions could be developed by targeting at the level of ligand binding, receptor expression/recycling, receptor activation and the proteins involved in the signaling events (Hanahan and Weinberg, Cell 2000. 100:57-70).

The type I insulin like growth factor receptor (IGF-1R, CD221) belongs to receptor tyrosine kinase (RTK) family, (Ullrich et al., Cell. 1990, 61:203-12). IGF-1R is widely expressed and its ligands, IGF-1 and IGF-2 play a significant role in pre- and post-natal development, growth hormone responsiveness, cell transformation, survival, and have been implicated in the acquisition of an invasive and metastatic tumor phenotype (Baserga, Cell. 1994. 79:927-30; Baserga et al., Exp. Cell Res. 1999. 253:1-6, Baserga et al., Int J. Cancer. 2003. 107:873-77). Immunohistochemical studies have shown that a number of human tumors express higher levels of IGF-1R.

The molecular architecture of IGF-1R comprises, two extra-cellular α subunits (130-135 kD) and two membrane spanning β subunits (95 kD) that contain the cytoplasmic catalytic kinase domain. IGF-1R, like the insulin receptor (InsR), differs from other RTK family members by having covalent dimeric (α2β2) structures. Structurally, IGF-1R is highly related to his R (Pierre De Meyts and Whittaker, Nature Reviews Drug Discovery. 2002, 1: 769-83). IGF-1R contains 84% sequence identity to InsR at the kinase domain, whereas the juxta-membrane and the N-terminal regions share 61% and 44% sequence identity, respectively (Ulrich et al., EMBO J., 1986, 5:2503-12; Blakesley et al., Cytokine Growth Factor Rev., 1996. 7:153-56).

The IGF-1 and IGF-2 are the two activating ligands of IGF-1R. The binding of IGF-1 and IGF-2 to the α chain induces conformational changes that result in auto-phosphorylation of each β-chain at specific tyrosine residues, converting the receptor from an unphoshorylated state to the active state. The activation of three tyrosine residues in the activation loop (Tyr residues at 1131,1135 and 1136) of the kinase domain leads to an increase in catalytic activity that triggers docking and phosphorylation of the substrates such as IRS-1 and Shc adaptor proteins. Activation of these substrates leads to phosphorylation of additional proteins involved in the signaling cascade of survival (PI3K, AKT, TOR, S6) and/or proliferation (mitogen-activated protein kinase, p42/p44) (Pollak et al., Nature Reviews Cancer. 2004. 4:505-516; Baserga et al., Biochem Biophys Act. 1997. 1332:F105-F126; Baserga et al., Int. J. Cancer. 2003. 107:873-77).

Despite the high degree of homology between IGF-1R and InsR, evidence suggests that the two receptors have distinct biological roles; InsR is a key regulator of physiological functions such as glucose transport and the biosynthesis of glycogen and fat, whereas IGF-1R is a potent regulator of cell growth and differentiation. In contrast to InsR, IGF-1R is ubiquitously expressed in tissues where it plays a role in tissue growth, under the control of growth hormone (GH), which modulates IGF-1. Although IGF-1R activation has been shown to promote normal cell growth, experimental evidence suggests that IGF-1R is not an absolute requirement (Baserga et al., Exp Cell Res. 1999. 253:1-6; Baserga et al., Int. J. Cancer. 2003. 107:873-77).

IGFs play a crucial role in regulating cell proliferation, differentiation and apoptosis. Inhibition of IGF-1R mediated signaling has been shown to reduce tumor growth rate, increase apoptosis, increase killing of tumors by chemotherapy and other molecular target therapies (reviewed in Pollak et al., Nature Reviews Cancer. 2004. 4:505-516; Zhang et al., Breast Cancer Res. 2000. 2:170-75; Chakravarti et al., Cancer Res. 2002. 62:200-07).

Experimental approaches undertaken to inhibit IGF-1R function in tumors have provided encouraging but limited success, and their effectiveness in treating cancer is yet to be determined in the clinic. The experimental approaches include; antibodies to IGF-1R (Kull et al., J. Biol. Chem. 1983, 258:6561-66; Kalebic et al., Cancer Res. 1994. 54:5531-4), neutralizing antibodies to IGF-1 or IGF-2 (Feng et al., Mol. Cancer Therapy. 2006. 5:114-20; Miyamoto et al., Clin. Cancer Res. 2005, 11:3494-502), small-molecule tyrosine kinase inhibitors (Garcia-Escheverria et al., Cancer Cell. 2004. 5:231-9; Scotlandi et al., Cancer Res. 2005. 65:3868-76), antisense oligonucleotides (Shapiro et al., J. Clin. Invest. 1994. 94:1235-42; Wraight et al. Nature Biotech. 2000. 18:521-26; Scotlandi et al., Cancer Gene Therapy. 2002. 9:296-07), dominant-negative mutants of IGF-1R (Prager et al., Proc. Natl. Acad. Sci. 1994, 91:2181-85; Kalebic et al., Int. J. Cancer 1998. 76:223-7; Scotlandi et al., Int. J. Cancer. 2002:101:11-6), analogues of the IGF ligand (Pietrzkowski et al., Mol. Cell. Biol. 1992. 12:3883-89), recombinant IGF binding proteins (Yee et al. Cell growth Differ. 1994. 5:73-77; Van Den Berg et al., Eur. J. Cancer. 1997, 33:1108-1113; Jerome et al. AACR 2004, Abstract # 5334), antagonists of GH-releasing hormone, GHRH (Szereday et al., Cancer Res. 2003. 63:7913-19; Letsh et al., Proc Natl. Acad. Sci. USA. 2003. 100:1250-55) and GH (Kopchick et al., 2002. Endocr. Rev. 23, 623-46).

The ability of an antibody to inhibit IGF-1R function was first demonstrated with a mouse monoclonal antibody (αIR3) targeting an unknown epitope in the α subunit of IGF-1R (Kull et al., J. Biol. Chem. 1983, 258:6561-66). Subsequently other antibodies developed to the α subunit of IGF-1R have been shown to inhibit IGF-1R function to varying degrees in different experimental cancer models (Maloney et al. Cancer Res. 2003. 63: 5073-83; Burtrum et al., Cancer Res. 2003. 63:8912-21; Sachdev D et al., Cancer Res. 2003. 63, 627-35; Wu., et al., Clin. Cancer Res. 2005. 11:3065-74; Goetsch et al., Intl. J. Cancer. 2005. 113:316-28. Lu et al., J. Biol. Chem. 2005. 280:19665-72).

In a cancer cell, in addition to pro-survival and proliferative signaling, activation of IGF-1R has also been shown to be involved in motility and invasion (Reiss et al., Oncogene 2001. 20:490-500, Nolan et al., Int. J. Cancer. 1997.72:828-34, Stracke et al.; J. Biol. Chem. 1989. 264:21544-49; Jackson et al., Oncogene, 2001. 20:7318-25).

Tumor cells have been shown to produce one or more of the components of the IGF system (IGF-1, IGF-2, IGF-1R, IGF-2R and IGF-BPs). Although in vitro studies have indicated that tumors can produce IGF-1 or IGF-2, translational studies indicate that IGF-2 is the more relevant and commonly expressed IGF in the tumors. This is due to loss of imprinting (LOI) of the silenced IGF-2 allele in the tumor by epigenetic alterations, resulting in biallelic expression of the IGF-2 gene (Fienberg et al., Nat. Rev. Cancer 2004. 4:143-53; Giovannucci et al., Horm. Metab. Res. 2003. 35:694-04; De Souza et al., FASEB J. et al., 1997. 11:60-7). This in turn results in increased IGF-2 supply to cancer cells and to the microenvironment supporting tumor growth.

IGF-1R sensitive tumors receive receptor activation signals of IGF-1 from the circulation (liver produced) and IGF-2 from the tumor, and thus approaches aimed at disrupting the biological activity mediated by both IGF-1 and IGF-2 should provide a better anti-tumor response. Therefore, anti-IGF-1R antibody methods that effectively block the biological functions mediated by both IGF-1 and IGF-2 may provide an improved efficacy over other approaches that do not efficiently block the biological functions of both IGF-1 and IGF-2 mediated IGF-1R signaling in tumor microenvironment.

With regard to safety, IGF-1R is ubiquitously expressed and thus antibodies targeting IGF-1R should have minimal or no effector functions to avoid toxicities resulting from ADCC and CDC activities in normal tissues. One possibility of developing such antibodies is to have the non-glycosylated form of the human gamma 4 Fc region, which does not mediate ADCC or CDC functions.

IGF-1R is involved in oncogene mediated cellular transformation.

IGF/IGF-1R activation mediates mitogenic and pro-survival signaling in cancer cell.

IGF-1R activation also promotes cell motility and metastasis.

IGF-1R is over expressed in many cancers.

Individuals with higher than normal circulating IGF levels have increased risk for developing cancer.

Increased plasma levels of IGF 1 & 2 found in many cancer patients.

Human tumors produce IGF-2 as an autocrine growth factor.

Inhibition of tumor growth has been demonstrated as single agent and in combination with chemotherapeutic and biological agents.

There remains a need in the art for IGF-1R antibodies with different or improved binding, efficacy, and safety characteristics for the treatment of various neoplastic diseases including cancer and metastases thereof.

### BRIEF SUMMARY OF THE INVENTION

The present invention is based on the important role of the IGF system in regulating cell proliferation, differentiation, apoptosis and transformation. In particular, type I insulin like growth factor receptor (IGF-1R) and its ligands, IGF-1 and IGF-2, play a significant role in pre- and post-natal development, growth hormone responsiveness, cell transformation, survival, and have been implicated in the acquisition of an invasive and metastatic tumor phenotype. The invention relates generally to IGF-1R antibodies, antigen binding fragments or derivatives thereof. Certain IGF-1R antibodies and antigen-binding fragments inhibit IGF-1R function or block the biological functions of IGF-1 and IGF-2 mediated IGF-1R signaling. Additionally, the invention generally relates to methods for treating various neoplastic diseases including cancer and metastases, as well as various hyperproliferative disease, disorders or injuries associated with IGF-1R signaling.

In some embodiments, the invention provides an isolated antibody or antigen-binding fragment thereof which specifically binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.

In some embodiments, the invention provides an isolated antibody or antigen-binding fragment thereof which specifically binds to IGF-1R, where the antibody or fragment competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8 from binding to IGF-1R.

In some embodiments, the invention provides an isolated antibody or antigen-binding fragment thereof which specifically binds to IGF-1R, where the antibody or fragment thereof comprises an antigen binding domain identical to that of a monoclonal Fab antibody fragment selected from the group consisting of M13-C06, M14-G11, M14-C03, M14-B01, M12-E01, and M12-G04, or a monoclonal antibody produced by a hybridoma selected from the group consisting of P2A7.3E11, 20C8.3B8, P1A2.2B11, 20D8.24B11, P1E2.3B12, and P1G10.2B8.

In some embodiments, the invention provides an isolated antibody or fragment thereof which specifically binds to IGF-1R, where the heavy chain variable region (VH) of the antibody or fragment thereof comprises an amino acid sequence at least 90% identical to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.

In some embodiments, the invention provides an isolated antibody or fragment thereof which specifically binds to IGF-1R, where the light chain variable region (VL) of the antibody or fragment thereof comprises an amino acid sequence at least 90% identical to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.

In some embodiments, the invention provides an isolated antibody or fragment thereof which specifically binds to IGF-1R, where the VH of the antibody or fragment thereof comprises an amino acid sequence identical, except for 20 or fewer conservative amino acid substitutions, to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.

In some embodiments, the invention provides an isolated antibody or fragment thereof which specifically binds to IGF-1R, where the VL of the antibody or fragment thereof comprises an amino acid sequence identical, except for 20 or fewer conservative amino acid substitutions, to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.

In some embodiments, the invention provides an isolated antibody or fragment thereof which specifically binds to IGF-1R, where the VH of the antibody or fragment thereof comprises an amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.

In some embodiments, the invention provides an isolated antibody or fragment thereof which specifically binds to IGF-1R, where the VL of the antibody or fragment thereof comprises an amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.

In some embodiments, the invention provides an isolated antibody or fragment thereof which specifically binds to IGF-1R, where the VH and VL of the antibody or fragment thereof comprise, respectively, amino acid sequences at least 90% identical to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118.

In some embodiments, the invention provides an isolated antibody or fragment thereof which specifically binds to IGF-1R, where the VH and VL of the antibody or fragment thereof comprise, respectively, amino acid sequences identical, except for 20 or fewer conservative amino acid substitutions each, to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118.

In some embodiments, the invention provides an isolated antibody or fragment thereof which specifically binds to IGF-1R, where the VH and VL of the antibody or fragment thereof comprise, respectively, amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118.

In some embodiments, the invention provides an isolated antibody or fragment thereof which specifically binds to IGF-1R, where the VH of the antibody or fragment thereof comprises a Kabat heavy chain complementarity determining region-1 (VH-CDR1) amino acid sequence identical, except for two or fewer amino acid substitutions, to a reference VH-CDR1 amino acid sequence selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64. In further embodiments, the VH-CDR1 amino acid sequence is selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64.

In some embodiments, the invention provides an isolated antibody or fragment thereof which specifically binds to IGF-1R, where the VH of the antibody or fragment thereof comprises a Kabat heavy chain complementarity determining region-2 (VH-CDR2) amino acid sequence identical, except for four or fewer amino acid substitutions, to a reference VH-CDR2 amino acid sequence selected from the group consisting of: SEQ ID NO: 6, SEQ ID NO: 1, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65. In further embodiments, the VH-CDR2 amino acid sequence is selected from the group consisting of: SEQ ID NO: 6, SEQ ID NO: 1, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65.

In some embodiments, the invention provides an isolated antibody or fragment thereof which specifically binds to IGF-1R, where the VH of the antibody or fragment thereof comprises a Kabat heavy chain complementarity determining region-3 (VH-CDR3) amino acid sequence identical, except for four or fewer amino acid substitutions, to a reference VH-CDR3 amino acid sequence selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66. In further embodiments, the VH-CDR3 amino acid sequence is selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66.

In some embodiments, the invention provides an isolated antibody or fragment thereof which specifically binds to IGF-1R, where the VL of the antibody or fragment thereof comprises a Kabat light chain complementarity determining region-1 (VL-CDR1) amino acid sequence identical, except for four or fewer amino acid substitutions, to a reference VL-CDR1 amino acid sequence selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119. In further embodiments, the VL-CDR1 amino acid sequence is selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119.

In some embodiments, the invention provides an isolated antibody or fragment thereof which specifically binds to IGF-1R, where the VL of the antibody or fragment thereof comprises a Kabat light chain complementarity determining region-2 (VL-CDR2) amino acid sequence identical, except for two or fewer amino acid substitutions, to a reference VL-CDR2 amino acid sequence selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 110, SEQ ID NO: 115, and SEQ ID NO: 120. In further embodiments, the VL-CDR2 amino acid sequence is selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 110, SEQ ID NO: 115, and SEQ ID NO: 120.

In some embodiments, the invention provides an isolated antibody or fragment thereof which specifically binds to IGF-1R, where the VL of the antibody or fragment thereof comprises a Kabat light chain complementarity determining region-3 (VL-CDR3) amino acid sequence identical, except for four or fewer amino acid substitutions, to a reference VL-CDR3 amino acid sequence selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO:1111, SEQ ID NO: 116, and SEQ ID NO: 121. In further embodiments, the VL-CDR3 amino acid sequence is selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO:1111, SEQ ID NO: 116, and SEQ ID NO: 121.

In some embodiments, the invention provides an isolated antibody or fragment thereof which specifically binds to IGF-1R, where the VH of the antibody or fragment thereof comprises VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66, except for one, two, three, or four amino acid substitutions in at least one of said VH-CDRs.

In some embodiments, the invention provides an isolated antibody or fragment thereof which specifically binds to IGF-1R, where the VH of the antibody or fragment thereof comprises VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66.

In some embodiments, the invention provides an isolated antibody or fragment thereof which specifically binds to IGF-1R, where the VL of the antibody or fragment thereof comprises VL-CDR1, VL-CDR2, and VL-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121, except for one, two, three, or four amino acid substitutions in at least one of said VL-CDRs.

In some embodiments, the invention provides an isolated antibody or fragment thereof which specifically binds to IGF-1R, where the VL of the antibody or fragment thereof comprises VL-CDR1, VL-CDR2, and VL-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121.

In various embodiments of the above-described antibodies or fragments thereof, the VH framework regions and/or VL framework regions are human, except for five or fewer amino acid substitutions.

In some embodiments, the above-described antibodies or fragments thereof bind to a linear epitope or a non-linear conformation epitope

In some embodiments, the above-described antibodies or fragments thereof are multivalent, and comprise at least two heavy chains and at least two light chains.

In some embodiments, the above-described antibodies or fragments thereof are multispecific. In further embodiments, the above-described antibodies or fragments thereof are bispecific.

In various embodiments of the above-described antibodies or fragments thereof, the heavy and light chain variable domains are fully human. In further embodiments, the heavy and light chain variable domains are from a monoclonal Fab antibody fragment selected from the group consisting of M13-C06, M14-G11, M14-C03, M14-B01, M12-E01, and M12-G04.

In various embodiments of the above-described antibodies or fragments thereof, the heavy and light chain variable domains are murine. In further embodiments, the heavy and light chain variable domains are from a monoclonal antibody produced by a hybridoma selected from the group consisting of P2A7.3E11, 20C8.3B8, P1A2.2B 11, 20D8.24B11, P1E2.3B12, and P1G10.2B8.

In various embodiments, the above-described antibodies or fragments thereof are humanized. In various embodiments, the above-described antibodies or fragments thereof are chimeric. In various embodiments, the above-described antibodies or fragments thereof are primatized. In various embodiments, the above-described antibodies or fragments thereof are fully human.

In certain embodiments, the above-described antibodies or fragments thereof are Fab fragments, Fab′ fragments, F(ab)₂ fragments, or Fv fragments.

In certain embodiments, the above-described antibodies are single chain antibodies.

In certain embodiments, the above-described antibodies or fragments thereof comprise light chain constant regions selected from the group consisting of a human kappa constant region and a human lambda constant region.

In certain embodiments, the above-described antibodies or fragments thereof comprise a heavy chain constant region or fragment thereof. In further embodiments, the heavy chain constant region or fragment thereof is human IgG4. In certain other embodiments, the IgG4 is mutagenized to remove glycosylation sites. In further embodiments, the IgG4 mutations comprise S241P and T318A, using the Kabat numbering system.

In some embodiments, the above-described antibodies or fragments thereof specifically bind to an IGF-1R polypeptide or fragment thereof, or an IGF-1R variant polypeptide, with an affinity characterized by a dissociation constant (K_{D}) which is less than the K_{D} for said reference monoclonal antibody. In further embodiments, the dissociation constant (K_{D}) is no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹¹ M, 5×10⁻¹⁴ M, 10⁻¹⁴ M, 5×10⁻¹⁵ M, or 10⁻¹⁵ M.

In some embodiments, the above-described antibodies or fragments thereof preferentially bind to a human IGF-1R polypeptide or fragment thereof, relative to a murine IGF-1R polypeptide or fragment thereof or a non-human primate IGF-1R polypeptide or fragment thereof.

In certain other embodiments, the above described antibodies or fragments thereof bind to human IGF-1R polypeptide or fragment thereof, and also binds to a non-human primate IGF-1R polypeptide or fragment thereof.

In some embodiments, the above described antibodies or fragments thereof bind to IGF-1R expressed on the surface of a cell. In further embodiments, the cell is a malignant cell, a neoplastic cell, a tumor cell, or a metastatic cell.

In some embodiments, the above described antibodies or fragments thereof block insulin growth factor from binding to IGF-1R. In further embodiments, the insulin growth factor is insulin growth factor-1 (IGF-1) or insulin growth factor-2 (IGF-2). In certain embodiments, the above described antibodies or fragments thereof block both IGF-1 and IGF-2 from binding to IGF-1R.

In some embodiments, the above described antibodies or fragments thereof inhibit IGF-1R-mediated cell proliferation, IGF-1 or IGF-2-mediated IGF-1R phosphorylation, tumor cell growth, or IGF-1R internalization.

In further embodiments, the above described antibodies or fragments thereof further comprise a heterologous polypeptide fused thereto.

In some embodiments, the above described antibodies or fragments thereof are conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In further embodiments, the detectable label is selected from the group consisting of an enzyme, a fluorescent label, a chemiluminescent label, a bioluminescent label, a radioactive label, or a combination of two or more of any said detectable labels.

In additional embodiments, the invention includes compositions comprising the above-described antibodies or fragments thereof, and a carrier.

Certain embodiments of the invention include an isolated polynucleotide comprising a nucleic acid which encodes an antibody VH polypeptide, where the amino acid sequence of the VH polypeptide is at least 90% identical to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63; and where an antibody or antigen binding fragment thereof comprising the VH polypeptide specifically binds to IGF-1R. In further embodiments, the amino acid sequence of the VH polypeptide is selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.

In certain embodiments, the nucleotide sequence encoding the VH polypeptide is optimized for increased expression without changing the amino acid sequence of the VH polypeptide. In further embodiments, the optimization comprises identification and removal of splice donor and splice acceptor sites and/or optimization of codon usage for the cells expressing the polynucleotide. In further embodiments, the nucleic acid comprises a nucleotide sequence selected from the group consisting of: SEQ ID NO: 3, SEQ ID NO: 8, SEQ ID NO: 13, SEQ ID NO: 18, SEQ ID NO: 19, SEQ ID NO: 24, SEQ ID NO: 25, SEQ ID NO: 30, SEQ ID NO: 31, SEQ ID NO: 36, SEQ ID NO: 37, SEQ ID NO: 42, SEQ ID NO: 47, SEQ ID NO: 52, SEQ ID NO: 57, and SEQ ID NO: 62.

In some embodiments, the invention provides an isolated polynucleotide comprising a nucleic acid which encodes an antibody VL polypeptide, where the amino acid sequence of the VL polypeptide is at least 90% identical to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118; and where an antibody or antigen binding fragment thereof comprising the VL polypeptide specifically binds to IGF-1R. In further embodiments, the amino acid sequence of the VL polypeptide is selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.

In certain embodiments, the nucleotide sequence encoding the VL polypeptide is optimized for increased expression without changing the amino acid sequence of said VL polypeptide. In further embodiments, the optimization comprises identification and removal of splice donor and splice acceptor sites and/or optimization of codon usage for the cells expressing the polynucleotide. In further embodiments, the nucleic acid comprises a nucleotide sequence selected from the group consisting of: SEQ ID NO: 67, SEQ ID NO: 72, SEQ ID NO: 77, SEQ ID NO: 82, SEQ ID NO: 87, SEQ ID NO: 92, SEQ ID NO: 97, SEQ ID NO: 102, SEQ ID NO: 107, SEQ ID NO: 112, and SEQ ID NO: 117.

In certain other embodiments, the invention provides an isolated polynucleotide comprising a nucleic acid which encodes an antibody VH polypeptide, where the amino acid sequence of the VH polypeptide is identical, except for 20 or fewer conservative amino acid substitutions, to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63; and where an antibody or antigen binding fragment thereof comprising said VH polypeptide specifically binds to IGF-1R.

In some embodiments, the invention provides an isolated polynucleotide comprising a nucleic acid which encodes an antibody VL polypeptide, where the amino acid sequence of the VL polypeptide is identical, except for 20 or fewer conservative amino acid substitutions, to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118; and wherein an antibody or antigen binding fragment thereof comprising said VL polypeptide specifically binds to IGF-1R.

In some embodiments, the invention provides an isolated polynucleotide comprising a nucleic acid which encodes a VH-CDR1 amino acid sequence identical, except for two or fewer amino acid substitutions, to a reference VH-CDR1 amino acid sequence selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64; and where an antibody or antigen binding fragment thereof comprising the VH-CDR1 specifically binds to IGF-1R. In further embodiments, the VH-CDR1 amino acid sequence is selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64.

In some embodiments, the invention provides an isolated polynucleotide comprising a nucleic acid which encodes a VH-CDR2 amino acid sequence identical, except for four or fewer amino acid substitutions, to a reference VH-CDR2 amino acid sequence selected from the group consisting of: SEQ ID NO: 6, SEQ ID NO: 1, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65; and where an antibody or antigen binding fragment thereof comprising the VH-CDR2 specifically binds to IGF-1R. In further embodiments, the VH-CDR2 amino acid sequence is selected from the group consisting of: SEQ ID NO: 6, SEQ ID NO: 11, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65.

In some embodiments, the invention provides an isolated polynucleotide comprising a nucleic acid which encodes a VH-CDR3 amino acid sequence identical, except for four or fewer amino acid substitutions, to a reference VH-CDR3 amino acid sequence selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66; and where an antibody or antigen binding fragment thereof comprising the VH-CDR3 specifically binds to IGF-1R. In further embodiments, the VH-CDR3 amino acid sequence is selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66.

In some embodiments, the invention provides an isolated polynucleotide comprising a nucleic acid which encodes a VL-CDR1 amino acid sequence identical, except for four or fewer amino acid substitutions, to a reference VL-CDR1 amino acid sequence selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119; and where an antibody or antigen binding fragment thereof comprising the VL-CDR1 specifically binds to IGF-1R. In further embodiments, the VL-CDR1 amino acid sequence is selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119.

In some embodiments, the invention provides an isolated polynucleotide comprising a nucleic acid which encodes a VL-CDR2 amino acid sequence identical, except for two or fewer amino acid substitutions, to a reference VL-CDR2 amino acid sequence selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 110, SEQ ID NO: 115, and SEQ ID NO: 120; and wherein an antibody or antigen binding fragment thereof comprising said VL-CDR2 specifically binds to IGF-1R. In further embodiments, the VL-CDR2 amino acid sequence is selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 110, SEQ ID NO: 115, and SEQ ID NO: 120.

In some embodiments, the invention provides an isolated polynucleotide comprising a nucleic acid which encodes a VL-CDR3 amino acid sequence identical, except for four or fewer amino acid substitutions, to a reference VL-CDR3 amino acid sequence selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO: 111, SEQ ID NO: 116, and SEQ ID NO: 121; and wherein an antibody or antigen binding fragment thereof comprising said VL-CDR3 specifically binds to IGF-1R. In further embodiments, the VL-CDR3 amino acid sequence is selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO: 11, SEQ ID NO: 116, and SEQ ID NO: 121.

In some embodiments, the invention provides an isolated polynucleotide comprising a nucleic acid which encodes an antibody VH polypeptide, where the VH polypeptide comprises VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66; and where an antibody or antigen binding fragment thereof comprising the VL-CDR3 specifically binds to IGF-1R.

In some embodiments, the invention provides an isolated polynucleotide comprising a nucleic acid which encodes an antibody VL polypeptide, wherein said VL polypeptide comprises VH-CDR1, VH-CDR2, and VH-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121; and wherein an antibody or antigen binding fragment thereof comprising said VL-CDR3 specifically binds to IGF-1R.

In some embodiments, the above-described polynucleotides further comprise a nucleic acid encoding a signal peptide fused to the antibody VH polypeptide or the antibody VL polypeptide.

In certain other embodiments, the above-described polynucleotides further comprise a nucleic acid encoding a heavy chain constant region CH1 domain fused to the VH polypeptide, encoding a heavy chain constant region CH2 domain fused to the VH polypeptide, encoding a heavy chain constant region CH3 domain fused to the VH polypeptide, or encoding a heavy chain hinge region fused to said VH polypeptide. In further embodiments, the heavy chain constant region is human IgG4. In certain other embodiments, the IgG4 is mutagenized to remove glycosylation sites. In further embodiments, the IgG4 mutations comprise S241P and T318A using the Kabat numbering system.

In some embodiments, the above-described polynucleotides comprise a nucleic acid encoding a light chain constant region domain fused to said VL polypeptide. In further embodiments, the light chain constant region is human kappa.

In various embodiments of the above-described polynucleotides, the antibody or antigen-binding fragment thereof comprising a polypeptide encoded by the nucleic acid specifically binds the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.

In various other embodiments of the above-described polynucleotides, the antibody or antigen-binding fragment thereof comprising a polypeptide encoded by the nucleic acid competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.

In various embodiments of the above-describe polynucleotides, the framework regions of the VH polypeptide or VL polypeptide are human, except for five or fewer amino acid substitutions.

In various embodiments of the above-described polynucleotides, the invention provides an antibody or antigen-binding fragment thereof comprising the polypeptide encoded by the nucleic acid, that binds to a linear epitope or a non-linear conformational epitope.

In various embodiments of the above-described polynucleotides, the antibody or antigen-binding fragment thereof comprising the polypeptide encoded by the nucleic acid is multivalent, and comprises at least two heavy chains and at least two light chains.

In certain embodiments of the above-described polynucleotides, the antibody or antigen-binding fragment thereof comprising the polypeptide encoded by the nucleic acid is multispecific. In further embodiments, the antibody or antigen-binding fragment thereof comprising the polypeptide encoded by the nucleic acid is bispecific.

In various embodiments of the above-described polynucleotides, the antibody or antigen-binding fragment thereof comprising the polypeptide encoded by the nucleic acid comprises heavy and light chain variable domains which are fully human. In further embodiments, the heavy and light chain variable domains are identical to those of a monoclonal Fab antibody fragment selected from the group consisting of M13-C06, M14-G11, M14-C03, M14-B01, M12-E01, and M12-G04.

In certain other embodiments of the above-described polynucleotides, the antibody or antigen-binding fragment thereof comprising the polypeptide encoded by the nucleic acid comprises heavy and light chain variable domains which are murine. In further embodiments, the heavy and light chain variable domains are identical to those of a monoclonal antibody produced by a hybridoma selected from the group consisting of P2A7.3E11, 20C8.3B8, P1A2.2B11, 20D8.24B11, P1E2.3B12, and P1G10.2B8.

In various embodiments of the above-described polynucleotides, the antibody or antigen-binding fragment thereof comprising the polypeptide encoded by the nucleic acid is humanized.

In various embodiments of the above-described polynucleotides, the antibody or antigen-binding fragment thereof comprising the polypeptide encoded by the nucleic acid is primatized.

In various embodiments of the above-described polynucleotides, the antibody or antigen-binding fragment thereof comprising the polypeptide encoded by the nucleic acid is chimeric.

In some embodiments of the above-described polynucleotides, the antibody or antigen-binding fragment thereof comprising the polypeptide encoded by the nucleic acid is fully human.

In various embodiments of the above-described polynucleotides, the antibody or antigen-binding fragment thereof comprising the polypeptide encoded by the nucleic acid is an Fab fragment, an Fab′ fragment, an F(ab)₂ fragment, or an Fv fragment. In certain embodiments of the above-described polynucleotides, the antibody or antigen-binding fragment thereof comprising the polypeptide encoded by the nucleic acid is a single chain antibody.

In some embodiments of the above-described polynucleotides, the antibody or antigen-binding fragment thereof comprising the polypeptide encoded by the nucleic acid specifically binds to an IGF-1R polypeptide or fragment thereof, or an IGF-1R variant polypeptide, with an affinity characterized by a dissociation constant (K_{D}) no greater than 5×10⁻² M, 10⁻² M, 5×10⁻³ M, 10⁻³ M, 5×10⁴ M, 10⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments of the above-described polynucleotides, the antibody or antigen-binding fragment thereof comprising the polypeptide encoded by the nucleic acid preferentially binds to a human IGF-1R polypeptide or fragment thereof, relative to a murine IGF-1R polypeptide or fragment thereof or a non-human primate IGF-1R polypeptide or fragment thereof.

In some embodiments of the above-described polynucleotides, the antibody or antigen-binding fragment thereof comprising the polypeptide encoded by the nucleic acid binds to a human IGF-1R polypeptide or fragment thereof, and also binds to a non-human primate IGF-1R polypeptide or fragment thereof.

In some embodiments of the above-described polynucleotides, the antibody or antigen-binding fragment thereof comprising the polypeptide encoded by the nucleic acid binds to IGF-1R expressed on the surface of a cell. In further embodiments, the cell is a malignant cell, a neoplastic cell, a tumor cell, or a metastatic cell.

In some embodiments of the above-described polynucleotides, the antibody or antigen-binding fragment thereof comprising the polypeptide encoded by said nucleic acid blocks insulin growth factor from binding to IGF-1R. In further embodiments, the insulin growth factor is insulin growth factor-1 (IGF-1) or insulin growth factor-2 (IGF-2). In certain other embodiments of the above-described polynucleotide, the antibody or antigen-binding fragment thereof blocks both IGF-1 and IGF-2 from binding to IGF-1R.

In some embodiments of the above-described polynucleotides, the antibody or antigen-binding fragment thereof comprising the polypeptide encoded by the nucleic acid inhibits IGF-1R-mediated cell proliferation, inhibits IGF-1 or IGF-2-mediated IGF-1R phosphorylation, inhibits tumor cell growth or inhibits IGF-1R internalization.

In some embodiments, the above-described polynucleotides further comprise a nucleic acid encoding a heterologous polypeptide.

In some embodiments of the above-described polynucleotides, the antibody or antigen-binding fragment thereof comprising the polypeptide encoded by the nucleic acid is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In certain other embodiments, the detectable label is selected from the group consisting of an enzyme, a fluorescent label, a chemiluminescent label, a bioluminescent label, a radioactive label, or a combination of two or more of any said detectable labels.

In some embodiments, the invention provides compositions comprising the above-described polynucleotides.

In certain other embodiments, the invention provides vectors comprising the above-described polynucleotides. In further embodiments, the polynucleotides are operably associated with a promoter. In additional embodiments, the invention provides host cells comprising such vectors. In further embodiments, the invention provides vectors where the polynucleotide is operably associated with a promoter.

In additional embodiments, the invention provides a method of producing an antibody or fragment thereof which specifically binds IGF-1R, comprising culturing a host cell containing a vector comprising the above-described polynucleotides, and recovering said antibody, or fragment thereof. In further embodiments, the invention provides an isolated polypeptide produced by the above-described method.

In some embodiments, the invention provides isolated polypeptides encoded by the above-described polynucleotides.

In further embodiments of the above-described polypeptides, the antibody or fragment thereof comprising the polypeptide specifically binds to IGF-1R. Other embodiments include the isolated antibody or fragment thereof comprising the above-described polypeptides.

In some embodiments, the invention provides a composition comprising an isolated VH encoding polynucleotide and an isolated VL encoding polynucleotide, where the VH encoding polynucleotide and the VL encoding polynucleotide, respectively, comprise nucleic acids encoding amino acid sequences at least 90% identical to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118; and where an antibody or fragment thereof encoded by the VH and VL encoding polynucleotides specifically binds IGF-1R. In further embodiments, the VH encoding polynucleotide and said VL encoding polynucleotide, respectively, comprise nucleic acids encoding amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118.

In certain other embodiments, the invention provides a composition comprising an isolated VH encoding polynucleotide and an isolated VL encoding polynucleotide, where the VH encoding polynucleotide and the VL encoding polynucleotide, respectively, comprise nucleic acids encoding amino acid sequences identical, except for less than 20 conservative amino acid substitutions, to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118; and where an antibody or fragment thereof encoded by the VH and VL encoding polynucleotides specifically binds IGF-1R. In further embodiments, the VH encoding polynucleotide encodes a VH polypeptide comprising VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66; where the VL encoding polynucleotide encodes a VL polypeptide comprising VL-CDR1, VL-CDR2, and VL-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121; and where an antibody or fragment thereof encoded by the VH and VL encoding polynucleotides specifically binds IGF-1R.

In various embodiments of the above-described compositions, the VH encoding polynucleotide further comprises a nucleic acid encoding a signal peptide fused to the antibody VH polypeptide.

In various embodiments of the above-described compositions, the VL encoding polynucleotide further comprises a nucleic acid encoding a signal peptide fused to the antibody VL polypeptide.

In some embodiments of the above-described compositions, the VH encoding polynucleotide further comprises a nucleic acid encoding a heavy chain constant region CH1 domain fused to the VH polypeptide, further comprises a nucleic acid encoding a heavy chain constant region CH2 domain fused to the VH polypeptide, further comprises a nucleic acid encoding a heavy chain constant region CH3 domain fused to the VH polypeptide, or further comprises a nucleic acid encoding a heavy chain hinge region fused to the VH polypeptide. In further embodiments, the heavy chain constant region is human IgG4. In certain other embodiments, the IgG4 is mutagenized to remove glycosylation sites. In further embodiments, the IgG4 mutations comprise S241P and T318A using the Kabat numbering system.

In some embodiments of the above-described compositions, the VL encoding polynucleotide further comprises a nucleic acid encoding a light chain constant region domain fused to the VL polypeptide. In further embodiments, the light chain constant region is human kappa.

In some embodiments of the above-described compositions, the antibody or fragment thereof encoded by the VH and VL encoding polynucleotides specifically binds the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.

In some embodiments of the above-described compositions, the antibody or fragment thereof encoded by the VH and VL encoding polynucleotides competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8 from binding to IGF-1R.

In some embodiments of the above-described compositions, the framework regions of the VH and VL polypeptides are human, except for five or fewer amino acid substitutions.

In some embodiments of the above-described compositions, the antibody or fragment thereof encoded by the VH and VL encoding polynucleotides binds to a linear epitope or a non-linear conformational epitope.

In some embodiments of the above-described compositions, the antibody or fragment thereof encoded by the VH and VL encoding polynucleotides is multivalent, and comprises at least two heavy chains and at least two light chains.

In some embodiments of the above-described compositions, the antibody or fragment thereof encoded by the VH and VL encoding polynucleotides is multispecific. In further embodiments, the antibody or fragment thereof encoded by the VH and VL encoding polynucleotides is bispecific.

In some embodiments of the above-described compositions, the antibody or fragment thereof encoded by the VH and VL encoding polynucleotides comprises heavy and light chain variable domains which are fully human. In further embodiments, the heavy and light chain variable domains are identical to those of a monoclonal Fab antibody fragment selected from the group consisting of M13-C06, M14-G11, M14-C03, M14-B0, M12-E01, and M12-G04.

In some embodiments of the above-described compositions, the antibody or fragment thereof encoded by the VH and VL encoding polynucleotides comprises heavy and light chain variable domains which are murine. In further embodiments, the heavy and light chain variable domains are identical to those of a monoclonal antibody produced by a hybridoma selected from the group consisting of P2A7.3E11, 20C8.3B8, P1A2.2B11, 20D8.24B11, P1E2.3B12, and P1G10.2B8.

In various embodiments of the above-described compositions, the antibody or antigen-binding fragment thereof comprising the polypeptide encoded by the nucleic acid is humanized.

In various embodiments of the above-described compositions, the antibody or antigen-binding fragment thereof comprising the polypeptide encoded by the nucleic acid is primatized.

In various embodiments of the above-described compositions, the antibody or antigen-binding fragment thereof comprising the polypeptide encoded by the nucleic acid is chimeric.

In some embodiments of the above-described compositions, the antibody or antigen-binding fragment thereof comprising the polypeptide encoded by the nucleic acid is fully human.

In various embodiments of the above-described compositions, the antibody or antigen-binding fragment thereof comprising the polypeptide encoded by the nucleic acid is an Fab fragment, an Fab′ fragment, an F(ab)₂ fragment, or an Fv fragment. In certain embodiments of the above-described compositions, the antibody or antigen-binding fragment thereof comprising the polypeptide encoded by the nucleic acid is a single chain antibody.

In some embodiments of the above-described compositions, the antibody or antigen-binding fragment thereof comprising the polypeptide encoded by the nucleic acid specifically binds to an IGF-1R polypeptide or fragment thereof, or an IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵-M.

In some embodiments of the above-described compositions, the antibody or antigen-binding fragment thereof comprising the polypeptide encoded by the nucleic acid preferentially binds to a human IGF-1R polypeptide or fragment thereof, relative to a murine IGF-1R polypeptide or fragment thereof or a non-human primate IGF-1R polypeptide or fragment thereof.

In some embodiments of the above-described compositions, the antibody or antigen-binding fragment thereof comprising the polypeptide encoded by the nucleic acid binds to a human IGF-1R polypeptide or fragment thereof, and also binds to a non-human primate IGF-1R polypeptide or fragment thereof.

In some embodiments of the above-described compositions, the antibody or antigen-binding fragment thereof comprising the polypeptide encoded by the nucleic acid binds to IGF-1R expressed on the surface of a cell. In further embodiments, the cell is a malignant cell, a neoplastic cell, a tumor cell, or a metastatic cell.

In some embodiments of the above-described compositions, the antibody or antigen-binding fragment thereof comprising the polypeptide encoded by said nucleic acid blocks insulin growth factor from binding to IGF-1R. In further embodiments, the insulin growth factor is insulin growth factor-1 (IGF-1) or insulin growth factor-2 (IGF-2). In certain other embodiments of the above-described compositions, the antibody or antigen-binding fragment thereof blocks both IGF-1 and IGF-2 from binding to IGF-1R.

In some embodiments of the above-described compositions, the an antibody or antigen-binding fragment thereof comprising the polypeptide encoded by the nucleic acid inhibits IGF-1R-mediated cell proliferation, inhibits IGF-1 or IGF-2-mediated IGF-1R phosphorylation, inhibits tumor cell growth or inhibits IGF-1R internalization.

In some embodiments, the above-described compositions, the VH encoding polynucleotide, the VL encoding polynucleotide, or both the VH and the VL encoding polynucleotides further comprise a nucleic acid encoding a heterologous polypeptide.

In some embodiments of the above-described compositions, the antibody or antigen-binding fragment thereof comprising the polypeptide encoded by the nucleic acid is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents. In further embodiments, the cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents. In certain other embodiments, the detectable label is selected from the group consisting of an enzyme, a fluorescent label, a chemiluminescent label, a bioluminescent label, a radioactive label, or a combination of two or more of any said detectable labels.

In some embodiments of the above-described compositions, the VH encoding polynucleotide is contained on a first vector and the VL encoding polynucleotide is contained on a second vector. In further embodiments, the VH encoding polynucleotide is operably associated with a first promoter and the VL encoding polynucleotide is operably associated with a second promoter. In certain other embodiments, the first and second promoters are copies of the same promoter. In further embodiments, the first and second promoters non-identical.

In various embodiments of the above-described compositions, the first vector and the second vector are contained in a single host cell.

In certain other embodiments of the above-described compositions, the first vector and the second vector are contained in a separate host cells.

In some embodiments, the invention provides a method of producing an antibody or fragment thereof which specifically binds IGF-1R, comprising culturing the above-described host cells, and recovering the antibody, or fragment thereof.

In other embodiments, the invention provides a method of producing an antibody or fragment thereof which specifically binds IGF-1R, comprising co-culturing separate host cells, and recovering the antibody, or fragment thereof. In further embodiments of the above-described method, the invention provides combining the VH and VL encoding polypeptides, and recovering the antibody, or fragment thereof.

In some embodiments, the invention provides an antibody or fragment thereof which specifically binds IGF-1R, produced by the above-described methods.

In some embodiments, the invention provides compositions, where the VH encoding polynucleotide and the VL encoding polynucleotide are on the same vector, as well as the vectors therein.

In various embodiments of the above described vectors, the VH encoding polynucleotide and the VL encoding polynucleotide are each operably associated with a promoter.

In various embodiments of the above described vectors, the VH encoding polynucleotide and the VL encoding polynucleotide are fused in frame, are co-transcribed from a single promoter operably associated therewith, and are cotranslated into a single chain antibody or antigen-binding fragment thereof.

In various embodiments of the above described vectors, the VH encoding polynucleotide and said VL encoding polynucleotide are co-transcribed from a single promoter operably associated therewith, but are separately translated. In further embodiments, the vectors further comprise an IRES sequence disposed between the VH encoding polynucleotide and the VL encoding polynucleotide. In certain other embodiments, the polynucleotide encoding a VH and the polynucleotide encoding a VL are separately transcribed, each being operably associated with a separate promoter. In further embodiments, the separate promoters are copies of the same promoter or the separate promoters are non-identical.

In some embodiments, the invention provides host cells comprising the above-described vectors.

In other embodiments, the invention provides a method of producing an antibody or fragment thereof which specifically binds IGF-1R, comprising culturing the above-described host cells, and recovering the antibody, or fragment thereof.

In some embodiments, the invention provides an antibody or fragment thereof which specifically binds IGF-1R, produced by the above-described methods.

In some embodiments, the invention provides a method for treating a hyperproliferative disorder in an animal, comprising administering to an animal in need of treatment a composition comprising: a) an isolated antibody or fragment as described above; and b) a pharmaceutically acceptable carrier. In further embodiments, the hyperproliferative disease or disorder is selected from the group consisting of cancer, a neoplasm, a tumor, a malignancy, or a metastasis thereof.

In various embodiments of the above-described methods, the antibody or fragment thereof specifically binds to IGF-1R expressed on the surface of a malignant cell. In further embodiments, the binding of the antibody or fragment thereof to the malignant cell results in growth inhibition of the malignant cell.

In various embodiments of the above-described methods, the antibody or fragment thereof inhibits IGF binding to the malignant cell. In further embodiments, the IGF is IGF-1 or IGF-2.

In various embodiments of the above-described methods, the antibody or fragment thereof inhibits IGF-1 from binding to said malignant cell but does not inhibit IGF-2. In certain other embodiments, the antibody or fragment thereof inhibits IGF-2 from binding to said malignant cell but does not inhibit IGF-1.

In various embodiments of the above-described methods, the antibody or fragment thereof promotes internalization of IGF-1R into the malignant cell.

In various embodiments of the above-described methods, the antibody or fragment thereof inhibits IGF-1R phosphorylation or inhibits tumor cell proliferation. In further embodiments, the tumor cell proliferation is inhibited through the prevention or retardation of metastatic growth.

In various embodiments of the above-described methods, the antibody or fragment thereof inhibits tumor cell migration. In further embodiments, the tumor cell proliferation is inhibited through the prevention or retardation of tumor spread to adjacent tissues.

In various embodiments of the above-described methods, the hyperproliferative disease or disorder is a neoplasm located in the: prostate, colon, abdomen, bone, breast, digestive system, liver, pancreas, peritoneum, adrenal gland, parathyroid gland, pituitary gland, testicles, ovary, thymus, thyroid, eye, head, neck, central nervous system, peripheral nervous system, lymphatic system, pelvis, skin, soft tissue, spleen, thoracic region, or urogenital tract.

In various embodiments of the above-described methods, the hyperproliferative disease is cancer, said cancer selected from the group consisting of: epithelial squamous cell cancer, melanoma, leukemia, myeloma, stomach cancer, brain cancer, lung cancer, pancreatic cancer, cervical cancer, ovarian cancer, liver cancer, bladder cancer, breast cancer, colon cancer, renal cancer, prostate cancer, testicular cancer, thyroid cancer, and head and neck cancer. In further embodiments, the cancer is selected from the group consisting of stomach cancer, renal cancer, brain cancer, bladder cancer, colon cancer, lung cancer, breast cancer, pancreatic cancer, ovarian cancer, and prostate cancer.

In various embodiments of the above-described methods, the animal is a mammal. In further embodiments, the mammal is a human.

### BRIEF DESCRIPTION OF THE DRAWINGS

FIG. 1: Binding activity of IGF-1R specific Fabs. (A) Binding of purified anti-IGF-1R Fab antibodies to recombinant IGF-1R-his and IGF1R-Fc protein by ELISA. (B) Binding of purified anti-IGF-1R Fab antibodies to human IGF-1R expressed on 3T3 by flow cytometry.

FIG. 2: Binding activity of Fabs to IGF-1R expressed on MCF-7 cells.

FIG. 3: Anti-IGF-1R Fabs inhibited the (A) IGF-1 and (B) IGF-2 induced phosphorylation in MCF7 cells

FIG. 4: Binding of IGF-1R Fab fragment antibodies to (A) soluble IGF-1R and (B) INSR by ELISA.

FIG. 5: The nucleotide and the amino acid sequence of the original and the modified versions of VH and VL chains of M13-C06, M14-G11, M14-C03 and M14-B01. (A) (SEQ ID NO:13) shows the single-stranded DNA sequence of heavy chain M13-C06. (B) (SEQ ID NO:77) shows the single-stranded DNA sequence of light chain M13-C06. (C) (SEQ ID NO:14) shows the amino acid sequence of heavy chain M13-C06. (D) (SEQ ID NO:78) shows the amino acid sequence of light chain M13-C06. (E) (SEQ ID NO:25) shows the single-stranded DNA sequence of heavy chain M14-C03. (F) (SEQ ID NO:87) shows the single-stranded DNA sequence of light chain M14-C03. (G) (SEQ ID NO:26) shows the amino acid sequence of heavy chain M14-C03. (H) (SEQ ID NO:88) shows the amino acid sequence of light chain M14-C03. (I) (SEQ ID NO:31) shows the single-stranded DNA sequence of heavy chain M14-G11. (J) (SEQ ID NO:92) shows the single-stranded DNA sequence of light chain M14-G11. (K) (SEQ ID NO:32) shows the amino acid sequence of heavy chain M14-G11. (L) (SEQ ID NO:93) shows the amino acid sequence of light chain M14-G11. (M) (SEQ ID NO: 19) shows the single-stranded DNA sequence of heavy chain M14-B01. (N) (SEQ ID NO:82) shows the single-stranded DNA sequence of light chain M14-B01. (O) (SEQ ID NO:20) shows the amino acid sequence of heavy chain M14-B01. (P) (SEQ ID NO:83) shows the amino acid sequence of light chain M14-B01. (Q) (SEQ ID NO:18) shows the single-stranded DNA sequence of sequence optimized heavy chain M13-C06. (R) (SEQ ID NO:14) shows the amino acid sequence of sequence optimized heavy chain M13-C06. (S) (SEQ ID NO:30) shows the single-stranded DNA sequence of sequence optimized heavy chain M14-C03. (T) (SEQ ID NO:26) shows the amino acid sequence of sequence optimized heavy chain M14-C03. (U) (SEQ ID NO:36) shows the single-stranded DNA sequence of sequence optimized heavy chain M14-G11. (V) (SEQ ID NO:32) shows the amino acid sequence of sequence optimized heavy chain M14-G11. (W) (SEQ ID NO:24) shows the single-stranded DNA sequence of sequence optimized heavy chain M14-B01. (X) (SEQ ID NO:20) shows the amino acid sequence of sequence optimized heavy chain M14-B01. (Y) (SEQ ID NO: 153) shows the single-stranded DNA sequence of light chain constant domain. (Z) (SEQ ID NO: 154) shows the amino acid sequence of light chain constant domain. (AA) (SEQ ID NO:155) shows the single-stranded DNA sequence of heavy chain agly.IgG4.P constant domains. (BB) (SEQ ID NO: 156) shows the amino acid sequence of heavy chain aglylgG4.P constant domains.

FIG. 6: Non-reduced and reduced SDS PAGE analysis of G4.P.agly versions of fully human M13-C06 and M14-C03 antibodies.

FIG. 7: The binding activity of fully human G4.P (A) and G4.P.agly (B) versions of anti-IGF-1R antibodies as determined by ELISA.

FIG. 8: The binding of fully human antibodies to IGF-1R expressed on (A) MCF-7, (B) IGF-1R/3T3 vs. 3T3 only cells was determined by flow cytometry. The binding EC50 on MCF-7 ranged between 2.7-12×10-10 nM.

FIG. 9: The ability of G4 versions of fully human antibodies to block (inhibit) (A) IGF-1 and (B) IGF-2 binding to IGF-1R was determined by an RIA.

FIG. 10: (A) Inhibition of H-23 tumor cell proliferation in response to IGF-1 by G4 versions of fully human antibodies; (B) Inhibition of H-23 tumor cell proliferation in response to IGF-2 by G4 versions of fully human antibodies; (C) Inhibition of Calu-6 tumor cell proliferation in response to IGF-1 by G4 versions of fully human antibodies.

FIG. 11: Inhibition of (A) IGF-1 and (B) IGF-2 driven receptor phosphorylation by M13.C06.G4.P.agly, M14.C03.G4.P.agly and M14.G11.P antibodies.

FIG. 12: Inhibition of downstream signaling by M13.C06.G4.P.agly. (A) Phospho Akt (Thr308) and total Akt have been shown in top and bottom rows respectively. (B) Top Phospho p44/42 MAPK and total p44/42 MAPK shown in top and bottom rows respectively.

FIG. 13: Inhibition of IGF-1 mediated tumor cell growth by selected IGF-1R mAbs. (A) H23; (B) Calu-6; (C) Panc-1; (D) BxPC3; (E) MaPaCa; and (F) Colo205. Bars show means and SD.

FIG. 14: Inhibition of IGF-1 and IGF-2 driven proliferation of H-23 cells by anti-IGF-1R antibodies.

FIG. 15: Inhibition of BxPC3 cell proliferation (driven with recombinant human IGF-1 and IGF-2) by M13-C06.G4.P.agly antibody.

FIG. 16: Inhibition of NCI-H23 cell proliferation (driven with recombinant human IGF-1 and IGF-2) by M13-C06.G4.P.agly antibody.

FIG. 17: Inhibition of A549 cell proliferation (driven with recombinant human IGF-1 and IGF-2) by M13-C06.G4.P.agly antibody.

FIG. 18: Inhibition of IGF-1 and IGF-2 induced phosphorylation of Akt at amino acid residue Ser-473 by a fully human IGF-1R antibody.

FIG. 19: Fully human M13.C06.G4.P.agly antibody exhibits in vivo dose dependent inhibition of tumor growth in a pancreatic cancer model.

FIG. 20: Fully human M13.C06.G4.P.agly antibody exhibits in vivo dose dependent inhibition of tumor growth in a lung cancer model.

FIG. 21: Fully human M13.C06.G4.P.agly antibody administered in combination with gemcitabine exhibits increased efficacy in inhibiting tumor growth.

FIG. 22: Fully human M13.C06.G4.P.agly antibody binds to IGF-1R expressed on an established cynomolgus fibroblast cell line.

FIG. 23: Cross-competition binding analysis of IGF-1R antibody binding epitopes.

FIG. 24: Co-immunoprecipitation of IRS-1 and p85 (regulatory subunit of PI3K) demonstrates M13-C06.G4.P.agly mediated inhibition of IGF-1R signal transduction.

FIG. 25: Immunoprecipitation of IGF-1R and INSR in mammalian cells demonstrates M13.C06.G4.P.agly antibody binding to IGF-1R but not insulin receptor. IGF-1R and INSR proteins were detected by immunoblot (Western blot) analysis with (A) mouse anti-human IR or (B) mouse anti-human IGF-1R.

FIG. 26: Relative binding affinity measurements of M13-C06 Fab for (A) hIGF-1R-Fc and (B) mIGF-1R-Fc. The x- and y-axis scales are identical for (A) and (B). Residuals for the binding fits are shown at the bottom of each panel to indicate the applicability of the 1:1 binding model in determining relative affinities of M13-C06 for each receptor.

FIG. 27: Examples of M13.C06 antibody binding to hIGF-1R-Fc and mIGF-1R-Fc controls in the SPR assay compared to antibody binding to IGF-1R mutant proteins SD006 (binding positive) and SD015 (binding negative). (A) M13-C06 Fab over WT hIGF-1R-Fc captured on M13-C06 surface; (A) M13-C06 Fab over WT hIGF-1R-Fc captured on M13-C06 surface; (B) M13-C06 Fab over mIGF-1R-Fc captured on M13-C06 surface; (C) M13-C06 Fab over SD006 (see Table 17) captured on M13-C06 surface; (D) M13-C06 Fab over SD015 (see Table 17) captured on M13-C06 surface.

FIG. 28: Structural representations of IGF-1R and INSR: A) Schematic diagram of the structure of IGF-1R. (A) FnIII-2 contains loop structure that is proteolytically processed in vivo as shown on the diagram. The transmembrane region is shown as a helical loop that traverses a schematic of a phospholipid bilayer. The location of the IGF-1/IGF-2 binding site within IGF-1R is shown by a star. It has been demonstrated that only one IGF-1/IGF-2 molecule binds to each IGF-1R heterodimeric molecule. (B & C) M13-C06IGF-1R binding epitope mapped to the surface of the structure of the homologous INSR. The M13-C06IGF-1R binding epitope was modeled based on the highly homologous INSR crystal structure. (B) Surface representation of the INSR structure with amino acid residue positions corresponding to the homologous positions of V462-H464 in IGF-1R (i.e., L472-K474 in INSR) are shaded black. The first three domains corresponding to IGF-1R (i.e., L1-CR-L2) (such as are included in the truncated IGF-1R(1-462)-Fc construct described herein) are shaded grey. (C) Surface representation of the INSR structure with those residues that expose surface area to solvent and that are within a 14 Å (angstrom) radius (or 28 Å diameter) of residues corresponding to 462-464 of IGF-1R (i.e., 472-474 of INSR) are shaded black. Residues corresponding to IGF-1R amino acids 462-464 are shaded grey to indicate the experimentally confirmed surface area of the proposed epitope.

FIG. 29: Immunoblot (Western blot) analysis of in vivo IGF-1R expression in mouse tumors treated with M13.C06.G4.P.agly antibody.

FIG. 30: In vivo anti-tumor activity of M13-C06.G4.P.agly in tumors generated from a primary human colon tumor.

FIG. 31: In vivo anti-tumor activity of M13-C06.G4.P.agly in tumors generated from breast carcinoma (MCF-7) cells.

FIG. 32: M13-C06 antibody does not exhibit in vitro ADCC activity.

FIG. 33: Inhibition of human IGF-1 His binding to biotinylated hIGF-1R-Fc by antibodies M13-C06, M14-C03, M14-G11, and αIR3.

FIG. 34: Inhibition of human IGF-2 His binding to biotinylated hIGF-1R-Fc by antibodies M13-C06, M14-C03, M14-G11, P1E2 and αIR3.

FIG. 35: ELISA assay for detecting human IGF-1 His binding to biotinylated hIGF-1R. Human IGF-1 His was serially diluted in PBST (circles) and PBST containing 2 μm M13-C06 (squares).

FIG. 36: Residues whose mutation affected the binding of M13-C06 to hIGF-1R-Fc were mapped to the structure of the homologous IR ectodomain. Mutation of IGF-1R amino acid residues 415, 427, 468, 478 and 532 had no detectable affect on M13-C06 antibody binding. Mutation of IGF-1R amino acid residues 466, 467, 533, 564 and 565 had a weak negative affect on M13-C06 antibody binding. Mutation of IGF-1R amino acid residues 459, 460, 461, 462, 464, 482, 483, 490, 570 and 571 had a strong negative affect on M13-C06 antibody binding. See, Table 20 for a compilation of mutation analysis results.

FIG. 37: Residues whose mutation affected the binding of M14-G11 to hIGF-1R-Fc were mapped to the structure of the first three ectodomains of human IGF-1R. Mutation of IGF-1R amino acid residues 28, 227, 237, 285, 286, 301, 327 and 412 had no detectable affect on M14-G11 antibody binding. Mutation of IGF-1R amino acid residues 257, 259, 260, 263 and 265 had a weak negative affect on M14-G11 antibody binding. Mutation of IGF-1R amino acid residue 254 had a moderate negative affect on M14-G11 antibody binding. Mutation of IGF-1R amino acid residues 248 and 250 had a strong negative affect on M14-G11 antibody binding. See, Table 20 for a compilation of mutation analysis results.

FIG. 38: Residues whose mutation affected the binding of αIR3 and P1E2 to hIGF-1R-Fc were mapped to the structure of the first three ectodomains of human IGF-1R. Mutation of IGF-1R amino acid residues 28, 227, 237, 250, 259, 260, 264, 285, 286, 306 and 412 had no detectable affect on antibody binding. Mutation of IGF-1R amino acid residues 257, 263, 301, 303, 308, 327 and 389 had a weak negative affect on antibody binding. Mutation of IGF-1R amino acid residue 248 and 254 had a moderate negative affect on M14-G11 antibody binding. Mutation of IGF-1R amino acid residue 265 had a strong negative affect on antibody binding. See, Table 20 for a compilation of mutation analysis results.

FIG. 39: Shows enhanced inhibition of BXPC3 (pancreatic cancer cell line) cell growth stimulated by IGF-1/IGF-2 under serum-free conditions through combined antibody targeting of distinct IGF-1R epitopes.

FIG. 40: Shows that the combination of equimolar amounts of M13.C06.G4.P.agly (C06) and M14.G11.G4.P.agly (G11) antibodies at concentrations between 500 nM and 5 nM resulted in significantly enhanced inhibition of cell growth compared to that observed with either antibody alone at the same corresponding antibody concentrations.

FIG. 41: Shows an example of the effects observed in H322 M grown under standard cell culture conditions in the presence of 10% fetal bovine serum, where a significantly greater inhibition of cell growth resulted from the C06/G11 antibody combination compared to either antibody alone.

FIG. 42: Shows discrimination of the allosteric or competitive IGF-1 and IGF-2 ligand inhibition properties of anti-IGF-1R antibodies.

FIGS. 43 and 44: Models of the surface of the L1/CRR/L2 domains of IGF-1R based on the published crystal structure (Garrett, et al., “Crystal structure of the first three domains of the type-1 insulin-like growth factor receptor,” Nature, (1998) Jul. 23; 394(6691):395-9). FIG. 43 shows the residues that have been described as important for IGF-1 binding (Whittaker et al., 2001). Surface diagrams in FIG. 44 show the positions of each IGF-1R mutant on the surface of the molecule and their effect on the binding of each of the 6 antibodies that bind to the CRR/L2 region. Mutations that had an effect on binding are shown in black and those that had no effect are shown in white.

FIG. 45: Binding of IGF-1 to IGF-1R monitored by isothermal titration calorimetry ITC. (A) Heat generated by 2 μL injections of 60 μM IGF-1 into an ˜200 μL solution of 5 μM sIGF-1R(1-903) measured by calorimetry. (B) ITC binding curves of IGF-1 binding to sIGF-1R(1-903) at 5° C., 25° C., and 37° C. Equilibrium dissociation constants (KD) for IGF-1 binding at the three separate temperatures are listed at the bottom of the graph.

FIG. 46: Dual injection cycles of the inhibitory MAbs followed by IGF-1. (A) Left panel: Calorimetric heat capacity measured at 37° C. during 2.0 μL injections of 60 μM IGF-1 to an ˜200 μL solution of 5 μM IGF-1R with (above) or without (below) previous 1.5 μL injections of 75 μM M13-C06. Right Panel Binding curves of IGF-1 binding to sIGF-1R(1-903) in the presence (♦) or absence () of M13-C06 as determined by changes in the enthalpy (ΔH°) of the system. (B) Same as in (A), however using 20C8 as the inhibitory antibody in the experiment at 25° C. (C) Same as (A), however with M14-G11 as the inhibitory antibody used in the experiment at 25° C.

FIG. 47: Solution-based binding of IGF-1 to sIGF-1R(1-903) in the presence and absence of MAbs. (A) Measurement of IGF-1 binding affinity to sIGF-1R(1-903) using varying concentrations of receptor ((▴)=0 nM (standard curve); (▪)=24 nM; (♦)=64 nM; and ()=240 nM sIGF-1R(1-903)). (B) Solution binding experiment using 240 nM sIGF-1R(1-903) in the absence (▪) and presence of saturating inhibitory anti IGF-1R MAbs M13-C06 (), 20C8 (▴), and G11 (♦). Overlaid on the experimental data with the inhibitory antibodies are theoretical curves for ligand binding to the receptor with a spectrum of affinities ranging from 20 μM to 6 nM (dotted lines). The theoretical curves provide a visual cues for the inhibitory effects of the MAbs.

### DETAILED DESCRIPTION OF THE INVENTION

The present application incorporates by reference herein, in their entirety, U.S. provisional patent application No. 60/786,347 (filed Mar. 28, 2006), U.S. provisional patent application No. 60/876,554 (filed Dec. 22, 2006), and U.S. patent application Ser. No. 11/727,887 (filed Mar. 28, 2007).

### I. Definitions

It is to be noted that the term “a” or “an” entity refers to one or more of that entity; for example, “an IGF-1R antibody,” is understood to represent one or more IGF-1R antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

As used herein the term “derived from” a designated protein refers to the origin of the polypeptide. In one embodiment, the polypeptide or amino acid sequence which is derived from a particular starting polypeptide is a variable region sequence (e.g. a VH or VL) or sequence related thereto (e.g. a CDR or framework region). In one embodiment, the amino acid sequence which is derived from a particular starting polypeptide is not contiguous. For example, in one embodiment, one, two, three, four, five, or six CDRs are derived from a starting antibody. In one embodiment, the polypeptide or amino acid sequence that is derived from a particular starting polypeptide or amino acid sequence has an amino acid sequence that is essentially identical to that of the starting sequence or a portion thereof, wherein the portion consists of at least 3-5 amino acids, 5-10 amino acids, at least 10-20 amino acids, at least 20-30 amino acids, or at least 30-50 amino acids, or which is otherwise identifiable to one of ordinary skill in the art as having its origin in the starting sequence.

Also included as polypeptides of the present invention are fragments, derivatives, analogs, or variants of the foregoing polypeptides, and any combination thereof. The terms “fragment,” “variant,” “derivative” and “analog” when referring to IGF-1R antibodies or antibody polypeptides of the present invention include any polypeptides which retain at least some of the antigen-binding properties of the corresponding native antibody or polypeptide. Fragments of polypeptides of the present invention include proteolytic fragments, as well as deletion fragments, in addition to specific antibody fragments discussed elsewhere herein. Variants of IGF-1R antibodies and antibody polypeptides of the present invention include fragments as described above, and also polypeptides with altered amino acid sequences due to amino acid substitutions, deletions, or insertions. Variants may occur naturally or be non-naturally occurring Non-naturally occurring variants may be produced using art-known mutagenesis techniques. Variant polypeptides may comprise conservative or non-conservative amino acid substitutions, deletions or additions. Derivatives of IGF-1R antibodies and antibody polypeptides of the present invention, are polypeptides which have been altered so as to exhibit additional features not found on the native polypeptide. Examples include fusion proteins. Variant polypeptides may also be referred to herein as “polypeptide analogs.” As used herein a “derivative” of an IGF-1R antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an IGF-1R antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an IGF-1R antibody or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The present invention is directed to certain IGF-1R antibodies, or antigen-binding fragments, variants, or derivatives thereof. Unless specifically referring to full-sized antibodies such as naturally-occurring antibodies, the term “IGF-1R antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table I as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambigously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an IGF-1R antibody or antigen-binding fragment, variant, or derivative thereof of the present invention are according to the Kabat numbering system.

In camelid species, the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH.

Antibodies or antigen-binding fragments, variants, or derivatives thereof of the invention include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to IGF-1R antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Antibodies or immunospecific fragments thereof of the present invention may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the invention comprises a polypeptide chain comprising a CH3 domain. Further, a binding polypeptide for use in the invention may lack at least a portion of a CH2 domain (e.g., all or part of a CH2 domain). As set forth above, it will be understood by one of ordinary skill in the art that these domains (e.g., the heavy chain portions) may be modified such that they vary in amino acid sequence from the naturally occurring immunoglobulin molecule.

In certain IGF-1R antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a VL or CL domain.

IGF-1R antibodies, or antigen-binding fragments, variants, or derivatives thereof disclosed herein may be described or specified in terms of the epitope(s) or portion(s) of an antigen, e.g., a target polypeptide (IGF-1R) that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or include non-polypeptide elements, e.g., an “epitope may include a carbohydrate side chain.

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitope recognized by IGF-1R antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of IGF-1R.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody or antigen-binding fragment, variant, or derivative disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁴ sec⁻¹, 10⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein may be said to bind a target polypeptide disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁻⁵ M⁻¹ sec⁻¹, 10⁻⁶ M⁻¹ sec⁻¹, or 5×10⁻⁶ M⁻¹ sec⁻¹ or 10⁷ M⁻¹ sec⁻.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

IGF-1R antibodies or antigen-binding fragments, variants or derivatives thereof of the invention may also be described or specified in terms of their cross-reactivity. As used herein, the term “cross-reactivity”refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

IGF-1R antibodies or antigen-binding fragments, variants or derivatives thereof of the invention may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

IGF-1R antibodies or antigen-binding fragments, variants or derivatives thereof of the invention may be “multispecific,” e.g., bispecific, trispecific or of greater multispecificity, meaning that it recognizes and binds to two or more different epitopes present on one or more different antigens (e.g., proteins) at the same time. Thus, whether an IGF-1R antibody is “monospecific” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in an IGF-1R antibody, binding polypeptide or antibody. Each binding domain specifically binds one epitope. When an IGF-1R antibody, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., IGF-1R antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as cancer, with a binding molecule which specifically binds a given target protein. As described in more detail herein, the binding molecule can be used in unconjugated form or can be conjugated, e.g., to a drug, prodrug, or an isotope.

As used herein, the term “binding molecule” refers to a molecule which binds (e.g., specifically binds or preferentially binds) to a target molecule of interest, e.g., an antigen. In particular embodiments, a binding molecule of the invention is a polypeptide which specifically or preferentially binds to at least one epitope of IGF-1R. Binding molecules within the scope of the invention also include small molecules, nucleic acids, peptides, peptidomimetics, dendrimers, non-immunoglobulin molecules, and other molecules with binding specificity for IGF-1R epitopes described herein.

### Non-Immunoglobulin Binding Molecules

In certain embodiments, the binding molecules of the invention are non-immunoglobulin binding molecules. As used herein, the term “non-immunoglobulin binding molecules” are binding molecules whose binding sites comprise a portion (e.g., a scaffold or framework) which are derived from a polypeptide other than an immunoglobulin, but which may be engineered (e.g., mutagenized) to confer a desired binding specificity.

Non-immunoglobulin binding molecules can comprise binding site portions that are derived from a member of the immunoglobulin superfamily that is not an immunoglobulin (e.g., a T-cell receptor or a cell-adhesion protein (e.g., CTLA-4, N-CAM, telokin)). Such binding molecules comprise a binding site portion which retains the conformation of an immunoglobulin fold and is capable of specifically binding an IGF1-R epitope. In other embodiments, non-immunoglobulin binding molecules of the invention also comprise a binding site with a protein topology that is not based on the immunoglobulin fold (e.g., such as ankyrin repeat proteins or fibronectins) but which nonetheless are capable of specifically binding to a target (e.g., an IGF-1R epitope).

Non-immunoglobulin binding molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in complex with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with the desired binding characteristics, e.g., specific binding to an IGF-1R epitope described supra. For example, selection can be achieved by art-recognized methods such as phage display, yeast display, or ribosome display.

In one embodiment, a binding molecule of the invention comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. The FnIII loops comprise regions that may be subjected to random mutation and directed evolutionary schemes of iterative rounds of target binding, selection, and further mutation in order to develop useful therapeutic tools. Fibronectin based “addressable” therapeutic binding molecules (“FATBIMs”) may developed to specifically or preferentially bind the IGF-1R epitopes described herein. FATBIMs include, for example, the species of fibronectin-based binding molecules termed Adnectins by Compound Therapeutics, Inc. Methods for making fibronectin binding polypeptides are described, for example, in WO 01/64942 and in U.S. Pat. Nos. 6,673,901, 6,703,199, 7,078,490, and 7,119,171, which are incorporated herein by reference.

In another embodiment, a binding molecule of the invention comprises a binding site from an affibody. Affibodies are derived from the immunoglobulin binding domains of staphylococcal Protein A (SPA) (see e.g., Nord et al., Nat. Biotechnol., 15: 772-777 (1997)). Affibody binding sites employed in the invention may be synthesized by mutagenizing an SPA-related protein (e.g., Protein Z) derived from a domain of SPA (e.g., domain B) and selecting for mutant SPA-related polypeptides having binding affinity for an IGF-1R epitope. Other methods for making affibody binding sites are described in U.S. Pat. Nos. 6,740,734 and 6,602,977 and in WO 00/63243, each of which is incorporated herein by reference.

In another embodiment, a binding molecule of the invention comprises a binding site from an anticalin. Anticalins (also known as lipocalins) are members of a diverse β-barrel protein family whose function is to bind target molecules in their barrel/loop region. Lipocalin binding sites may be engineered to bind an IGF-1R epitope by randomizing loop sequences connecting the strands of the barrel (see e.g., Schlehuber et al., Drug Discov. Today, 10: 23-33 (2005); Beste et al., PNAS, 96: 1898-1903 (1999). Anticalin binding sites employed in the binding molecules of the invention may be obtainable starting from polypeptides of the lipocalin family which are mutated in four segments that correspond to the sequence positions of the linear polypeptide sequence comprising amino acid positions 28 to 45, 58 to 69, 86 to 99 and 114 to 129 of the Bilin-binding protein (BBP) of Pieris brassica. Other methods for making anticalin binding sites are described in WO99/16873 and WO 05/019254, each of which is incorporated herein by reference.

In another embodiment, a binding molecule of the invention comprises a binding site from a cysteine-rich polypeptide. Cysteine-rich domains employed in the practice of the present invention typically do not form an α-helix, a β sheet, or a β-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g. Sortilin-related receptor, LDL-receptor, VLDLR, LRP1, LRP2, and ApoER2). Methods for making A domain proteins of a desired binding specificity are disclosed, for example, in WO 02/088171 and WO 04/044011, each of which is incorporated herein by reference.

In other embodiments, a binding molecule of the invention comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include designed ankyrin repeat proteins (i.e., a DARPins) (see e.g., Binz et al., Nat. Biotechnol., 22: 575-582 (2004)) or leucine-rich repeat proteins (i.e., LRRPs) (see e.g., Pancer et al., Nature, 430: 174-180 (2004)). All so far determined tertiary structures of ankyrin repeat units share a characteristic composed of a β-hairpin followed by two antiparallel α-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Other non-immunoglobulin binding sites which may be employed in binding molecules of the invention include binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Panni et al., J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, and other such domains known to those of ordinary skill in the art, as well as derivatives and/or variants thereof. Exemplary non-immunoglobulin binding molecules, and methods of making the same, can also be found in Stemmer et al., “Protein scaffolds and uses thereof”, U.S. Patent Publication No. 20060234299 (Oct. 19, 2006) and Hey, et al., Artificial, Non-Antibody Binding Proteins for Pharmaceutical and Industrial Applications, TRENDS in Biotechnology, vol. 23, No. 10, Table 2 and pp. 514-522 (October 2005); see also, references provided therein.

As used herein, the term “block IGF-1R-mediated signaling to a greater extent” with respect to the binding of a binding molecule to IGF-1R, refers to a situation where the binding of a first binding moiety that binds to a first epitope of IGF-1R (that blocks the binding of at least one of IGF-1 and IGF-2 to IGF-1R) and a second binding moiety that binds to a second, different epitope of IGF-1R (that blocks the binding of at least one of IGF-1 and IGF-2 to IGF-1R to IGF-1R) blocks IGF-1R-mediated signaling more than the binding of the first or second moiety alone. Inhibition of IGF-1R-mediated signaling can be measured in a number of different ways, e.g., downmodulation of tumor growth (e.g. tumor growth delay), reduction in tumor size or metastasis, the amelioration or minimization of the clinical impairment or symptoms of cancer, an extension of the survival of the subject beyond that which would otherwise be expected in the absence of such treatment, and the prevention of tumor growth in an animal lacking any tumor formation prior to administration, i.e., prophylactic administration. As used herein, the terms “downmodulate”, “downmodulating” or “downmodulation” refer to decreasing the rate at which a particular process occurs, inhibiting a particular process, reversing a particular process, and/or preventing the initiation of a particular process. Accordingly, if the particular process is tumor growth or metastasis, the term “downmodulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

In one embodiment, when IGF-1R-mediated signaling is blocked to a greater extent, an additive effect is observed. The term “additive effect”, as used herein refers to the scenario wherein sum effect of the binding of a first and second binding moiety in combination is approximately equal to the effect observed when the first or second binding moieties bind alone. An additive effect is typically measured under conditions where the molar ratio of the first or second binding moiety (alone) to IGF-1R is approximately the same as the molar ratio of the first and second binding-moiety (together) to IGF-1R.

In one embodiment, when IGF-1R-mediated signaling is blocked to a greater extent, a synergistic effect is observed. The term “synergistic effect”, as used herein, refers to a greater-than-additive effect which is produced upon binding of the first and second binding moieties, and which exceeds that which would otherwise result from individual administration of either the first or second binding moieties alone. A synergistic effect is typically measured under conditions where the molar ratio of the first or second binding moiety (alone) to IGF-1R is approximately the same as the molar ratio of the first and second binding moiety (together) to IGF-1R. Embodiments of the invention include methods of producing a synergistic effect in downmodulating IGF-1R-mediated signaling via use of said first and second IGF-1R binding moieties, wherein said effect is at least 5%, 10%, 15%, 20%, 25%, 30%, 40%, 50%, 60%, 70%, 80%, 90% or 100% greater than the corresponding additive effect.

In one embodiment, a synergistic effect is measured using the combination index (CI) method of Chou and Talalay (see Chang et al., Cancer Res. 45: 2434-2439, (1985)) which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. In another embodiment, a synergistic effect is determined by using the fractional inhibitory concentration (FIC). This fractional value is determined by expressing the IC₅₀ of a drug acting in combination, as a function of the IC₅₀ of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

In certain alternative embodiments, a synergistic effect is observed when greater modulation occurs upon combination of two separate compounds (e.g. separate binding moieties) than what is possible when using saturating concentrations or doses of each of the compounds. This form of synergy may occur where the single binding moieties themselves are not capable of leading to a complete effect (e.g., 100% downmodulation is not reached regardless of how high the concentration of the drug is used). In this situation, synergistic effects are not adequately captured by analysis of EC₅₀ or IC₅₀ values. If the combination of two compounds (e.g. binding moieties) leads to a greater downmodulation than what is possible for the single compounds, this is recognized as a powerful synergistic effect.

### Hyperproliferative Disease or Disorders

By “hyperproliferative disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.” In certain embodiments of the present invention, the hyperproliferative disease or disorder, e.g., the precancerous lesion, abnormal cell growth, benign tumor, malignant tumor, or “cancer” comprises cells which express, over-express, or abnormally express IGF-1R.

Additional examples of hyperproliferative diseases, disorders, and/or conditions include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract. Such neoplasms, in certain embodiments, express, over-express, or abnormally express IGF-1R.

Other hyperproliferative disorders include, but are not limited to: hypergammaglobulinemia, lymphoproliferative disorders, paraproteinemias, purpura, sarcoidosis, Sezary Syndrome, Waldenstron's macroglobulinemia, Gaucher's Disease, histiocytosis, and any other hyperproliferative disease, besides neoplasia, located in an organ system listed above. In certain embodiments of the present invention the diseases involve cells which express, over-express, or abnormally express IGF-1R.

As used herein, the terms “tumor” or “tumor tissue” refer to an abnormal mass of tissue that results from excessive cell division, in certain cases tissue comprising cells which express, over-express, or abnormally express IGF-1R. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Cancers conducive to treatment methods of the present invention involves cells which express, over-express, or abnormally express IGF-1R.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The method of the present invention may be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W.B. Saunders Co., Philadelphia, pp. 68-79 (1976). Such conditions in which cells begin to express, over-express, or abnormally express IGF-1R, are particularly treatable by the methods of the present invention.

Hyperplasia is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated by the method of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, menangioma, melanoma, neuroblastoma, and retinoblastoma.

### II. IGF-1R

Naturally occurring insulin-like growth factor receptor-1 (IGF-1R) IGF-1R is a heterotetrameric plasma membrane glycoprotein composed of two α-subunits (130 kDa each) and two β-subunits (90 kDa each) linked by disulfide bonds. Massagué, J. and Czech, M. P. J. Biol. Chem. 257:5038-5045 (1992). IGF-1R is also known in the art by the names CD221 and JTK13. The nucleic acid sequence of the human IGF-1R mRNA is available under GenBank Accession Number NM_{—}000875, and is presented herein as SEQ ID NO: 1.

SEQ ID NO:1
>gi|11068002|ref|NM_000875.2|
Homo sapiens insulin-like growth factor 1
receptor (IGF1R), mRNA
TTTTTTTTTTTTTTGAGAAAGGGAATTTCATCCCAAATAAAAGGAATGAA
GTCTGGCTCCGGAGGAGGGTCCCCGACCTCGCTGTGGGGGCTCCTGTTTC
TCTCCGCCGCGCTCTCGCTCTGGCCGACGAGTGGAGAAATCTGCGGGCCA
GGCATCGACATCCGCAACGACTATCAGCAGCTGAAGCGCCTGGAGAACTG
CACGGTGATCGAGGGCTACCTCCACATCCTGCTCATCTCCAAGGCCGAGG
ACTACCGCAGCTACCGCTTCCCCAAGCTCACGGTCATTACCGAGTACTTG
CTGCTGTTCCGAGTGGCTGGCCTCGAGAGCCTCGGAGACCTCTTCCCCAA
CCTCACGGTCATCCGCGGCTGGAAACTCTTCTACAACTACGCCCTGGTCA
TCTTCGAGATGACCAATCTCAAGGATATTGGGCTTTACAACCTGAGGAAC
ATTACTCGGGGGGCCATCAGGATTGAGAAAAATGCTGACCTCTGTTACCT
CTCCACTGTGGACTGGTCCCTGATCCTGGATGCGGTGTCCAATAACTACA
TTGTGGGGAATAAGCCCCCAAAGGAATGTGGGGACCTGTGTCCAGGGACC
ATGGAGGAGAAAGCCGATGTGTGAGAAGACCACCATCAACAATGAGTACA
ACTACCGCTGCTGGACCACAAACCGCTGCCAGAAAATGTGCCCAAGCACG
TGTGGGAAGCGGGCGTGCACCGAGAACAATGAGTGCTGCCACCCCGAGTG
CCTGGGCAGCTGCAGCGCGCCTGACAACGACACGGCCTGTGTAGCTTGCC
GCCACTACTACTATGCCGGTGTCTGTGTGCCTGCCTGCCCGCCCAACACC
TACAGGTTTGAGGGCTGGCGCTGTGTGGACCGTGACTTCTGCGCCAACAT
CCTCAGCGCCGAGAGCAGCGACTCCGAGGGGTTTGTGATCCACGACGGCG
AGTGCATGCAGGAGTGCCCCTCGGGCTTCATCCGCAACGGCAGCCAGAGC
ATGTACTGCATCCCTTGTGAAGGTCCTTGCCCGAAGGTCTGTGAGGAAGA
AAAGAAAACAAAGACCATTGATTCTGTTACTTCTGCTCAGATGCTCCAAG
GATGCACCATCTTCAAGGGCAATTTGCTCATTAACATCCGACGGGGGAAT
AACATTGCTTCAGAGCTGGAGAACTTCATGGGGCTCATCGAGGTGGTGAC
GGGCTACGTGAAGATCCGCCATTCTCATGCCTTGGTCTCCTTGTCCTTCC
TAAAAAACCTTCGCCTCATCCTAGGAGAGGAGCAGCTAGAAGGGAATTAC
TCCTTCTACGTCCTCGACAACCAGAACTTGCAGCAACTGTGGGACTGGGA
CCACCGCAACCTGACCATCAAAGCAGGGAAAATGTACTTTGCTTTCAATC
CCAAATTATGTGTTTCCGAAATTTACCGCATGGAGGAAGTGACGGGGACT
AAAGGGCGCCAAAGCAAAGGGGACATAAACACCAGGAACAACGGGGAGAG
AGCCTCCTGTGAAAGTGACGTCCTGCATTTCACCTCCACCACCACGTCGA
AGAATCGCATCATCATAACCTGGCACCGGTACCGGCCCCCTGACTACAGG
GATCTCATCAGCTTCACCGTTTACTACAAGGAAGCACCCTTTAAGAATGT
CACAGAGTATGATGGGCAGGATGCCTGCGGCTCCAACAGCTGGAACATGG
TGGACGTGGACCTCCCGCCCAACAAGGACGTGGAGCCCGGCATCTTACTA
CATGGGCTGAAGCCCTGGACTCAGTACGCCGTTTACGTCAAGGCTGTGAC
CCTCACCATGGTGGAGAACGACCATATCCGTGGGGCCAAGAGTGAGATCT
TGTACATTCGCACCAATGCTTCAGTTCCTTCCATTCCCTTGGACGTTCTT
TCAGCATCGAACTCCTCTTCTCAGTTAATCGTGAAGTGGAACCCTCCCTC
TCTGCCCAACGGCAACCTGAGTTACTACATTGTGCGCTGGCAGCGGCAGC
CTCAGGACGGCTACCTTTACCGGCACAATTACTGCTCCAAAGACAAAATC
CCCATCAGGAAGTATGCCGACGGCACCATCGACATTGAGGAGGTCACAGA
GAACCCCAAGACTGAGGTGTGTGGTGGGGAGAAAGGGCCTTGCTGCGCCT
GCCCCAAAACTGAAGCCGAGAAGCAGGCCGAGAAGGAGGAGGCTGAATAC
CGCAAAGTCTTTGAGAATTTCCTGCACAACTCCATCTTCGTGCCCAGACC
TGAAAGGAAGCGGAGAGATGTCATGCAAGTGGCCAACACCACCATGTCCA
GCCGAAGCAGGAACACCACGGCCGCAGACACCTACAACATCACCGACCCG
GAAGAGCTGGAGACAGAGTACCCTTTCTTTGAGAGCAGAGTGGATAACAA
GGAGAGAACTGTCATTTCTAACCTTCGGCCTTTCACATTGTACCGCATCG
ATATCCACAGCTGCAACCACGAGGCTGAGAAGCTGGGCTGCAGCGCCTCC
TACTTCGTCTTTGCAAGGACTATGCCCGCAGAAGGAGCAGATGACATTCC
GGGGCCAGTGACCTGGGAGCCAAGGCCTGAAAACTCCATCTTTTTAAAGT
AGCCGGAACCTGAGAATCCCAATGGATTGATTCTAATGTATGAAATAAAA
TACGGATCACAAGTTGAGGATCAGCGAGAATGTGTGTCCAGACAGGAATA
CAGGAAGTATGGAGGGGCCAAGCTAAACCGGCTAAACCCGGGGAACTACA
CAGCCCGGATTCAGGCCACATCTCTCTCTGGGAATGGGTCGTGGACAGAT
CCTGTGTTCTTCTATGTCCAGGCCAAAACAGGATATGAAAACTTCATCCA
TCTGATCATCGCTCTGCCCGTCGCTGTCCTGTTGATCGTGGGAGGGTTGG
TGATTATGCTGTACGTCTTCCATAGAAAGAGAAATAACAGCAGGCTGGGG
AATGGAGTGCTGTATGCCTCTGTGAACCCGGAGTACTTCAGCGCTGCTGA
TGTGTACGTTCCTGATGAGTGGGAGGTGGCTCGGGAGAAGATCACCATGA
GCCGGGAACTTGGGCAGGGGTCGTTTGGGATGGTCTATGAAGGAGTTGCC
AAGGGTGTGGTGAAAGATGAACCTGAAACCAGAGTGGCCATTAAAACAGT
GAACGAGGCCGCAAGCATGCGTGAGAGGATTGAGTTTCTCAACGAAGCTT
CTGTGATGAAGGAGTTCAATTGTCACCATGTGGTGCGATTGCTGGGTGTG
GTGTCCCAAGGCCAGCCAACACTGGTCATCATGGAACTGATGACACGGGG
CGATCTCAAAAGTTATCTCCGGTCTCTGAGGCCAGAAATGGAGAATAATC
CAGTCCTAGCACCTCCAAGCCTGAGCAAGATGATTCAGATGGCCGGAGAG
ATTGCAGACGGCATGGCATACCTCAACGCCAATAAGTTCGTCCACAGAGA
CCTTGCTGCCCGGAATTGCATGGTAGCCGAAGATTTCACAGTCAAAATCG
GAGATTTTGGTATGACGCGAGATATCTATGAGACAGACTATTACCGGAAA
GGAGGCAAAGGGCTGCTGCCCGTGCGCTGGATGTCTCCTGAGTCCCTCAA
GGATGGAGTCTTCACCACTTACTCGGACGTCTGGTCCTTCGGGGTCGTCC
TCTGGGAGATCGCCACACTGGCCGAGCAGCCCTACCAGGGCTTGTCCAAC
GAGCAAGTCCTTCGCTTCGTCATGGAGGGCGGCCTTCTGGACAAGCCAGA
CAACTGTCCTGACATGCTGTTTGAACTGATGCGCATGTGCTGGCAGTATA
ACCCCAAGATGAGGCCTTCCTTCCTGGAGATCATCAGCAGCATCAAAGAG
GAGATGGAGCCTGGCTTCCGGGAGGTCTCCTTCTACTACAGCGAGGAGAA
CAAGCTGCCCGAGCCGGAGGAGCTGGACCTGGAGCCAGAGAACATGGAGA
GCGTCCCCCTGGACCCCTCGGCCTCCTCGTCCTCCCTGCCACTGCCCGAC
AGACACTCAGGACACAAGGCCGAGAACGGCCCCGGCCCTGGGGTGCTGGT
CCTCCGCGCCAGCTTCGACGAGAGACAGCCTTACGCCCACATGAACGGGG
GCCGCAAGAACGAGCGGGCCTTGCCGCTGCCCCAGTCTTCGACCTGCTGA
TCCTTGGATCCTGAATCTGTGCAAACAGTAACGTGTGCGCACGCGCAGCG
GGGTGGGGGGGGAGAGAGAGTTTTAACAATCCATTCACAAGCCTCCTGTA
CCTCAGTGGATCTTCAGTTCTGCCCTTGCTGCCCGCGGGAGACAGCTTCT
CTGCAGTAAAACACATTTGGGATGTTCCTTTTTTCAATATGCAAGCAGCT
TTTTATTCCCTGCCCAAACCCTTAACTGACATGGGCCTTTAAGAACCTTA
ATGACAACACTTAATAGCAACAGAGCACTTGAGAACCAGTCTCCTCACTC
TGTCCCTGTCCTTCCCTGTTCTCCCTTTCTCTCTCCTCTCTGCTTCATAA
CGGAAAAATAATTGCCACAAGTCCAGCTGGGAAGCCCTTTTTATCAGTTT
GAGGAAGTGGCTGTCCCTGTGGCCCCATCCAACCACTGTACACACCCGCC
TGACACCGTGGGTCATTACAAAAAAACACGTGGAGATGGAAATTTTTACC
TTTATCTTTCACCTTTCTAGGGACATGAAATTTACAAAGGGCCATCGTTC
ATCCAAGGCTGTTACCATTTTAACGCTGCCTAATTTTGCCAAAATCCTGA
ACTTTCTCCCTCATCGGCCCGGCGCTGATTCCTCGTGTCCGGAGGCATGG
GTGAGCATGGCAGCTGGTTGCTCCATTTGAGAGACACGCTGGCGACACAC
TCCGTCCATCCGACTGCCCCTGCTGTGCTGCTCAAGGCCACAGGCACACA
GGTCTCATTGCTTCTGACTAGATTATTATTTGGGGGAACTGGACACAATA
GGTCTTTCTCTCAGTGAAGGTGGGGAGAAGCTGAACCGGC

The precursor polypeptide sequence is available under GenBank Accession Number NP_{—}000866, and is presented herein as SEQ ID NO:2.

SEQ ID NO:2
>gi|4557665|ref|NP_00866.1|
insulin-like growth factor 1 receptor precursor
[Homo sapiens]
MKSGSGGGSPTSLWGLLFLSAALSLWPTSGEICGPGIDIRNDYQQLKRLE
NCTVIEGYLHILLISKAEDYRSYRFPKLTVITEYLLLFRVAGLESLGDLF
PNLTVIRGWKLFYNYALVIFEMTNLKDIGLYNLRNITRGAIRIEKNADLC
YLSTVDWSLTLDAVSNNYIVGNKPPKECGDLCPGTMEEKPMCEKTTINNE
YNYRCWTTNRCQKMCPSTCGKRACTENNECCHPECLGSCSAPDNDTACVA
CRHYYYAGVCVPACPPNTYRFEGWRCVDRDFCANILSAESSDSEGFVIHD
GECMQECPSGFIRNGSQSMYCIPCEGPCPKVCEEEKKTKTIDSVTSAQML
QGCTIFKGNLLINIRRGNNIASELENFMGLIEVVTGYVKIRHSHALVSLS
FLKNLRLILGEEQLEGNYSFYVLDNQNLQQLWDWDHRNLTIKAGKMYFAF
NPKLCVSEIYRMEEVTGTKGRQSKGDINTRNNGERASCESDVLHFTSTTT
SKNRIIITWHRYRPPDYRDLISFTVYYKEAPFKNVTEYDGQDACGSNSWN
MVDVDLPPNKDVEPGILLHGLKPWTQYAVYVKAVTLTMVENDHIRGAKSE
ILYIRTNASVPSIPLDVLSASNSSSQLIVKWNPPSLPNGNLSYYIVRWQR
QPQDGYLYRHNYCSKDKIPIRKYADGTIDIEEVTENPKTEVCGGEKGPCC
ACPKTEAEKQAEKEEAEYRKVFENFLHNSIFVPRPERKRRDVMQVANTTM
SSRSRNTTAADTYNITDPEELETEYPFFESRVDNKERTVISNLRPFTLYR
IDIHSCNHEAEKLGCSASNFVFARTMPAEGADDIPGPVTWEPRPENSIFL
KWPEPENPNGLILMYEIKYGSQVEDQRECVSRQEYRKYGGAKLNRLNPGN
YTARIQATSLSGNGSWTDPVFFYVQAKTGYENFIHLIIALPVAVLLIVGG
LVIMLYVFHRKRNNSRLGNGVLYASVNPEYFSAADVYVPDEWEVAREKIT
MSRELGQGSFGMVYEGVAKGVVKDEPETRVAIKTVNEAASMRERIEFLNE
ASVMKEFNCHHVVRLLGVVSQGQPTLVIMELMTRGDLKSYLRSLRPEMEN
NPVLAPPSLSKMIQMAGEIADGMAYLNANKFVHRDLAARNCMVAEDFTVK
IGDFGMTRDIYETDYYRKGGKGLLPVRWMSPESLKDGVFTTYSDVWSFGV
VLWEIATLAEQPYQGLSNEQVLRFVMEGGLLDKPDNCPDMLFELMRMCWQ
YNPKMRPSFLEIISSIKEEMEPGFREVSFYYSEENKLPEPEELDLEPENM
ESVPLDPSASSSSLPLPDRHSGHKAENGPGPGVLVLRASFDERQPYAHMN
GGRKNERALPLPQSSTC

Amino acids 1 to 30 of SEQ ID NO:2 are reported to encode the IGF-1R signal peptide, amino acids 31 to 740 of SEQ ID NO:2 are reported to encode the IGF-1R α-subunit, and amino acids 741 to 1367 of SEQ ID NO:2 are reported to encode the IGF-1R β-subunit. These and other features of human IGF-1R reported in the NP_{—}000866 GenBank entry are presented in Table 2.

TABLE 2

| SEQ ID NO: 2 | Feature (from NP_000866) |
|---|---|
| 1 to 30 | signal peptide |
| 31 to 740 | insulin-like growth factor 1 receptor alpha chain |
| 51 to 161 | Receptor L domain |
| 230 to 277 | Furin-like repeats |
| 372 to 467 | Receptor L domain |
| 494 to 606 | Fibronectin type 3 domain |
| 611 to >655 | Fibronectin type 3 domain |
| 741 to 1367 | insulin-like growth factor 1 receptor beta |
| 835 to 924 | Fibronectin type 3 domain |
| 931 to 955 | transmembrane region |
| 973 | Phosphorylation |
| 980 | Phosphorylation |
| 991 to 1268 | Tyrosine kinase, catalytic domain |
| 1161 | Phosphorylation |
| 1165 | Phosphorylation |
| 1166 | Phosphorylation |

The present invention is also directed to IGF-1R antibodies, or antigen-binding fragments, variants, or derivatives thereof which bind specifically, preferentially, and/or competitively to non-human IGF-1R proteins, e.g., IGF-1R from rodents or non-human primates.

IGF-1R is expressed in a large number of tumor cells, including, but not limited to certain of the following: bladder tumors (Ouban et al., Hum. Pathol. 34:803 (2003)); brain tumors (Del Valle et al., Clinical Cancer Res. 8:1822 (2002)); breast tumors (Railo, et al., Eur. J. Cancer 30:307 (1994) and Altundag et al., Hum Pathol. 36:448-449 (2005)); colon tumors, e.g., adenocarcinomas, metastases, and adenomas (Hakam et al., Human Pathol. 30:1128 (1999), Gongoll et al., Virchows. Arc. 443:139 (2003), and Nakamura et al., Clin. Cancer Res. 10: 8434-8441 (2004); gastric tumors (Jiang et al., Clin. Exp. Metastasis 21:755 (2004)); kidney tumors, e.g., clear cell, chromophobe and papillary RCC (Schips et al., Am. J. Clin. Pathol. 122:931-937 (2004)); lung tumors (Ouban et al., Hum. Pathol. 34:803-808 (2003)) and Kaiser, et al., J. Cancer Res. Clinical Oncol. 119:665-668 (1993)); ovarian tumors (Ouban et al., Hum. Pathol. 34:803-808 (2003)); pancreatic tumors, e.g., ductal adenocarcinoma (Hakam et al., Digestive Diseases. Sci. 48:1972-1978 (2003) and Furukawa et al., Clinical Cancer Res. 11:3233-3242 (2005)); and prostate tumors (Hellawell et al., Cancer Res. 62:2942-2950 (2002)).

### III. IGF-1R Antibodies

In one embodiment, the present invention is directed to IGF-1R antibodies, or antigen-binding fragments, variants, or derivatives thereof. For example, the present invention includes at least the antigen-binding domains of certain monoclonal antibodies, and fragments, variants, and derivatives thereof shown in Tables 3 and 4. Table 3 lists human anti-human IGF-1R Fab regions identified from a phage display library and various binding properties of the antibodies, described in more detail in the Examples. Table 4 lists murine anti-human IGF-1R monoclonal antibodies identified by hybridoma technology, and various binding properties of the antibodies, described in more detail in the Examples.

TABLE 3
Functional properties of IGF-1R specific Fabs.

| | | | | | | | | | Inhibition of | |
|---|---|---|---|---|---|---|---|---|---|---|
| | | ELISA Binding | | | FACS Binding | | IGF | | IGF-1R | |
| | | IGF- | IGF- | | IGF-1R | MCF-7 | Blocking | | Phosphorylation | |
| | Fabs | 1R-His | 1R-Fc | InsR | 3T3 | EC50nM | IGF-1 | IGF-2 | IGF-1 | IGF-2 |
| 1 | M13-C06 | + | +++ | − | +++ | 8.8 | + | ++ | ++ | ++ |
| 2 | M14-G11 | ++ | +++ | − | +++ | 39.8 | ++ | ++ | + | +++ |
| 3 | M14-C03 | ++ | +++ | − | +++ | 25.4 | − | + | ++ | ++ |
| 4 | M14-B01 | +++ | +++ | − | +++ | 29.4 | ++ | ++ | ++ | ++ |
| 5 | M12-E01 | +++ | +++ | − | +++ | 7.4 | − | ++ | ++ | + |
| 6 | M12-G04 | + | ++ | − | ++ | 25.0 | + | + | + | + |

| | | | | | |
|---|---|---|---|---|---|
| pTy-IGF-1R | >30% @0.1 ug/ml | +++ | Ligand | >30% @ 0.1 ug/ml | +++ |
| | >30% @1 ug/ml | ++ | Blocking | >30% @ 1 ug/ml | ++ |
| | >30% @10 ug/ml | + | | >30% @ 10 ug/ml | + |
| | > OD 2x bkg | | | | |
| ELISA | @0.1 ug/ml | +++ | | | |
| | > OD 2x bkg | | | | |
| | @1 ug/ml | ++ | | | |
| | > OD 2x bkg | | | | |
| | @10 ug/ml | + | | | |

TABLE 4
Functional properties of murine monoclonal antibodies

| | | | Binding | | | | | | | | | | | |
|---|---|---|---|---|---|---|---|---|---|---|---|---|---|---|
| | | | (EC₅₀nM) | | | IGF | | Inhibition of | | Proliferation of Tumor | | | | |
| | | | Protein | Tumor | | Blocking | | IGF-1R.pTy | | Cells¹ | | | | |
| | Hybridoma # | Isotype | ELISA | MCF-7 | InsR ELISA | IGF-1 | IGF-2 | IGF-1 | IGF-2 | MCF-7* | H-23 | Calu-6 | Panc-1 | Colo205 |
| 1 | P2A7.3E11 | IgG2a/k | 0.011 | 0.447 | − | +++ | − | +++ | ++ | ++ | ++++ | +++ | ++++ | +++ |
| 2 | 20C8.3B8 | IgG1/k | 0.085 | 1.228 | − | +++ | +++ | +++ | ++ | +++ | +++ | +++ | +++ | +++ |
| 3 | P1A2.2B11 | IgG2b/k | 0.023 | 1.103 | − | +++ | − | +++ | +++ | ++ | +++ | ++ | +++ | +++ |
| 4 | 20D8.24B11 | IgG1/k | 0.042 | 1.296 | − | +++ | +++ | +++ | ++ | ++ | ++++ | +++ | +++ | +++ |
| 5 | P1E2.3B12 | IgG2b/k | 0.016 | 0.391 | − | +++ | − | +++ | +++ | ++ | ++++ | ++ | ++ | ++ |
| 6 | P1G10.2B8 | IgG1/k | 0.075 | 2.059 | − | +++ | − | +++ | +++ | +++ | +++ | ++ | + | ++ |

¹MCF-7 = breast cancer cell; H-23 and Calu-6 = lung cancer cells; Panc-1 = pancreatic cancer cell; Colo205 = colon cancer cell

| pTy-IGF-1R | | Ligand Blocking | | *Ki67 Inhibit. (MCF-7) | | Prolif. Inhibition | |
|---|---|---|---|---|---|---|---|
| >30% @ | +++ | >40% @ | +++ | >50% @ | ++++ | >30% @ | ++++ |
| 0.1 μg/ml | | 0.1 μg/ml | | 0.01 μg/ml | | 0.01 μg/ml | |
| >30% @ | ++ | >40% @ | ++ | >50% @ | +++ | >30% @ | +++ |
| 1 μg/ml | | 1 μg/ml | | 0.1 μg/ml | | 0.1 μg/ml | |
| >30% @ | + | >40% @ | + | >50% @ | ++ | >30% @ | ++ |
| 10 μg/ml | | 10 μg/ml | | 1 μg/ml | | 1 μg/ml | |
| | | | | >50% @ | + | >30% @ | + |
| | | | | 10 μg/ml | | 10 μg/ml | |

Chinese Hamster Ovary cell lines which express full-length antibody of M13-C06 and M14-C03 were deposited with the American Type Culture Collection (“ATCC”) on Mar. 28, 2006, and were given ATCC Deposit Numbers PTA-7444 and PTA-7445, respectively. Chinese Hamster Ovary cell lines which express Fab antibody fragment M14-G11 were deposited with the American Type Culture Collection (“ATCC”) on Aug. 29, 2006, and were given ATCC Deposit Number PTA-7855.

Hybridoma cell line which express full-length human antibodies P2A7.3E11, 20C8.3B8, and P1A2.2B11 were deposited with the ATCC on Mar. 28, 2006, Jun. 13, 2006, and Mar. 28, 2006, respectively, and were given the ATCC Deposit Numbers PTA-7458, PTA-7732, and, PTA-7457, respectively. Hybridoma cell lines which express full-length human antibodies 20D8.24B11, P1E2.3B12, and P1G10.2B8 were deposited with the ATCC on Mar. 28, 2006, Jul. 11, 2006, and Jul. 11, 2006, respectively, and were given the ATCC Deposit Numbers PTA-7456, PTA-7730, and PTA-7731, respectively. See, ATCC Deposit Table (below) for correlation of antibodies and deposited cell lines.

The ATCC is located at 10801 University Boulevard, Manassas, Va. 20110-2209, USA. The ATCC deposits were made pursuant to the terms of the Budapest Treaty on the international recognition of the deposit of microorganisms for purposes of patent procedure.

Certain embodiments of the invention were deposited with the American Type Culture Collection as shown in the following table (“ATCC Deposit Table”).

ATCC Deposit Table

Chinese Hamster Ovary (CHO) Cells

| Name of cell line (“as | Date of deposit (ATCC | Cell line | |
|---|---|---|---|
| indicated on ATCC | deposit | referred to | Antibody |
| deposit receipt”): | number) | herein as: | produced: |
| “Chinese Hamster Ovary | Mar. 28, 2006 | M13-C06 | M13-C06.G4.P.agly |
| (CHO): C06-40B5; CHO | (PTA-7444) | | |
| DG44Biogen Idec | | | |
| EAMar. 14, 2006” | | | |
| “Chinese Hamster Ovary | Mar. 28, 2006 | M14-C03 | M14-C03.G4.P.agly |
| (CHO): C03-2 CHO | (PTA-7445) | | |
| DG44Biogen Idec DA | | | |
| Mar. 14, 2006” | | | |
| “Chinese hamster ovary cell | Aug. 29, | M14-G11 | M14-G11.G4.P.agly |
| line: G11 70 8e6 cells | 2006 | | |
| Aug. 09, 2006” | (PTA-7855) | | |
| Hybridomas | | | |
| | Date of | | |
| Name of cell line (“as | deposit (ATCC | Cell line | |
| indicated on ATCC | deposit | referred to | |
| deposit receipt”): | number) | herein as: | Antibody isotype: |
| “Hybridoma 8.P2A7.3D11” | Mar. 28, 2006 | P2A7.3E11 | IgG2a/k |
| | (PTA-7458) | | |
| “Hybridoma cell line: | Jun. 13, 2006 | 20C8.3B8 | IgG1/k |
| 7.20C8.3B8” | (PTA-7732) | | |
| “Hybridoma: 5.P1A2.2B11” | Mar. 28, 2006 | P1A2.2B11 | IgG2b/k |
| | (PTA-7457) | | |
| “Hybridoma: 7.20D8.24.B11” | Mar. 28, 2006 | 20D8.24B11 | IgG1/k |
| | (PTA-7456) | | |
| “Hybridoma Cell Line: | Jul. 11, 2006 | P1E2.3B12 | IgG2b/k |
| 9.P1E2.3B12” | (PTA-7730) | | |
| “Hybridoma Cell Line: | Jul. 11, 2006 | P1G10.2B8 | IgG1/k |
| 5P1G10.2B8” | (PTA-7731) | | |

As used herein, the term “antigen binding domain” includes a site that specifically binds an epitope on an antigen (e.g., an epitope of IGF-1R). The antigen binding domain of an antibody typically includes at least a portion of an immunoglobulin heavy chain variable region and at least a portion of an immunoglobulin light chain variable region. The binding site formed by these variable regions determines the specificity of the antibody.

The present invention is more specifically directed to an IGF-1R antibody, or antigen-binding fragment, variant or derivatives thereof, where the IGF-1R antibody specifically binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.

The invention is further drawn to an IGF-1R antibody, or antigen-binding fragment, variant or derivatives thereof, where the IGF-1R antibody competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8 from binding to IGF-1R.

The invention is also drawn to an IGF-1R antibody, or antigen-binding fragment, variant or derivatives thereof, where the IGF-1R antibody comprises an antigen binding domain identical to that of a monoclonal Fab antibody fragment selected from the group consisting of M13-C06, M14-G11, M14-C03, M14-B01, M12-E01, and M12-G04, or a monoclonal antibody produced by a hybridoma selected from the group consisting of P2A7.3E11, 20C8.3B8, P1A2.2B11, 20D8.24B11, P1E2.3B12, and P1G10.2B8.

Methods of making antibodies are well known in the art and described herein. Once antibodies to various fragments of, or to the full-length IGF-1R without the signal sequence, have been produced, determining which amino acids, or epitope, of IGF-1R to which the antibody or antigen binding fragment binds can be determined by epitope mapping protocols as described herein as well as methods known in the art (e.g. double antibody-sandwich ELISA as described in “Chapter 11—Immunology,” Current Protocols in Molecular Biology, Ed. Ausubel et al., v. 2, John Wiley & Sons, Inc. (1996)). Additional epitope mapping protocols may be found in Morris, G. Epitope Mapping Protocols, New Jersey: Humana Press (1996), which are both incorporated herein by reference in their entireties. Epitope mapping can also be performed by commercially available means (i.e. ProtoPROBE, Inc. (Milwaukee, Wis.)).

Additionally, antibodies produced which bind to any portion of IGF-1R can then be screened for their ability to act as an antagonist of IGF-1R for example, to inhibit binding of insulin growth factor, e.g., IGF-1, IGF-2, or both IGF-1 and IGF-2 to IGF-1R, to promote internalization of IGF-1R, to inhibit phosphorylation of IGF-1R, to inhibit downstream phosphorylation, e.g., of Akt or p42/44 MAPK, or to inhibit tumor cell proliferation, motility or metastasis. Antibodies can be screened for these and other properties according to methods described in detail in the Examples. Other functions of antibodies of the present invention can be tested using other assays as described in the Examples herein.

In other embodiments, the present invention includes an antibody, or antigen-binding fragment, variant, or derivative thereof which specifically or preferentially binds to at least one epitope of IGF-1R, where the epitope comprises, consists essentially of, or consists of at least about four to five amino acids of SEQ ID NO:2, at least seven, at least nine, or between at least about 15 to about 30 amino acids of SEQ ID NO:2. The amino acids of a given epitope of SEQ ID NO:2 as described may be, but need not be contiguous or linear. In certain embodiments, at least one epitope of IGF-1R comprises, consists essentially of, or consists of a non-linear epitope formed by the extracellular domain of IGF-1R as expressed on the surface of a cell or as a soluble fragment, e.g., fused to an IgG Fc region. Thus, in certain embodiments at least one epitope of IGF-1R comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, where non-contiguous amino acids form an epitope through protein folding.

In other embodiments, the present invention includes an antibody, or antigen-binding fragment, variant, or derivative thereof which specifically or preferentially binds to at least one epitope of IGF-1R, where the epitope comprises, consists essentially of, or consists of, in addition to one, two, three, four, five, six or more contiguous or non-contiguous amino acids of SEQ ID NO:2 as described above, and an additional moiety which modifies the protein, e.g., a carbohydrate moiety may be included such that the IGF-1R antibody binds with higher affinity to modified target protein than it does to an unmodified version of the protein. Alternatively, the IGF-1R antibody does not bind the unmodified version of the target protein at all.

In certain aspects, the present invention is directed to an antibody, or antigen-binding fragment, variant, or derivative thereof which specifically binds to a IGF-1R polypeptide or fragment thereof, or an IGF-1R variant polypeptide, with an affinity characterized by a dissociation constant (K_{D}) which is less than the K_{D} for a given reference monoclonal antibody.

In certain embodiments, an antibody, or antigen-binding fragment, variant, or derivative thereof of the invention binds specifically to at least one epitope of IGF-1R or fragment or variant described above, i.e., binds to such an epitope more readily than it would bind to an unrelated, or random epitope; binds preferentially to at least one epitope of IGF-1R or fragment or variant described above, i.e., binds to such an epitope more readily than it would bind to a related, similar, homologous, or analogous epitope; competitively inhibits binding of a reference antibody which itself binds specifically or preferentially to a certain epitope of IGF-1R or fragment or variant described above; or binds to at least one epitope of IGF-1R or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻⁴ M, about 10⁻¹⁴ M, about 5×10⁻¹⁵ M, or about 10⁻¹⁵ M. In a particular aspect, the antibody or fragment thereof preferentially binds to a human IGF-1R polypeptide or fragment thereof, relative to a murine IGF-1R polypeptide or fragment thereof. In another particular aspect, the antibody or fragment thereof preferentially binds to one or more IGF-1R polypeptides or fragments thereof, e.g., one or more mammalian IGF-1R polypeptides, but does not bind to insulin receptor (InsR) polypeptides. While not being bound by theory, insulin receptor polypeptides are known to have some sequence similarity with IGF-1R polypeptides, and antibodies which cross react with InsR may produce unwanted side effects in vivo, e.g., interfering with glucose metabolism.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, an antibody, or antigen-binding fragment, variant, or derivative thereof of the invention binds IGF-1R polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. Alternatively, an antibody, or antigen-binding fragment, variant, or derivative thereof of the invention binds IGF-1R polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻.

In other embodiments, an antibody, or antigen-binding fragment, variant, or derivative thereof of the invention binds IGF-1R polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, an antibody, or antigen-binding fragment, variant, or derivative thereof of the invention binds IGF-1R polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M⁻¹ sec⁻¹.

In various embodiments, an IGF-1R antibody, or antigen-binding fragment, variant, or derivative thereof as described herein is an antagonist of IGF-1R activity. In certain embodiments, for example, binding of an antagonist IGF-1R antibody to IGF-1R as expressed on a tumor cell inhibits binding of insulin growth factor, e.g., IGF-1, IGF-2, or both IGF-1 and IGF-2 to IGF-1R, promotes internalization of IGF-1R thereby inhibiting its signal transduction capability, inhibits phosphorylation of IGF-1R, inhibits phosphorylation of molecules downstream in the signal transduction pathway, e.g., Akt or p42/44 MAPK, or inhibits tumor cell proliferation, motility or metastasis.

Unless it is specifically noted, as used herein a “fragment thereof” in reference to an antibody refers to an antigen-binding fragment, i.e., a portion of the antibody which specifically binds to the antigen. In one embodiment, an IGF-1R antibody, e.g., an antibody of the invention is a bispecific IGF-1R antibody, e.g., a bispecific antibody, minibody, domain deleted antibody, or fusion protein having binding specificity for more than one epitope, e.g., more than one antigen or more than one epitope on the same antigen. In one embodiment, a bispecific IGF-1R antibody has at least one binding domain specific for at least one epitope on a target polypeptide disclosed herein, e.g., IGF-1R. In another embodiment, a bispecific IGF-1R antibody has at least one binding domain specific for an epitope on a target polypeptide and at least one target binding domain specific for a drug or toxin. In yet another embodiment, a bispecific IGF-1R antibody has at least one binding domain specific for an epitope on a target polypeptide disclosed herein, and at least one binding domain specific for a prodrug. A bispecific IGF-1R antibody may be a tetravalent antibody that has two target binding domains specific for an epitope of a target polypeptide disclosed herein and two target binding domains specific for a second target. Thus, a tetravalent bispecific IGF-1R antibody may be bivalent for each specificity.

IGF-1R antibodies, or antigen-binding fragments, variants, or derivatives thereof of the invention, as known by those of ordinary skill in the art, can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Accordingly, certain embodiments of the invention include an IGF-1R antibody, or antigen-binding fragment, variant, or derivative thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted. In other embodiments, certain antibodies for use in the diagnostic and treatment methods described herein have s constant region, e.g., an IgG4 heavy chain constant region, which is altered to eliminate glycosylation, referred to elsewhere herein as “agly” antibodies. While not being bound by theory, it is believed that “agly” antibodies may have an improved safety and stability profile in vivo.

In certain IGF-1R antibodies, or antigen-binding fragments, variants, or derivatives thereof described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of IGF-1R antibodies, or antigen-binding fragments, variants, or derivatives thereof of the invention can be made from whole precursor or parent antibodies using techniques known in the art. Exemplary techniques are discussed in more detail herein.

In certain embodiments both the variable and constant regions of IGF-1R antibodies, or antigen-binding fragments, variants, or derivatives thereof are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

IGF-1R antibodies, or antigen-binding fragments, variants, or derivatives thereof of the invention can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

IGF-1R antibodies, or antigen-binding fragments, variants, or derivatives thereof of the invention also include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In certain embodiments, IGF-1R antibodies, or antigen-binding fragments, variants, or derivatives thereof of the invention will not elicit a deleterious immune response in the animal to be treated, e.g., in a human. In one embodiment, IGF-1R antibodies, or antigen-binding fragments, variants, or derivatives thereof of the invention are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1989); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, VH and VL sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., IGF-1R-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

IGF-1R antibodies, or antigen-binding fragments, variants, or derivatives thereof of the invention may be generated by any suitable method known in the art. Polyclonal antibodies to an antigen of interest can be produced by various procedures well known in the art. For example, an IGF-1R antibody, e.g., a binding polypeptide, e.g., an IGF-1R-specific antibody or immunospecific fragment thereof can be administered to various host animals including, but not limited to, rabbits, mice, rats, chickens, hamsters, goats, donkeys, etc., to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using IGF-1R knockout mice to increase the regions of epitope recognition. Monoclonal antibodies can be prepared using a wide variety of techniques known in the art including the use of hybridoma and recombinant and phage display technology as described elsewhere herein.

Using art recognized protocols, in one example, antibodies are raised in mammals by multiple subcutaneous or intraperitoneal injections of the relevant antigen (e.g., purified IGF-1R or cells or cellular extracts comprising IGF-1R) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Antibody fragments that recognize specific epitopes may be generated by known techniques. For example, Fab and F(ab′)₂ fragments may be produced recombinantly or by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Those skilled in the art will also appreciate that DNA encoding antibodies or antibody fragments (e.g., antigen binding sites) may also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv OE DAB (individual Fv region from light or heavy chains) or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. For example, DNA sequences encoding VH and VL regions are amplified or otherwise isolated from animal cDNA libraries (e.g., human or murine cDNA libraries of lymphoid tissues) or synthetic cDNA libraries. In certain embodiments, the DNA encoding the VH and VL regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., an IGF-1R polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1994). See also, U.S. Pat. No. 5,565,332.)

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7(5):437-444 (1989) and Nisinoff, J. Immunol. 147(8):2429-2438 (1991)). For example, antibodies which bind to and competitively or allosterically inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as, but not limited to, E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In one embodiment, an IGF-1R antibody of the invention comprises at least one heavy or light chain CDR of an antibody molecule. In another embodiment, an IGF-1R antibody of the invention comprises at least two CDRs from one or more antibody molecules. In another embodiment, an IGF-1R antibody of the invention comprises at least three CDRs from one or more antibody molecules. In another embodiment, an IGF-1R antibody of the invention comprises at least four CDRs from one or more antibody molecules. In another embodiment, an IGF-1R antibody of the invention comprises at least five CDRs from one or more antibody molecules. In another embodiment, an IGF-1R antibody of the invention comprises at least six CDRs from one or more antibody molecules. Exemplary antibody molecules comprising at least one CDR that can be included in the subject IGF-1R antibodies are described herein.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., IGF-1R. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies of the present invention can be produced by any method known in the art for the synthesis of antibodies, in particular, by chemical synthesis or preferably, by recombinant expression techniques as described herein.

In one embodiment, an IGF-1R antibody, or antigen-binding fragment, variant, or derivative thereof of the invention comprises a synthetic constant region wherein one or more domains are partially or entirely deleted (“domain-deleted antibodies”). In certain embodiments compatible modified antibodies will comprise domain deleted constructs or variants wherein the entire CH2 domain has been removed (ΔCH2 constructs). For other embodiments a short connecting peptide may be substituted for the deleted domain to provide flexibility and freedom of movement for the variable region. Those skilled in the art will appreciate that such constructs are particularly preferred due to the regulatory properties of the CH2 domain on the catabolic rate of the antibody. Domain deleted constructs can be derived using a vector encoding an IgG₁ human constant domain (see, e.g., WO 02/060955A2 and WO02/096948A2). This vector is engineered to delete the CH2 domain and provide a synthetic vector expressing a domain deleted IgG₁ constant region.

In certain embodiments, IGF-1R antibodies, or antigen-binding fragments, variants, or derivatives thereof of the invention are minibodies. Minibodies can be made using methods described in the art (see e.g., U.S. Pat. No. 5,837,821 or WO 94/09817A1).

In one embodiment, an IGF-1R antibody, or antigen-binding fragment, variant, or derivative thereof of the invention comprises an immunoglobulin heavy chain having deletion or substitution of a few or even a single amino acid as long as it permits association between the monomeric subunits. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides antibodies that comprise, consist essentially of, or consist of, variants (including derivatives) of antibody molecules (e.g., the VH regions and/or VL regions) described herein, which antibodies or fragments thereof immunospecifically bind to an IGF-1R polypeptide or fragment or variant thereof. Standard techniques known to those of skill in the art can be used to introduce mutations in the nucleotide sequence encoding an IGF-1R antibody, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VH-CDR1, VH-CDR2, VH-CDR3, VL region, VL-CDR1, VL-CDR2, or VL-CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an IGF-1R polypeptide).

For example, it is possible to introduce mutations only in framework regions or only in CDR regions of an antibody molecule. Introduced mutations may be silent or neutral missense mutations, i.e., have no, or little, effect on an antibody's ability to bind antigen, indeed some such mutations do not alter the amino acid sequence whatsoever. These types of mutations may be useful to optimize codon usage, or improve a hybridoma's antibody production. Codon-optimized coding regions encoding IGF-1R antibodies of the present invention are disclosed elsewhere herein. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of an IGF-1R polypeptide) can be determined using techniques described herein or by routinely modifying techniques known in the art.

### IV. Polynucleotides Encoding IGF-1R Antibodies

The present invention also provides for nucleic acid molecules encoding IGF-1R antibodies, or antigen-binding fragments, variants, or derivatives thereof of the invention.

In one embodiment, the present invention provides an isolated polynucleotide comprising, consisting essentially of, or consisting of a nucleic acid encoding an immunoglobulin heavy chain variable region (VH), where at least one of the CDRs of the heavy chain variable region or at least two of the VH-CDRs of the heavy chain variable region are at least 80%, 85%, 90% or 95% identical to reference heavy chain VH-CDR1, VH-CDR2, or VH-CDR3 amino acid sequences from monoclonal IGF-1R antibodies disclosed herein. Alternatively, the VH-CDR1, VH-CDR2, and VH-CDR3 regions of the VH are at least 80%, 85%, 90% or 95% identical to reference heavy chain VH-CDR1, VH-CDR2, and VH-CDR3 amino acid sequences from monoclonal IGF-1R antibodies disclosed herein. Thus, according to this embodiment a heavy chain variable region of the invention has VH-CDR1, VH-CDR2, or VH-CDR3 polypeptide sequences related to the polypeptide sequences shown in Table 5:

TABLE 5

Reference VH-CDR1, VH-CDR2, and
VH-CDR3 amino acid sequences*

| | VH SEQUENCE PN/PP | | | |
|---|---|---|---|---|
| | (VH-CDR1, VH-CDR2, | | | |
| Antibody | and VH-CDR3 underlined) | VH CDR1 | VH CDR2 | VH CDR3 |
| M12-E01 | GAAGTTCAATTGTTAGAGTCTGGTGGCGG | PYSML | SIGSSGGS | VRGILHYD |
| | TCTTGTTCAGCCTGGTGGTTCTTTACGTC | (SEQ ID | TRYADSVK | ILIGRNLY |
| | TTTCTTGCGCTGCTTCCGGATTCACTTTC | NO:5) | G | YYYMDV |
| | TCTCCTTACTCTATGCTTTGGGTTCGCCA | | (SEQ ID | (SEQ ID |
| | AGCTCCTGGTAAAGGTTTGGAGTGGGTTT | | NO:6) | NO:7) |
| | CTTCTATCGGTTCTTCTGGTGGCTCTACT | | | |
| | CGTTATGCTGACTCCGTTAAAGGTCGCTT | | | |
| | CACTATCTCTAGAGACAACTCTAAGAATA | | | |
| | CTCTCTACTTGCAGATGAACAGCTTAAGG | | | |
| | GCTGAGGACACCGCCATGTATTACTGTGC | | | |
| | ACGGGTACGGGGGATCCTTCATTACGATA | | | |
| | TTTTGATTGGTAGAAATCTCTACTACTAC | | | |
| | TACATGGACGTCTGGGGCAAAGGGACCAC | | | |
| | GGTCACCGTCTCAAGC | | | |
| | (SEQ ID NO:3) | | | |
| | EVQLLESGGGLVQPGGSLRLSCAASGFTF | | | |
| | SPYSMLWVRQAPGKGLEWVSSIGSSGGST | | | |
| | RYADSVKGRFTISRDNSKNTLYLQMNSLR | | | |
| | AEDTANYYCARVRGILHYDILIGRNLYYY | | | |
| | YMDVWGKGTTVTVSS | | | |
| | (SEQ ID NO:4) | | | |
| M12-G04 | GAAGTTCAATTGTTAGAGTCTGGTGGCGG | KYTMH | SIVSSGGW | DRSIAAAG |
| | TCTTGTTCAGCCTGGTGGTTCTTTACGTC | (SEQ ID | TDYADSVK | TGWSVSFV |
| | TTTCTTGCGCTGCTTCCGGATTCACTTTC | NO:10) | G | DWFDP |
| | TCTAAGTACACTATGCATTGGGTTCGCCA | | (SEQ ID | (SEQ ID |
| | AGCTCCTGGTAAAGGTTTGGAGTGGGTTT | | NO:11) | NO:12) |
| | CTTCTATCGTTTCTTCTGGTGGCTGGACT | | | |
| | GATTATGCTGACTCCGTTAAAGGTCGCTT | | | |
| | CACTATCTCTAGAGACAACTCTAAGAATA | | | |
| | CTCTCTACTTGCAGATGAACAGCTTAAGG | | | |
| | GCTGAGGACACGGCCGTGTATTACTGTGC | | | |
| | GAGAGATCGGAGTATAGCAGCAGCTGGTA | | | |
| | CCGGTTGGTCTGTGAGTTTTGTGGACTGG | | | |
| | TTCGACCCCTGGGGCCAGGGAACCCTGGT | | | |
| | CACCGTCTCAAGC | | | |
| | (SEQ ID NO:8) | | | |
| | EVQLLESGGGLVQPGGSLRLSCAASGFTF | | | |
| | SKYTMHWVRQAPGKGLEWVSSIVSSGGWT | | | |
| | DYADSVKGRFTISRDNSKNTLYLQMNSLR | | | |
| | AEDTAVYYCARDRSIAAAGTGWSVSFVDW | | | |
| | FDPWGQGTLVTVSS | | | |
| | (SEQ ID NO:9) | | | |
| M13-C06 | GAAGTTCAATTGTTAGAGTCTGGTGGCGG | IYRMQ | GISPSGGT | WSGGSGYA |
| | TCTTGTTCAGCCTGGTGGTTCTTTACGTC | (SEQ ID | TWYADSVK | FDI |
| | TTTCTTGCGCTGCTTCCGGATTCACTTTC | NO:15) | G | (SEQ ID |
| | TCTATTTACCGTATGCAGTGGGTTCGCCA | | (SEQ ID | NO:17) |
| | AGCTCCTGGTAAAGGTTTGGAGTGGGTTT | | NO:16) | |
| | CTGGTATCTCTCCTTCTGGTGGCACTACT | | | |
| | TGGTATGCTGACTCCGTTAAAGGTCGCTT | | | |
| | CACTATCTCTAGAGACAACTCTAAGAATA | | | |
| | CTCTCTACTTGCAGATGAACAGCTTAAGG | | | |
| | GCTGAGGACACGGCCGTGTATTACTGTGC | | | |
| | GAGATGGAGCGGGGGTTCGGGCTATGCTT | | | |
| | TTGATATCTGGGGCCAAGGGACAATGGTC | | | |
| | ACCGTCTCAAGC | | | |
| | (SEQ ID NO:13) | | | |
| | EVQLLESGGGLVQPGGSLRLSCAASGFTF | | | |
| | SIYRMQWVRQAPGKGLEWVSGISPSGGTT | | | |
| | WYADSVKGRFTISRDNSKNTLYLQMNSLR | | | |
| | AEDTAVYYCARWSGGSGYAFDIWGQGTMV | | | |
| | TVSS | | | |
| | (SEQ ID NO:14) | | | |
| M13-C06 | GAGGTCCAGCTGTTGGAGTCCGGCGGTGG | IYRMQ | GISPSGGT | WSGGSGYA |
| Optimized | CCTGGTGCAGCCTGGGGGGTCCCTGAGAC | (SEQ ID | TWYADSVK | FDI |
| | TCTCCTGCGCAGCTAGCGGCTTCACCTTC | NO:15) | G | (SEQ ID |
| | AGCATTTACCGTATGCAGTGGGTGCGCCA | | SEQ ID | NO:17) |
| | GGCTCCTGGAAAGGGGCTGGAGTGGGTTT | | NO:16) | |
| | CCGGTATCTCTCCCTCTGGTGGCACGACG | | | |
| | TGGTATGCTGACTCCGTGAAGGGCCGGTT | | | |
| | CACAATCTCCAGAGACAATTCCAAGAACA | | | |
| | CTCTGTACCTGCAAATGAACAGCCTGAGA | | | |
| | GCTGAGGATACTGCAGTGTACTACTGCGC | | | |
| | CAGATGGTCCGGGGGCTCCGGATACGCCT | | | |
| | TCGACATCTGGGGACAGGGAACCATGGTC | | | |
| | ACCGTCTCAAGC | | | |
| | (SEQ ID NO:18) | | | |
| | EVQLLESGGGLVQPGGSLRLSCAASGFTF | | | |
| | SIYRMQWVRQAPGKGLEWVSGISPSGGTT | | | |
| | WYADSVKGRFTISRDNSKNTLYLQMNSLR | | | |
| | AEDTAVYYCARWSGGSGYAFDIWGQGTMV | | | |
| | TVSS | | | |
| | (SEQ ID NO:14) | | | |
| M14-B01 | GAAGTTCAATTGTTAGAGTCTGGTGGCGG | NYHMA | VISPTGGR | AGYSYGYG |
| | TCTTGTTCAGCCTGGTGGTTCTTTACGTC | (SEQ ID | TTYADSVK | YFDY |
| | TTTCTTGCGCTGCTTCCGGATTCACTTTC | NO:21) | (SEQ ID | (SEQ ID |
| | TCTAATTACCATATGGCTTGGGTTCGCCA | | NO:22) | NO:23) |
| | AGCTCCTGGTAAAGGTTTGGAGTGGGTTT | | | |
| | CTGTTATCTCTCCTACTGGTGGCCGTACT | | | |
| | ACTTATGCTGACTCCGTTAAAGGTCGCTT | | | |
| | CACTATCTCTAGAGACAACTCTAAGAATA | | | |
| | CTCTCTACTTGCAGATGAACAGCTTAAGG | | | |
| | GCTGAGGACACAGCCACATATTACTGTGC | | | |
| | GAGAGCGGGGTACAGCTATGGTTATGGCT | | | |
| | ACTTTGACTACTGGGGCCAGGGAACCCTG | | | |
| | GTCACCGTCTCAAGC | | | |
| | (SEQ ID NO:19) | | | |
| | EVQLLESGGGLVQPGGSLRLSCAASGFTF | | | |
| | SNYHMAWVRQAPGKGLEWVSVISPTGGRT | | | |
| | TYADSVKGRFTISRDNSKNTLYLQMNSLR | | | |
| | AEDTATYYCARAGYSYGYGYFDYWGQGTL | | | |
| | VTVSS | | | |
| | (SEQ ID N0:20) | | | |
| M14-B01 | GAGGTCCAGCTGTTGGAGTCCGGCGGTGG | NYHMA | VISPTGGR | AGYSYGYG |
| Optimized | CCTGGTGCAGCCTGGGGGGTCCCTGAGAC | (SEQ ID | TTYADSVK | YFDY |
| | TCTCCTGCGCAGCTAGCGGCTTCACCTTC | NO:21) | G | (SEQ ID |
| | AGCAATTACCACATGGCCTGGGTGCGCCA | | SEQ ID | NO:23) |
| | GGCTCCTGGAAAGGGGCTGGAGTGGGTTT | | NO:22) | |
| | CCGTGATCTCTCCTACCGGTGGCAGGACC | | | |
| | ACTTACGCTGACTCCGTGAAGGGCCGGTT | | | |
| | CACAATCTCCAGAGACAATTCCAAGAACA | | | |
| | CTCTGTACCTGCAAATGAACAGCCTGAGA | | | |
| | GCTGAGGATACTGCAACATACTACTGCGC | | | |
| | CAGAGCCGGGTACTCCTACGGCTACGGAT | | | |
| | ACTTCGACTACTGGGGACAGGGAACCCTG | | | |
| | GTCACCGTCTCAAGC | | | |
| | (SEQ ID NO:24) | | | |
| | EVQLLESGGGLVQPGGSLRLSCAASGFTF | | | |
| | SNYHMAWVRQAPGKGLEWVSVISPTGGRT | | | |
| | TYADSVKGRFTISRDNSKNTLYLQNNSLR | | | |
| | AEDTATYYCARAGYSYGYGYFDYWGQGTL | | | |
| | VTVSS | | | |
| | (SEQ ID NO:20) | | | |
| M14-C03 | GAAGTTCAATTGTTAGAGTCTGGTGGCGG | KYMMS | YISPSGGL | DGARGYGM |
| | TCTTGTTCAGCCTGGTGGTTCTTTACGTC | (SEQ ID | TWYADSVK | DV |
| | TTTCTTGCGCTGCTTCCGGATTCACTTTC | NO:27) | G | (SEQ ID |
| | TCTAAGTACATGATGTCTTGGGTTCGCCA | | NO:28) | (SEQ ID |
| | AGCTCCTGGTAAAGGTTTGGAGTGGGTTT | NO:29) | | |
| | CTTATATCTCTCCTTCTGGTGGCCTTACT | | | |
| | TGGTATGCTGACTCCGTTAAAGGTCGCTT | | | |
| | CACTATCTCTAGAGACAACTCTAAGAATA | | | |
| | CTCTCTACTTGCAGATGAACAGCTTAAGG | | | |
| | GCTGAGGACACGGCCGTGTATTACTGTGC | | | |
| | GAGAGATGGAGCTAGAGGCTACGGTATGG | | | |
| | ACGTCTGGGGCCAAGGGACCACGGTCACC | | | |
| | GTCTCAAGC | | | |
| | (SEQ ID NO:25) | | | |
| | EVQLLESGGGLVQPGGSLRLSCAASGFTF | | | |
| | SKYMMSWVRQAPGKGLEWVSYISPSGGLT | | | |
| | WYADSVKGRFTISRDNSKNTLYLQMNSLR | | | |
| | AEDTAVYYCARDGARGYGMDVWGQGTTVT | | | |
| | VSS | | | |
| | (SEQ ID NO:26) | | | |
| M14-C03 | GAGGTCCAGCTGTTGGAGTCCGGCGGTGG | KYMMS | YISPSGGL | DGARGYGM |
| Optimized | CCTGGTGCAGCCTGGGGGGTCCCTGAGAC | (SEQ ID | TWYADSVK | DV |
| | TCTCCTGCGCAGCTAGCGGCTTCACCTTC | NO:27) | G | (SEQ ID |
| | AGCAAGTACATGATGTCTTGGGTGCGCCA | | (SEQ ID | NO:29) |
| | GGCTCCTGGAAAGGGGCTGGAGTGGGTTT | NO:28) | | |
| | CCTATATCTCTCCCTCTGGTGGCCTGACG | | | |
| | TGGTATGCTGACTCCGTGAAGGGCCGGTT | | | |
| | CACAATCTCCAGAGACAATTCCAAGAACA | | | |
| | CTCTGTACCTGCAAATGAACAGCCTGAGA | | | |
| | GCTGAGGATACTGCAGTGTACTACTGCGC | | | |
| | CAGAGATGGGGCTAGAGGATACGGAATGG | | | |
| | ACGTCTGGGGACAGGGAACCACCGTCACC | | | |
| | GTCTCAAGC | | | |
| | (SEQ ID NO:30) | | | |
| | EVQLLESGGGLVQPGGSLRLSCAASGFTF | | | |
| | SKYMMSWVRQAPGKGLEWVSYISPSGGLT | | | |
| | WYADSVKGRFTISRDNSKNTLYLQMNSLR | | | |
| | AEDTAVYYCARDGARGYGMDVWGQGTTVT | | | |
| | VSS | | | |
| | (SEQ ID NO:26) | | | |
| M14-G11 | GAAGTTCAATTGTTAGAGTCTGGTGGCGG | NYPMY | RISSSGGR | DRWSRSAA |
| | TCTTGTTCAGCCTGGTGGTTCTTTACGTC | (SEQ ID | TVYADSVK | EYGLGGY |
| | TTTCTTGCGCTGCTTCCGGATTCACTTTC | NO:33) | G | (SEQ ID |
| | TCTAATTACCCTATGTATTGGGTTCGCCA | | (SEQ ID | NO:35) |
| | AGCTCCTGGTAAAGGTTTGGAGTGGGTTT | | NO:34) | |
| | CTCGTATCTCTTCTTCTGGTGGCCGTACT | | | |
| | GTTTATGCTGACTCCGTTAAAGGTCGCTT | | | |
| | CACTATCTCTAGAGACAACTCTAAGAATA | | | |
| | CTCTCTACTTGCAGATGAACAGCTTAAGG | | | |
| | GCTGAGGACACGGCCGTGTATTACTGTGC | | | |
| | GAGAGATCGATGGTCCAGATCTGCAGCTG | | | |
| | AATATGGGTTGGGTGGCTACTGGGGCCAG | | | |
| | GGAACCCTGGTCACCGTCTCAAGC | | | |
| | (SEQ ID NO:31) | | | |
| | EVQLLESGGGLVQPGGSLRLSCAASGFTF | | | |
| | SNYPMYWVRQAPGKGLEWVSRISSSGGRT | | | |
| | VYADSVKGRFTISRDNSKNTLYLQMNSLR | | | |
| | AEDTAVYYCARDRWSRSAAEYGLGGYWGQ | | | |
| | GTLVTVSS | | | |
| | (SEQ ID NO:32) | | | |
| M14-G11 | GAGGTCCAGCTGTTGGAGTCCGGCGGTGG | NYPMY | RISSSGGR | DRWSRSAA |
| Optimized | CCTGGTGCAGCCTGGGGGGTCCCTGAGAC | (SEQ ID | TVYADSVK | EYGLGGY |
| | TCTCCTGCGCAGCTAGCGGCTTCACCTTC | NO:33) | G | (SEQ ID |
| | AGCAATTACCCCATGTACTGGGTGCGCCA | | (SEQ ID | NO:35) |
| | GGCTCCTGGAAAGGGGCTGGAGTGGGTTT | | NO:34) | |
| | CCAGGATCTCTAGCAGCGGTGGCAGGACC | | | |
| | GTGTACGCTGACTCCGTGAAGGGCCGGTT | | | |
| | CACAATCTCCAGAGACAATTCCAAGAACA | | | |
| | CTCTGTACCTGCAAATGAACAGCCTGAGA | | | |
| | GCTGAGGATACTGCAGTGTACTACTGCGC | | | |
| | CAGAGATAGGTGGTCCAGATCTGCAGCCG | | | |
| | AGTACGGACTGGGGGGCTACTGGGGACAG | | | |
| | GGAACCCTGGTCACCGTCTCAAGC | | | |
| | (SEQ ID NO:36) | | | |
| | EVQLLESGGGLVQPGGSLRLSCAASGFTF | | | |
| | SNYPMYWVRQAPGKGLEWVSRISSSGGRT | | | |
| | VYADSVKGRFTISRDNSKNTLYLQMNSLR | | | |
| | AEDTAVYYCARDRWSRSAAEYGLGGYWGQ | | | |
| | GTLVTVSS | | | |
| | (SEQ ID NO:32) | | | |
| P2A7.3E11 | CAGGTTCAGCTGCAGCAGTCTGGACCTGA | DYVIN | IYPGNENT | GIYYYGSR |
| | GCTAGTGAAGCCTGGGGCTTCAGTGAAGA | (SEQ ID | YYNEKFKG | TRTMDY |
| | TGTCCTGCAAGGCTTCTGGAAACACATTC | NO:39) | (SEQ ID | (SEQ ID |
| | ACTGACTATGTTATAAACTGGGTGAAGCA | | NO:40) | NO:41) |
| | GAGAACTGGACAGGGCCTTGAGTGGATTG | | | |
| | GAGAGATTTATCCTGGAAATGAAAATACT | | | |
| | TATTACAATGAGAAGTTCAAGGGCAAGGC | | | |
| | CACACTGACTGCAGACAAATCCTCCAACA | | | |
| | CAGCCTACATGCAGCTCAGTAGCCTGACA | | | |
| | TCTGAGGACTCTGCGGTCTATTTCTGTGC | | | |
| | AAGAGGGATTTATTACTACGGTAGTAGGA | | | |
| | CGAGGACTATGGACTACTGGGGTCAAGGA | | | |
| | ACCTCAGTCACCGTCTCCTCA | | | |
| | (SEQ ID NO:37) | | | |
| | QVQLQQSGPELVKPGASVKMSCKASGNTF | | | |
| | TDYVINWVKQRTGQGLEWIGEIYPGNENT | | | |
| | YYNEKFKGKATLTADKSSNTAYMQLSSLT | | | |
| | SEDSAVYFCARGIYYYGSRTRTMDYWGQG | | | |
| | TSVTVSS | | | |
| | (SEQ ID NO:38) | | | |
| 20C8.3B8 | GACGTCCAACTGCAGGAGTCTGGACCTGA | SGYSWH | YIHYSGGT | SGYGYRSA |
| | CCTGGTGAAACCTTCTCAGTCACTTTCAC | (SEQ ID | NYNPSLKS | YYFDY |
| | TCACCTGCACTGTCACTGGCTACTCCATC | NO:44) | (SEQ ID | (SEQ ID |
| | ACCAGTGGTTATAGCTGGCACTGGATCCG | | NO:45) | NO:46) |
| | GCAGTTTCCAGGAAACAAACTGGAATGGA | | | |
| | TGGGCTACATACACTACAGTGGTGGCACT | | | |
| | AACTACAACCCATCTCTCAAAAGTCGAAT | | | |
| | CTCTATCACTCGAGACACATCCAAGAACC | | | |
| | AGTTCTTCCTCCAGTTGAATTCTGTGACT | | | |
| | ACTGAGGACACAGCCACATATTACTGTGC | | | |
| | AAGATCGGGGTACGGCTACAGGAGTGCGT | | | |
| | ACTATTTTGACTACTGGGGCCAAGGGACC | | | |
| | ACGGTCACCGTCTCCTCA | | | |
| | (SEQ ID NO:42) | | | |
| | DVQLQESGPDLVKPSQSLSLTCTVTGYSI | | | |
| | TSGYSWHWIRQFPGNKLEWMGYIHYSGGT | | | |
| | NYNPSLKSRISITRDTSKNQFFLQLNSVT | | | |
| | TEDTATYYCARSGYGYRSAYYFDYWGQGT | | | |
| | TVTVSS | | | |
| | (SEQ ID N0:43) | | | |
| P1A2.2B11 | CAAATACAGTTGGTTCAGAGCGGACCTGA | NHGMN | NTSTGEPT | PLYYMYGR |
| | GCTGAAGAAGCCTGGAGAGACAGTCAAGA | (SEQ ID | YADDFKG | YIDV |
| | TCTCCTGCAAGGCTTCTGGGTATACCTTC | NO:49) | (SEQ ID | (SEQ ID |
| | ACAAACCATGGAATGAACTGGGTGAAGCA | | NO:50) | NO:51) |
| | GGCTCCAGGAAAGGGTTTAAAGTGGATGG | | | |
| | GCTGGATAAACACCTCCACTGGAGAGCCA | | | |
| | ACATATGCTGATGACTTCAAGGGACGTTT | | | |
| | TGCCTTCTCTTTGGAAACCTCTGCCAGCA | | | |
| | CTGCCTTTTTGCAGATCAACAACCTCAAA | | | |
| | AATGAGGACACGGCTTCATATTTCTGTGC | | | |
| | AAGTCCCCTCTACTATATGTACGGGCGGT | | | |
| | ATATCGATGTCTGGGGCGCAGGGACCGCG | | | |
| | GTCACCGTCTCCTCA | | | |
| | (SEQ ID NO:47) | | | |
| | QIQLVQSGPELKKPGETVKISCKASGYTF | | | |
| | TNHGMNWVKQAPGKGLKWMGWNTSTGEPT | | | |
| | YADDFKGRFAFSLETSASTAFLQINNLKN | | | |
| | EDTASYFCASPLYYMYGRYIDVWGAGTAV | | | |
| | TVSS | | | |
| | (SEQ ID NO:48) | | | |
| 20D8.24B11 | ACGTCCAACTGCAGGAGTCTGGACCTGAC | SGYSWH | YIHYSGGT | SGYGYRSA |
| | CTGGTGAAACCTTCTCAGTCACTTTCACT | (SEQ ID | NYNPSLKS | YYFDY |
| | CACCTGCACTGTCACTGGCTACTCCATCA | NO:54) | (SEQ ID | (SEQ ID |
| | CCAGTGGTTATAGCTGGCACTGGATCCGG | | NO:55) | NO:56) |
| | CAGTTTCCAGGAAACAAACTGGAATGGAT | | | |
| | GGGCTACATACACTACAGTGGTGGCACTA | | | |
| | ACTACAACCCATCTCTCAAAAGTCGAATC | | | |
| | TCTATCACTCGAGACACATCCAAGAACCA | | | |
| | GTTCTTCCTCCAGTTGAATTCTGTGACTA | | | |
| | CTGAGGACACAGCCACATATTACTGTGCA | | | |
| | AGATCGGGGTACGGCTACAGGAGTG | | | |
| | (SEQ ID NO:52) | | | |
| | DVQLQESGPDLVKPSQSLSLTCTVTGYSI | | | |
| | TSGYSWHWIRQFPGNKLEWMGYIHYSGGT | | | |
| | NYNPSLKSRISITRDTSKNQFFLQLNSVT | | | |
| | TEDTATYYCARSGYGYRSAYYFDYWGQGT | | | |
| | TLTVSS | | | |
| | (SEQ ID NO:53) | | | |
| P1G10.2B8 | CAGATCCAGTTGGTGCAGTCTGGACCTGA | NHGMN | WINTNTGE | PLYYRNGR |
| | CCTGAAGAAGCCTGGAGAGACAGTCAAGA | (SEQ ID | PTYADDFK | YFDV |
| | TCTCCTGCAAGGCTTCTGGGTATACCTTC | NO:59) | G | (SEQ ID |
| | ACAAACCATGGAATGAACTGGGTGAAGCA | | (SEQ ID | NO:61) |
| | GGCTCCAGGAAAGGATTTAAAGTGGATGG | | NO:60) | |
| | GCTGGATAAACACCAACACTGGAGAGCCA | | | |
| | ACATATGCTGATGACTTCAAGGGACGGTT | | | |
| | TGCCTTCTCTTTGGAAACCTCTGCCAGCA | | | |
| | CTGCCTATTTGCAGATCAACAACCTCAAA | | | |
| | AATGAGGACACGGCTACATATTTCTGTGC | | | |
| | AAGTCCCCTCTACTATAGGAACGGGCGAT | | | |
| | ACTTCGATGTCTGGGGCGCAGGGACCACG | | | |
| | GTCACCGTCTCC | | | |
| | (SEQ ID NO:57) | | | |
| | QIQLVQSGPDLKKPGETVKISCKASGYTF | | | |
| | TNHGMNWVKQAPGKDLKWMGWINTNTGEP | | | |
| | TYADDFKGRFAFSLETSASTAYLQINNLK | | | |
| | NEDTATYFCASPLYYRNGRYFDVWGAGTT | | | |
| | VTVSS | | | |
| | (SEQ ID NO:58) | | | |
| P1E2.3B12 | CAGGTCCAACTGCAGCAGCCTGGGGCTGA | SYWMH | EINPTYGR | LVRLRYFD |
| | ACTGGTGAAGCCTGGGGCTTCAGTGAAGC | (SEQ ID | SNYNEKFK | V |
| | TGTCCTGTAAGGCTTCTGGCTACACCTTC | NO:64) | S | SEQ ID |
| | ACCAGCTACTGGATGCACTGGGTGAAGCA | | (SEQ ID | NO:66) |
| | GAGGCCTGGACAAGGCCTTGAGTGGATTG | | NO:65) | |
| | GAGAGATTAATCCTACCTACGGTCGTAGT | | | |
| | AATTACAATGAGAAGTTCAAGAGTAAGGC | | | |
| | CACACTGACTGTAGACAAATCCTCCAGCA | | | |
| | CAGCCTACATGCAACTCAGCAGCCTGACA | | | |
| | TCTGAGGACTCTGCGGTCTATTACTGTGC | | | |
| | AAGATTAGTACGCCTACGGTACTTCGATG | | | |
| | TCTGGGGCGCAGGGACCACGGTCACCGTC | | | |
| | TCCTCA | | | |
| | (SEQ ID NO:62) | | | |
| | QVQLQQPGAELVKPGASVKLSCKASGYTF | | | |
| | TSYWMHWVKQRPGQGLEWIGEINPTYGRS | | | |
| | NYNEKFKSKATLTVDKSSSTAYMQLSSLT | | | |
| | SEDSAVYYCARLVRLRYFDVWGAGTTVTV | | | |
| | SS | | | |
| | (SEQ ID NO:63) | | | |

*Determined by the Kabat system (see supra).
N = nucleotide sequence, P = polypeptide sequence.

As known in the art, “sequence identity” between two polypeptides or two polynucleotides is determined by comparing the amino acid or nucleic acid sequence of one polypeptide or polynucleotide to the sequence of a second polypeptide or polynucleotide. When discussed herein, whether any particular polypeptide is at least about 40%, 45%, 50%, 55%, 60%, 65%, 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In certain embodiments, an antibody or antigen-binding fragment comprising the VH encoded by the polynucleotide specifically or preferentially binds to IGF-1R. In certain embodiments the nucleotide sequence encoding the VH polypeptide is altered without altering the amino acid sequence encoded thereby. For instance, the sequence may be altered for improved codon usage in a given species, to remove splice sites, or the remove restriction enzyme sites. Sequence optimizations such as these are described in the examples and are well known and routinely carried out by those of ordinary skill in the art.

In another embodiment, the present invention provides an isolated polynucleotide comprising, consisting essentially of, or consisting of a nucleic acid encoding an immunoglobulin heavy chain variable region (VH) in which the VH-CDR1, VH-CDR2, and VH-CDR3 regions have polypeptide sequences which are identical to the VH-CDR1, VH-CDR2, and VH-CDR3 groups shown in Table 5. In certain embodiments, an antibody or antigen-binding fragment comprising the VH encoded by the polynucleotide specifically or preferentially binds to IGF-1R.

In certain embodiments, an antibody or antigen-binding fragment thereof comprising, consisting essentially of, or consisting of a VH encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R.

In certain embodiments, an antibody or antigen-binding fragment thereof comprising, consisting essentially of, or consisting of a VH encoded by one or more of the polynucleotides described above specifically or preferentially binds to an IGF-1R polypeptide or fragment thereof, or a IGF-1R variant polypeptide, with an affinity characterized by a dissociation constant (KD) no greater than 5×10⁻² M, 10⁻² M, 5×10⁻³ M, 10⁻³ M, 5×10⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻³ M, 5×10⁻¹⁴ M, 10⁻¹⁴ M, 5×10⁻¹⁵ M, or 10⁻¹⁵ M.

In another embodiment, the present invention provides an isolated polynucleotide comprising, consisting essentially of, or consisting of a nucleic acid encoding an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2, or VL-CDR3 amino acid sequences from monoclonal IGF-1R antibodies disclosed herein. Alternatively, the VL-CDR1, VL-CDR2, and VL-CDR3 regions of the VL are at least 80%, 85%, 90% or 95% identical to reference light chain VL-CDR1, VL-CDR2, and VL-CDR3 amino acid sequences from monoclonal IGF-1R antibodies disclosed herein. Thus, according to this embodiment a light chain variable region of the invention has VL-CDR1, VL-CDR2, or VL-CDR3 polypeptide sequences related to the polypeptide sequences shown in Table 6:

TABLE 6

Reference VL-CDR1, VL-CDR2, and
VL-CDR3 amino acid sequences*

| | VL SEQUENCE PN/PP | | | |
|---|---|---|---|---|
| | (VL-CDR1, VL-CDR2, and | | | |
| Antibody | VL-CDR3 sequences underlined) | VL CDR1 | VL CDR2 | VL CDR3 |
| M12-E01 | CAGTACGAATTGACTCAGCCGCCCTCGG | SGSSSNIGN | YDDLLPS | AAWDDNLN |
| | TGTCTGAGGCCCCCCGGCAGAGGGTCAC | NAIN | (SEQ ID | GVI |
| | CATCTCCTGTTCTGGAAGCAGCTCCAAC | (SEQ ID | NO:70) | (SEQ ID |
| | ATCGGAAATAATGCTATAAACTGGTACC | NO:69) | | NO:71) |
| | AGCAACTCCCAGGAAAGCCTCCCAAACT | | | |
| | CCTCATCTATTATGATGATCTGTTGCCC | | | |
| | TCAGGGGTCTCTGACCGATTCTCTGGCT | | | |
| | CCAAGTCTGGCACCTCAGGCTCCCTGGC | | | |
| | CATCAGTGGGCTGCAGTCTGAGGATGAG | | | |
| | GCTGATTATTACTGTGCAGCATGGGATG | | | |
| | ACAACCTGAATGGTGTGATTTTCGGCGG | | | |
| | AGGGACCAAGCTGACCGTCCTA | | | |
| | (SEQ ID NO:67) | | | |
| | QYELTQPPSVSEAPRQRVTISCSGSSSN | | | |
| | IGNNAINWYQQLPGKPPKLLIYYDDLLP | | | |
| | SGVSDRFSGSKSGTSGSLAISGLQSEDE | | | |
| | ADYYCAAWDDNLNGVIFGGGTKLTVL | | | |
| | (SEQ ID NO:68) | | | |
| M12-G04 | GACATCCAGATGACCCAGTCTCCACTCT | PASQSINGY | ATSSLQS | QQSYSTPP |
| | CCCTGTCTGCATCTGTAGGAGACAGAGT | LN | (SEQ ID | YT |
| | CACCATCACTTGCCGGGCAAGTCAGAGC | (SEQ ID | NO:75) | (SEQ ID |
| | ATTAACGGCTACTTAAATTGGTATCAGC | NO:74) | | NO:76) |
| | AGAAACCAGGGAAAGCCCCTAACCTCCT | | | |
| | GATCTACGCTACATCCAGTTTGCAAAGT | | | |
| | GGGGTCCCATCAAGGTTCAGTGGCAGTG | | | |
| | GATCTGGGACAGATTTCACTCTCACCAT | | | |
| | CAGCAGTCTGCAACCTGAAGATTTTGCA | | | |
| | ACTTACTACTGTCAACAGAGTTACAGTA | | | |
| | CCCCCCCGTACACTTTTGGCCAGGGGAC | | | |
| | CAAGCTGGAGATCAAA | | | |
| | (SEQ ID NO:72) | | | |
| | DIQMTQSPLSLSASVGDRVTITCRASQS | | | |
| | INGYLNWYQQKPGKAPNLLIYATSSLQS | | | |
| | GVPSRFSGSGSGTDFTLTISSLQPEDFA | | | |
| | TYYCQQSYSTPPYTFGQGTKLEIK | | | |
| | (SEQ ID NO:73) | | | |
| M13-C06 | GACATCCAGATGACCCAGTCTCCACTCT | QASRDIRNY | DASSLQT | QQFDSLPH |
| | CCCTGTCTGCATCTGTAGGAGACAGAGT | LN | (SEQ ID | T |
| | CACCATCACTTGCCAGGCGAGTCGGGAC | (SEQ ID | NO:80) | (SEQ ID |
| | ATTAGAAACTATTTAAATTGGTATCAAC | NO:79) | | NO:81) |
| | AAAAACCAGGGAAAGCCCCGAAGCTCCT | | | |
| | GATCTACGATGCATCCAGTTTGCAAACA | | | |
| | GGGGTCCCATCAAGGTTCGGTGGCAGTG | | | |
| | GATCTGGGACAGACTTTAGTTTCACCAT | | | |
| | CGGCAGCCTGCAGCCTGAAGATATTGCA | | | |
| | ACATATTACTGTCAACAGTTTGATAGTC | | | |
| | TCCCTCACACTTTTGGCCAGGGGACCAA | | | |
| | ACTGGAGATCAAA | | | |
| | (SEQ ID NO:77) | | | |
| | DIQMTQSPLSLSASVGDRVTITCQASRD | | | |
| | IRNYLNWYQQKPGKAPKLLIYDASSLQT | | | |
| | GVPSRFGGSGSGTDFSFTIGSLQPEDIA | | | |
| | TYYCQQFDSLPHTFGQGTKLEIK | | | |
| | (SEQ ID NO:78) | | | |
| M14-B01 | GACATCCAGATGACCCAGTTTCCAGCCA | RASQSVMRN | GASKRAT | HQRSTWPL |
| | CCCTGTCTGTGTCTCCAGGGGAAAGAGC | LA | (SEQ ID | GT |
| | CACCCTCTCCTGCAGGGCCAGTCAGAGT | (SEQ ID | NO:85) | (SEQ ID |
| | GTTATGAGGAACTTAGCCTGGTACCAGC | NO:84) | | NO:86) |
| | AGAAACCTGGCCAGCCTCCCAGGCTCCT | | | |
| | CATCTATGGTGCATCCAAAAGGGCCACT | | | |
| | GGCATCCCAGCCAGGTTCAGTGGCAGTG | | | |
| | GGTCTGGGACAGCCTTCACTCTCACCAT | | | |
| | CAGCAACCTAGAGCCTGAAGATTTTGCA | | | |
| | GTTTATTACTGTCACCAACGTAGCACCT | | | |
| | GGCCTCTGGGGACTTTCGGCCCTGGGAC | | | |
| | CAAACTGGAGGCCAAA | | | |
| | (SEQ ID NO:82) | | | |
| | DIQMTQFPATLSVSPGERATLSCRASQS | | | |
| | VMRNLAWYQQKPGQPPRLLIYGASKRAT | | | |
| | GIPARFSGSGSGTAFTLTISNLEPEDFA | | | |
| | VYYCHQRSTWPLGTFGPGTKTLEAK | | | |
| | (SEQ ID NO:83) | | | |
| M14-C03 | GACATCCAGATGACCCAGTCTCCAGCCA | RASQSVSSY | DASNRAT | QQRSNWPP |
| | CCCTGTCTTTGTCTCCAGGGGAAAGAGC | LA | (SEQ ID | EVT |
| | CACCCTCTCCTGCAGGGCCAGTCAGAGT | (SEQ ID | NO:90) | (SEQ ID |
| | GTTAGCAGCTACTTAGCCTGGTACCAAC | NO:89) | | NO:91) |
| | AGAAACCTGGCCAGGCTCCCAGGCTCCT | | | |
| | CATCTATGATGCATCCAACAGGGCCACT | | | |
| | GGCATCCCAGCCAGGTTCAGTGGCAGTG | | | |
| | GGTCTGGGACAGACTTCACTCTCACCAT | | | |
| | CAGCAGCCTAGAGCCTGAAGATTTTGCA | | | |
| | GTTTATTACTGTCAGCAGCGTAGCAACT | | | |
| | GGCCTCCGGAGGTCACTTTCGGCCCTGG | | | |
| | GACCAAAGTGGATATCAAA | | | |
| | (SEQ ID NO:87) | | | |
| | DIQMTQSPATLSLSPGERATLSCRASQS | | | |
| | VSSYLAWYQQKPGQAPRLLIYDASNRAT | | | |
| | GIPARFSGSGSGTDFTLTISSLEPEDFA | | | |
| | VYYCQQRSNWPPEVTFGPGTKVDIK | | | |
| | (SEQ ID NO:88) | | | |
| M14-G11 | GACATCCAGATGACCCAGTCTCCAGACT | KSSQSVLYS | LASTRES | QQYYSTWT |
| | CCCTGGCTGTGTCTCTGGGCGAGAGGGC | SNNKNYLA | (SEQ ID | (SEQ ID |
| | CACCATCAACTGCAAGTCCAGCCAGAGT | (SEQ ID | NO:95) | NO:96) |
| | GTTTTATACAGCTCCAACAATAAGAACT | NO:94) | | |
| | ACTTAGCTTGGTACCAGCAGAAACCAGG | | | |
| | ACAGCCTCCTAAGCTGCTCATTTACTTG | | | |
| | GCATCTACCCGGGAATCCGGGGTCCCTG | | | |
| | ACCGATTCAGTGGCAGCGGGTCTGGGAC | | | |
| | AGATTTCACTCTCACCATCAGCAGCCTG | | | |
| | CAGGCTGAAGATGTGGCAGTTTATTACT | | | |
| | GTCAGCAATATTATAGTACTTGGACGTT | | | |
| | CGGCCAAGGGACCAAGGTGGAAATCAAA | | | |
| | (SEQ ID NO:92) | | | |
| | DIQMTQSPDSLAVSLGERATINCKSSQS | | | |
| | VLYSSNNKNYLAWYQQKPGQPPKLLIYL | | | |
| | ASTRESGVPDRFSGSGSGTDFTLTISSL | | | |
| | QAEDVAVYYCQQYYSTWTFGQGTKVEIK | | | |
| | (SEQ ID NO:93) | | | |
| P2A7.3E11 | GAAGTTGTGCTCACCCAGTCTCCAACCG | SASSTLSSN | RTSNLAS | QQGSSIPL |
| | CCATGGCTGCATCTCCCGGGGAGAAGAT | YLH | (SEQ ID | T |
| | CACTATCACCTGCAGTGCCAGCTCAACT | (SEQ ID | NO:100) | (SEQ ID |
| | TTAAGTTCCAATTACTTGCATTGGTATC | NO:99) | | NO:101) |
| | AGCAGAAGCCAGGATTCTCCCCTAAACT | | | |
| | CTTGATTTATAGGACATCCAATCTGGCC | | | |
| | TCTGGAGTCCCAGGTCGCTTCAGTGGCA | | | |
| | GTGGGTCTGGGACCTCTTACTCTCTCAC | | | |
| | AATTGGCACCATGGAGGCTGAAGATGTT | | | |
| | GCCACTTACTACTGCCAGCAGGGTAGTA | | | |
| | GTATACCGCTCACGTTCGGTGCTGGGAC | | | |
| | CAAGCTGGAGCTGAAG | | | |
| | (SEQ ID NO:97) | | | |
| | EVVLTQSPTAMAASPGEKITITCSASST | | | |
| | LSSNYLHWYQQKPGFSPKLLIYRTSNLA | | | |
| | SGVPGRFSGSGSGTSYSLTIGTMEAEDV | | | |
| | ATYYCQQGSSIPLTFGAGTKLELK | | | |
| | (SEQ ID NO:98) | | | |
| 20C8.3B8 | GACATTGTGCTGACACAGTCTCCTGCTT | RASKSVSTS | LASNLES | QHSRELPY |
| | CCTTAGCTGTATCTCTGGGGCAGAGGGC | AYSYMH | (SEQ ID | T |
| | CACCATCTCATGCAGGGCCAGCAAAAGT | (SEQ ID | NO:105) | (SEQ ID |
| | GTCAGTACATCTGCCTATAGTTATATGC | NO:104) | | NO:106) |
| | ACTGGTACCAACAGAAACCAGGACAGCC | | | |
| | ACCCAAACTCCTCATCTATCTTGCATCC | | | |
| | AACCTAGAATCTGGGGTCCCTGCCAGGT | | | |
| | TCAGTGGCAGTGGGTCTGGGACAGACTT | | | |
| | CACCCTCAACATCCATCCTGTGGAGGAG | | | |
| | GAGGATGCTGCAACCTATTACTGTCAGC | | | |
| | ACAGTAGGGAGCTTCCGTATACGTTCGG | | | |
| | AGGGGGGACCAAGCTGGAAATC | | | |
| | (SEQ ID NO:102) | | | |
| | DIVLTQSPASLAVSLGQRATISCRASKS | | | |
| | VSTSAYSYMHWYQQKPGQPPKLLIYLAS | | | |
| | NLESGVPARFSGSGSGTDFTLNIHPVEE | | | |
| | EDAATYYCQHSRELPYTFGGGTKLEIK | | | |
| | (SEQ ID NO:103) | | | |
| P1A2.2B11 | GATATCCAGATGACACAGACTACATCCT | RASQDISNY | TSRLHS | QQGKTLPW |
| | CCCTATCTGCCTCTCTGGGAGACAGAGT | LN | (SEQ ID | T |
| | CACCATCAGTTGCAGGGCAAGTCAGGAC | (SEQ ID | NO:110) | (SEQ ID |
| | ATTAGCAATTATTTAAACTGGTATCAGC | NO:109) | | NO:111) |
| | AGAAACCAGATGGAACTATTAAACTCCT | | | |
| | GATCTACTACACATCAAGATTACACTCA | | | |
| | GGAGTCCCATCAAGGTTCAGTGGCAGTG | | | |
| | GGTCTGGAACAGATTATTCTCTCACCAT | | | |
| | TAGCAACCTGGAACAAGAAGATTTTGCC | | | |
| | ACTTACTTTTGCCAACAGGGTAAAACGC | | | |
| | TTCCGTGGACGTTCGGTGGAGGCACCAA | | | |
| | GCTGGAAATCAAA | | | |
| | (SEQ ID NO:107) | | | |
| | DIQMTQTTSSLSASLGDRVTISCPASQD | | | |
| | ISNYLNWYQQKPDGTIKLLIYYTSRLHS | | | |
| | GVPSRFSGSGSGTDYSLTTSNLEQEDFA | | | |
| | TYFCQQGKTLPWTFGGGTKLEIK | | | |
| | (SEQ ID NO:108) | | | |
| 20D8.24B11 | SAME AS 20C8 | | | |
| P1G10.2B8 | GATATCCAGATGACACAGACTACATCCT | RASQDISNY | TSRLH | QQGKTLPW |
| | CCCTGTCTGCCTCTCTGGGAGACAGAGT | LN | (SEQ ID | T |
| | CACCATCAGTTGCAGGGCAAGTCAGGAC | (SEQ ID | NO:115) | (SEQ ID |
| | ATTAGTAATTATTTAAATTGGTATCAGC | NO:114) | | NO:116) |
| | AGAAACCAGATGGATCTGTTAAACTCCT | | | |
| | GATCTACTACACATCAAGATTACACTCA | | | |
| | GGAGTCCCATCAAGGTTCAGTGGCAGTG | | | |
| | GGTCTGGAACAGATTATTCTCTCACCAT | | | |
| | TAGCAACCTGGAACAAGAAGATATTGCC | | | |
| | ACTTACTTTTGCCAACAGGGAAAGACGC | | | |
| | TTCCGTGGACGTTCGGTGGAGGCACCAA | | | |
| | CTGGAAATCAAA | | | |
| | (SEQ ID NO:112) | | | |
| | DIQMTQTTSSLSASLGDRVTISCRASQD | | | |
| | ISNYLNWYQQKPDGSVKLLIYYTSRLHS | | | |
| | GVPSRFSGSGSGTDYSLTISNLEQEDIA | | | |
| | TYFCQQGKTLPWTFGGGTKLEIK | | | |
| | (SEQ ID NO:113) | | | |
| P1E2.3B12 | GATATTGTGATGACGCAGGCTGCATTCT | RSSKSLLHS | QMSNLAS | AQNLELPY |
| | CCAATCCAGTCACTCTTGGAACATCAGC | NGITYLY | (SEQ ID | T |
| | TTCCATCTCCTGCAGGTCTAGTAAGAGT | (SEQ ID | NO:120) | (SEQ ID |
| | CTCCTACATAGTAATGGCATCACTTATT | NO:119) | | NO:121) |
| | TGTATTGGTATCTGCAGAAGCCAGGCCA | | | |
| | GTCTCCTCAGCTCCTGATTTATCAGATG | | | |
| | TCCAACCTTGCCTCAGGAGTCCCAGACA | | | |
| | GGTTCAGTAGCAGTGGGTCAGGAACTGA | | | |
| | TTTCACACTGAGAATCAGCAGAGTGGAG | | | |
| | GCTGAGGATGTGGGTGTTTATTACTGTG | | | |
| | CTCAAAATCTAGAACTTCCGTACACGTT | | | |
| | CGGAGGGGGGACCAAGCTGGAAATCAAA | | | |
| | (SEQ ID NO:117) | | | |
| | DIVMTQAAFSNPVTLGTSASISCRSSKS | | | |
| | LLHSNGITYLYWYLQKPGQSPQLLIYQM | | | |
| | SNLASGVPDRFSSSGSGTDFTLRISRVE | | | |
| | AEDVGVYYCAQNLELPYTFGGGTKLEIK | | | |
| | (SEQ ID NO:118) | | | |

*Deteimined by the Kabat system (see supra).
PN = nucleotide sequence, PP = polypeptide sequence.

In certain embodiments, an antibody or antigen-binding fragment comprising the VL encoded by the polynucleotide specifically or preferentially binds to IGF-1R.

In another embodiment, the present invention provides an isolated polynucleotide comprising, consisting essentially of, or consisting of a nucleic acid encoding an immunoglobulin light chain variable region (VL) in which the VL-CDR1, VL-CDR2, and VL-CDR3 regions have polypeptide sequences which are identical to the VL-CDR1, VL-CDR2, and VL-CDR3 groups shown in Table 6. In certain embodiments, an antibody or antigen-binding fragment comprising the VL encoded by the polynucleotide specifically or preferentially binds to IGF-1R.

In a further aspect, the present invention provides an isolated polynucleotide comprising, consisting essentially of, or consisting of a nucleic acid encoding an immunoglobulin light chain variable region (VL) in which the VL-CDR1, VL-CDR2, and VL-CDR3 regions are encoded by nucleotide sequences which are identical to the nucleotide sequences which encode the VL-CDR1, VL-CDR2, and VL-CDR3 groups shown in Table 6. In certain embodiments, an antibody or antigen-binding fragment comprising the VL encoded by the polynucleotide specifically or preferentially binds to IGF-1R.

In certain embodiments, an antibody or antigen-binding fragment thereof comprising, consisting essentially of, or consisting of a VL encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R.

In certain embodiments, an antibody or antigen-binding fragment thereof comprising, consisting essentially of, or consisting of a VL encoded by one or more of the polynucleotides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In a further embodiment, the present invention includes an isolated polynucleotide comprising, consisting essentially of, or consisting of a nucleic acid encoding a VH at least 80%, 85%, 90% 95% or 100% identical to a reference VH polypeptide sequence selected from the group consisting of SEQ ID NOs: 4, 9, 14, 20, 26, 32, 38, 43, 48, 53, 58, and 63. In certain embodiments, an antibody or antigen-binding fragment comprising the VH encoded by the polynucleotide specifically or preferentially binds to IGF-1R.

In another aspect, the present invention includes an isolated polynucleotide comprising, consisting essentially of, or consisting of a nucleic acid sequence encoding a VH having a polypeptide sequence selected from the group consisting of SEQ ID NOs: 4, 9, 14, 20, 26, 32, 38, 43, 48, 53, 58, and 63. In certain embodiments, an antibody or antigen-binding fragment comprising the VH encoded by the polynucleotide specifically or preferentially binds to IGF-1R.

In a further embodiment, the present invention includes an isolated polynucleotide comprising, consisting essentially of, or consisting of a VH-encoding nucleic acid at least 80%, 85%, 90% 95% or 100% identical to a reference nucleic acid sequence selected from the group consisting of SEQ ID NOs: 3, 8, 13, 18, 19, 24, 25, 30, 31, 36, 37, 42, 47, 52, 57, and 62. In certain embodiments, an antibody or antigen-binding fragment comprising the VH encoded by such polynucleotides specifically or preferentially binds to IGF-1R.

In another aspect, the present invention includes an isolated polynucleotide comprising, consisting essentially of, or consisting of a nucleic acid sequence encoding a VH of the invention, where the amino acid sequence of the VH is selected from the group consisting of SEQ ID NOs: 4, 9, 14, 20, 26, 32, 38, 43, 48, 53, 58, and 63. The present invention further includes an isolated polynucleotide comprising, consisting essentially of, or consisting of a nucleic acid sequence encoding a VH of the invention, where the sequence of the nucleic acid is selected from the group consisting of SEQ ID NOs: 3, 8, 13, 18, 19, 24, 25, 30, 31, 36, 37, 42, 47, 52, 57, and 62. In certain embodiments, an antibody or antigen-binding fragment comprising the VH encoded by such polynucleotides specifically or preferentially binds to IGF-1R.

In certain embodiments, an antibody or antigen-binding fragment thereof comprising, consisting essentially of, or consisting of a VH encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R, or will competitively inhibit such a monoclonal antibody from binding to IGF-1R.

In certain embodiments, an antibody or antigen-binding fragment thereof comprising, consisting essentially of, or consisting of a VH encoded by one or more of the polynucleotides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In a further embodiment, the present invention includes an isolated polynucleotide comprising, consisting essentially of, or consisting of a nucleic acid encoding a VL at least 80%, 85%, 90% 95% or 100% identical to a reference VL polypeptide sequence having an amino acid sequence selected from the group consisting of SEQ ID NOs: 68, 73, 78, 83, 88, 93, 98, 103, 108, 113, and 118. In a further embodiment, the present invention includes an isolated polynucleotide comprising, consisting essentially of, or consisting of a VL-encoding nucleic acid at least 80%, 85%, 90% 95% or 100% identical to a reference nucleic acid sequence selected from the group consisting of SEQ ID NOs: 67, 72, 77, 82, 87, 92, 97, 102, 107, 112, and 117. In certain embodiments, an antibody or antigen-binding fragment comprising the VL encoded by such polynucleotides specifically or preferentially binds to IGF-1R.

In another aspect, the present invention includes an isolated polynucleotide comprising, consisting essentially of, or consisting of a nucleic acid sequence encoding a VL having a polypeptide sequence selected from the group consisting of SEQ ID NOs: 68, 73, 78, 83, 88, 93, 98, 103, 108, 113, and 118. The present invention further includes an isolated polynucleotide comprising, consisting essentially of, or consisting of a nucleic acid sequence encoding a VL of the invention, where the sequence of the nucleic acid is selected from the group consisting of SEQ ID NOs: 67, 72, 77, 82, 87, 92, 97, 102, 107, 112, and 117. In certain embodiments, an antibody or antigen-binding fragment comprising the VL encoded by such polynucleotides specifically or preferentially binds to IGF-1R.

In certain embodiments, an antibody or antigen-binding fragment thereof comprising, consisting essentially of, or consisting of a VL encoded by one or more of the polynucleotides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R.

In certain embodiments, an antibody or antigen-binding fragment thereof comprising, consisting essentially of, or consisting of a VL encoded by one or more of the polynucleotides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polynucleotides described above may further include additional nucleic acids, encoding, e.g., a signal peptide to direct secretion of the encoded polypeptide, antibody constant regions as described herein, or other heterologous polypeptides as described herein.

Also, as described in more detail elsewhere herein, the present invention includes compositions comprising the polynucleotides comprising one or more of the polynucleotides described above. In one embodiment, the invention includes compositions comprising a first polynucleotide and second polynucleotide wherein said first polynucleotide encodes a VH polypeptide as described herein and wherein said second polynucleotide encodes a VL polypeptide as described herein. Specifically a composition which comprises, consists essentially of, or consists of a VH polynucleotide, and a VL polynucleotide, wherein the VH polynucleotide and the VL polynucleotide encode polypeptides, respectively at least 80%, 85%, 90% 95% or 100% identical to reference VL and VL polypeptide amino acid sequences selected from the group consisting of SEQ ID NOs: 4 and 68, 8 and 73, 14 and 78, 20 and 83, 26 and 88, 32 and 93, 38 and 98, 43 and 103, 48 and 108, 53 and 103, 58 and 113, and 63 and 118. Or alternatively, a composition which comprises, consists essentially of, or consists of a VH polynucleotide, and a VL polynucleotide at least 80%, 85%, 90% 95% or 100% identical, respectively, to reference VL and VL nucleic acid sequences selected from the group consisting of SEQ ID NOs: 3 and 67, 8 and 72, 13 and 77, 18 and 77, 19 and 82, 24 and 82, 25 and 87, 30 and 87, 31 and 92, 36 and 92, 37 and 97, 42 and 102, 47 and 107, 58 and 102, 57 and 112, and 62 and 117. In certain embodiments, an antibody or antigen-binding fragment comprising the VH and VL encoded by the polynucleotides in such compositions specifically or preferentially binds to IGF-1R.

The present invention also includes fragments of the polynucleotides of the invention, as described elsewhere. Additionally polynucleotides which encode fusion polynucleotides, Fab fragments, and other derivatives, as described herein, are also contemplated by the invention.

The polynucleotides may be produced or manufactured by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an IGF-1R antibody, or antigen-binding fragment, variant, or derivative thereof may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+ RNA, isolated from, any tissue or cells expressing the antibody or other IGF-1R antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other IGF-1R antibody. Amplified nucleic acids generated by PCR may then be cloned into replicable cloning vectors using any method well known in the art.

Once the nucleotide sequence and corresponding amino acid sequence of the IGF-1R antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding an IGF-1R antibody, or antigen-binding fragment, variant, or derivative thereof can be composed of any polyribonucleotide or polydeoxyribonucleotide, which may be unmodified RNA or DNA or modified RNA or DNA. For example, a polynucleotide encoding IGF-1R antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an IGF-1R antibody, or antigen-binding fragment, variant, or derivative thereof can be composed of triple-stranded regions comprising RNA or DNA or both RNA and DNA. A polynucleotide encoding an IGF-1R antibody, or antigen-binding fragment, variant, or derivative thereof may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

### V. IGF-1R Antibody Polypeptides

The present invention is further directed to isolated polypeptides which make up IGF-1R antibodies, and polynucleotides encoding such polypeptides. IGF-1R antibodies of the present invention comprise polypeptides, e.g., amino acid sequences encoding IGF-1R-specific antigen binding regions derived from immunoglobulin molecules. A polypeptide or amino acid sequence “derived from” a designated protein refers to the origin of the polypeptide having a certain amino acid sequence. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the present invention provides an isolated polypeptide comprising, consisting essentially of, or consisting of an immunoglobulin heavy chain variable region (VH), where at least one of VH-CDRs of the heavy chain variable region or at least two of the VH-CDRs of the heavy chain variable region are at least 80%, 85%, 90% or 95% identical to reference heavy chain VH-CDR1, VH-CDR2 or VH-CDR3 amino acid sequences from monoclonal IGF-1R antibodies disclosed herein. Alternatively, the VH-CDR1, VH-CDR2 and VH-CDR3 regions of the VH are at least 80%, 85%, 90% or 95% identical to reference heavy chain VH-CDR1, VH-CDR2 and VH-CDR3 amino acid sequences from monoclonal IGF-1R antibodies disclosed herein. Thus, according to this embodiment a heavy chain variable region of the invention has VH-CDR1, VH-CDR2 and VH-CDR3 polypeptide sequences related to the groups shown in Table 5, supra. While Table 5 shows VH-CDRs defined by the Kabat system, other CDR definitions, e.g., VH-CDRs defined by the Chothia system, are also included in the present invention. In certain embodiments, an antibody or antigen-binding fragment comprising the VH specifically or preferentially binds to IGF-1R.

In another embodiment, the present invention provides an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 5. In certain embodiments, an antibody or antigen-binding fragment comprising the VH specifically or preferentially binds to IGF-1R.

In another embodiment, the present invention provides an isolated polypeptide comprising, consisting essentially of, or consisting of an immunoglobulin heavy chain variable region (VH) in which the VH-CDR1, VH-CDR2 and VH-CDR3 regions have polypeptide sequences which are identical to the VH-CDR1, VH-CDR2 and VH-CDR3 groups shown in Table 5, except for one, two, three, four, five, or six amino acid substitutions in any one VH-CDR. In larger CDRs, e.g., VH-CDR-3, additional substitutions may be made in the CDR, as long as the a VH comprising the VH-CDR specifically or preferentially binds to IGF-1R. In certain embodiments the amino acid substitutions are conservative. In certain embodiments, an antibody or antigen-binding fragment comprising the VH specifically or preferentially binds to IGF-1R.

In a further embodiment, the present invention includes an isolated polypeptide comprising, consisting essentially of, or consisting of a VH polypeptide at least 80%, 85%, 90% 95% or 100% identical to a reference VH polypeptide amino acid sequence selected from the group consisting of SEQ ID NOs: SEQ ID NOs: 4, 9, 14, 20, 26, 32, 38, 43, 48, 53, 58, and 63. In certain embodiments, an antibody or antigen-binding fragment comprising the VH polypeptide specifically or preferentially binds to IGF-1R.

In another aspect, the present invention includes an isolated polypeptide comprising, consisting essentially of, or consisting of a VH polypeptide selected from the group consisting of SEQ ID NOs: SEQ ID NOs: 4, 9, 14, 20, 26, 32, 38, 43, 48, 53, 58, and 63. In certain embodiments, an antibody or antigen-binding fragment comprising the VH polypeptide specifically or preferentially binds to IGF-1R.

In certain embodiments, an antibody or antigen-binding fragment thereof comprising, consisting essentially of, or consisting of a one or more of the VH polypeptides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R.

In certain embodiments, an antibody or antigen-binding fragment thereof comprising, consisting essentially of, or consisting of one or more of the VH polypeptides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another embodiment, the present invention provides an isolated polypeptide comprising, consisting essentially of, or consisting of an immunoglobulin light chain variable region (VL), where at least one of the VL-CDRs of the light chain variable region or at least two of the VL-CDRs of the light chain variable region are at least 80%, 85%, 90% or 95% identical to reference light chain VL-CDR1, VL-CDR2 or VL-CDR3 amino acid sequences from monoclonal IGF-1R antibodies disclosed herein. Alternatively, the VL-CDR1, VL-CDR2 and VL-CDR3 regions of the VL are at least 80%, 85%, 90% or 95% identical to reference light chain VL-CDR1, VL-CDR2 and VL-CDR3 amino acid sequences from monoclonal IGF-1R antibodies disclosed herein. Thus, according to this embodiment a light chain variable region of the invention has VL-CDR1, VL-CDR2 and VL-CDR3 polypeptide sequences related to the polypeptides shown in Table 6, supra. While Table 6 shows VL-CDRs defined by the Kabat system, other CDR definitions, e.g., VL-CDRs defined by the Chothia system, are also included in the present invention. In certain embodiments, an antibody or antigen-binding fragment comprising the VL polypeptide specifically or preferentially binds to IGF-1R.

In another embodiment, the present invention provides an isolated polypeptide comprising, consisting essentially of, or consisting of an immunoglobulin light chain variable region (VL) in which the VL-CDR1, VL-CDR2 and VL-CDR3 regions have polypeptide sequences which are identical to the VL-CDR1, VL-CDR2 and VL-CDR3 groups shown in Table 6. In certain embodiments, an antibody or antigen-binding fragment comprising the VL polypeptide specifically or preferentially binds to IGF-1R.

In another embodiment, the present invention provides an isolated polypeptide comprising, consisting essentially of, or consisting of an immunoglobulin heavy chain variable region (VL) in which the VL-CDR1, VL-CDR2 and VL-CDR3 regions have polypeptide sequences which are identical to the VL-CDR1, VL-CDR2 and VL-CDR3 groups shown in Table 6, except for one, two, three, four, five, or six amino acid substitutions in any one VL-CDR. In larger CDRs, additional substitutions may be made in the VL-CDR, as long as the a VL comprising the VL-CDR specifically or preferentially binds to IGF-1R. In certain embodiments the amino acid substitutions are conservative. In certain embodiments, an antibody or antigen-binding fragment comprising the VL specifically or preferentially binds to IGF-1R.

In a further embodiment, the present invention includes an isolated polypeptide comprising, consisting essentially of, or consisting of a VL polypeptide at least 80%, 85%, 90% 95% or 100% identical to a reference VL polypeptide sequence selected from the group consisting of SEQ ID NOs: 68, 73, 78, 83, 88, 93, 98, 103, 108, 113, and 118. In certain embodiments, an antibody or antigen-binding fragment comprising the VL polypeptide specifically or preferentially binds to IGF-1R.

In another aspect, the present invention includes an isolated polypeptide comprising, consisting essentially of, or consisting of a VL polypeptide selected from the group consisting of S SEQ ID NOs: 68, 73, 78, 83, 88, 93, 98, 103, 108, 113, and 118. In certain embodiments, an antibody or antigen-binding fragment comprising the VL polypeptide specifically or preferentially binds to IGF-1R.

In certain embodiments, an antibody or antigen-binding fragment thereof comprising, consisting essentially of, one or more of the VL polypeptides described above specifically or preferentially binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8, or will competitively inhibit such a monoclonal antibody or fragment from binding to IGF-1R.

In certain embodiments, an antibody or antigen-binding fragment thereof comprising, consisting essentially of, or consisting of a one or more of the VL polypeptides described above specifically or preferentially binds to an IGF-1R polypeptide or fragment thereof, or a IGF-1R variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In other embodiments, an antibody or antigen-binding fragment thereof comprises, consists essentially of or consists of a VH polypeptide, and a VL polypeptide, where the VH polypeptide and the VL polypeptide, respectively are at least 80%, 85%, 90% 95% or 100% identical to reference VL and VL polypeptide amino acid sequences selected from the group consisting of SEQ ID NOs: 4 and 68, 8 and 73, 14 and 78, 20 and 83, 26 and 88, 32 and 93, 38 and 98, 43 and 103, 48 and 108, 53 and 103, 58 and 113, and 63 and 118. In certain embodiments, an antibody or antigen-binding fragment comprising these VH and VL polypeptides specifically or preferentially binds to IGF-1R.

Any of the polypeptides described above may further include additional polypeptides, e.g., a signal peptide to direct secretion of the encoded polypeptide, antibody constant regions as described herein, or other heterologous polypeptides as described herein. Additionally, polypeptides of the invention include polypeptide fragments as described elsewhere. Additionally polypeptides of the invention include fusion polypeptide, Fab fragments, and other derivatives, as described herein.

Also, as described in more detail elsewhere herein, the present invention includes compositions comprising the polypeptides described above.

It will also be understood by one of ordinary skill in the art that IGF-1R antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In other embodiments, a polypeptide or amino acid sequence derived from a designated protein may be identical to the starting sequence except for two or fewer, three or fewer, four or fewer, five or fewer, six or fewer, seven or fewer, eight or fewer, nine or fewer, ten or fewer, fifteen or fewer, or twenty or fewer individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Certain IGF-1R antibody polypeptides of the present invention comprise, consist essentially of, or consist of an amino acid sequence derived from a human amino acid sequence. However, certain IGF-1R antibody polypeptides comprise one or more contiguous amino acids derived from another mammalian species. For example, an IGF-1R antibody of the present invention may include a primate heavy chain portion, hinge portion, or antigen binding region. In another example, one or more murine-derived amino acids may be present in a non-murine antibody polypeptide, e.g., in an antigen binding site of an IGF-1R antibody. In another example, the antigen binding site of an IGF-1R antibody is fully murine. In certain therapeutic applications, IGF-1R-specific antibodies, or antigen-binding fragments, variants, or analogs thereof are designed so as to not be immunogenic in the animal to which the antibody is administered.

In certain embodiments, an IGF-1R antibody polypeptide comprises an amino acid sequence or one or more moieties not normally associated with an antibody. Exemplary modifications are described in more detail below. For example, a single-chain fv antibody fragment of the invention may comprise a flexible linker sequence, or may be modified to add a functional moiety (e.g., PEG, a drug, a toxin, or a label).

An IGF-1R antibody polypeptide of the invention may comprise, consist essentially of, or consist of a fusion protein. Fusion proteins are chimeric molecules which comprise, for example, an immunoglobulin antigen-binding domain with at least one target binding site, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an IGF-1R antibody, or an antigen-binding fragment, variant, or analog thereof is derived from a non-immunoglobulin polypeptide of the same species, or an immunoglobulin or non-immunoglobulin polypeptide of a different species.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into IGF-1R antibodies for use in the diagnostic and treatment methods disclosed herein and screened for their ability to bind to the desired antigen, e.g., IGF-1R.

### VI. IGF-1R Epitopes

A. Epitopes Resulting in Competitive Inhibition of Binding

In certain embodiments, an IGF-1R binding moiety may bind to a competitive epitope of IGF-1R such that it competitively blocks binding of a ligand (e.g. IGF1 and/or IGF2) to IGF-1R. Such binding specificities are referred to herein as “competitive binding moieties.” In one embodiment, the competitive binding moiety competitively blocks binding of IGF-1 (but not IGF-2) to IGF-1R. In another embodiment, the competitive binding moiety competitively blocks binding of IGF-2 (but not IGF-1) to IGF-1R. In yet another embodiment, the competitive binding moiety competitively blocks binding of both IGF-1 and IGF-2 to IGF-1R.

A binding molecule is said to “competitively inhibit” or “competitively block” binding of the ligand if it specifically or preferentially binds to the epitope to the extent that binding of the ligand (e.g. IGF) to IGF-1R is inhibited or blocked (e.g. sterically blocked) in a manner that is dependent on the concentration of the ligand. For example, when measured biochemically, competitive inhibition at a given concentration of binding molecule can be overcome by increasing the concentration of ligand in which case the ligand will outcompete the binding molecule for binding to the target molecule (e.g., IGF-1R). Without being bound to any particular theory, competition is thought to occur when the epitope to which the binding molecule binds is located at or near the binding site of the ligand, thereby preventing binding of the ligand. Competitive inhibition may be determined by methods well known in the art and/or described in the Examples, including, for example, competition ELISA assays. In one embodiment, a binding molecule of the invention competitively inhibits binding of the ligand to a given epitope by at least 90%, at least 80%, at least 70%, at least 60%, or at least 50%.

An exemplary competitive epitope is situated within a region encompassing the mid and C-terminal regions of the CRR domain at residues 248-303 of IGF-1R. This epitope of IGF-1R is adjacent (in 3-dimensional space) to the IGF-1/IGF-2 ligand binding site of the L1 domain. An exemplary antibody which competitively binds to this epitope is the human antibody designated M14-G11. The M14-G11 antibody has been shown to competitively block binding of both IGF-1 and IGF-2 to IGF-1R. Chinese Hamster Ovary cell lines which express the Fab antibody fragment of M14-G11 were deposited with the American Type Culture Collection (“ATCC”) on Aug. 29, 2006, and were given ATCC Deposit Number PTA-7855.

Accordingly, in certain embodiments, a binding moiety employed in the compositions of the invention may bind to the same competitive epitope as the M14-G11 antibody. For example, a binding moiety may be derived from an antibody which cross-blocks (i.e., competes for binding with) an M14-G11 antibody or otherwise interferes with the binding of the M14-G11 antibody. In other embodiments, the binding moiety may comprise the M14-G11 antibody itself, or a fragment, variant, or derivative thereof. In other embodiments, a binding moiety may comprise an antigen binding domain, variable region (VL or VH), or CDR therefrom. For example, a competitive binding moiety may comprise all six CDRs (i.e., CDRs 1-6) of a M14-G11 antibody or it may comprise fewer than all six CDRs (e.g., one, two, three, four, or five CDRs) from the M14-G11 antibody. In one exemplary embodiment, the competitive binding specificity comprises CDR-H3 from the M14-G11 antibody.

Other antibodies which bind to a competitive epitope of IGF-1R may be identified using art-recognized methods. For example, once antibodies to various fragments of, or to the full-length IGF-1R without the signal sequence, have been produced, determining which amino acids, or epitope, of IGF-1R to which the antibody or antigen binding fragment binds can be determined by epitope mapping protocols as described herein as well as methods known in the art (e.g. double antibody-sandwich ELISA as described in “Chapter II—Immunology,” Current Protocols in Molecular Biology, Ed. Ausubel et al., v. 2, John Wiley & Sons, Inc. (1996)). Additional epitope mapping protocols may be found in Morris, G. Epitope Mapping Protocols, New Jersey: Humana Press (1996), which are both incorporated herein by reference in their entireties. Epitope mapping can also be performed by commercially available means (i.e. ProtoPROBE, Inc. (Milwaukee, Wis.)). Additionally, antibodies produced which bind to a competitive epitope of IGF-1R can then be screened for their ability to competitively inhibit binding of insulin growth factor, e.g., IGF-1, IGF-2, or both IGF-1 and IGF-2 to IGF-1R. Antibodies can be screened for these and other properties according to methods described in detail in the Examples.

In other embodiments, a competitive IGF-1R binding moiety specifically or preferentially binds to a competitive epitope which comprises, consists essentially of, or consists of at least about four to five amino acids of the sequence spanning residues 248-303 of IGF-1R, inclusive. For example, in one embodiment, a competitive IGF-1R binding moiety comprises, at least seven, at least nine, or between at least about 15 to about 30 amino acids of the sequence spanning residues 248-303 of IGF-1R. The amino acids of a given epitope may be, but need not be contiguous or linear. In certain embodiments, the competitive epitope comprises, consists essentially of, or consists of a non-linear epitope formed by the CRR and L2 domain interface of IGF-1R as expressed on the surface of a cell or as a soluble fragment, e.g., fused to an IgG Fc region. Thus, in certain embodiments a competitive epitope of IGF-1R comprises, consists essentially of, or consists of at least 3, at least 4, at least 5, at least 6, at least 7, at least 8, at least 9, at least 10, at least 15, at least 20, at least 25, between about 15 to about 30, or at least 10, 15, 20, 25, 30, 35, 40, or 45 contiguous or non-contiguous amino acids of the sequence spanning residues 248-303 of IGF-1R. In the case of non-contiguous amino acids, the amino acids form an epitope through protein folding.

In other embodiments, the competitive epitope to which the binding moiety binds comprises, consists essentially of, or consists of at least 3, at least 4, at least 5, at least 6, at least 7, at least 8, at least 9, at least 10, at least 15, at least 20, at least 25, between about 15 to about 30, contiguous or non-contiguous amino acids of IGF-1R and at least one of the amino acids of the epitope is selected from the group consisting of amino acid number 248, 250, 254, 257, 259, 260, 263, 265, 301, and 303 of IGF-1R.

In other embodiments, the amino acids bound by a binding moiety of the invention are present in the epitope spanning amino acids 248-303 of IGF-1R. In one embodiment, the epitope bound by a binding moiety of the invention includes at least one amino acid that, when mutated, leads to ablation or large decreases in antibody affinity (e.g., >100-fold decrease in affinity), e.g. IGF-1R residues 248 and/or 250. In another embodiment, the epitope may comprise one or more amino acids of IGF-1R which, when mutated, leads to a moderate decrease in antibody affinity towards the receptor (10≧KD≧100-fold above that of wild-type IGF-1R). In yet other embodiments, the epitope may comprise an amino acid of IGF-1R which, when mutated, leads to small decreases in antibody affinity (e.g., 2.5≧KD≧10 nM) compared to wild-type human IGF-1R, e.g. one or more of residues 254, 257, 259, 260, 263, 265, 301, or 303 of IGF-1R. In a preferred embodiment, the epitope bound by a binding moiety of the invention comprises any one, any two, or all three of IGF-1R residues 248, 250, and/or 254. In a particularly preferred embodiment, a competitive binding moiety binds to an epitope comprising all three amino acids 248, 250, and 254 and simultaneously recognizes these amino acid residues.

B. Epitopes Resulting in Allosteric Inhibition of Binding

In certain embodiments, a binding moiety may bind to an allosteric epitope such that it allosterically blocks binding of an IGF ligand to IGF-1R. Such binding specificities are referred to herein as “allosteric binding moieties”. In one embodiment, the allosteric binding moiety allosterically blocks binding of IGF-1 (but not IGF-2) to IGF-1R. In another embodiment, the allosteric binding moiety allosterically blocks binding of IGF-2 (but not IGF-1) to IGF-1R. In yet another embodiment, an allosteric binding moiety allosterically blocks binding of both IGF-1 and IGF-2 to IGF-1R.

A binding molecule is said to “allosterically inhibit” or “allosterically block” binding of the ligand if it specifically or preferentially binds to the epitope to the extent that binding of the ligand (e.g. IGF1 and/or IGF2) to IGF-1R is inhibited or blocked in a manner that is independent of the concentration of the binding molecule. For example, increases in the concentration of ligand will not effect the potency of inhibition (e.g., IC50 or concentration at which the binding molecule leads to a 50% reduction in its maximal ligand inhibition). Without being bound to any particular theory, allosteric inhibition is thought to occur when there is a conformational or dynamic change in the target molecule (e.g. IGF-1R) that is induced by binding of the binding molecule to the allosteric epitope, such that the affinity of the ligand for the target is reduced. Allosteric inhibition may be determined by methods well known in the art or described in the Examples, including, for example, competition ELISA assays. In one embodiment, a binding molecule may allosterically inhibit binding of the ligand to a given epitope by at least 90%, at least 80%, at least 70%, at least 60%, or at least 50%.

(i) Epitopes Resulting in Allosteric Blocking of IGF-1 and IGF-2

In certain exemplary embodiments, a binding molecule of the invention comprises a binding moiety which binds an allosteric epitope located within a region spanning the entire FnIII-1 domain of IGF-1R and comprising residues 440-586 of IGF-1R. Exemplary antibodies which allosterically bind to an epitope within this region are the human antibodies designated M13-C06 and M14-C03. Both the M13-C06 antibody and the M14-C03 antibody have been shown in the Examples to allosterically block binding of both IGF-1 and IGF-2 to IGF-1R. Chinese Hamster Ovary cell lines which express full-length antibody of M13-C06 and M14-C03 were deposited with the American Type Culture Collection (“ATCC”) on Mar. 28, 2006, and were given ATCC Deposit Numbers PTA-7444 and PTA-7445, respectively. Accordingly, in certain embodiments, a binding moiety employed in the compositions of the invention may bind to the same allosteric epitope as the M13-C06 antibody or the M14-C03 antibody. For example, a binding specificity may be derived from an antibody which cross-blocks (competes with) the M13-C06 antibody or the M14-C03 antibody or otherwise interferes with the binding of the M13-C06 antibody or the M14-C03 antibody. In other embodiments, the binding moiety may comprise either of the M13-C06 or the M14-C03 antibodies themselves, or a fragment, variant, or derivative thereof. In other embodiments, a binding moiety may comprise an antigen binding domain, variable region (VL and/or VH), or CDR therefrom. For example, an allosteric binding moiety may comprise all six CDRs of the M13-C06 antibody or the M14-C03 antibody or it may comprise fewer than all six CDRs (e.g., one, two, three, four, or five CDRs) from the M13-C06 antibody or the M14-C03 antibody. In one exemplary embodiment, the allosteric binding specificity comprises CDR-H3 from the M13-C06 antibody or the M14-C03 antibody.

In certain embodiments, an allosteric IGF-1R binding moiety specifically or preferentially binds to an allosteric epitope which comprises, consists essentially of, or consists of at least about four to five amino acids of the sequence spanning residues 440-586 of IGF-1R, at least seven, at least nine, or between at least about 15 to about 30 amino acids of the sequence spanning residues 440-586 of IGF-1R. The amino acids of a given epitope may be, but need not be, contiguous or linear.

In certain embodiments, the allosteric epitope comprises, consists essentially of, or consists of a non-linear epitope located in L2 and/or FnIII-1 domain of IGF-1R as expressed on the surface of a cell or as a soluble fragment, e.g., fused to an IgG Fc region. Thus, in certain embodiments the allosteric epitope comprises, consists essentially of, or consists of at least 3, at least 4, at least 5, at least 6, at least 7, at least 8, at least 9, at least 10, at least 15, at least 20, at least 25, between about 15 to about 30, or at least 10, 15, 20, 25, 30, or more contiguous or non-contiguous amino acids of the sequence spanning amino acid positions 440-586 of IGF-1R, where the non-contiguous amino acids form an epitope through protein folding.

In another embodiment, the allosteric epitope to which the binding moiety binds comprises, consists essentially of, or consists of at least 3, at least 4, at least 5, at least 6, at least 7, at least 8, at least 9, at least 10, at least 15, at least 20, at least 25, between about 15 to about 30, contiguous or non-contiguous amino acids of IGF-1R and at least one of the amino acids of the epitope is selected from the group consisting of amino acid number 437, 438, 459, 460, 461, 462, 464, 466, 467, 469, 470, 471, 472, 474, 476, 477, 478, 479, 480, 482, 483, 488, 490, 492, 493, 495, 496, 509, 513, 514, 515, 533, 544, 545, 546, 547, 548, 551, 564, 565, 568, 570, 571, 572, 573, 577, 578, 579, 582, 584, 585, 586, and 587 of IGF-1R.

In other embodiments, the epitope bound by a binding moiety of the invention comprises at least one amino acid of IGF-1R selected from residues on the surface of the FnIII-1 domain of IGF-1R within a 14 A radius of residues 462-464, for example, residues S437, E438, E469, N470, E471, L472, K474, S476, Y477, 1478, R479, R488, E490, Y492, W493, P495, D496, E509, Q513, N514, V515, K544, S545, Q546, N547, H548, W551, R577, T578, Y579, K582, D584, I585, I586, and Y587. In other embodiments, a binding moiety of the invention binds to at least one amino acid selected from residues within positions 440-586 of IGF-1R which, when mutated, leads to ablation or large decreases in antibody affinity (e.g., >100-fold decrease in affinity), e.g. IGF-1R residues 459, 460, 461, 462, 464, 480, 482, 483, 490, 533, 570, or 571. In yet other embodiments, the epitope may comprise an amino acid of IGF-1R which, when mutated, leads to small decreases in antibody affinity (e.g., 2.5≧KD≧10 nM) compared to wild-type human IGF-1R, e.g. at residues 466, 467, 478, 533, 564, 565, or 568 of IGF-1R. In a particular preferred embodiment, the epitope bound by a binding moiety of the invention comprises any one, any two, or all three of IGF-1R residues 461, 462, and 464.

(ii) Epitopes Resulting in Allosteric Blocking of IGF-1 and not IGF-2

Another exemplary allosteric epitope is located on the surface of the CRR domain of IGF-1R on a face of the receptor rotated slightly away from the IGF-1/IGF-2 binding pocket. The epitope may span large regions of both the CRR and L2 domains. In one embodiment, the allosteric epitope is located within a region that comprises residues 241-379 of IGF-1R. In certain embodiments, the allosteric epitope is located within a region that includes residues 241-266 of the CRR domain IGF-1R or residues 301-308 and 327-379 of the L2 domain of IGF-1R. Exemplary antibodies which allosterically bind to this epitope include the antibodies designated P1E2 and αIR3. Both the P1E2 antibody and the αIR3 antibody have been shown in the Examples to allosterically block binding of IGF-1 (but not IGF-2) to IGF-1R. In one embodiment, a P1E2 antibody is a chimeric antibody that contains the mouse VH and VL derived from the mouse antibody expressed by the P1E2.3B12 mouse hybridoma) and fused to a human IgG4 Palgy/kappa constant domains (e.g., IgG4 constant domains comprising substitutions S228P and T299A (EU numbering convention)). A hybridoma cell line which expresses a full-length mouse antibody P1E2.3B12 was deposited with the ATCC on Jul. 11, 2006 and given the ATCC Deposit Number PTA-7730.

Accordingly, in certain embodiments, a binding moiety employed in the compositions of the invention may bind to the same allosteric epitope as the P1E2 antibody or the αIR3 antibody. For example, a binding specificity may be derived from an antibody which cross-blocks (competes with) the P1E2 antibody or the αIR3 antibody or otherwise interferes with the binding of the P1E2 antibody or the αIR3 antibody. In other embodiments, the binding specificity may comprise either of the P1E2 or αIR3 antibodies themselves, or a fragment, variant, or derivative thereof. In other embodiments, a binding moiety may comprise an antigen binding domain, variable region (VL and/or VH), or CDR therefrom. For example, an allosteric binding moiety may comprise all six CDRs of the P1E2 antibody or the αIR3antibody or it may comprise fewer than all six CDRs (e.g., one, two, three, four, or five CDRs) from the P1E2 antibody or the αIR3 antibody. In one exemplary embodiment, the allosteric binding specificity comprises CDR-H3 from the P1E2 antibody or the αIR3 antibody.

Other antibodies which bind to an allosteric epitope of IGF-1R may be identified using art-recognized methods such as those described above. Additionally, antibodies produced which bind to an allosteric epitope of IGF-1R can then be screened for their ability to allosterically block binding of an insulin growth factor, e.g., IGF-1, IGF-2, or both IGF-1 and IGF-2 to IGF-1R. Antibodies can be screened for these and other properties according to methods described in detail in the Examples.

In yet other embodiments, an allosteric IGF-1R binding moiety specifically or preferentially binds to an allosteric epitope which comprises, consists essentially of, or consists of at least about four to five amino acids of the sequence spanning residues 241-266 of IGF-1R, at least seven, at least nine, or between at least about 15 to about 25 amino acids of the sequence spanning amino acid residues 241-266 of IGF-1R. The amino acids of the epitope may be, but need not be contiguous or linear. In certain embodiments, the allosteric epitope comprises, consists essentially of, or consists of a non-linear epitope present on the extracellular surface of the CRR domain of IGF-1R as expressed on the surface of a cell or as a soluble fragment, e.g., fused to an IgG Fc region. Thus, in certain embodiments the allosteric epitope comprises, consists essentially of, or consists of at least 3, at least 4, at least 5, at least 6, at least 7, at least 8, at least 9, at least 10, at least 15, at least 20, at least 25, between about 15 to about 25, or at least 10, 11, 12, 13, 14, 15, 16, 17, 18, 19, 20, 21, 22, 23, 24, or 25 contiguous or non-contiguous amino acids of the sequence spanning amino acid residues about 241 to about 379 (e.g. residues 241-266 or 301-308 or 327-379) of IGF-1R, where the non-contiguous amino acids form an epitope through protein folding.

In other embodiments, the allosteric epitope to which the binding moiety binds comprises, consists essentially of, or consists of at least 3, at least 4, at least 5, at least 6, at least 7, at least 8, at least 9, at least 10, at least 15, at least 20, at least 25, between about 15 to about 30, contiguous or non-contiguous amino acids wherein at least one of the amino acids of the epitope (preferably all of the amino acids of the epitope) is selected from the group consisting of 241, 248, 250, 251, 254, 257, 263, 265, 266, 301, 303, 308, 327, and 379.

In other embodiments, the epitope recognized by a binding moiety of the invention comprises one or more of amino acids 241-266 of IGF-1R which, when mutated, lead to ablation or large decreases in antibody affinity (e.g., >100-fold decrease in affinity), e.g. at least one or all of IGF-1R residues 248, 254, or 265. In another embodiment, the epitope may comprise at least one amino acid which, when mutated, causes a moderate reduction in binding affinity (e.g. 10≧KD≧100-fold above that of wild-type IGF-1R), for example, IGF-1R residues 254 and/or 257. In yet other embodiments, the epitope may comprise an amino acid of IGF-1R which, when mutated, leads to small decreases in antibody affinity (e.g., 2.5≧KD≧10 nM) compared to wild-type human IGF-1R, e.g. at one or more of IGF-1R residues 248, 263, 301, 303, 308, 327, or 379. In a particular preferred embodiment, the epitope comprises any one, any two, any three, any four, any five, or all six of IGF-1R residues 241, 242, 251, 257, 265, and 266.

C. Other IGF-1R Epitopes

In certain embodiments, an IGF-1R binding moiety may bind to the same epitope as an antibody selected from the group consisting of P2A7.3E11, 20C8.3B8, P1A2.2B11, 20D8.24B11, and P1G10.2B8. In one exemplary embodiment of the invention, an IGF-1R binding moiety of a binding molecule of the invention is derived from a parental murine antibody selected from the group consisting of P2A7.3E11, 20C8.3B8, P1A2.2B11, 20D8.24B11, and P1G10.2B8. Hybridoma cell lines which express antibodies P2A7.3E11, 20C8.3B8, and P1A2.2B11 were deposited with the ATCC on Mar. 28, 2006, Jun. 13, 2006, and Mar. 28, 2006, respectively, and were given the ATCC Deposit Numbers PTA-7458, PTA-7732, and, PTA-7457, respectively. Hybridoma cell lines which express full-length antibodies 20D8.24B11 and P1G10.2B8 were deposited with the ATCC on Mar. 28, 2006, and Jul. 11, 2006, respectively, and were given the ATCC Deposit Numbers PTA-7456 and PTA-7731, respectively.

In yet other embodiments, a binding moiety employed in the compositions of the invention may be derived from an antibody which cross-blocks (competes with) with an antibody selected from the group consisting of any antibody selected from the group consisting of P2A7.3E11, 20C8.3B8, P1A2.2B11, 20D8.24B11, and P1G10.2B8 or otherwise interferes with the binding of selected from the group consisting of P2A7.3E11, 20C8.3B8, P1A2.2B11, 20D8.24B11, and P1G10.2B8. In other embodiments, the binding moiety may comprise an antibody selected from the group consisting of P2A7.3E11, 20C8.3B8, P1A2.2B11, 20D8.24B11, and P1G10.2B8, or a fragment, variant, or derivative thereof. In other embodiments, a binding moiety may comprise an antigen binding domain, variable region (VL and/or VH), or CDR therefrom. For example, a binding moiety may comprise all six CDRs of an antibody selected from the group consisting of P2A7.3E11, 20C8.3B8, P1A2.2B11, 20D8.24B11, and P1G10.2B8 or it may comprise fewer than all six CDRs (e.g., one, two, three, four, or five CDRs) from an antibody selected from the group consisting of P2A7.3E11, 20C8.3B8, P1A2.2B11, 20D8.24B11, and P1G10.2B8. In one exemplary embodiment, the binding specificity comprises CDR-H3 from an antibody selected from the group consisting of P2A7.3E11, 20C8.3B8, P1A2.2B11, 20D8.24B11, and P1G10.2B8.

### VII. Fusion Proteins and Antibody Conjugates

As discussed in more detail elsewhere herein, IGF-1R antibodies, or antigen-binding fragments, variants, or derivatives thereof of the invention may further be recombinantly fused to a heterologous polypeptide at the N- or C-terminus or chemically conjugated (including covalent and non-covalent conjugations) to polypeptides or other compositions. For example, IGF-1R-specific IGF-1R antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

IGF-1R antibodies, or antigen-binding fragments, variants, or derivatives thereof of the invention include derivatives that are modified, i.e., by the covalent attachment of any type of molecule to the antibody such that covalent attachment does not prevent the antibody binding IGF-1R. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

IGF-1R antibodies, or antigen-binding fragments, variants, or derivatives thereof of the invention can be composed of amino acids joined to each other by peptide bonds or modified peptide bonds, i.e., peptide isosteres, and may contain amino acids other than the 20 gene-encoded amino acids. IGF-1R-specific antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the IGF-1R-specific antibody, including the peptide backbone, the amino acid side-chains and the amino or carboxyl termini, or on moieties such as carbohydrates. It will be appreciated that the same type of modification may be present in the same or varying degrees at several sites in a given IGF-1R-specific antibody. Also, a given IGF-1R-specific antibody may contain many types of modifications. IGF-1R-specific antibodies may be branched, for example, as a result of ubiquitination, and they may be cyclic, with or without branching. Cyclic, branched, and branched cyclic IGF-1R-specific antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W.H. Freeman and Company, New York 2nd Ed., (1993); Posttranslational Covalent Modification Of Proteins, B. C. Johnson, Ed., Academic Press, New York, pgs. 1-12 (1983); Seifter et al., Meth. Enzymol. 182:626-646 (1990); Rattan et al., Ann. NY Acad. Sci. 663:48-62 (1992)).

The present invention also provides for fusion proteins comprising an IGF-1R antibody, or antigen-binding fragment, variant, or derivative thereof, and a heterologous polypeptide. The heterologous polypeptide to which the antibody is fused may be useful for function or is useful to target the IGF-1R polypeptide expressing cells. In one embodiment, a fusion protein of the invention comprises, consists essentially of, or consists of, a polypeptide having the amino acid sequence of any one or more of the VH regions of an antibody of the invention or the amino acid sequence of any one or more of the VL regions of an antibody of the invention or fragments or variants thereof, and a heterologous polypeptide sequence. In another embodiment, a fusion protein for use in the diagnostic and treatment methods disclosed herein comprises, consists essentially of, or consists of a polypeptide having the amino acid sequence of any one, two, three of the VH-CDRs of an IGF-1R-specific antibody, or fragments, variants, or derivatives thereof, or the amino acid sequence of any one, two, three of the VL-CDRs of an IGF-1R-specific antibody, or fragments, variants, or derivatives thereof, and a heterologous polypeptide sequence. In one embodiment, the fusion protein comprises a polypeptide having the amino acid sequence of a VH-CDR3 of an IGF-1R-specific antibody of the present invention, or fragment, derivative, or variant thereof, and a heterologous polypeptide sequence, which fusion protein specifically binds to at least one epitope of IGF-1R. In another embodiment, a fusion protein comprises a polypeptide having the amino acid sequence of at least one VH region of an IGF-1R-specific antibody of the invention and the amino acid sequence of at least one VL region of an IGF-1R-specific antibody of the invention or fragments, derivatives or variants thereof, and a heterologous polypeptide sequence. Preferably, the VH and VL regions of the fusion protein correspond to a single source antibody (or scFv or Fab fragment) which specifically binds at least one epitope of IGF-1R. In yet another embodiment, a fusion protein for use in the diagnostic and treatment methods disclosed herein comprises a polypeptide having the amino acid sequence of any one, two, three or more of the VH CDRs of an IGF-1R-specific antibody and the amino acid sequence of any one, two, three or more of the VL CDRs of an IGF-1R-specific antibody, or fragments or variants thereof, and a heterologous polypeptide sequence. Preferably, two, three, four, five, six, or more of the VH-CDR(s) or VL-CDR(s) correspond to single source antibody (or scFv or Fab fragment) of the invention. Nucleic acid molecules encoding these fusion proteins are also encompassed by the invention.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Linsley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

As discussed elsewhere herein, IGF-1R antibodies, or antigen-binding fragments, variants, or derivatives thereof of the invention may be fused to heterologous polypeptides to increase the in vivo half life of the polypeptides or for use in immunoassays using methods known in the art. For example, in one embodiment, PEG can be conjugated to the IGF-1R antibodies of the invention to increase their half-life in vivo. Leong, S. R., et al., Cytokine 16:106 (2001); Chapman et al., “PEGylated antibodies and antibody fragments for improved therapy: a review”, Adv. in Drug Deliv. Rev. 54:531 (June 2002); or Weir et al., Biochem. Soc. Transactions 30:512 (2002).

Moreover, IGF-1R antibodies, or antigen-binding fragments, variants, or derivatives thereof of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). The precise site at which the fusion is made may be selected empirically to optimize the secretion or binding characteristics of the fusion protein. DNA encoding the fusion protein is then transfected into a host cell for expression.

IGF-1R antibodies of the present invention may be used in non-conjugated form or may be conjugated to at least one of a variety of molecules, e.g., to improve the therapeutic properties of the molecule, to facilitate target detection, or for imaging or therapy of the patient. IGF-1R antibodies, or antigen-binding fragments, variants, or derivatives thereof of the invention can be labeled or conjugated either before or after purification, when purification is performed.

In particular, IGF-1R antibodies, or antigen-binding fragments, variants, or derivatives thereof of the invention may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the IGF-1R antibodies, or antigen-binding fragments, variants, or derivatives thereof of the invention are prepared in an analogous manner.

The present invention further encompasses IGF-1R antibodies, or antigen-binding fragments, variants, or derivatives thereof of the invention conjugated to a diagnostic or therapeutic agent. The IGF-1R antibodies can be used diagnostically to, for example, monitor the development or progression of a neurological disease as part of a clinical testing procedure to, e.g., determine the efficacy of a given treatment and/or prevention regimen. Detection can be facilitated by coupling the IGF-1R antibody, or antigen-binding fragment, variant, or derivative thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

An IGF-1R antibody, or antigen-binding fragment, variant, or derivative thereof also can be detectably labeled by coupling it to a chemiluminescent compound. The presence of the chemiluminescent-tagged IGF-1R antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an IGF-1R antibody, or antigen-binding fragment, variant, or derivative thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the IGF-1R antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the IGF-1R antibody, or antigen-binding fragment, variant, or derivative thereof, it is possible to detect the antibody through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

An IGF-1R antibody, or antigen-binding fragment, variant, or derivative thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to an IGF-1R antibody, or antigen-binding fragment, variant, or derivative thereof are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

In particular, binding molecules, e.g., binding polypeptides, e.g., IGF-1R-specific antibodies or immunospecific fragments thereof for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG. In another embodiment, a binding molecule, e.g., a binding polypeptide, e.g., a IGF-1R-specific antibody or immunospecific fragment thereof for use in the diagnostic and treatment methods disclosed herein can be conjugated to a molecule that decreases vascularization of tumors. In other embodiments, the disclosed compositions may comprise binding molecules, e.g., binding polypeptides, e.g., IGF-1R-specific antibodies or immunospecific fragments thereof coupled to drugs or prodrugs. Still other embodiments of the present invention comprise the use of binding molecules, e.g., binding polypeptides, e.g., IGF-1R-specific antibodies or immunospecific fragments thereof conjugated to specific biotoxins or their cytotoxic fragments such as ricin, gelonin, pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in solid tumors as well as lymphomas/leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

With respect to the use of radiolabeled conjugates in conjunction with the present invention, binding molecules, e.g., binding polypeptides, e.g., IGF-1R-specific antibodies or immunospecific fragments thereof may be directly labeled (such as through iodination) or may be labeled indirectly through the use of a chelating agent. As used herein, the phrases “indirect labeling” and “indirect labeling approach” both mean that a chelating agent is covalently attached to a binding molecule and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to a polypeptide (typically via an amino acid residue). More specifically, these linking technologies include random labeling and site-directed labeling. In the latter case, the labeling is directed at specific sites on the polypeptide, such as the N-linked sugar residues present only on the Fc portion of the conjugates. Further, various direct labeling techniques and protocols are compatible with the instant invention. For example, Technetium-99 labeled polypeptides may be prepared by ligand exchange processes, by reducing pertechnate (TcO₄⁻) with stannous ion solution, chelating the reduced technetium onto a Sephadex column and applying the binding polypeptides to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I covalently attached via tyrosine residues. Binding molecules, e.g., binding polypeptides, e.g., IGF-1R-specific antibodies or immunospecific fragments thereof for use in the diagnostic and treatment methods disclosed herein may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation U.S. Pat. Nos. 6,682,134, 6,399,061, and 5,843,439, incorporated herein by reference in their entireties, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, binding molecules may be conjugated to different radiolabels for diagnostic and therapeutic purposes. To this end the aforementioned U.S. Pat. Nos. 6,682,134, 6,399,061, and 5,843,439 disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, et al, J. Nucl. Med. 28: 25-33 (1987) and Carraguillo et al., J. Nuc. Med. 26: 67 (1985).

As indicated above, a variety of radionuclides are applicable to the present invention and those skilled in the can readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target molecule.

Additional preferred agents for conjugation to binding molecules, e.g., binding polypeptides, e.g., IGF-1R-specific antibodies or immunospecific fragments thereof are cytotoxic drugs, particularly those which are used for cancer therapy. As used herein, “a cytotoxin or cytotoxic agent” means any agent that is detrimental to the growth and proliferation of cells and may act to reduce, inhibit or destroy a cell or malignancy. Exemplary cytotoxins include, but are not limited to, radionuclides, biotoxins, enzymatically active toxins, cytostatic or cytotoxic therapeutic agents, prodrugs, immunologically active ligands and biological response modifiers such as cytokines. Any cytotoxin that acts to retard or slow the growth of immunoreactive cells or malignant cells is within the scope of the present invention.

Exemplary cytotoxins include, in general, cytostatic agents, alkylating agents, anti-metabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the maytansinoid family of drugs. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, caminomycin, daunorubicin (daunomycin), doxorubicin, aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. One skilled in the art may make chemical modifications to the desired compound in order to make reactions of that compound more convenient for purposes of preparing conjugates of the invention.

One example of particularly preferred cytotoxins comprise members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the constructs. Such site-directed linking methods have the advantage of reducing the possible effects of linkage on the binding properties of the constructs.

As previously alluded to, compatible cytotoxins for preparation of conjugates may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that binding molecules, e.g., binding polypeptides, e.g., IGF-1R-specific antibodies or immunospecific fragments thereof disclosed herein can also be associated with or conjugated to a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the binding molecules, e.g., binding polypeptides, e.g., IGF-1R-specific antibodies or immunospecific fragments thereof disclosed herein comprise cytokines such as lymphokines and interferons. In view of the instant disclosure it is submitted that one skilled in the art could readily form such constructs using conventional techniques.

Another class of compatible cytotoxins that may be used in association with or conjugated to the disclosed binding molecules, e.g., binding polypeptides, e.g., IGF-1R-specific antibodies or immunospecific fragments thereof, are radiosensitizing drugs that may be effectively directed to tumor or immunoreactive cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked binding molecules of the invention would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

In certain embodiments, a moiety that enhances the stability or efficacy of a binding molecule, e.g., a binding polypeptide, e.g., a IGF-1R-specific antibody or immunospecific fragment thereof can be conjugated. For example, in one embodiment, PEG can be conjugated to the binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

The present invention further encompasses the use of binding molecules, e.g., binding polypeptides, e.g., IGF-1R-specific antibodies or immunospecific fragments conjugated to a diagnostic or therapeutic agent. The binding molecules can be used diagnostically to, for example, monitor the development or progression of a tumor as part of a clinical testing procedure to, e.g., determine the efficacy of a given treatment and/or prevention regimen. Detection can be facilitated by coupling the binding molecule, e.g., binding polypeptide, e.g., IGF-1R-specific antibody or immunospecific fragment thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

A binding molecule, e.g., a binding polypeptide, e.g., a IGF-1R-specific antibody or immunospecific fragment thereof also can be detectably labeled by coupling it to a chemiluminescent compound. The presence of the chemiluminescent-tagged binding molecule is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which a binding molecule, e.g., a binding polypeptide, e.g., a IGF-1R-specific antibody or immunospecific fragment thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the binding molecule will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the binding molecule, e.g., binding polypeptide, e.g., IGF-1R-specific antibody or immunospecific fragment thereof, it is possible to detect cancer antigens through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

A binding molecule, e.g., a binding polypeptide, e.g., a IGF-1R-specific antibody or immunospecific fragment thereof can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to a binding molecule, e.g., a binding polypeptide, e.g., a IGF-1R-specific antibody or immunospecific fragment thereof are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); Order, S. E., “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982).

### VII. Expression of Antibody Polypeptides

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo-dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Following manipulation of the isolated genetic material to provide IGF-1R antibodies, or antigen-binding fragments, variants, or derivatives thereof of the invention, the polynucleotides encoding the IGF-1R antibodies are typically inserted in an expression vector for introduction into host cells that may be used to produce the desired quantity of IGF-1R antibody.

Recombinant expression of an antibody, or fragment, derivative or analog thereof, e.g., a heavy or light chain of an antibody which binds to a target molecule described herein, e.g., IGF-1R, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes (preferably human) synthetic as discussed above. In one embodiment, this is effected using a proprietary expression vector of Biogen IDEC, Inc., referred to as NEOSPLA (disclosed in U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression of antibodies upon incorporation of variable and constant region genes, transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the IGF-1R antibodies, or antigen-binding fragments, variants, or derivatives thereof of the invention may be expressed using polycistronic constructs such as those disclosed in United States Patent Application Publication No. 2003-0157641 A1, filed Nov. 18, 2002 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of IGF-1R antibodies, e.g., binding polypeptides, e.g., IGF-1R-specific antibodies or immunospecific fragments thereof in eukaryotic host cells. Compatible IRES sequences are disclosed in U.S. Pat. No. 6,193,980 which is also incorporated herein. Those skilled in the art will appreciate that such expression systems may be used to effectively produce the full range of IGF-1R antibodies disclosed in the instant application.

More generally, once the vector or DNA sequence encoding a monomeric subunit of the IGF-1R antibody has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990) 8(7):662-667).

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CV1 (monkey kidney line), COS (a derivative of CV1 with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:2026-2034 (1992)), and adenine phosphoribosyltransferase (Lowy et al., Cell 22:817 1980) genes can be employed in tk-, hgprt- or aprt-cells, respectively. Also, anti-metabolite resistance can be used as the basis of selection for the following genes: dhfr, which confers resistance to methotrexate (Wigler et al., Natl. Acad. Sci. USA 77:3567-3570 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Godspiel et al., Clinical Pharmacy 12:488-505 (1993)); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding IGF-1R antibodies, or antigen-binding fragments, variants, or derivatives thereof of the invention can also be expressed non-mammalian cells such as bacteria or insect or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschumper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85(1): 23-33 (1977)). The presence of the trpl lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002 0123057 A1.

### VIII. Treatment Methods Using Therapeutic IGF-1R-Specific Antibodies, or Immunospecific Fragments Thereof

One embodiment of the present invention provides methods for treating a hyperproliferative disease or disorder, e.g., cancer, a malignancy, a tumor, or a metastasis thereof, in an animal suffering from such disease or predisposed to contract such disease, the method comprising, consisting essentially of, or consisting of administering to the animal an effective amount of an antibody or immunospecific fragment thereof, that binds to IGF-1R or a variant of IGF-1R. Suitable antibodies include all antibodies and antigen-specific fragments thereof described herein. Examples include, but are not limited to, an isolated antibody or antigen-binding fragment thereof which specifically binds to the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B1, P1E2.3B12, and P1G10.2B8, an isolated antibody or antigen-binding fragment thereof which specifically binds to IGF-1R, where the antibody or fragment thereof competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B 8 from binding to IGF-1R, or an isolated antibody or antigen-binding fragment thereof which specifically binds to IGF-1R, where the antibody or fragment thereof comprises an antigen binding domain identical to that of a monoclonal Fab antibody fragment selected from the group consisting of M13-C06, M14-G11, M14-C03, M14-B01, M12-E01, and M12-G04, or a monoclonal antibody produced by a hybridoma selected from the group consisting of P2A7.3E11, 20C8.3B8, P1A2.2B11, 20D8.24B11, P1E2.3B12, and P1G10.2B8.

In certain embodiments an antibody of the present invention which specifically binds to IGF-1R or a variant thereof inhibits one or more insulin growth factors, e.g., IGF-1, IGF-2 or both IGF-1 and IGF-1 from binding to IGF-1R. In other embodiments, an antibody of the present invention which specifically binds to IGF-1R or a variant thereof inhibits phosphorylation of IGF-1R upon binding of one or more insulin growth factors. In a further embodiment, an antibody of the present invention which specifically binds to IGF-1R or a variant thereof expressed on a cell, in particular, a tumor cell. inhibits phosphorylation of downstream signal transduction molecules involved in cell proliferation, motility and/or metastasis. Such molecules include, but are not limited to Akt and p42/44 MAPK. In a further embodiment, an antibody of the present invention which specifically binds to IGF-1R or a variant thereof expressed on a cell promotes internalization of surface-expressed IGF-1R, limiting its availability to interact with IGF. In yet a further embodiment, an antibody of the present invention which specifically binds to IGF-1R or a variant thereof expressed on a cell, in particular, a tumor cell, inhibits cell proliferation, motility, and/or metastasis.

An antibody of the present invention which specifically binds to IGF-1R or a variant thereof, to be used in treatment methods disclosed herein can be prepared and used as a therapeutic agent that stops, reduces, prevents, or inhibits cellular activities involved in cellular hyperproliferation, e.g., cellular activities that induce the altered or abnormal pattern of vascularization that is often associated with hyperproliferative diseases or disorders.

Antibodies or immunospecific fragments thereof of the present invention include, but are not limited to monoclonal, chimeric or humanized antibodies, and fragments of antibodies that bind specifically to tumor-associated proteins such as IGF-1R. The antibodies may be monovalent, bivalent, polyvalent, or bifunctional antibodies, and the antibody fragments include Fab F(ab′)₂, and Fv.

Therapeutic antibodies according to the invention can be used in unlabeled or unconjugated form, or can be coupled or linked to cytotoxic moieties such as radiolabels and biochemical cytotoxins to produce agents that exert therapeutic effects.

In certain embodiments, an antibody, or immunospecific fragment thereof of the invention includes an antigen binding domain. An antigen binding domain is formed by antibody variable regions that vary from one antibody to another. Naturally occurring antibodies comprise at least two antigen binding domains, i.e., they are at least bivalent. As used herein, the term “antigen binding domain” includes a site that specifically binds an epitope on an antigen (e.g., a cell surface or soluble antigen). The antigen binding domain of an antibody typically includes at least a portion of an immunoglobulin heavy chain variable region and at least a portion of an immunoglobulin light chain variable region. The binding site formed by these variable regions determines the specificity of the antibody.

The present invention provides methods for treating various hyperproliferative disorders, e.g., by inhibiting tumor growth, in a mammal, comprising, consisting essentially of, or consisting of administering to the mammal an effective amount of a antibody or antigen-binding fragment thereof which specifically or preferentially binds to IGF-1R, e.g., human IGF-1R.

The present invention is more specifically directed to a method of treating a hyperproliferative disease, e.g., inhibiting or preventing tumor formation, tumor growth, tumor invasiveness, and/or metastasis formation, in an animal, e.g., a mammal, e.g., a human, comprising, consisting essentially of, or consisting of administering to an animal in need thereof an effective amount of a an antibody or immunospecific fragment thereof, which specifically or preferentially binds to one or more epitopes of IGF-1R.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, an antibody, or immunospecific fragment thereof, which specifically binds to at least one epitope of IGF-1R, where the epitope comprises, consists essentially of, or consists of at least about four to five amino acids amino acids of SEQ ID NO:2, at least seven, at least nine, or between at least about 15 to about 30 amino acids of SEQ ID NO:2. The amino acids of a given epitope of SEQ ID NO:2 as described may be, but need not be contiguous. In certain embodiments, the at least one epitope of IGF-1R comprises, consists essentially of, or consists of a non-linear epitope formed by the extracellular domain of IGF-1R as expressed on the surface of a cell. Thus, in certain embodiments the at least one epitope of IGF-1R comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, where non-contiguous amino acids form an epitope through protein folding.

In other embodiments, the present invention includes a method for treating a hyperproliferative disease, e.g., inhibiting tumor formation, tumor growth, tumor invasiveness, and/or metastasis formation in an animal, e.g., a human patient, where the method comprises administering to an animal in need of such treatment an effective amount of a composition comprising, consisting essentially of, or consisting of, in addition to a pharmaceutically acceptable carrier, an antibody, or immunospecific fragment thereof, which specifically binds to at least one epitope of IGF-1R, where the epitope comprises, consists essentially of, or consists of, in addition to one, two, three, four, five, six or more contiguous or non-contiguous amino acids of SEQ ID NO:2 as described above, and an additional moiety which modifies the protein, e.g., a carbohydrate moiety may be included such that the binding molecule binds with higher affinity to modified target protein than it does to an unmodified version of the protein. Alternatively, the binding molecule does not bind the unmodified version of the target protein at all.

More specifically, the present invention provides a method of treating cancer in a human, comprising administering to a human in need of treatment a composition comprising an effective amount of an IGF-1R-specific antibody or immunospecific fragment thereof, and a pharmaceutically acceptable carrier. Types of cancer to be treated include, but are not limited to, stomach cancer, renal cancer, brain cancer, bladder cancer, colon cancer, lung cancer, breast cancer, pancreatic cancer, ovarian cancer, and prostate cancer.

In certain embodiments, an antibody or fragment thereof binds specifically to at least one epitope of IGF-1R or fragment or variant described above, i.e., binds to such an epitope more readily than it would bind to an unrelated, or random epitope; binds preferentially to at least one epitope of IGF-1R or fragment or variant described above, i.e., binds to such an epitope more readily than it would bind to a related, similar, homologous, or analogous epitope; competitively inhibits binding of a reference antibody which itself binds specifically or preferentially to a certain epitope of IGF-1R or fragment or variant described above; or binds to at least one epitope of IGF-1R or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M. In certain embodiments, antibodies and fragments thereof of the present invention cross-react with IGF-1R proteins of other species from which they were raised, e.g., an antibody or fragment thereof which specifically binds to human IGF-1R also binds to primate IGF-1R and/or murine IGF-1R. Other suitable antibodies or fragments thereof of the present invention include those that are highly species specific.

In specific embodiments, antibodies or immunospecific fragments thereof disclosed herein bind IGF-1R polypeptides or fragments or variants thereof with an off rate (k(off)) of less than or equal to 5×10⁻² sec⁻¹, 10⁻² sec⁻³, 5×10⁻³ sec¹ or 10⁻³ sec⁻¹. Other antibodies or immunospecific fragments thereof disclosed herein bind IGF-1R polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, antibodies or immunospecific fragments thereof disclosed herein bind IGF-1R polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Other antibodies or immunospecific fragments thereof for use in the diagnostic and treatment methods disclosed herein bind IGF-1R polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

In various embodiments, one or more binding molecules as described above is an antagonist of IGF-1R activity, for example, binding of an antagonist IGF-1R antibody to IGF-1R as expressed on a tumor cell inhibits binding of insulin growth factor, e.g., IGF-1, IGF-2, or both IGF-1 and IGF-2 to IGF-1R, promotes internalization of IGF-1R thereby inhibiting its signal transduction capability, inhibits phosphorylation of IGF-1R, inhibits phosphorylation of molecules downstream in the signal transduction pathway, e.g., Akt or p42/44 MAPK, or inhibits tumor cell proliferation, motility or metastasis.

### IX. Diagnostic or Prognostic Methods Using IGF-1R-Specific Binding Molecules and Nucleic Acid Amplification Assays

IGF-1R-specific antibodies, or fragments, derivatives, or analogs thereof, can be used for diagnostic purposes to detect, diagnose, or monitor diseases, disorders, and/or conditions associated with the aberrant expression and/or activity of IGF-1R. IGF-1R expression is increased in tumor tissue and other neoplastic conditions.

IGF-1R-specific antibodies or fragments thereof, are useful for diagnosis, treatment, prevention and/or prognosis of hyperproliferative disorders in mammals, preferably humans. Such disorders include, but are not limited to, cancer, neoplasms, tumors and/or as described under elsewhere herein, especially IGF-1R-associated cancers such as stomach cancer, renal cancer, brain cancer, bladder cancer, colon cancer, lung cancer, breast cancer, pancreatic cancer, ovarian cancer, and prostate cancer.

For example, as disclosed herein, IGF-1R expression is associated with at least stomach, renal, brain, bladder, colon, lung, breast, pancreatic, ovarian, and prostate tumor tissues. Accordingly, antibodies (and antibody fragments) directed against IGF-1R may be used to detect particular tissues expressing increased levels of IGF-1R. These diagnostic assays may be performed in vivo or in vitro, such as, for example, on blood samples, biopsy tissue or autopsy tissue.

Thus, the invention provides a diagnostic method useful during diagnosis of a cancers and other hyperproliferative disorders, which involves measuring the expression level of IGF-1R protein or transcript in tissue or other cells or body fluid from an individual and comparing the measured expression level with a standard IGF-1R expression levels in normal tissue or body fluid, whereby an increase in the expression level compared to the standard is indicative of a disorder.

One embodiment provides a method of detecting the presence of abnormal hyperproliferative cells, e.g., precancerous or cancerous cells, in a fluid or tissue sample, comprising assaying for the expression of IGF-1R in tissue or body fluid samples of an individual and comparing the presence or level of IGF-1R expression in the sample with the presence or level of IGF-1R expression in a panel of standard tissue or body fluid samples, where detection of IGF-1R expression or an increase in IGF-1R expression over the standards is indicative of aberrant hyperproliferative cell growth.

More specifically, the present invention provides a method of detecting the presence of abnormal hyperproliferative cells in a body fluid or tissue sample, comprising (a) assaying for the expression of IGF-1R in tissue or body fluid samples of an individual using IGF-1R-specific antibodies or immunospecific fragments thereof of the present invention, and (b) comparing the presence or level of IGF-1R expression in the sample with a the presence or level of IGF-1R expression in a panel of standard tissue or body fluid samples, whereby detection of IGF-1R expression or an increase in IGF-1R expression over the standards is indicative of aberrant hyperproliferative cell growth.

With respect to cancer, the presence of a relatively high amount of IGF-1R protein in biopsied tissue from an individual may indicate the presence of a tumor or other malignant growth, may indicate a predisposition for the development of such malignancies or tumors, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

IGF-1R-specific antibodies of the present invention can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin. Suitable assays are described in more detail elsewhere herein.

One aspect of the invention is a method for the in vivo detection or diagnosis of a hyperproliferative disease or disorder associated with aberrant expression of IGF-1R in an animal, preferably a mammal and most preferably a human. In one embodiment, diagnosis comprises: a) administering (for example, parenterally, subcutaneously, or intraperitoneally) to a subject an effective amount of a labeled antibody or fragment thereof of the present invention, which specifically binds to IGF-1R; b) waiting for a time interval following the administering for permitting the labeled binding molecule to preferentially concentrate at sites in the subject where IGF-1R is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of IGF-1R. Background level can be determined by various methods including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of, e.g., ⁹⁹Tc. The labeled binding molecule, e.g., antibody or antibody fragment, will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 7 to 10 days.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the binding molecule is labeled with a radioisotope and is detected in the patient using a radiation responsive surgical instrument (Thurston et al., U.S. Pat. No. 5,441,050). In another embodiment, the binding molecule is labeled with a fluorescent compound and is detected in the patient using a fluorescence responsive scanning instrument. In another embodiment, the binding molecule is labeled with a positron emitting metal and is detected in the patent using positron emission-tomography. In yet another embodiment, the binding molecule is labeled with a paramagnetic label and is detected in a patient using magnetic resonance imaging (MRI).

Antibody labels or markers for in vivo imaging of IGF-1R expression include those detectable by X-radiography, nuclear magnetic resonance imaging (NMR), MRI, CAT-scans or electron spin resonance imaging (ESR). For X-radiography, suitable labels include radioisotopes such as barium or cesium, which emit detectable radiation but are not overtly harmful to the subject. Suitable markers for NMR and ESR. include those with a detectable characteristic spin, such as deuterium, which may be incorporated into the antibody by labeling of nutrients for the relevant hybridoma. Where in vivo imaging is used to detect enhanced levels of IGF-1R expression for diagnosis in humans, it may be preferable to use human antibodies or “humanized” chimeric monoclonal antibodies as described elsewhere herein.

In a related embodiment to those described above, monitoring of an already diagnosed disease or disorder is carried out by repeating any one of the methods for diagnosing the disease or disorder, for example, one month after initial diagnosis, six months after initial diagnosis, one year after initial diagnosis, etc.

Where a diagnosis of a disorder, including diagnosis of a tumor, has already been made according to conventional methods, detection methods as disclosed herein are useful as a prognostic indicator, whereby patients continuing to exhibiting enhanced IGF-1R expression will experience a worse clinical outcome relative to patients whose expression level decreases nearer the standard level.

By “assaying the expression level of the tumor associated IGF-1R polypeptide” is intended qualitatively or quantitatively measuring or estimating the level of IGF-1R polypeptide in a first biological sample either directly (e.g., by determining or estimating absolute protein level) or relatively (e.g., by comparing to the cancer associated polypeptide level in a second biological sample). Preferably, IGF-1R polypeptide expression level in the first biological sample is measured or estimated and compared to a standard IGF-1R polypeptide level, the standard being taken from a second biological sample obtained from an individual not having the disorder or being determined by averaging levels from a population of individuals not having the disorder. As will be appreciated in the art, once the “standard” IGF-1R polypeptide level is known, it can be used repeatedly as a standard for comparison.

By “biological sample” is intended any biological sample obtained from an individual, cell line, tissue culture, or other source of cells potentially expressing IGF-1R. As indicated, biological samples include body fluids (such as sera, plasma, urine, synovial fluid and spinal fluid), and other tissue sources which contain cells potentially expressing IGF-1R. Methods for obtaining tissue biopsies and body fluids from mammals are well known in the art.

In an additional embodiment, antibodies, or immunospecific fragments of antibodies directed to a conformational epitope of IGF-1R may be used to quantitatively or qualitatively detect the presence of IGF-1R gene products or conserved variants or peptide fragments thereof. This can be accomplished, for example, by immunofluoresence techniques employing a fluorescently labeled antibody coupled with light microscopic, flow cytometric, or fluorimetric detection.

Cancers that may be diagnosed, and/or prognosed using the methods described above include but are not limited to, stomach cancer, renal cancer, brain cancer, bladder cancer, colon cancer, lung cancer, breast cancer, pancreatic cancer, ovarian cancer, and prostate cancer.

### X. Immunoassays

IGF-1R-specific antibodies or immunospecific fragments thereof disclosed herein may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4.degree. C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10. 16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32 p or 125 l) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8. 1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

IGF-1R-specific antibodies may, additionally, be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a patient, and applying thereto a labeled IGF-1R-specific antibody or fragment thereof, preferably applied by overlaying the labeled antibody (or fragment) onto a biological sample. Through the use of such a procedure, it is possible to determine not only the presence of IGF-1R protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for IGF-1R gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to IGF-1R or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled IGF-1R-specific antibody. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of IGF-1R-specific antibody may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon resonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE code No. BR-1001-86; BIAtechnology Handbook, version AB (reprinted 1998), BIACORE code No. BR-1001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a monoclonal antibody. Epitope mapping with BIAcore, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIAcore are straightforward to carry out.

For example, one can use a capture molecule to bind the first Mab, followed by addition of antigen and second MAb sequentially. The sensorgrams will reveal: 1. how much of the antigen binds to first Mab, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

### XI. Pharmaceutical Compositions and Administration Methods

Methods of preparing and administering IGF-1R-specific antibodies or immunospecific fragments thereof to a subject in need thereof are well known to or are readily determined by those skilled in the art. The route of administration of the binding molecule, e.g., binding polypeptide, e.g., IGF-1R-specific antibody or immunospecific fragment thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules, e.g., binding polypeptides, e.g., IGF-1R-specific antibodies or immunospecific fragments thereof can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1 M and preferably 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule, e.g., a binding polypeptide, e.g., a IGF-1R-specific antibody or immunospecific fragment thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 (US-2002-0102208 A1), which is incorporated herein by reference in its entirety. Such articles of manufacture will preferably have labels or package inserts indicating that the associated compositions are useful for treating a subject suffering from, or predisposed to autoimmune or neoplastic disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or fragment thereof, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

IGF-1R-specific antibodies or immunospecific fragments thereof disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moiety, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules, e.g., binding polypeptides, e.g., IGF-1R-specific antibodies or immunospecific fragments thereof for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding an IGF-1R-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where IGF-1R-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

IGF-1R antibodies or fragments thereof of the invention can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled binding polypeptides range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-à-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, 47Sc, ¹⁰⁵Rh, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not IGF-1R-specific antibodies or immunospecific fragments thereof disclosed herein are used in a conjugated or unconjugated form, it will be appreciated that a major advantage of the present invention is the ability to use these molecules in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the molecules makes them particularly useful for treating patients that have reduced red marrow reserves and are sensitive to myelotoxicity. In this regard, the unique delivery profile of the molecules make them very effective for the administration of radiolabeled conjugates to myelosuppressed cancer patients. As such, the IGF-1R-specific antibodies or immunospecific fragments thereof disclosed herein are useful in a conjugated or unconjugated form in patients that have previously undergone adjunct therapies such as external beam radiation or chemotherapy. In other preferred embodiments, binding molecules, e.g., binding polypeptides, e.g., IGF-1R-specific antibodies or immunospecific fragments thereof (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies or other binding molecules and one or more chemotherapeutic agents. Particularly preferred embodiments of this aspect of the invention will comprise the administration of a radiolabeled binding polypeptide.

While IGF-1R-specific antibodies or immunospecific fragments thereof may be administered as described immediately above, it must be emphasized that in other embodiments conjugated and unconjugated binding molecules may be administered to otherwise healthy patients as a first line therapeutic agent. In such embodiments binding molecules may be administered to patients having normal or average red marrow reserves and/or to patients that have not, and are not, undergoing adjunct therapies such as external beam radiation or chemotherapy.

However, as discussed above, selected embodiments of the invention comprise the administration of IGF-1R-specific antibodies or immunospecific fragments thereof to myelosuppressed patients or in combination or conjunction with one or more adjunct therapies such as radiotherapy or chemotherapy (i.e. a combined therapeutic regimen). As used herein, the administration of IGF-1R-specific antibodies or immunospecific fragments thereof in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates described herein. Conversely, cytotoxin-conjugated binding molecules could be administered intravenously followed by tumor localized external beam radiation. In yet other embodiments, binding molecules may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of a binding molecule (with or without cytotoxin) and the chemotherapeutic agent may be administered in any order and within any time frame that provides a therapeutic benefit to the patient. That is, the chemotherapeutic agent and IGF-1R-specific antibody or immunospecific fragment thereof, may be administered in any order or concurrently. In selected embodiments IGF-1R-specific antibodies or immunospecific fragments thereof of the present invention will be administered to patients that have previously undergone chemotherapy. In yet other embodiments, IGF-1R-specific antibodies or immunospecific fragments thereof of the present invention will be administered substantially simultaneously or concurrently with the chemotherapeutic treatment. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the binding molecule will be administered within 4, 3, 2 or 1 week of any chemotherapeutic agent or treatment. In yet other embodiments the binding molecule will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppression and one can easily measure the extent to which myelosuppression is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated IGF-1R-specific antibodies or immunospecific fragments thereof of the present invention may be used to effectively treat patients having ANCs lower than about 2000/mm³ or platelet counts lower than about 150,000/mm³. More preferably IGF-1R-specific antibodies or immunospecific fragments thereof of the present invention may be used to treat patients having ANCs of less than about 1500/mm³, less than about 1000/mm³ or even more preferably less than about 500/mm³. Similarly, IGF-1R-specific antibodies or immunospecific fragments thereof of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, IGF-1R-specific antibodies or immunospecific fragments thereof of the present invention may be used to treat disorders in patients exhibiting myelosuppression regardless of the cause.

In this regard it will further be appreciated that IGF-1R-specific antibodies or immunospecific fragments thereof of the present invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other embodiments, radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medicine 1774-1788 (Kurt J. Isselbacher et al., eds., 13^{th} ed. 1994) and V. T. DeVita et al., J. Clin. Oncol., 15: 867-869 (1997) and the references cited therein for standard dosing and scheduling. These therapies can be used unchanged, or altered as needed for a particular patient, in combination with one or more IGF-1R-specific antibodies or immunospecific fragments thereof of the present invention.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade malignancies, who fail to achieve remission or relapse, salvage therapy is used. Salvage therapies employ drugs such as cytosine arabinoside, cisplatin, carboplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the IGF-1R-specific antibodies or immunospecific fragments thereof of the present invention may vary by subject or may be administered according to what is known in the art. See for example, Bruce A Chabner et al., Antineoplastic Agents, in Goodman & Gilman's The Pharmacological Basis of Therapeutics 1233-1287 (Joel G. Hardman et al., eds., 9^{th} ed. (1996)).

In another embodiment, an IGF-1R-specific antibody or immunospecific fragment thereof of the present invention is administered in conjunction with a biologic. Biologics useful in the treatment of cancers are known in the art and a binding molecule of the invention may be administered, for example, in conjunction with such known biologics.

For example, the FDA has approved the following biologics for the treatment of breast cancer: Herceptin® (trastuzumab, Genentech Inc., South San Francisco, Calif.; a humanized monoclonal antibody that has anti-tumor activity in HER2-positive breast cancer); Faslodex® (fulvestrant, AstraZeneca Pharmaceuticals, LP, Wilmington, Del.; an estrogen-receptor antagonist used to treat breast cancer); Arimidex® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); Femara® (letrozole, Novartis Pharmaceuticals, East Hanover, N.J.; a nonsteroidal aromatase inhibitor approved by the FDA to treat breast cancer); and Nolvadex® (tamoxifen, AstraZeneca Pharmaceuticals, LP; a nonsteroidal anti-estrogen approved by the FDA to treat breast cancer). Other biologics with which the binding molecules of the invention may be combined include: Avastin™ (bevacizumab, Genentech Inc.; the first FDA-approved therapy designed to inhibit angiogenesis); and Zevalin® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: Avastin™; Erbitux™ (cetuximab, ImClone Systems Inc., New York, N.Y., and Bristol-Myers Squibb, New York, N.Y.; is a monoclonal antibody directed against the epidermal growth factor receptor (EGFR)); Gleevec® (imatinib mesylate; a protein kinase inhibitor); and Ergamisol® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For use in treatment of Non-Hodgkin's Lymphomas currently approved therapies include: Bexxar® (tositumomab and iodine 1-131 tositumomab, GlaxoSmithKline, Research Triangle Park, N.C.; a multi-step treatment involving a mouse monoclonal antibody (tositumomab) linked to a radioactive molecule (iodine I-131)); Intron® A (interferon alfa-2b, Schering Corporation, Kenilworth, N.J.; a type of interferon approved for the treatment of follicular non-Hodgkin's lymphoma in conjunction with anthracycline-containing combination chemotherapy (e.g., cyclophosphamide, doxorubicin, vincristine, and prednisone [CHOP])); Rituxan® (rituximab, Genentech Inc., South San Francisco, Calif., and Biogen Idec, Cambridge, Mass.; a monoclonal antibody approved for the treatment of non-Hodgkin's lymphoma; Ontak® (denileukin diftitox, Ligand Pharmaceuticals Inc., San Diego, Calif.; a fusion protein consisting of a fragment of diphtheria toxin genetically fused to interleukin-2); and Zevalin® (ibritumomab tiuxetan, Biogen Idec; a radiolabeled monoclonal antibody approved by the FDA for the treatment of B-cell non-Hodgkin's lymphomas).

For treatment of Leukemia, exemplary biologics which may be used in combination with the binding molecules of the invention include Gleevec®; Campath®-1H (alemtuzumab, Berlex Laboratories, Richmond, Calif.; a type of monoclonal antibody used in the treatment of chronic Lymphocytic leukemia). In addition, Genasense (oblimersen, Genta Corporation, Berkley Heights, N.J.; a BCL-2 antisense therapy under development to treat leukemia may be used (e.g., alone or in combination with one or more chemotherapy drugs, such as fludarabine and cyclophosphamide) may be administered with the claimed binding molecules.

For the treatment of lung cancer, exemplary biologics include Tarceva™ (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include Velcade® Velcade (bortezomib, Millennium Pharmaceuticals, Cambridge Mass.; a proteasome inhibitor). Additional biologics include Thalidomid® (thalidomide, Clegene Corporation, Warren, N.J.; an immunomodulatory agent and appears to have multiple actions, including the ability to inhibit the growth and survival of myeloma cells and anti-angiogenesis).

Other exemplary biologics include the MOAB IMC-C225, developed by ImClone Systems, Inc., New York, N.Y.

As previously discussed, IGF-1R-specific antibodies or immunospecific fragments thereof of the present invention, or recombinants thereof may be administered in a pharmaceutically effective amount for the in vivo treatment of mammalian hyperproliferative disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of IGF-1R-specific antibodies or immunospecific fragments thereof of the present invention, or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the binding molecule will be preferably be capable of interacting with selected immunoreactive antigens on neoplastic or immunoreactive cells, or on non neoplastic cells, e.g., vascular cells associated with neoplastic cells. and provide for an increase in the death of those cells. Of course, the pharmaceutical compositions of the present invention may be administered in single or multiple doses to provide for a pharmaceutically effective amount of the binding molecule.

In keeping with the scope of the present disclosure, IGF-1R-specific antibodies or immunospecific fragments thereof of the present invention may be administered to a human or other animal in accordance with the aforementioned methods of treatment in an amount sufficient to produce a therapeutic or prophylactic effect. The IGF-1R-specific antibodies or immunospecific fragments thereof of the present invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Maniatis et al., ed., Cold Springs Harbor Laboratory, New York (1982), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C.A.K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, M A (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical—Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein, are incorporated herein by reference in their entireties.

### EXAMPLES

### Example 1

Selection of IGF-1R Specific Fabs from Phage Libraries

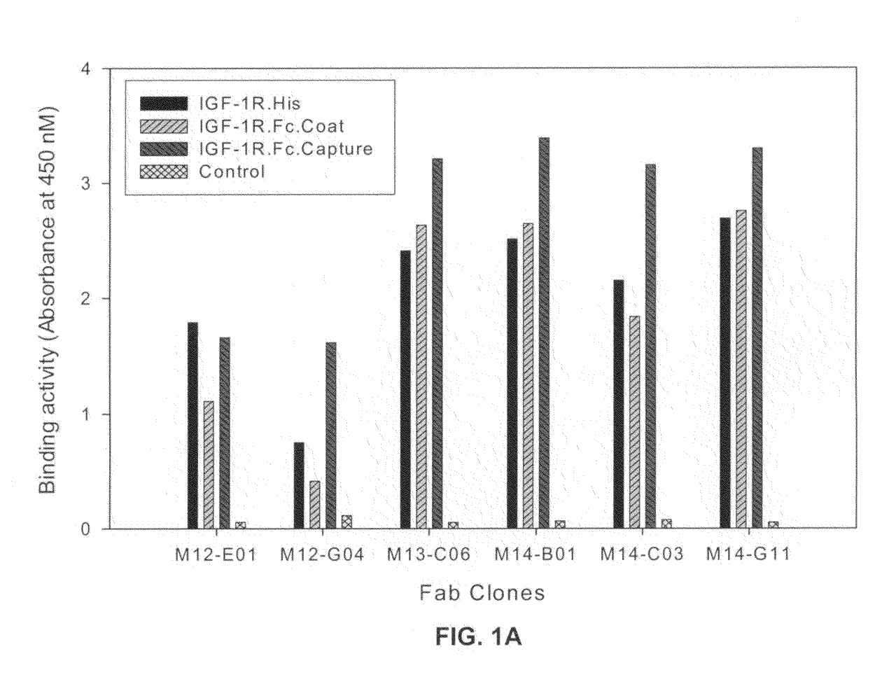

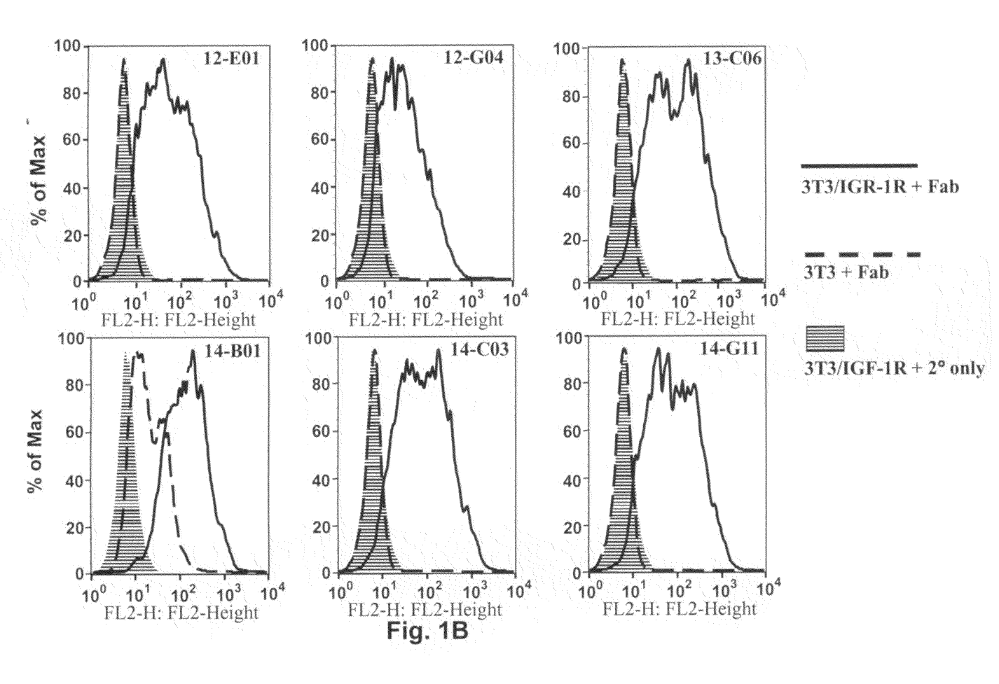

Recombinant human IGF-1R ectodomain was used to screen a human naïve phagemid Fab library containing 3.5×10¹⁰ unique clones (Hoet, R. M., et al. Nat. Biotechnol. 23(3):344-8 (2005), (“Hoet et al.”) which is incorporated herein by reference in its entirety). Two distinct panning arms were followed using biotinylated IGF1R-his and IGF1R-Fc protein. Proteins were captured on steptavidin-coated magnetic beads prior to incubation with the phage library. In the case of IGF1R-Fc, a biotinylated anti-Fc antibody was captured on the magnetic beads, followed by captured of the Fc fusion protein. Selections were performed as described in Hoet et al. After 3 rounds of panning, the 479 bp gene III stump was removed by MluI digestion, and the vector was religated for soluble Fab expression in TG1 cells. ELISA analysis of 920 clones from the biotinylated IGF1R-his arm yielded 593 positive clones, containing 33 unique sequences. ELISA analysis of 920 clones from the IGF1R-Fc arm yielded 163 positive clones, containing 12 unique sequences. Sequence analysis of all clones determined 12 clones were isolated in both arms of the panning strategy. Unique clones were purified and binding was reconfirmed to recombinant human IGF-1R ectodomain by ELISA as well as 3T3 cells stably transfected with full-length human IGF-1R (FIGS. 1A & 1B). Based on binding data, 6 of the 12 unique clones isolated in both arms were selected for further analysis.

### Example 2

### Binding Activity of Fabs to IGF-1R Expressed on Tumor Cells

The ability of Fabs to bind to the wild type IGF-1R was determined by flow cytometry using MCF-7 tumor cell line.

MCF-7 cells (Human Breast Adenocarcinoma from NCI) were split 24 hours prior to the setup of the assay to obtain 70% confluent monolayer. Routinely, MCF-7 cell line was maintained within 20 passages. Cells were lifted with cell dissociation buffer (Gibco catalog #13151-014), counted, washed and adjusted to 1×10⁶ cells/ml and one ml of cells were then added to each tube (12×75 mm tube Falcon catalog# 352054). Cells were pelleted and supernatant removed by centrifugation at 1200 rpm for 5 min and 100 μl of diluted antibodies were then added to the cell pellet. Purified Fabs were tested at a starting concentration of either 210 or 60 μg/ml with 1:3 dilutions in FACS buffer, down to 0.001 μg/ml. FACS buffer used throughout the assay was PBS (without Ca++/Mg++) containing 1% BSA (Sigma catalog# A-7906; Sigma-Aldrich Corp. (St. Louis, Mo., USA)) and 0.1% Sodium Azide (Sigma catalog #S2002). As a positive control IR3 a murine antibody (Ab-1; Calbiochem #GR11L) was used. Samples were allowed to incubate on ice for 1 hour and 15 minutes then were washed with 2 ml FACS buffer and centrifuged at 1200 rpm for 5 minutes at 4° C. The supernatant was aspirated and 100 μl of the secondary detection antibody was added to each corresponding tube in FACS buffer. Samples were then incubated for 30 minutes on ice, in the dark. Cells were washed as described above, then, re-suspended in 250 μl FACS buffer per tube/sample.

Cell bound Fabs were detected using FITC-conjugated affinity-purified F(ab′)₂ Fragment specific goat anti-human-IgG (Jackson ImmunoResearch Lab catalog #109-096-006; use at 5 μg/ml), while positive murine control antibody was detected using the F(ab′)₂ FITC conjugated goat anti-mouse IgG (H+L) (Jackson ImmunoResearch, catalog# 115-096-062; used at 5 μg/ml). Cells were stained for live cell determination with Propidium Iodide staining solution (PI for dead cell exclusion; BD Pharmingen catalog# 51-66211E or 556463; use at 1:500 final in FACS buffer). Samples were run on the FACSCalibur instrument (Becton Dickinson) with 10,000 live events collected per sample. Data analysis was done using GraphPad Prism version 4.0 software (www.graphpad.com) (GraphPad Software, Inc., 11452 E1 Camino Real, #215, San Diego, Calif. 92130 USA).

Once samples have been run and geometric means determined, antibody concentration (X axis) vs. geometric mean (Y axis) was graphed to the log10, using Graphpad Prism (Prism Graph) graphing program. Data sets were then transformed (X value data set=antibody concentration) to X=Log(X) and graphed using a nonlinear regression curve fit, Sigmoidal dose-response. EC₅₀ values and R² values were generated using the Prism Graph software.

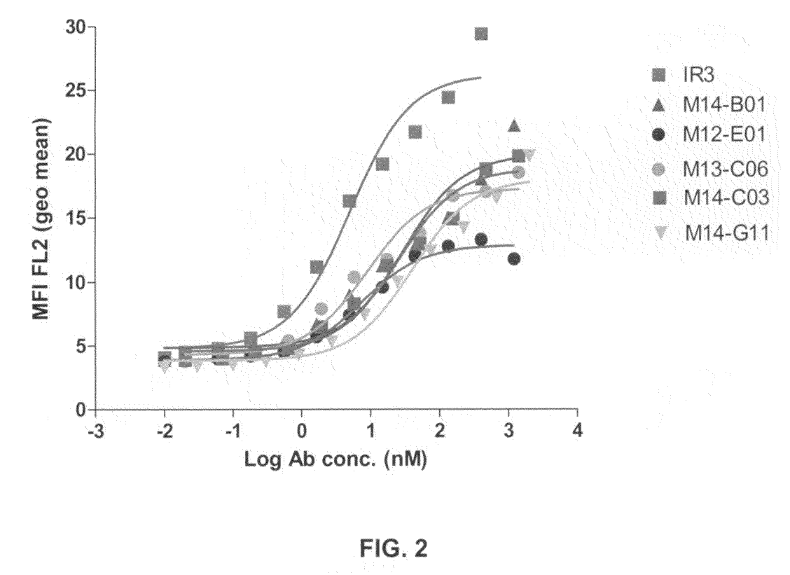

All 6 Fabs showed good binding activity to wild type IGF-1R expressed on MCF-7 tumor cells (FIG. 2). The EC₅₀ of binding ranged between 9 to 42 nM (Table 3).

### Example 3

### Inhibition of Ligand Binding to IGF-1R by Fabs

The ability of Fabs to block the binding of IGF-1 and IGF-2 ligands to IGF-1R was determined using a radioimmunoassay (RIA).

Ligand blocking assay (RIA). Recombinant human IGF-1 (Cat #291-G1), IGF-2 (Cat #292-G2), insulin (Cat # Custom02) human Insulin Receptor (Cat #1544-1R) were purchased from R&D Systems, Inc., Minneapolis, Minn. Insulin (Arg-Insulin, Cat #01-207) was purchased from Upstate Cell Signaling Solutions (Lake Placid, N.Y. (now part of Millipore, Concord, Mass. (USA)). ¹²⁵I-rhIGF-1 (Cat # 1 M1±72), ¹²⁵I-rhIGF-2 (Cat# IM238) and ¹²⁵I-rhInsulin (Cat# IM166) were purchased from Amersham Biosciences (Piscataway, N.J.). AffiPure goat anti-human IgG, Fcγ fragment specific antibodies (Cat #109-005-098, Jackson ImmunoResearch, West Grove, Pa.) was used for IGF-1R-Fc capture. As detection antibody, goat anti-mouse IgG HRP (Cat #1030-05, Southern Biotech Birmingham, Ala.) was used.

As positive controls for IGF-1 and IGF-2 blocking, IR3 (Ab-1, Cat. #GR11LSP5, Calbiochem, La Jolla, Calif.) and 1H7 (Mouse Monoclonal specific to IGF-1R α-chain, sc-461, IgG₁ Santa Cruz Biotechnology, Santa Cruz, Calif.) were used respectively. Human insulin receptor α-subunit specific antibodies, Clone 83-14, (Cat #AHR0221, Biosource International, Inc., Camarillo, Calif.) and the 47-9 (Cat #E55502 M, Biodesign International, Saco, Me.) were used as positive controls blocking of insulin-insulin receptor binding experiments. Recombinant IGF-1R-Fc fusion protein was produced at Biogen Idec (Cambridge, Mass.).

As isotype matched mouse negative control antibodies, 2B8 (murine α-CD20.IgG₁) and 2B8 mkm.G₂ₐ (murine α-CD20 MAb, IgG₂ₐ, Biogen Idec, Lot #NB3304-87, San Diego, Calif.) were used. The negative control for Fabs was R001-1B provided by Christilyn Graff (Biogen Idec, Cambridge, Mass.). PBS used in buffers was from BioWhittaker (Cat. # 17-513F, Walkersville, Md.).

Recombinant human IGF-1R (Histidine tagged version) or IGF-1R-Fc was coated onto IMMULON2 HB (high binding) Removawell strips (Dynex Technologies, Inc., cat. #6302) diluted with carbonate coating buffer pH 9.5 to a concentration of 250 ng/well. After overnight incubation at 4° C., the wells were washed three times with washing buffer (0.05% Tween 20/PBS) then blocked with blocking buffer (3% BSA/PBS) for one hour at room temperature. The blocking buffer was removed and the wells washed three more times. Antibody, Fab, or ligand preparations were diluted to desired concentration with dilution buffer (1% BSA/0.05% Tween 20/PBS) and plated at 50 μl per well in duplicate. After 45 minutes at room temperature, 100,000 cpm of either [125I] rhIGF-1 or [125I] rhIGF-2 in 50 μl dilution buffer was added per well. This was incubated at room temperature for one more hour. The wells were washed again three more times and left liquid free after the last wash. The air-dried wells were counted with the Isodata Gamma Counter.

Alternatively, Fabs were evaluated by a modified capture assay, where the IGF-1R-Fc was captured using anti-human IgG immobilized to a plate. Immobilization was carried out by overnight incubation of goat anti-human IgG, Fcγ fragment specific antibody (200 ng/well) in carbonate coating buffer. The wells were washed, blocked and 250 ng of IGF-1R-Fc was added per well.

The ability of 6 different Fabs to block the binding of IGF-1 or IGF-2, or both ligands is shown in Table 3. The top 6 Fabs with different blocking activity were selected for further analysis.

### Example 4

### Fabs Inhibited IGF-1 and IGF-2 Mediated IGF-1R Phosphorylation

Cell lines: IGF1R expressing human breast carcinoma cell line MCF-7 (NCI) were maintained at 37° C. and 5% CO₂ in MEM eagle (ATCC) containing 10% FBS, 1× non-essential amino acids, 2 mM L-glutamine, 1 mM sodium pyruvate and 1000 U/ml penicillin and streptomycin. Cells were sub-cultured twice weekly for maintenance and assay, and used with a maximum of 12 passages.

MCF-7 cells were plated in 2 ml growth media at 2×10⁵ to 4.0×10⁵ cells/well in Ploy-D-Lysine coated 12 well plates (BD Biosciences, #35-6470) and cultured at 37° C., 5% CO₂. At 48 hours, media removed and cells serum starved overnight at 37° C., 5% CO₂. Serum free media was removed and control or test antibodies at indicated concentration were added in 350 ul of fresh serum free media and incubated for 1 hour at room temperature, or alternately at 37° C. Fabs were tested at 200 nM, 20 nM and 2 nM concentration and the mAbs were tested at 67, 6.7 and 0.67 nM. The commercial anti-IGF-1R control antibody used was αIR3 (EMD biosciences, Oncogene Research products, #D27249). Human recombinant IGF-1 at 13 nM or IGF-2 at 27 nM (R & D Systems, #291-G1, #292-G2) added to wells in 35 ul serum free media and incubated at 37° C. for 15 minutes. Ligand was incubated at room temperature for 37° C. antibody experiments. Cells were lysed in 1× cell lysis buffer (Cell Signal technologies, #9803) with 1 mM PMSF for 1 hour at room temperature.

Cell lysates were added to ELISA plates pre-coated with IGF-1R, antibody (Clone 1-2, Biosource International, #AHR0361) and incubated for 2 hours. Following which plates were washed and the plate bound phosphorylated receptor was detected with the biotin labeled anti-phosphotyrosine antibody 4G10 (Catalog #16-103, Upstate Cell Signaling Solutions (Lake Placid, N.Y. (now part of Millipore, Concord, Mass. (USA)) and streptavidin-HRP (BD Pharmingen, #554066). Assay is developed by addition of TMB substrate (Kierkegaard & Perry, #50-76-00) and color stopped by addition of 4N H₂S₄O₄₋₄ (LabChem, Cat#LC25830-1). Optical density is measured at 450 nm using a Molecular Devices plate reader and percent inhibition over the ligand control is calculated for each antibody-ligand sample.

Table 3 summarizes the inhibition of IGF-1 and IGF-2 mediated phosphorylation of IGF-1R in MCF-7 cells by Fabs. A total of 16 IGF-1R Fabs were screened for inhibition of receptor phosphorylation by ELISA. Nine antibodies showed positive response of “+” or better at a concentration of 200 nM against IGF-1, IGF-2 or both. These antibodies were selected for scale up quantities and tested again for dose dependent inhibitory response. Based on the ability to inhibit ligand binding and receptor phosphorylation, four Fabs were selected as lead candidates for full-length antibody conversion (see, Example 6).

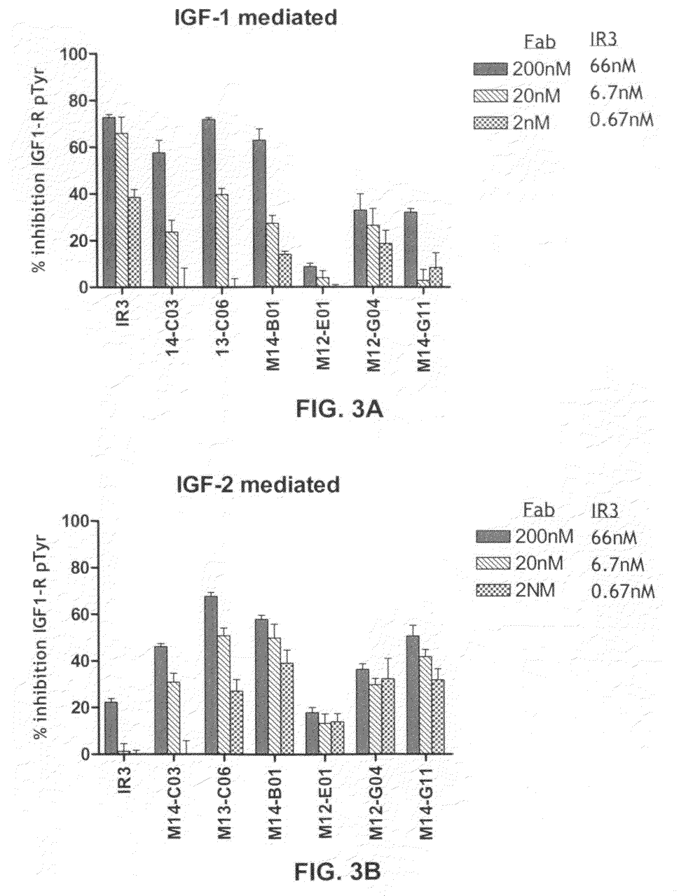

FIG. 3 (A & B), shows the Inhibition of IGF-1R phosphorylation of the scaled up material of the top 6 IGF-1R Fabs.

### Example 5

### Antibody Binding Specificities and Affinities for IGF-1R versus INSR

Part I: Analysis of Antibody Binding to Soluble IGF-1R Versus Soluble INSR Using Enzyme-Linked Immunosorbent Assays (ELISA)

ELISA assays were performed to determine specific binding of the Fab fragment antibodies to soluble IGF-1R over the insulin receptor. Plates were coated with 10 ug/ml of rh-IGF-1R (R & D Systems, #305-GR) or rh-INSR(R & D Systems, #1544-IR) overnight and blocked with 5% milk. The antibodies were added at a range of 2 μM-0.2 nM for Fabs or 667-0.067 nM for murine MAbs in a 1:10 serial dilution and incubated 1 hour at room temperature. Bound antibody was detected with HRPO labeled goat α-human kappa (Southern Biotechnology Associates, #2060-05) for Fabs and goat α-mouse IgG Fcγ (Jackson Immunoresearch, # 115-035-164) for murine MAbs. Color development was stopped by addition of 4NH₂SO₄ and optical density is measured at 450 nm using a Molecular Devices plate reader and binding curves are generated.

IGF-1R Fabs showed no specific binding to soluble insulin receptor at any concentration (Table 3) while, as expected they showed good binding to IGF-1R-Fc.

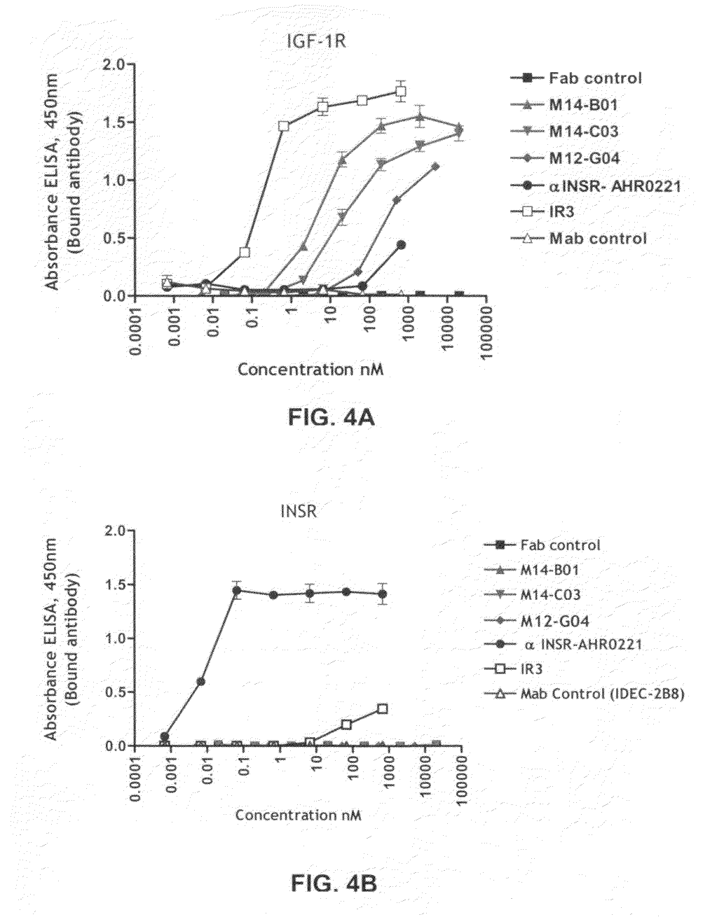

FIG. 4 (A & B) illustrates the representative binding curves obtained with Fabs M14-B01, M14-C03 and M12-G04. Similar binding patterns were observed for M13-C06, M14-G1 and M12-E01 (data not shown).

Part H: Analysis of Antibody Binding to Soluble IGF-1R Versus Soluble INSR Using Surface Plasmon Resonance (SPR) and Time-Resolved Fluorescence Resonance Energy Transfer (tr-FRET)

Binding affinities of M13-C06, M14-C03, and M14-G11 antibodies to soluble human IGF-1R and insulin receptor ectodomains were compared using surface plasmon resonance (Biacore) and time-resolved fluorescence resonance energy transfer (tr-FRET); further demonstrating that M13-C06 antibody does not exhibit significant cross-reactivity with insulin receptor, murine IGF-1R, or a truncated version of human IGF-1R (i.e., hIGF-1R amino acid residues 1-462 containing only the first and second leucine rich repeat domains as well as the cysteine rich repeat domain, but lacking IGF-1R's three fibronectin type III domains).

Surface Plasmon Resonance (SPR) Analyses

SPR analyses were performed using a Biacore3000. The instrument was set to 25° C. and assays performed with running buffer HBS-EP pH 7.2 purchased from Biacore (Biacore, Cat. No. BR-1001-88). The fully human antibodies, M13-C06, M14-C03, and M14-G11 were immobilized to ˜10,000 RU on Biacore CM5 Research Grade Sensor Chip surfaces using the standard NHS/EDC-amine reactive chemistry according to protocols supplied by Biacore. For immobilization, the antibodies were diluted to 40 μg/mL in a 10 mM Acetate pH 4.0 buffer. To investigate the relative kinetics of association and dissociation of the full-length ectodomains of human IGF-1R(1-902)-His₁₀ (hIGF-1R-His₁₀ (R&D systems)) and human INSR(28-956)-His₁₀ (INSR(R&D systems)) to each of the human antibodies, increasing concentrations of hIGF-1R-His₁₀ or INSR were injected over the sensorchip surfaces. The hIGF-1R-His₁₀ concentration series ranged from 1.0 nM to 250 nM while the INSR concentrations ranged from 1.0 nM to 2 μM. All antibody surfaces were reliably regenerated with 100 mM Glycine, pH 2.0. Repeated regenerations did not lead to activity losses for any of the antibody surfaces. Flow rates were 20 μl/min. (“His₁₀” denotes a 10-residue histidine tag on the C-terminus of the constructs.)

Time-Resolved Fluorescence Resonance Energy Transfer (tr-FRET) Assay

hIGF-1R-His₁₀ and M13-C06 were covalently conjugated to Cy5 and a Europium chelate, respectively, using standard NHS chemistry according to the dye manufacturer's protocols. Serial dilutions of several unlabeled soluble ectodomain receptor competitors, (1) hIGF-1R-His₁₀, (2) human IGF-1R(1-903)-FlagHis₁₀ (hIGF-1R-FlagHis₁₀, Biogen Idec), (3) human IGF-1R(1-903)-Fc (hIGF-1R-Fc, Biogen Idec), (4) human IGF-1R(1462)-Fc (hIGF-1R(1-462)-Fc, Biogen Idec), (5) murine IGF-1R(1-903)-Fc (mIGF-1R-Fc, Biogen Idec) or (6) INSR, starting at 6.25 μg (50 μl of 125 μg/ml stock solution) were mixed with 0.1 μg hIGF1R-His₁₀-Cy5 (25 μl of 4 μg/ml stock solution) and 0.075 μg Eu—C06 (25 μl of 3 g/ml stock solution) in 96-well microtiter plates (black from Costar). The conjugation levels for hIGF-1R-His₁₀-Cy5 were 6.8:1 (Cy5:IGF-1R-His₁₀), and for Eu—C06 were 10.3:1 (Eu:C06) as determined by the absorbance of each dye with respect to the protein concentration. The total volume was 100 μl for each sample. Plates were incubated for 1 hr at room temperature on a plate agitator. Fluorescence measurements were carried out on a Wallac Victor² fluorescent plate reader (Perkin Elmer) using the LANCE protocol with the excitation wavelength at 340 nm and emission wavelength at 665 nm. All constructs were sampled with at least two replicates.

All Biogen Idec derived soluble IGF-1R receptor ectodomain constructs were subcloned into Biogen Idec PV-90 vectors for CHO expression using described methodology (Brezinsky et al., 2003). Each receptor containing a C-terminal IgG-Fc tag was affinity purified using a single protein A SEPHAROSE-FF™ (GE Healthcare) step as described previously. hIGF-1R-FlagHis₁₀ was purified using Ni²⁺-agarose (Qiagen) as described previously (Demarest et al., 2006).

Results: The fully human anti-IGF-1R antibodies, M13-C06, M14-C03, and M14-G11, were evaluated for their comparative binding activities towards soluble IGF-1R and INSR ectodomain constructs using surface plasmon resonance (SPR). hIGF-1R-His₁₀ and INSR were injected over immobilized antibody surfaces using identical protocols. hIGF-1R-His₁₀ demonstrated binding to all three anti-IGF-1R antibodies even at the lowest concentration, 0.5 nM (data not shown: concentrations ranged from 1 to 250 nM and the receptor injection phase was 400-2200 seconds followed by a buffer dissociation phase and subsequent regeneration with glycine, pH 2.0). hIGF-1R-His₁₀ binding was strongest for the M13-C06 surface. In contrast, INSR demonstrated little activity towards the M13-C06 surface even at a concentration as high as 2 μM receptor (>1000 higher than what was observed for IGF-1R binding (data not shown: concentrations ranged from 1.0 nM to 2 μM and the receptor injection phase was 500-1000 seconds followed by a buffer dissociation phase). The M14-C03 and M14-G11 surfaces also demonstrated little binding activity towards INSR.

Next, the affinities of various recombinant IGF-1R and INSR constructs for M13-C06 were determined using a competition-based tr-FRET assay. Best fit binding curves for all recombinant receptor constructs (described below) were determined (data not shown). All data were fitted to a one-site binding model from which the corresponding IC₅₀ values were determined. The three full-length human IGF-1R ectodomain constructs (hIGF-1R-Fc, hIGF-1R-His₁₀, and hIGF-1R-FlagHis₁₀) all competed in a concentration dependent manner with IC₅₀ values of 2.9, 2.0, 5.2 μg/ml, respectively. The truncated human IGF-1R(1-462)-Fc construct, the full-length mouse IGF-1R-Fc construct, and the full-length human INSR-His₁₀ construct did not inhibit Cy5-labeled hIGF-1R-His₁₀ at concentrations 100-fold above the IC₅₀ of the recombinant full-length human IGF-1R constructs, suggesting these former constructs do not exhibit significant binding reactivity for M13-C06 compared to the latter full-length human IGF-1R.

Part III: Relative Binding Affinity of M13-C06 Antibody for Soluble Human Versus Murine IGF-1R.

The relative binding affinity of M13-C06 for murine versus human IGF-1R were compared. Surface plasmon resonance (SPR) was used to determine the affinity of M13-C06 for murine IGF-1R Fc and human IGF-1R Fc. Experiments were performed on a Biacore 3000 set to 25° C. using HBS-EP (Biacore, Cat. No. BR-1001-88) as the running buffer. An anti-human IgG-Fc antibody (2C11 from Biogenesis, Cat. No. 5218-9850) was immobilized to saturation on a Biacore CM5 chip (Cat. No. BR-1000-14) surface by injection at 500 nM in HBS-EP buffer. mIGF-1R-Fc or hIGF-1R-Fc was captured on the chip surface by injecting 40 μL of 20 nM receptor at 3 μL/min. Following capture of receptor, 40 μL of M13-C06 Fab was injected at 3 μL/min. Dissociation of Fab was measured for ˜27 minutes. Fab was serially diluted from 25 to 0.4 nM to obtain concentration dependent kinetic binding curves. Regeneration of the surface chip between each injection series was performed using 3×10 μL injections of 100 mM glycine pH 2.0 at 60 μL/min. Each curve was double referenced using (1) data obtained from a CM5 chip surface devoid of the anti-IgG antibody 2C11 and (2) data from a primary injection of receptor followed by a secondary injection of HBS-EP buffer. The concentration series of M13-C06 Fab for each receptor was fit to the 1:1 binding model provided within the BiaEvaluation software of the manufacturer. To obtain the k_{d} of M13-C06 binding to mIGF-1R-Fc, the experiment was repeated with M13-C06 Fab at 25 nM and mIGF-1R-Fc at 20 nM with the only change in the original protocol being an extension of the dissociation period to three hours.

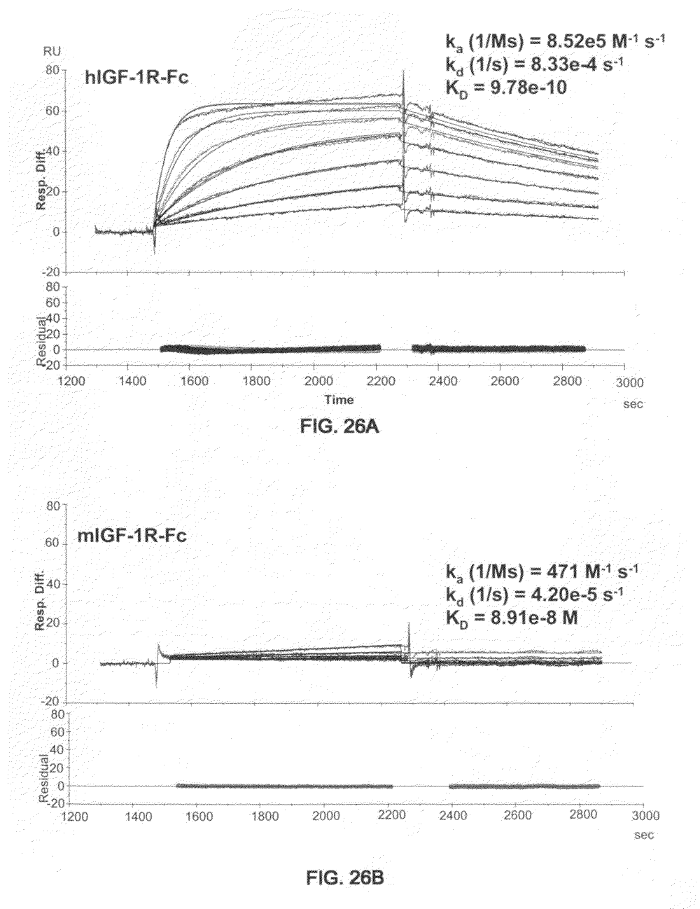

Results: M13-C06 Fab was applied to Biacore surfaces containing hIGF-1R-Fc or mIGF-1R-Fc to determine the relative affinity of the antibody to the two species of receptor. The presence of the C-terminal IgG1-Fc tag results in additional multimerization of the IGF-1R-Fc receptor constructs (data not shown); therefore, the binding model fits provide a measure of the relative or apparent affinities of M13-C06 for each receptor. The affinity of M13-C06 Fab for human and murine IGF-1R Fc was found to be 0.978 nM and 89.1 nM, respectively. The 100-fold decrease in binding to murine IGF-1R is readily apparent when comparing FIG. 26 A & B, which display the association and dissociation curves, kinetic rate constants, and equilibrium dissociation constants. FIG. 26A shows the concentration dependent binding characteristics of M13-C06 Fab for human IGF-1R (kₐ (1/Ms)=8.52e5 M⁻¹ s⁻¹; k_{d} (1/s)=8.33e4 s⁻¹; and, K_{D}=9.78e-10 M). FIG. 26B shows the slow association and dissociation binding characteristics of M13-C06 for mIGF-1R-Fc (kₐ (1/Ms)=471 M⁻¹ s⁻¹; k_{d}(1/s)=4.20e-5 s⁻; K_{D}=8.91e-8 M). Due to the extremely slow dissociation of M13-C06 Fab from mIGF-1R-Fc, the kinetic dissociation rate constant, k_{d}, could not be determined using the initial data set. A second experiment was performed using a 3 hr dissociation period to obtain the dissociation rate constant, k_{d} of 4.20e-5 s⁻¹, which was used to obtain the equilibrium dissociation constant, K_{D}, (described above) from the original dataset. The presence of the C-terminal IgG1-Fc tag results in additional multimerization of the IGF-1R-Fc receptor constructs (data not shown); therefore, the binding model fits provide a measure of the relative or apparent affinities of M13-C06 for each receptor.

Part IV: M13-C06 Full-Length Antibody Specifically Binds IGF-1R but not INSR Expressed in Mammalian Cells.

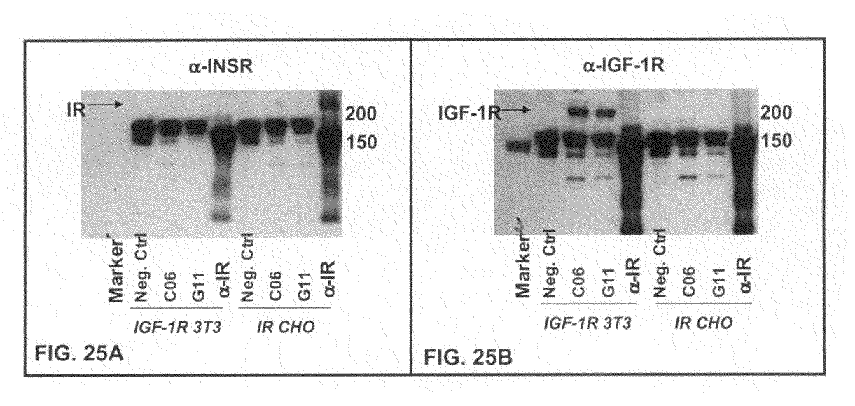

Recombinant IGF-1R and insulin receptor (IR) were independently expressed in mammalian cells (3T3 or CHO). Cells were solubilized with 1% Triton X-100 and the receptor was immunoprecipitated with protein-A/G beads coupled to a negative control antibody (IDEC-151), M13.C06.G4.P.agly antibody (C06), M14-G11.G4.P.agly antibody (G11), or an INSR antibody (α-IR). Antibody/antigen complexes were released from the beads by acid treatment, applied to Tris-Glycine SDS-PAGE gels and blotted to nitrocellulose membranes. Detection was performed using mouse anti-human IR (FIG. 25A) or mouse anti-human IGF-1R (FIG. 25B) and goat α-mouse IgG. Results: M13.C06.G4.P.agly antibody binds to IGF-1R but not to INSR expressed in mammalian cells.

### Example 6

### Construction of Full-Length Anti-IGF-1R IgGs

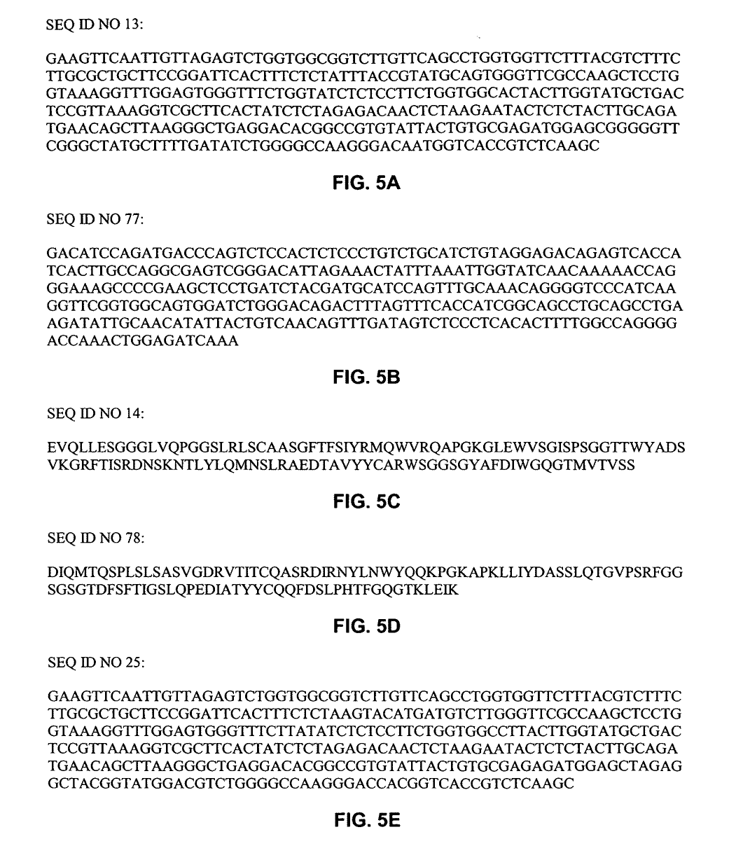

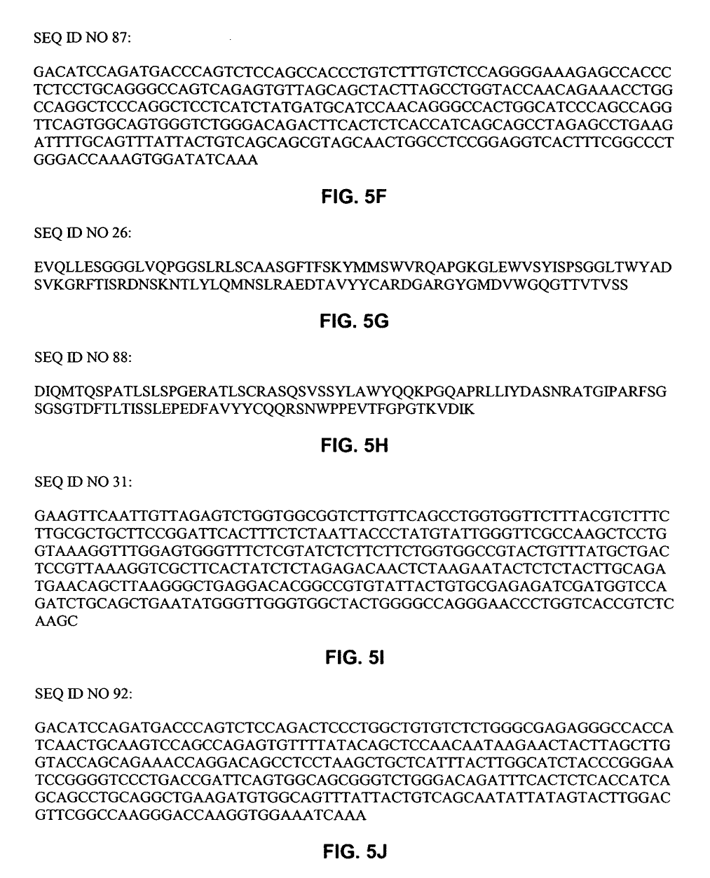

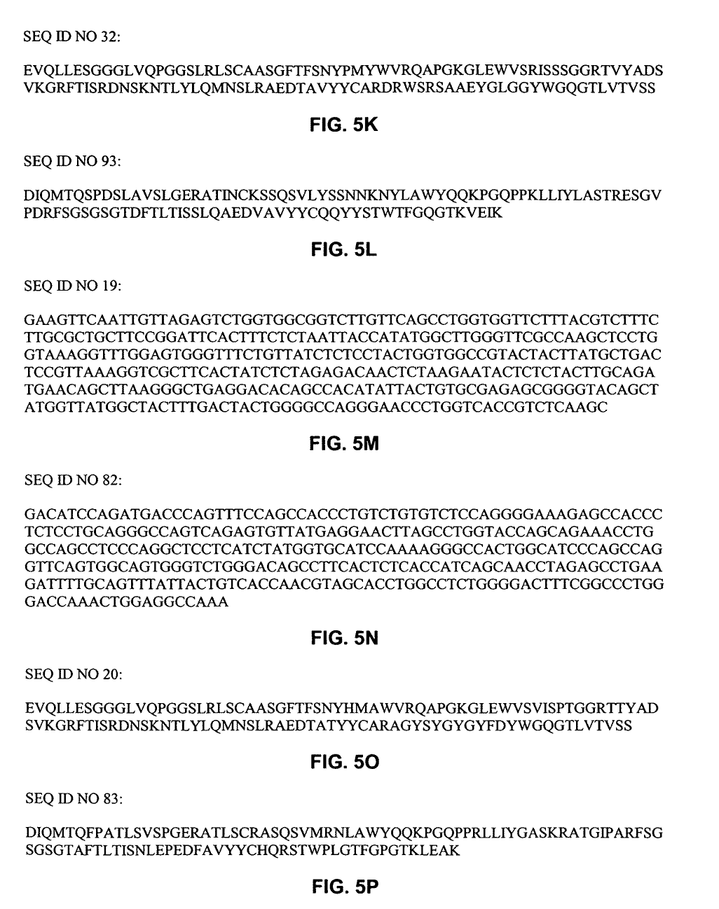

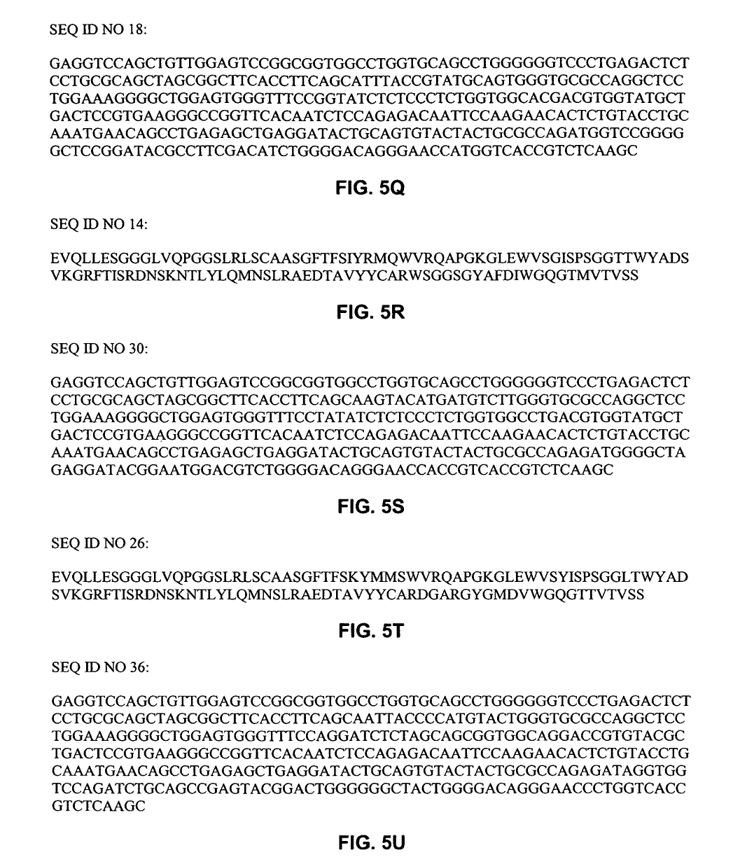

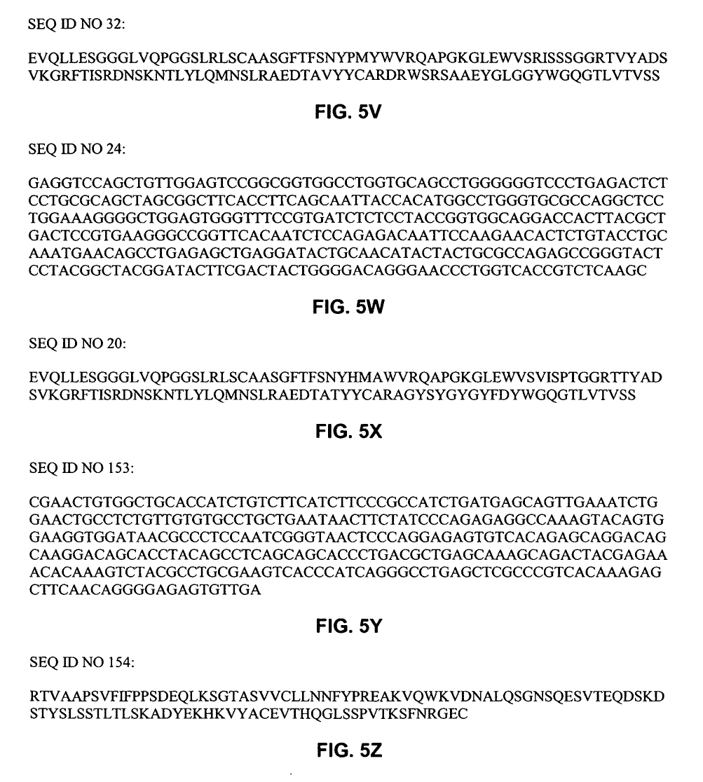

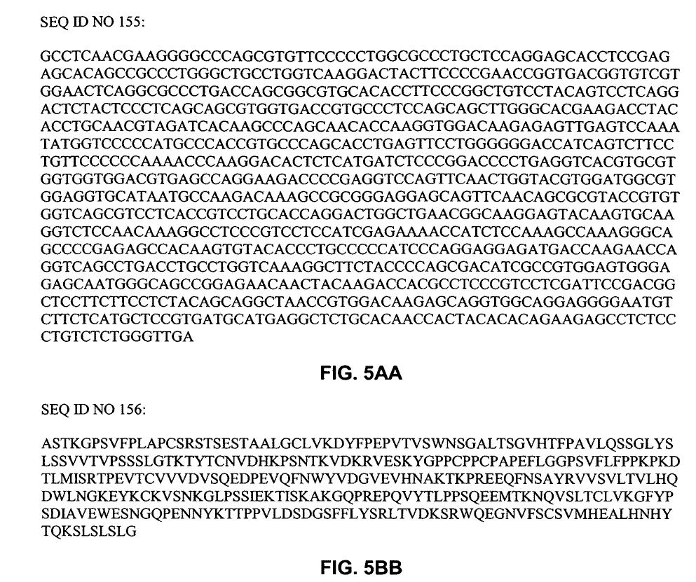

Four Fabs were converted to IgG4.P.agly version and expressed in CHO cells. DNA sequences encoding four distinct anti-IGF-1R Fabs-M13-C06 (FIGS. 5 (A)-(D)), M14-C03 (FIGS. 5(E)-(H)), M14-G11 (FIGS. 5(I)-(L)), and M14-B01 (FIGS. 5(M)-(P)) were selected from a human antibody phage library (Dyax Corp) by biopanning against a recombinant human IGF-1R ectodomain-Fc fusion protein. Each of the four anti-IGF-1R Fabs contained the V_{H}3-23 human heavy chain germline framework and were kappa light chains. The Fab gene sequences were used to construct expression plasmids encoding full-length anti-IGF-1R antibodies using the pV90AS expression vector system for antibody production in mammalian cells. pV90AS is a modified pV90 expression vector designed to generate two transcripts from a single promoter through alternate splicing of a primary transcript (Reference: USPTO Application WO2005/089285). The natural CMV splice donor is spliced either to a partially impaired splice acceptor to generate an antibody light chain-encoding transcript, or to a natural CMV splice acceptor to generate the antibody heavy chain-coding transcript. The partially impaired splice acceptor has been engineered to result in similar amounts of both heavy and light chain transcripts. Light chain Variable (VL) and Constant (CL) regions (SEQ ID NOs:153 and 154, FIG. 5(Y)-(Z)) of each anti-IGF-1R Fab (M13-C06; M14-C03; M14-G11 and M14-B01) were amplified by PCR. (Table 7). The 5′ light chain PCR primer IGF1R-FK included a Sfi I restriction endonuclease site followed by sequence encoding an immunoglobulin light chain signal peptide MDMRVPAQLLGLLLLWLPGARC (SEQ ID NO:157) in frame to sequences corresponding to the amino-terminus of the VL region according to the methods described in Nakamura T, et al., Int J Immunopharmacol. 22:13141 (2000), which is incorporated herein by reference in its entirety. All four of the mature IGF1R light chain sequences had identical amino-termini. The 3′ light chain PCR primer IGF1R-RK included sequence corresponding to the carboxyl-terminus of the CL region and an Asc I site. The PCR product was purified by agarose gel electrophoresis and extraction using the QIAquick GelExtration kit protocol (QIAGEN CA), digested with restriction endonucleases Sfi I and Asc I and ligated with the Sfi I/Asc I digested pHLP025 vector (Holly Prentice). The pHLP025 vector contains Sfi I/Asc I restriction endonuclease sites for receiving antibody light chain (signal peptide-VL-CL) as a Sfi I/Asc I digested PCR fragment in addition to the natural CMV splice donor site sequence, a partially impaired splice acceptor site sequence, and a poly A signal sequence (Reference: USPTO Application WO2005/089285).

The heavy chain Variable (VH) region of each anti-IGF-1R Fab (M13-C06; M14-C03; M14-G11 and M14-B01) was amplified by PCR. The 5′ heavy chain VH PCR primer IGF1R-FH included a Nco I restriction endonuclease site followed by sequence encoding synthetic heavy chain signal peptide MGWSLILLFLVAVATRVLS (SEQ ID NO: 122)) in frame to sequences corresponding to the amino-terminus of the VH region as described above. The 3′ heavy chain VH PCR primer IGF1R-RH included sequence corresponding to the carboxyl-terminus of the VH region and an Sfi I site. The PCR product was purified by agarose gel electrophoresis and extraction using the QIAquick GelExtration kit protocol (QIAGEN, CA), digested with restriction endonucleases Nco I and Sfi I and ligated with the Nco I/Sfi I digested pHLP029 vector (Holly Prentice). The pHLP029 vector contains Nco I/Sfi I sites for receiving the antibody signal peptide-VH sequence as a Nco I/Sfi I digested PCR fragment in addition to an upstream poly A signal sequence, a natural CMV splice acceptor site sequence, and a downstream poly A signal sequence (Reference: USPTO Application WO2005/089285).

The gene sequences coding for (Sfi I site-light chain signal peptide-anti-IGF-1R VL and CL) in pHLP025 and (heavy chain signal peptide-anti-IGF-1R VH-Sfi I site) in pHLP029 were assembled into a single DNA fragment by PCR amplification through common overlapping sequences present in both vectors using the 5′ light chain IGF1R-FK and 3′ heavy chain VH IGF1R-RH PCR primers described above. The resulting PCR product was purified by agarose gel electrophoresis and extraction using the QIAquick GelExtration kit protocol (QIAGEN, CA), digested with restriction endonuclease Sfi I and ligated with the Dra III digested pXWU007 vector. Briefly, pXWU007 was first constructed by subcloning an Age I/BamHI human IgG4 constant region fragment containing a S228P mutation in the IgG4 hinge region and a T299A mutation in the C_{H}2 domain, EU numbering system (Kabat, E, Wu, TT, Perry, H M, Gottesman, K S, Foeller, C: Sequences of Proteins of Immunological Interest. Bethesda, US Department of Health and Human Services, NIH, 1991) (SEQ ID NOs: 155 and 156, FIG. 5 (AA)-(BB)) from plasmid pEAG1808 (provided by Ellen Garber) into Age I/BamHI digested pHLP028 vector. pHLP028 is a pV90 IgG4 vector modified to contain a Dra III site for receiving the single Sfi I-digested PCR product described above (Reference: USPTO Application WO2005/089285).

The resulting plasmid produces a bi-cistronic precursor transcript that upon alternative splicing results in translationally active antibody heavy and light chain mRNAs in approximately stoichiometric quantities. Intermediate and expression vectors for producing full-length aglycosylated human anti-IGF-1R IgG4.P antibodies are shown in Table 8. Correct sequences were confirmed by DNA sequence analysis. Expression of full-length antibodies from plasmids pXWU020, pXWU022, pXWU024, and pXWU025 in mammalian cells results in production of stable, aglycosylated human IgG4.P antibodies.

TABLE 7

Oligonucleotides for PCR amplification
of human antibody domains.

| | |
|---|---|
| LC Primers | |
| IGF1R-FK | 5′- CGAACAGGCCCAGCTGGCCACCATGGACATGAGGG |
| | TCCCCGCTCAGCTCCTGGGGCTCCTTCTGCTCTGGCTCC |
| | CAGGTGCCAGATGTGACATCCAGATGACCCAG -3′ |
| | (SEQ ID NO:123) |
| IGF1R-RK | 5′- TCGCACGGCGCGCCTCAACACTCTCCCCTGTTGAA |
| | GC -3′ |
| | (SEQ ID NO:124) |
| VH Primers | |
| IGF1R-FH | 5′- CGGCCACCATGGGTTGGAGCCTCATCTTGCTCTTC |
| | CTTGTCGCTGTTGCTACGCGTGTCCTGTCCGAAGTTCAA |
| | TTGTTAGAG -3′ |
| | (SEQ ID NO:125) |
| IGF1R-RH | 5′- GGGATCGGCCAGCTGGGCCCCTTCGTTGAGGCGCT |
| | TGAGACGGTGAC -3′ |
| (SEQ ID NO: 126) | |

Forward 5′ light chain PCR primer includes a Sfi I restriction endonuclease site (underlined) and sequence encoding the light chain signal peptide;
Reverse 3′ light chain PCR primer includes an Asc I site (underlined).
Forward 5′ heavy chain variable PCR primer includes a Nco I restriction endonuclease site (underlined) and sequence encoding the heavy chain signal peptide.
Reverse 3′ heavy chain variable PCR primer includes an Sfi I site (underlined).

TABLE 8
Intermediate and expression plasmids encoding anti-IGF-1R antibodies.

| Vector | Composition | Antibody chain(s) |
|---|---|---|
| pXWU008 | pHLP025 + C03 L | C03 VL-CL |
| pXWU010 | pHLP025 + C06 L | C06 VL-CL |
| pXWU012 | pHLP025 + G11 L | G11 VL-CL |
| pXWU013 | pHLP025 + B01 L | B01 VL-CL |
| pXWU014 | pHLP029 + C03 VH | C03 VH |
| pXWU016 | pHLP029 + C06 VH | C06 VH |
| pXWU018 | pHLP029 + G11 VH | G11 VH |
| pXWU019 | pHLP029 + B01 VH | B01 VH |
| pXWU020 | pXWU007 + C03 L-VH | C03 VL-CL + C03 VH-agly γ4.P |
| pXWU022 | pXWU007 + C06 L-VH | C06 VL-CL + C06 VH-agly γ4.P |
| pXWU024 | pXWU007 + G11 L-VH | G11 VL-CL + G11 VH-agly γ4.P |
| pXWU025 | pXWU007 + B01 L-VH | B01 VL-CL + B01 VH-agly γ4.P |

### Example 7

### Construction of Full-Length Anti-IGF-1R IgGs for Improved Expression in Mammalian Cells

To improve antibody expression yields and product quality the original VH gene sequences from anti-IGF-1R Fabs M13-C06, M14-C03, M14-G11, and M14-B01 were modified. First, anti-IGF-1R VH sequences were analyzed for sequences containing putative splice sites with public sequence recognition programs (www.tigr.org/tdb/GeneSplicer/gene_spl.html (The Institute for Genomic Research, 9712 Medical Center Drive, Rockville, Md. 20850), www.fruitfly.org/seq_tools/splice.html). (Martin G. Reese and Frank H. Eeckman, Lawrence Berkeley National Laboratory, Genome Informatics Group, 1 Cyclotron Road, Berkeley, Calif., 94720; see also, Reese M G, Eeckman, F H, Kulp, D, Haussler, D, 1997. “Improved Splice Site Detection in Genie”. J Comp Biol 4(3), 311-23.). Second, codons in the heavy chain variable region of the anti-IGF-1R Fabs were replaced with codons corresponding to the identical Kabat positions from antibodies that have been successfully expressed in CHO cells without encountering any changes in the original anti-IGF-1R VH polypeptide sequence. This second step mostly removes putative splice sites but an additional splice site analysis followed by synonymous codon exchange was performed to reduce the predicted likelihood of a putative splice site being present.

DNA fragments encoding synthetic heavy chain leader in frame with sequence-optimized VH sequences of anti-IGF-1R Fabs-M13-C06 (SEQ ID NO:18, FIG. 5(O)), M14-C03 (SEQ ID NO:30, FIG. 5(S)), M14-G11 (SEQ ID NO:36, FIG. 5(U)), and M14-B01 (SEQ ID NO:24, FIG. 5(W)) were obtained as chemically synthesized double-stranded DNA sequences from a commercial provider (Blue Heron Biotechnology, Inc. Bothell Wash.). The Nco I and Sfi I restriction endonuclease sites at 5′ and 3′ were included in the synthesized fragments. The leader and anti-IGF1R sequence-optimized VH region fragments were cloned into the Nco I/Sfi I digested the pHLP029 vector as described in Example 6 above. Recombination with the appropriate corresponding light chains in pHLP025 and subsequent cloning of the single fragment into pXWU007 is as described in Example 6 above. Expression constructs producing the sequence-optimized full-length aglycosylated human anti-IGF-1R IgG4.P antibodies are shown in Table 9. Correct sequences were confirmed by DNA sequence analysis. Expression of full-length antibodies from the plasmid series pXWU029-pXWU032 in mammalian cells results in production of stable, aglycosylated human IgG4.P antibodies.

TABLE 9
Sequence-optimized expression plasmids encoding anti-IGF-1R
antibodies. Optimized heavy chain sequences are preceded
with an “m”.

| Vector | Composition | Antibody chain(s) |
|---|---|---|
| pXWU029 | pXWU007 + C03 L-mVH | C03 VL-CL + |
| | | mC03 VH-agly γ4.P |
| pXWU030 | pXWU007 + C06 L-mVH | C06 VL-CL + |
| | | mC06 VH-agly γ4.P |
| pXWU031 | pXWU007 + G11 L-mVH | G11 VL-CL + |
| | | mG11 VH-agly γ4.P |
| pXWU032 | pXWU007 + B01 L-mVH | B01 VL-CL + |
| | | mB01 VH-agly γ4.P |

### Example 8

### Transient Expression and Characterization of IGF-1R Antibodies

Plasmid DNAs were used to transform CHO DG44 cells for transient production of antibody protein. 20 μg of plasmid DNA was combined with 4×10⁶ cells in a volume of 0.4 mL of 1×PBS. The mixture was added to a 0.4 cm cuvette (BioRad) and placed on ice for 15 min. The cells were electroporated at 600 uF and 350 volts with a Gene Pulser electroporator (BioRad). The cells were placed into a T-25 flask containing CHO-SSFM II media plus 100 uM Hypoxanthine and 16 uM Thymidine and incubated at 37° for 4 days. Supernatants were harvested and biochemically characterized by Western Blot and tested for antigen binding by ELISA.

Alternatively, selected Fabs also converted to full-length human IgG4.P version and expressed using a different vector system by a method described below. DNA sequences encoding five distinct anti-IGF1R Fab antibodies, M12-E01, M12-G04, M13-C06, M14-C03, and M14-G11 were transferred into vectors for expression of full-length human IgG4.P. All five antibodies use the V_{H}3-23 human heavy chain germline fragment. The variable heavy chain was removed from the soluble Fab expression vector by digestion with restriction enzymes MfeI and BstEII. The resulting fragment was purified by agarose gel electrophoresis using the QIAquick Gel Extraction Kit (Qiagen, CA) and ligated into the MfeI/BstEII digested pRR253 vector (Rachel Rennard). The resulting plasmid contains the heavy chain signal peptide (MGWSCIILFLVATATGAHS, SEQ ID NO: 127) followed by the anti-IGF1R VH and constant regions for human IgG4.P.

Four of the five antibodies, M12-G04, M13-C06, M14-C03, and M14-G11, contain kappa light chains. The variable light chain was amplified by PCR with primers to introduce an EcoRV site 5′ and a BsgI 3′ to the variable region. The resulting PCR fragment was purified by agarose gel electrophoresis using the QIAquick Gel Extraction Kit (Qiagen, CA) and ligated into TOPO2.1 TA vector (Invitrogen, CA). The variable kappa light chain was removed from the TOPO vector by digestion with restriction enzymes EcoRV and BsgI and purified. The fragment was ligated into EcoRV/BsgI digested pRR237 vector, which contains the immunoglobulin light chain signal peptide (MDMRVPAQLLGLLLLWLRGARC, SEQ ID NO:128) and the constant kappa domain. The resulting vector was digested with BamHI and NotI and the entire expression cassette (signal sequence, variable and constant kappa domains) was purified and ligated into BamHI/NotI digested pRR223.

The M12-E01 antibody contains a lambda light chain. The variable light chain was amplified by PCR with primers to introduce an AgeI site 5′ of the variable region. The resulting PCR fragment was purified by agarose gel electrophoresis using the QIAquick Gel Extraction Kit (Qiagen, CA) and ligated into TOPO2.1 TA vector (Invitrogen, CA). The variable lambda light chain was removed from the TOPO vector by digestion with restriction enzymes AgeI and AvrII and purified. The fragment was ligated into AgeI/AvrII digested pXW347 vector (Xin Wang), which contains the immunoglobulin light chain signal peptide (METDTLLLWVLLLWVPGSTG, SEQ ID NO: 129) and the constant lambda domain. The resulting vector was digested with NotI and the entire expression cassette (signal sequence, variable and constant lambda domains) was purified and ligated into NotI digested pRR223.

Plasmid DNA was used to transfect 293E cells for transient expression of antibody protein. 1.2 μg of each (heavy and light) plasmid DNA was transfected into 2×10⁶ cells with Qiagen's Effectene Transfection Protocol (Qiagen, CA). Cells were incubated at 37° C. for 3 days. Supernatant was harvested and full-length antibody confirmed by both Western Blot and ELISA methods. The ability of full.IgG4.P to bind to IGF-1R was confirmed by ELISA.

### Example 9

### Development of Anti-IGF-1R Antibody Producing CHO Cell Line

This example gives a detailed description of expression of the anti-IGF-1R antibody comprising the binding domain of the Fab M13-C06 as full-length hinged-modified agly gamma 4, kappa (referred to herein as “agly.IgG4.P” or “G4.P.agly”) antibody. The other Fabs described herein, i.e., those listed Table 3, were expressed in a similar manner. The variable and constant regions of M13-C06 are of human sequence origin. The entire light chain and heavy chain variable regions are derived from a Fab generated against human IGF-1R by the DYAX phage display technology. The variable, as well as the light chain constant regions were subcloned into an alternate splice expression vector. The alternate splice configuration links the light and heavy chain through the usage of a single splice donor with two splice acceptors where each splice acceptor generates a transcript encoding one of the two chains. The expression vector DNA encoding the immunoglobulin genes was electroporated into insulin independent Chinese hamster ovary cells (CHO DG44i). A CHO transfectoma (cell line 40B5) was selected for production purposes.

pXWU007—an “empty” expression vector contains a human gamma 4 constant region (heavy chain) as well as separate promoter-enhancers and polyadenylation regions for gene expression in mammalian cells, but does not contain variable domains. When expressed and translated the heavy chain polypeptide contains two amino acid substitutions, S228P and T299A, to reduce “half-antibody” formation and eliminate N-linked glycosylation, respectively.

Complementary DNA from the corresponding variable (VL) and constant (CL) domains of the light chain gene of M13-C06 and the variable (VH) domain of the heavy chain gene of M13-C06 was cloned into the expression vector pXWU007. The pXWU007 vector contains cloning sites for inserting the entire light chain and variable heavy cDNAs directly upstream of the human heavy chain constant region. In addition to the Ig genes, this expression vector contains a dihydrofolate reductase (DHFR) gene that can be used for selection in mammalian cells.

The resulting expression vector was then transfected into CHO cells to initiate the generation of the anti-IGF-1R secreting CHO cell lines (40B5).

PXWU022 was electroporated into CHO cells. Immunoglobulin light chain specific PCR primers were used to PCR amplify the Fab light chain cDNA. The 5′ specific oligo sequence included the native signal peptide from the light chain of the Biogen Idec anti-CD23 molecule. The 5′ and 3′ oligos contain Sfi I and Asc I restriction endonuclease recognition sequences, respectively, for subcloning into an intermediate vector (pHLP025). The VH cDNA was PCR amplified using a 5′ oligo that included a synthetic heavy chain signal peptide. The 5′ and 3′ oligos contain Nco I and Sfi I restriction endonuclease recognition sequences, respectively, for subcloning into an intermediate vector (pHLP029).

Overlapping PCR using the light chain 5′ and VH 3′ oligos and pHLP025 and pHLP029 as templates was employed to combine the light chain and the VH region as one cDNA segment. The resultant product was subcloned into the Dra III site of pXWU007 thus creating the final alternate splice expression vector, pXWU022. The alternate splice configuration generates two transcripts from a single promoter through alternate splicing of the primary transcript. The natural CMV splice donor is spliced either to a suboptimal splice acceptor to generate a light chain-encoding transcript, or to a natural CMV splice acceptor to generate the heavy chain-coding transcript. The sub-optimal splice acceptor has been designed to generate similar amounts of both transcripts.

The DNA vector (pXWU022) was prepared in HEBS buffer at a concentration of ˜700 ng/μL prior to electroporation in to CHO cells. Five electroporations were performed using various concentrations of DNA (15, 20, 30, 40, and 45 μg). Each electroporation was done in a disposable 0.4 cm cuvette (Invitrogen) containing 4×10⁶ log phase CHO cells in 0.7 ml sterile HEBS buffer and DNA in 0.1 mL HEBS (0.8 mL total volume). Cells were shocked using a Bio-Rad Gene Pulser XCELL, set at 290 volts, 950 micro Faradays. Shocked cells were then allowed to stand at room temperature for 10 minutes then mixed with 10 mL room temp insulin free CHOM16 medium, centrifuged (3′ @ 1000 rpm), and aspirated. Cells were then resuspended in 12 mL (room temp.) insulin free CHOM16 medium and transferred to a T-75 tissue culture flask.

Cells and Media: prior to electroporation the CHO cells were grown in serum free media (CHOM24) with the addition of 1× nucleosides. CHOM24 is a chemically defined in-house media formulation that does not contain any animal components. Methotrexate selection was performed in nucleoside free CHOM16 and CHOM24 chemically defined media.

Following electroporation, 4×10⁶ CHO cells were pooled into a T-75 flask. Selection for DHFR expression began immediately as the cells were inoculated in nucleoside free medium. Cells were eventually expanded to 125 mL shake flasks in CHOM24 (˜3 weeks). To isolate clonal cell lines, the transfected stable pools were diluted and plated at 1 cell/well in 200 μL CHOM16 on four 96-well plates. Plates were maintained at 36° C. until they were screened for antibody titer.

CHO colonies were screened for immunoglobulin production by assaying cell supernatants using an ELISA specific for the human kappa chain (day 21 to day 28 after plating). The capture antibody used in the ELISA was a polyclonal goat anti-human IgG (SouthernBiotech) and the detection antibody was a polyclonal goat anti-human kappa conjugated to horseradish peroxidase (SouthernBiotech). Colonies secreting the highest amount of immunoglobulin were expanded.

A total of 381 nearly confluent wells of the 1920 wells seeded were assayed. Of the 381 wells, 60 were expanded for further study and of these 60, 4 were selected for amplification (15A7, 40B3, 40B5, 40F6).

### Example 10

### Purification and Characterization of Fully Human Anti-IGF-1R IgG4.P.agly Antibodies

The antibody produced in CHO cells were purified and characterized by methods described below.

Protein A Capture: Pre-equilibrate the Protein A column with 1×PBS (equilibration buffer) at 100-150 cm/hr with 3 column volumes. Load the supernatant at 150 cm/hr with a maximum of 10 mg of αIGF-1R per milliliter of resin. After loading, wash the column with 5 column volumes of equilibration buffer. Then, step elute in an upflow direction with 100 mM Glycine, pH 3.0. Collect desired fractions and titrate to neutral pH with 2 M Tris base. Dialyze collected fractions against 1×PBS and concentrate material to prepare for the size exclusion step.

SUPERDEX™ 200 (Size Exclusion) aggregate removal step involved equilibration of SUPERDEX™ 200 with 1×PBS with 1.5 column volumes at a flow rate of 36 cm/hr followed by loading of protein and collecting desired fractions.

### Identity Testing Performed as Follows

1). Intact mass analysis by mass spectrometry where molecular mass measurements were performed on an electrospray mass spectrometer (ESI-MSD). Prior to analysis, the sample was reduced to remove disulfide bonds. The deconvoluted mass spectrum represents the masses of the heavy and light chains.

2). N-terminal sequence analysis was performed by Edman degradation using an ABI protein sequencer equipped with an on-line PTH analyzer. The sequences for the initial amino acids of the light chain and heavy chain were identified.

3). Peptide mapping with mass spectrometric analysis: tryptic or/and EndoLysC peptide maps were performed to obtain complete sequence coverage by analysis of the LC/MS data generated from each peptide. In addition, determination of sites and amounts of oxidation and deamidation were detected.

Purity testing was performed by; 1) SDS-Page or CE-SDS: Reduced and non-reduced samples, this technique is used to measure antibody fragmentation, aggregation and impurities, 2) SEC-HPLC with LS and RI technique was used to measure aggregation and fragmentation and light scattering determines the molar mass of sample components. 3) SDS gel or capillary IEF method was used to determine the isoelectric focusing pattern and pI distribution of charge isoforms that can result from C- and N-terminal heterogeneity and/or deamidation.

Finally, endotoxin concentrations were measured by the Limulus amoebocyte lysate (LAL) kinetic turbidometric method.

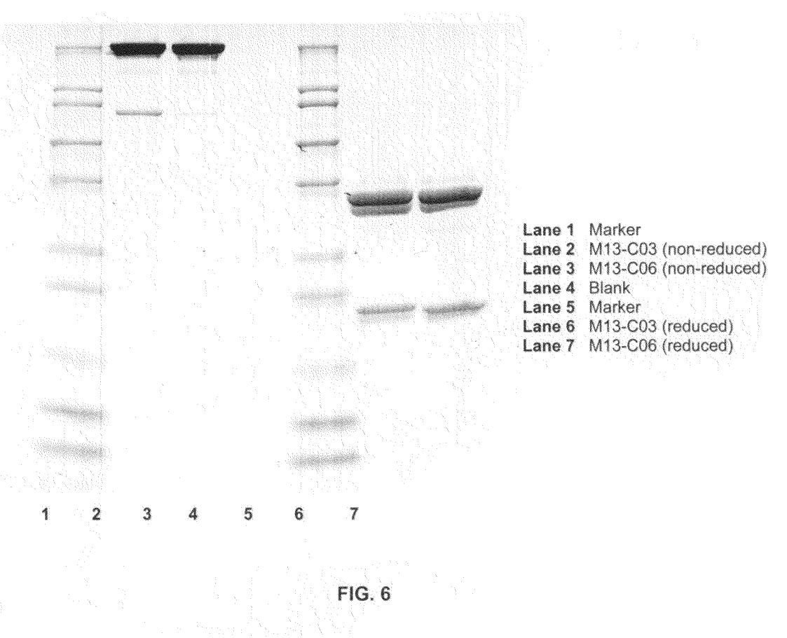

FIG. 6 shows non-reduced and reduced SDS PAGE analysis of G4.P.agly versions of fully human M13-C06 and M14-C03 antibodies. Both G4.P and G4.P.agly versions of antibodies M13-C06, M14-C03, M14-B01, and M14-G11 were produced. M12-E01 and M12-G04 were produced on as the G4.P version.

### Example 11

### Binding Activity of Fully Human Anti-IGF-1R Antibodies

The binding activity to soluble IGF-1R of the G4.P.agly and G4.P versions of antibodies tested by ELISA. Soluble IGF-1 receptor fusion protein (Biogen Idec) at 2.5 μg/ml in 0.025 M carbonate buffer, pH 9.6 was coated at 50 μl/well in a 96-well (IMMULON2 HB, Dynex Technologies, Inc., Cat. #3455) plate and incubated overnight at 4° C. The plate was washed with phosphate-buffered saline (PBS, Irvine Scientific, Cat#9240), pH 7.4 plus 0.025% Tween 20 in the Skan Washer 300 (Skatron Instruments), blocked with buffer containing 1% nonfat milk, 0.05% Tween 20 in PBS, pH 7.4, and then incubated at room temperature for 1 hour. After incubation plate was washed with PBS plus 0.025% Tween 20 in the Skan Washer 300. For the assay, the soluble IGF-1 receptor-coated plate was next incubated with the control and test antibodies of varied concentrations, diluted in 1% nonfat milk, 0.05% Tween 20 in PBS at 50 μl/well. Following a one hour incubation at room temperature, plate was washed with PBS plus 0.025% Tween 20 in the Skan Washer 300. A 2000-fold dilution in 1% nonfat milk, 0.05% Tween 20 in PBS of goat anti-human Kappa—HRP (Southern Biotech Cat#2060-05) was added 50 μl/well to detect bound antibody. Plate incubated for 1 hour at room temperature was washed with PBS plus 0.025% Tween 20 in the Skan Washer 300. TMB solution (KIRKEGAARD & PERRY LABS, INC. cat: 50-76-00) was added 101l/well, and the reaction was stopped with 50 ul/well of 4NH₂SO₄ (LabChem, Cat#LC25830-1) after two minutes. The absorbance was measured at 450 nm, background 540 nm for TMB using the Molecular Devices plate reader. Data was analyzed using the SOFTMAX PRO software package version 4.3 LS (Molecular Devices Corp.).

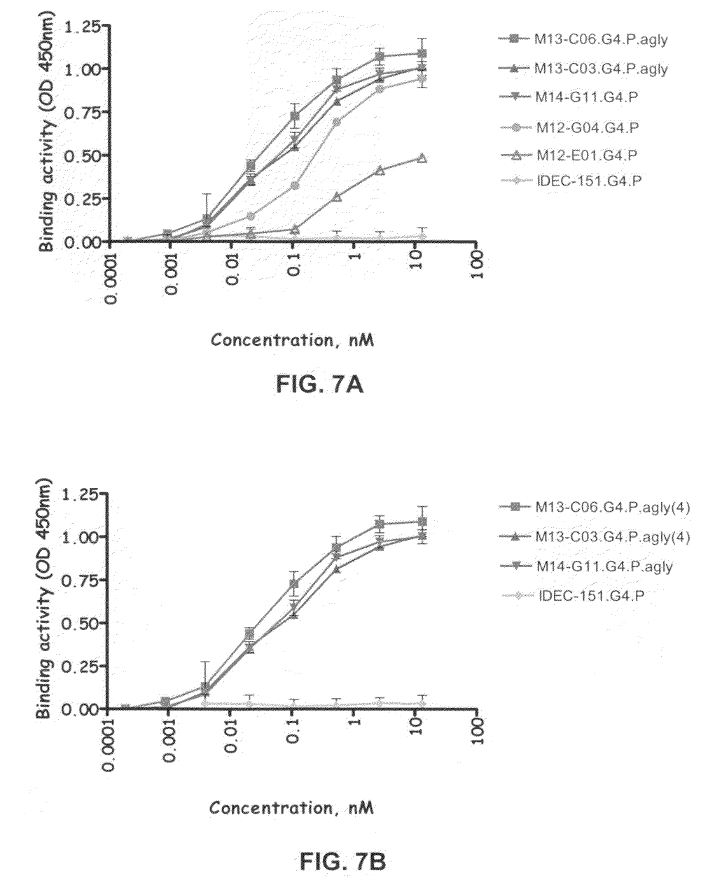

FIG. 7 (A) shows the concentration dependent binding of G4 version of M13-C06, M14-C03, M14-G11, M12-E01 and M12-G04, whereas the control antibody, IDEC-151 (G4.P) again did not show any binding to IGF-1R.Fc.

FIG. 7 (B) shows the concentration dependent binding of G4.P.agly version of M13-C06, M14-C03 and M14-B01 to soluble IGF-1R.Fc by ELISA. A G4.P antibody of irrelevant specificity (IDEC-151) used as a negative control did not show any binding to IGF-1R.Fc.

The binding activity of human antibodies to wild type IGF-1R expressed on tumor cells was determined by flow cytometry. Tumor cell lines MCF-7 and Calu-6 were cultured in Minimum Essential Medium Eagle (ATCC, Cat#30-2003) supplemented with 10% fetal bovine serum (FBS) (Irvine Scientific, Cat#3000A) and 50μ/ml gentamicin (Gibco Invitrogen, Cat#15750-060). Panc-1, Colo-205, NCI-H23 and ZR-75 were cultured in RPMI-1640 (ATCC, Cat#30-2001) supplemented with 10% FBS and 50 μg/ml gentamicin. Trypsin-EDTA (Sigma, Cat#T4049; Sigma-Aldrich Corp. (St. Louis, Mo., USA)) solution was used for removal of adherent cells from culture vessels.

Cells were rinsed twice with phosphate buffered saline (PBS) (Irvine Scientific, Cat# 9240), pH 7.4, trypsinized and washed once in PBS and 10% FBS. Cells were adjusted to 10⁷ cells/ml in FACS buffer (0.05% sodium azide, 2% FBS, 10% normal goat serum and 100 μg/ml normal goat IgG in PBS) and put on ice for at least 15 minutes. Control and test antibodies were aliquoted into a Corning 3790 plate. Cells at 50 μl/well were added to a Corning 3799 plate. Primary antibodies from Corning 3790 plate were added at 50 μl/well to respective wells of Corning 3799 plate. Next, cells (0.5×10⁶ cells/sample) were incubated 45 min on ice. Following incubation plates were centrifuged at 1500 rpm for 4 minutes and then supernatants were aspirated. Cells were resuspended in 150 μl of FACS buffer. Plates were centrifuged at 1500 rpm for 4 minutes and supernatants were aspirated. A 750-fold dilution in FACS buffer of goat anti-human IgG-RPE (Southern Biotech Cat#2040-09) was added 100 μl/well. Next, cells (0.5×10⁶ cells/secondary antibody) were incubated 45 min on ice. A 500-fold dilution in FACS buffer of 7AAD (Molecular Probes, Cat#A1310) was added 50 μl/well and incubated for 5 minutes on ice. Following incubation plates are spun at 1500 rpm for 4 minutes and then supernatants were aspirated. Cells were resuspended in 150 μl of FACS buffer. Plates were centrifuged at 1500 rpm for 4 minutes and supernatants were aspirated. Cells were resuspended in 100 μl/well of FACS buffer. Cells were transferred to 12×75 mm FACS tubes with 200 μl of FACS buffer. Finally, cells were examined for fluorescence intensity on a FACSCalibur using CellQuest software (both from Becton Dickinson).

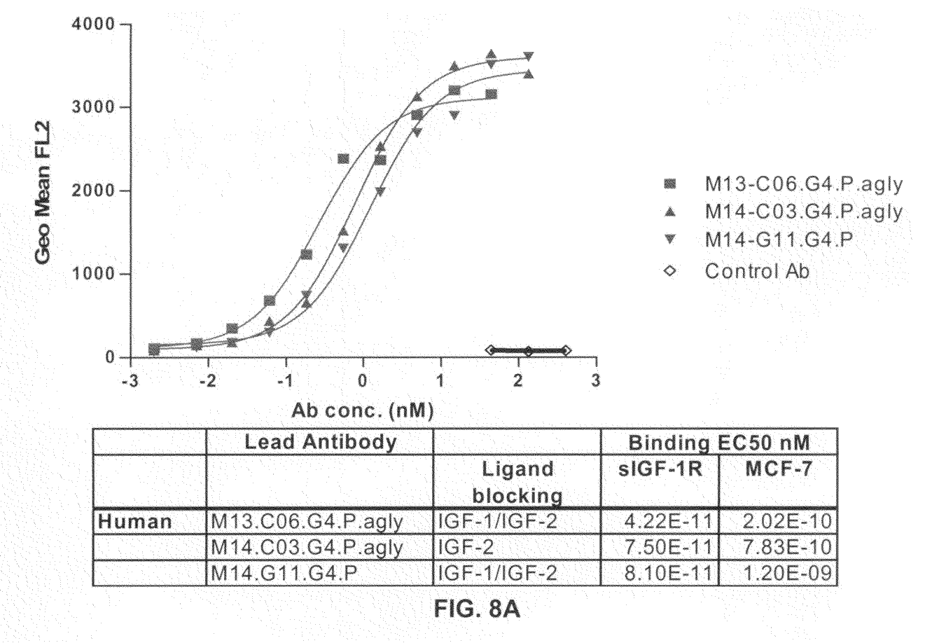

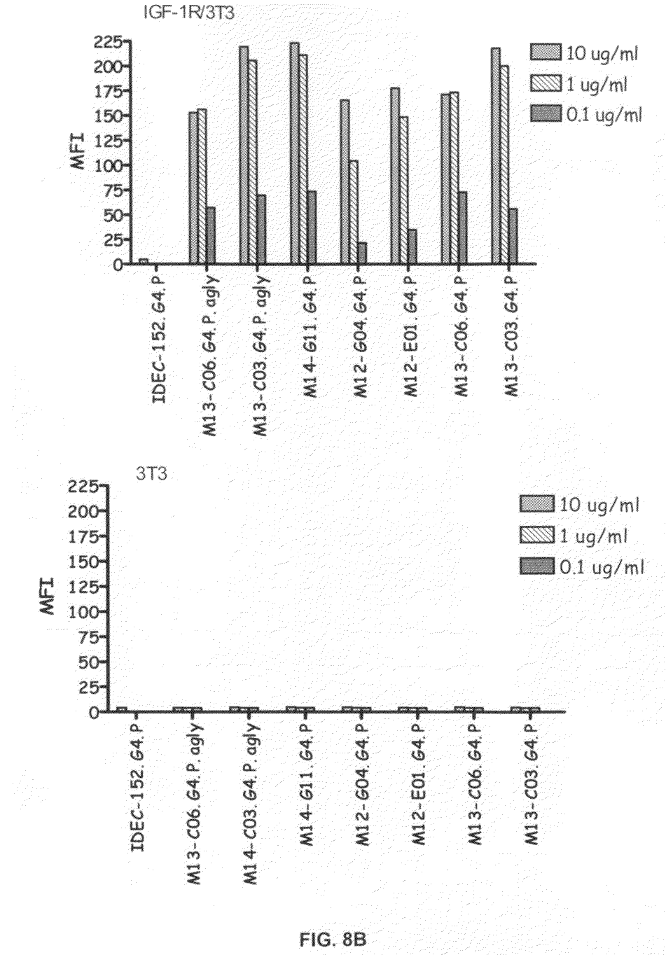

FIG. 8 shows the concentration dependent binding of M13-C06.G4.P.agly, M14-C03.G4.P.agly and M14-G11.G4.P to IGF-1R expressed on MCF-7 cells (FIG. 8(A)). The cell-surface binding specificity of antibodies was confirmed by testing binding to IGF-1R/3T3 transfectants and 3T3 parent cells. All of the lead antibodies showed specific reactivity to IGF-1R expressing 3T3 but not to 3T3 cells (FIG. 8(B)).

### Example 12

### Inhibition of Ligand Binding to IGF-1R by Fully Human Antibodies

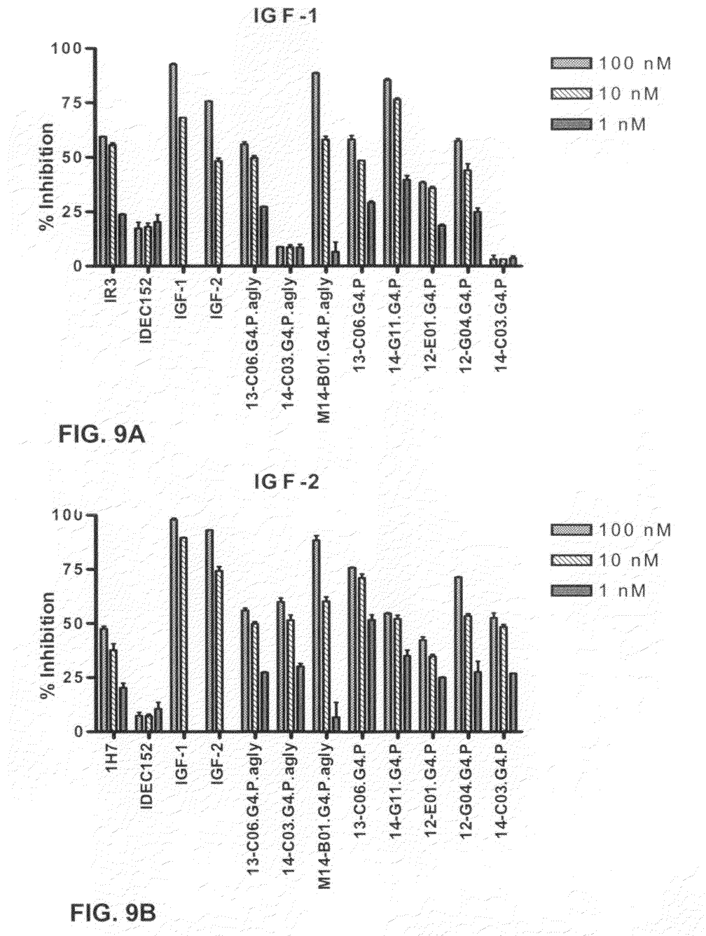

The ability of the G4.P.agly and G4.P versions of human antibodies to block IGF-1 and IGF-2 binding to soluble IGF-1R-Fc was determined. The IgG4 versions of M13-C06, M14-G11, M14-B01, M12-E01 and M12-G04 blocked both IGF-1 and IGF-2 binding to IGF-1R, whereas in this experiment M14-C03 only blocked IGF-2 (FIG. 9 (A) and (B)).

The ligand blocking ability of the anti-IGF-1R antibody was determined by a solid phase RIA capture method as described in Example 3. Briefly, the antibodies at varying concentrations were (100 nM-0.01 nM) co-incubated with 100,000 cpm of ¹²⁵I-labeled IGF-1 or ¹²⁵I-IGF-2 in the wells of a 96-well IMMULON2 plate, wherein human IGF-1R-Fc was previously immobilized (200 ng/well). After 1 hour of incubation at room temperature, the wells were washed and counted for bound radioactivity by a Gamma Counter. An isotype matched negative antibody control, IDEC-151 (human G4), was used. Percent (%) inhibition was calculated as=[1-(Ave.CPM with Ab)/(Ave.CPM with buffer)]×100%.

The result demonstrate that fully human antibodies M13-C06.G4.P, M13-C06.G4.P.agly, M14-G11.G4.P, M14-G11.G4.P.agly, M14-B01.G4.P.agly, M12-E01.G4.P, and M12-G04.G4.P block both IGF-1 and IGF-2 binding to IGF-1R, whereas in this experiment, the antibodies M14-C03.G4.P and M14-C03.G4.P.agly blocked only IGF-2 binding to IGF-1R. See, FIG. 9(A)-(B).

### Example 13

### Inhibition of Tumor Cell Growth by Fully Human Anti-IGF-1R Antibodies

The ability of antibodies to block IGF-1 and IGF-2 driven tumor cell growth was tested using a cell viability assay.

NCI-H23, Calu-6, Colo-205, Panc-1, BxPC-3 (ATCC) tumor lines were purchased from ATCC. Cell lines were grown in complete growth medium containing RPMI-1640 (ATCC), 10% fetal bovine serum (Irvine Scientific Inc.) and 50 μg/ml of Gentamycin (Gibco, Invitrogen). Trypsin-EDTA solution (Sigma) was used for removal of adherent cells from culture vessels. Phosphate buffered saline, pH 7.2, was from MediaTech Inc The 96-well clear bottom plates for luminescent assay was purchased from Wallac Inc.

Cells grown to 80% monolayers were, trypsinized, washed, resuspended and plated into 96-well plates in 200 μl of 2% growth medium at 8×10³ cells/well for NCI-H23 and Colo-205 cells; and 5×10³ cells/well for Calu-6, Panc-1 and BxPC-3 cells. After 24 hours, the culture medium was replaced with 100 μl of serum free medium (SFM), and 50 μl of serially diluted antibodies at 4× concentration was added. Following another hour of incubation at 37° C., 50 μl of IGF-1 or IGF-2 at 4× concentration was added and incubated at 37° C. until 48 hours to measure cell growth. All treatments were done in triplicates. Cell growth was measured using the CELL TITER-GLO™ Luminescent Cell Viability Assay (Promega, Madison, Wis.). The 1:1 mixture of reagent and SFM was added at 200 μl/well. Luminescence was detected on Wallac (Boston, Mass.) plate reader.

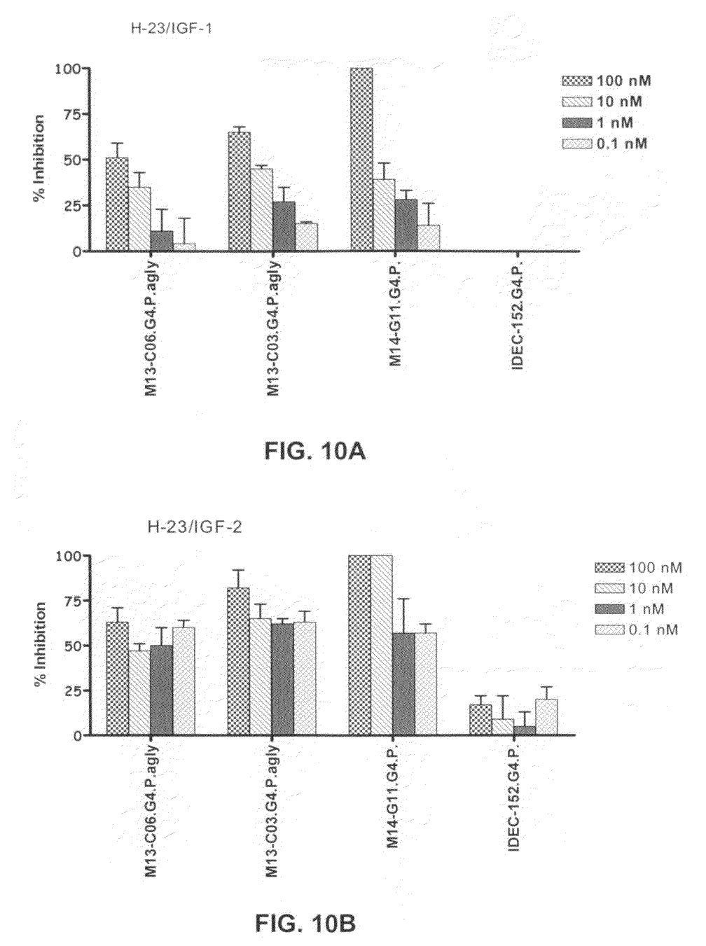

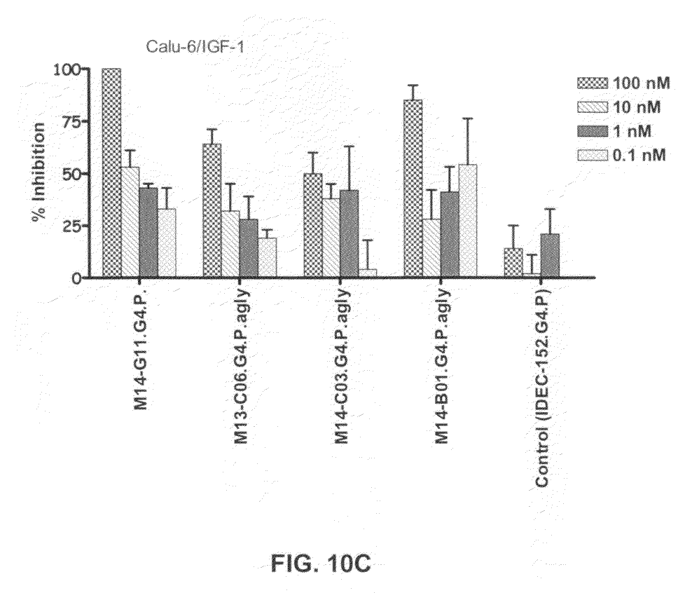

The various human IgG4 versions of the anti-IGF-1R antibodies exhibited inhibition of IGF-1 and IGF-2 driven cell proliferation in H-23 (IGF-1 and IGF-2) Calu-6 (IGF-2) cells (FIG. 10(A)-(C)). Other cell lines exhibited comparable trends (see e.g., Example 20).

### Example 14

### Internalization of IGF-1R by Fully Human Anti-IGF-1R Antibodies

MCF-7 cells were seeded at 50,000 cells per well into 8 well chamber slides (Becton Dickinson Collagen Type I coated culture slides, BD BioCoat™ #354630) 48 hours prior to staining procedures. Cells were routinely maintained below 20 passages. On day of staining procedures, culture media was discarded from each well and replaced with 500 μl cold incubation buffer (MEM Eagle ATCC #30-2003+1% BSA). Cells were washed 2× with this buffer for 3 min each wash. 250 μl of each mAb or human G4.P.agly antibody to be tested was then added to the appropriate well at a concentration of 10 μg/ml, diluted in incubation media, and incubated on ice for 1 hour. A murine anti-human-IGF-1R antibody (Lab Vision/NeoMarkers, clone 24-31 cat# MS-641) was used as a positive control antibody to compare degree of internalization. After the 1 hour incubation on ice, the time zero (t=0′) slide was washed 3× with 500 μl of cold wash buffer (PBS+1% BSA+2% Goat serum) for 3 min each wash (slides always kept on ice!). The t=0 slide was then fixed with 500 μl 4% paraformaldehyde (diluted with PBS from 16% stock; EMS #15710) for 15 minutes at room temperature. The t=0 slide was then washed again 3× with cold wash buffer for 3 minutes each wash, then left on ice. Meanwhile, the remaining slides were put into a 37° C. incubator for their designated time points (15 and 60 minutes). At the end of their incubation time each slide followed the same procedures as above—washes and fixation, and put on ice. All slides were then permeabilized with 200%1 cold permeabilization buffer (Wash buffer+0.5% Triton-X) for 10 minutes on ice. All slides were then washed 3× with 500 μl cold wash buffer for 3 minutes each wash. The secondary antibody was prepared at a 1:1000 dilution (AlexaFluor 488 Goat-anti-mouse IgG (H+L), Molecular Probes #A 11029 for the mAbs and AlexaFluor 488 Goat-anti-human IgG (H+L), Molecular Probes #A 11013 for G4 antibodies) in wash buffer, after an initial spin of the stock vial at 10,000 rpm for 10 min at 4° C. 250 μl of the diluted secondary antibody was added to each well and incubated for 40 min at room temperature in the dark (covered). Slides were again washed 3× with 500 μl cold wash buffer. On the final wash, the buffer was discarded and all wells were left empty. The chambers were then disassembled from the slide using the provided disassembly tool, and cover slips were mounted with Vectashield mounting medium containing DAPI (Vector #H-1500, Hard Set™). Slides were stored at 4° C. in the dark overnight to allow the mounting medium to dry.

Pictures of the slides were taken with a confocal microscope using the LaserSharp 2000 program (BioRad v5.2) and represented as a merge of blue and green components from Kalman 10 average. The internalization of IGF-1R by M13-C06.G4.P.agly antibody was observed at time 0, 15 and 60 min by confocal microscopy.

M13-C06.G4.P.agly showed rapid internalization of IGF-1R in 60 min (data not shown). Both M14-C03.G4.P.agly and M14-G11.4.P all showed internalization property similar to M13-C06.G4.P.agly antibody (data not shown). As expected the positive control, clone 24-31, also internalized the receptor whereas isotype matched negative controls (mouse 7F2 and human G4, IDEC-152.G.P (primatized antibody)) did not bind or internalize (data not shown).

In addition, the rate of receptor internalization was measured by a FACS based method for certain of the murine monoclonal antibodies. MCF-7 cells grown to 70% confluent monolayers were lifted off the flask with cell dissociation buffer (Gibco catalog #13151-014). Cells resuspended in media and 5×10⁶ cells were added into 12×75 mm tube (Falcon catalog# 352054), where each tube represents a different mAb to be tested. 10 μg/ml mAb was added to its corresponding tube in 0.5 ml FACS buffer containing no azide (PBS+1% BSA) as well as a control tube with no antibody for measuring experimental internalization error. Tubes were incubated on ice for 1 hour 15 minutes then washed and reconstituted in 1 ml FACS buffer. 100 μl of each sample was removed into 1 well of a 96 well u-bottom plate (NUNC #163320) kept on ice to prevent internalization and termed time zero (t=0). This was used as a 100% Ab bound control. Tubes were then transferred to a 37° C. water bath and 100 μl samples removed at time (t)=5, 10, 20, 40, and 60 minutes (later changed to 5, 10, 15, 30 and 60 minutes) and placed into separate wells of a 96 well u-bottom plate on ice. Once all samples were collected, the plates were spun at 1200 rpm in a 4° C. centrifuge to pellet cells. Antibody added to detect internalization of receptor was either anti-CD221-PE (BD Pharmingen cat# 555999-anti-IGF-1R; 10μ/100 μl sample) to detect receptors remaining on cell surface, or Goat-anti-mouse-PE (Jackson ImmunoResearch Lab cat#115-116-146; 5 μg/ml) to detect antibody remaining on cell surface. Samples were incubated 1 hour in FACS buffer containing 0.1% Sodium Azide, washed x1, and brought to a final volume of 200 μl in FACS buffer containing azide. Samples were then run and collected using a FACSArray (BD) and geometric means determined. Also run PE-labeled Quantibrite beads (BD #340495) to quantitate the number of PE molecules bound to the cell surface, where the Quantibrite bead are run on the same FL2 setting as samples. The number of PE molecules bound to the bead is given in their packaging, allowing the quantitation of the number of PE molecules bound to the cell surface using geometric means of the sample and of the beads. The FACS assay showed that the murine monoclonal antibodies tested promoted internalization of IGF-1R (data not shown).

### Example 15

### Inhibition of IGF-1R Mediated Signaling by Fully Human Antibodies

Part I: Inhibition of Signal Transduction in MCF-7Cells

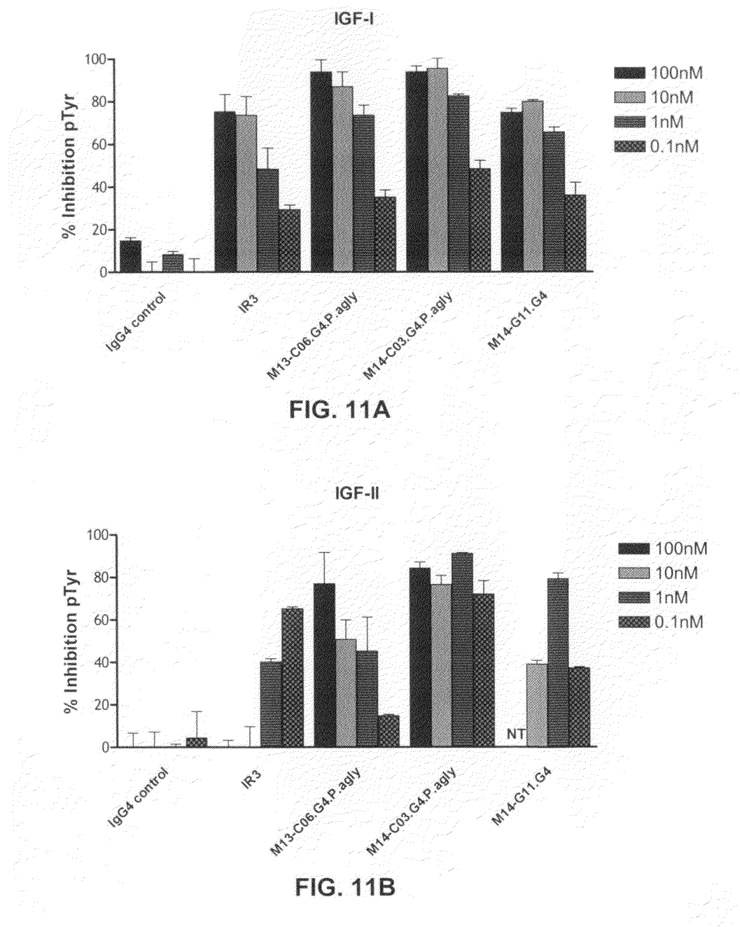

The effect of human anti-IGF-1R antibodies on IGF-1R signaling was evaluated using MCF-7 cells (human breast adenocarcinoma cells). The ability of antibodies to block IGF-1 and IGF-2 mediated IGF-1R receptor phosphorylation was determined as described in Example 4. All of the IgG4 versions of the fully human antibodies showed good inhibition (EC₅₀<1 nM) and inhibited the phosphorylation of IGF-1R (FIG. 11 (A & B).

To detect the effect on downstream signaling, cell lysates were generated as described in Example 4. For signaling experiments control and test antibodies were added after serum starvation at 100 nM, 15 nM, 5 nM and 1 nM in 350 μl of fresh serum free media and incubated for 1 hour at 37° C. Human recombinant IGF-1 at 13 nM or IGF-II at 27 nM (R & D Systems, #291-G1 and #292-G2) was added to wells in 35 μl serum free media and incubated at room temperature for 15 minutes. Cells were lysed and recovered sample separated using a 4-12% Bis-Tris gel and immobilized to nitrocellulose (Invitrogen Corp.). The IGF-1R signaling pathway was detected with phospho-Akt at site Thr308 (Cell signaling Technologies, #4056) and phospho-p44/42 MAPK at site Thr202/Tyr204 (Cell signaling Technologies, #9101) and anti-rabbit IgG-HRP (Cell Signaling Technologies, #7071). Bands were visualized using ECL luminol reagent (Amersham Biosciences, #RPN2109) and autoradiography. Each blot was stripped of antibody and re-probed respectively for total Akt (Cell signaling Technologies, #9272) or total p44/42 MAPK (Cell signaling Technologies, #9102) and anti-rabbit IgG-HRP. Bands visualized using ECL luminol reagent and autoradiography.

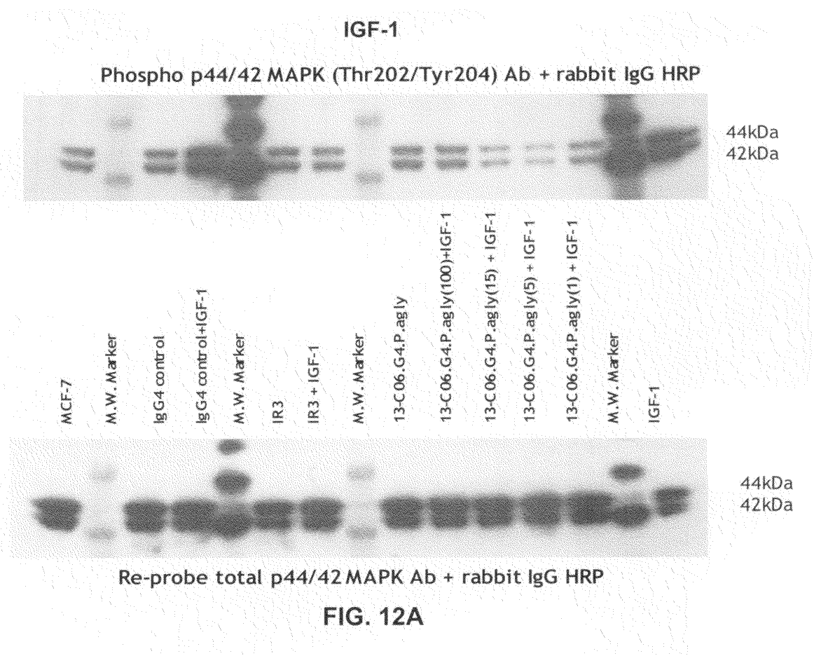

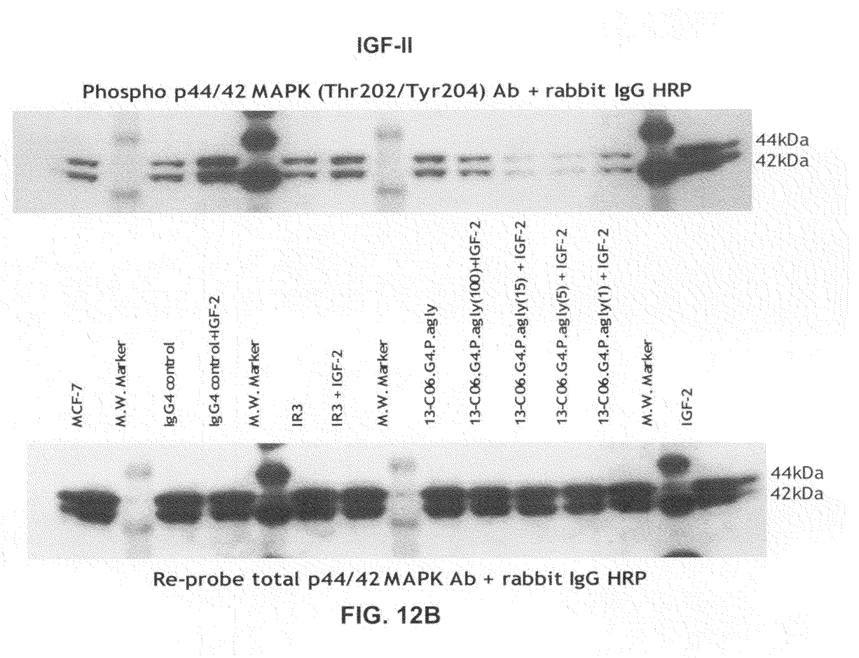

The effect of antibody on down stream signaling events such as Akt and MAPK phosphorylation was determined. Cell lysates from autophosphorylation were immunoprecipitated with polyclonal IGF-1Rβ antibody-agarose conjugate (Santa Cruz Biotechnology, #SC-713). Recovered receptor protein was separated using a 4-12% Tris-Glycine gel and immobilized to nitrocellulose (Invitrogen Corp.). Receptor was detected with anti-phospho-IGF-1R site Tyr1131 (Cell Signaling Technologies, #3021) or anti-IGF-1Rβ (Santa Cruz Biotechnology, #SC-9038) and anti-rabbit IgG-HRP (Cell Signaling Technologies, #7071). Bands were visualized using ECL luminol reagent (Amersham Biosciences, #RPN2109) and autoradiography. (FIGS. 12A and 12B).

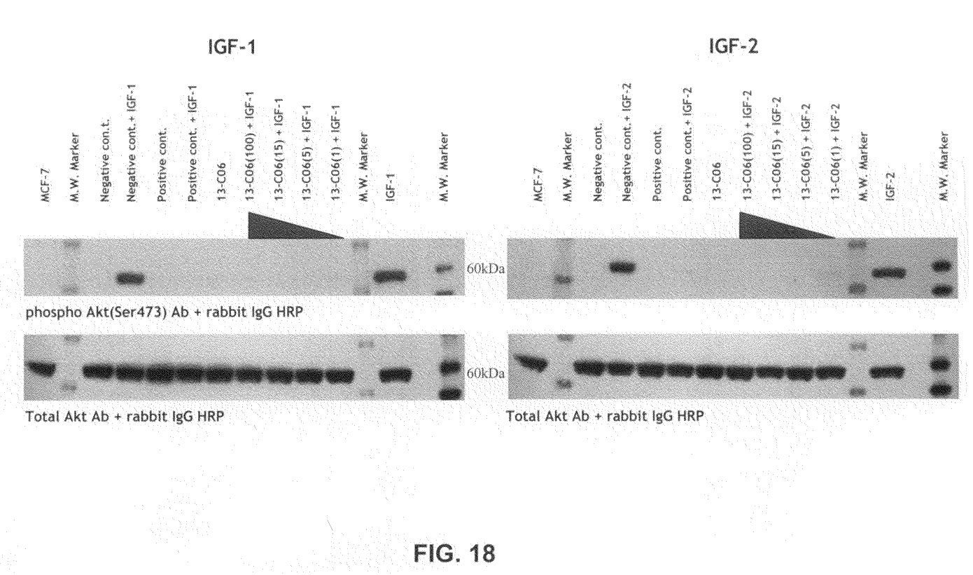

FIGS. 12 A & B show that M13.C06.G4.P.agly inhibited IGF-1 and IGF-2 mediated phosphorylation of Akt and p42/44 MAPK in a dose dependent manner. In particular, the M13-C06.G4.P.agly IGF-1R antibody inhibited ligand induced Akt signaling in MCF7 cells at all concentrations tested (i.e., 1-100 nM), as demonstrated by inhibition of IGF-1 and IGF-2 induced phosphorylation of Akt at amino acid residue Ser473 (FIG. 18). Control antibodies were tested at 100 nM, whereas M13-C06.G4.p.agly was tested at 100, 15, 5 and 1 nM. Antibody IDEC-152, a human G4 version of an antibody of irrelevant specificity, was used as a negative control. Antibody IR3, a murine monoclonal antibody to IGF-1R, was used as a positive control. In addition, M14-C03.G4.P.agly and M14.G11.G4.P full-length antibodies also inhibited IGF-1 and IGF-2 driven signaling of Akt and p42/44 MAPK activation (data not shown).

Part II: Inhibition of Signal Transduction in A549, Calu-6, and H1299 Cells

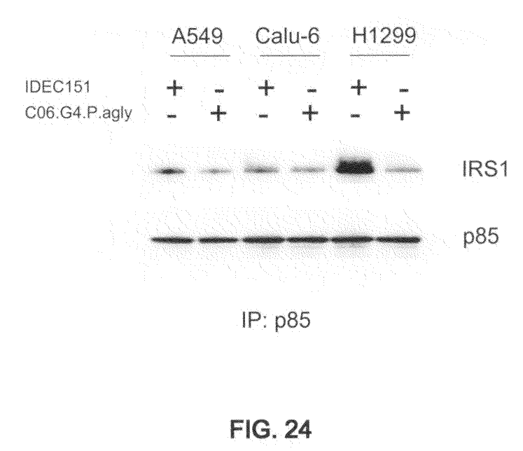

The ability of M13-C06.G4.P.agly to disrupt the association of insulin receptor substrate (IRS-1) with p85 the regulatory subunit of phosphoinositide 3-kinase (PI3K) was determined in tumor cell lines by a co-immunoprecipitation assay. In particular, IRS-1 binds to PI3K subunit p85 in an IGF-1R-dependent manner in NSCLC cell lines sensitive to M13-C06.G4.P.agly antibody. Thus, two non-small cell lung carcinoma cell lines (NSCLC) A549 and H1299 (responsive to M13-C06.G4.P.agly) and one NSCLC cell line, Calu-6 (less responsive to M13-C06.G4.P.agly) were grown in the presence of M13.C06.G4.P.agly or control antibody (IDEC-151) for 24 hours. Cell lysates were immunoprecipitated with an anti-p85 antibody and subjected to western blot analysis with anti-IRS-1 (top blot) and anti-p85 (bottom blot) antibodies (FIG. 24).

For this assay, human lung tumor cell lines A549, Calu-6, and NCI-1299 cells were purchased from ATCC and maintained in RPMI medium 1640 containing 10% fetal bovine serum (FBS). Cells were seeded at 3×10⁶ cells per dish in 100 mm dishes, cultured for 24 hours, and then treated with 100 nM of M13-C06.G4.P.agly or IDEC-151 (human G4.β isotype matched negative control antibody) for 24 hours in the presence of 5% FBS. Cell lysates were prepared in 1% Triton X-100 lysis buffer from Cell Signaling Technology, Inc. (Danvers, Mass. USA)). For immunoprecipitation, anti-p85 antibody (Cat #06-649, Upstate Cell Signaling Solutions (now part of Millipore, Concord, Mass. (USA) was added to the lysate (4 ug of antibody per 1-2 mg of lysate) and incubated at 4° C. overnight. The immunocomplex was then captured by mixing with protein-G agarose beads for 2 hours at 4° C. The immunoprecipitates were washed with ice-cold lysis buffer and boiled in 2×LDS (Lithium Dodecyl Sulfate) sample buffer before separation by NuPAGE® Novex 4-12% Bis-Tris Gel electrophoresis (Invitrogen Corp., Carlsbad, Calif. (USA)), and transfer to nitrocellulose membranes. IRS-1 (Cat # 06-248, Upstate) and p85 (Cat # 06-649, Upstate) antibodies were purchased from Millipore and immunoblotting was performed according to the manufacturer's protocols.

Result: M13-C06.G4.P.agly inhibited the association of IRS-1 with the p85 regulatory subunit of PI3K in the presence of serum in A549 and H1299 cell lines but not in Calu-6 (FIG. 24).

### Example 16

### Antibody Cross-Reactivity to Non-Human Primate IGF-1R

The ability of anti-human IGF-1R antibodies to recognize the IGF-1R from non-human primates was tested. First Rhesus and cynomolgus monkey IGF-1R was cloned and expressed in CHO cells. The binding of all antibodies was determined by flow cytometry and confirmed by confocal microscopy. M13.C06.G4.P.agly, M14.C03.G4.P.agly and M14.G11.G4.P all showed specific binding activity to both Rhesus and cynomolgus IGF-1R (data not shown). Further species cross-reactivity studies showed binding of M14.G11.G4.P and M14.C03.G4.P.agly to murine IGF-1R expressing CHO cells (data not shown).

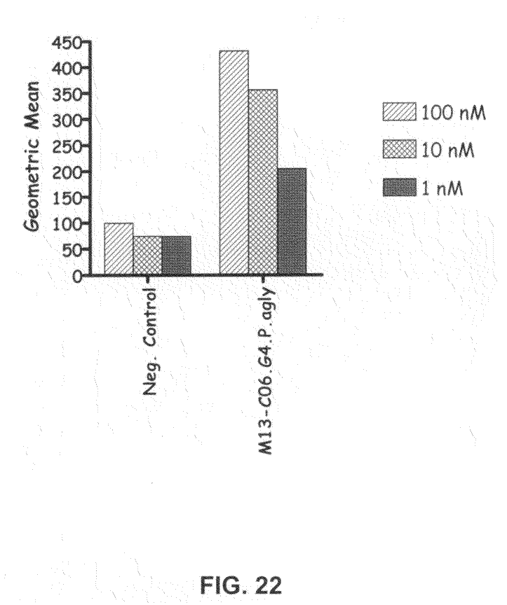

In addition to cynomolgus IGF-1R expressed on CHO cells, the M13.C06.G4.P.agly antibody also cross-reacts with cynomolgus macaque IGF-1R expressed on granulocytes and monocytes from this species. (Specificity of binding was demonstrated by the ability of soluble recombinant human IGF-1R to block M13.C06.G4.P.agly antibody binding (data not shown)). Similarly, the M13.C06.G4.P.agly antibody also binds to an established cynomolgus fibroblast cell line. (See, Example 26, FIG. 22). These results indicate that cynomolgus macaque is an ideal non-rodent species in which toxicity testing has been performed.

In contrast to results with the IGF-1R receptor in primates, M13.C06.G4.P.agly did not show cross-reactivity to rat or mouse IGF-1R expressed on immune cells (granulocytes, monocytes, lymphocytes) as assessed by FACS analysis.

### Example 17

### Generation of IGF-1R Specific Murine Mabs

Murine monoclonal antibodies specific to human IGF-1R were generated by standard hybridoma technology. Splenocytes from Balb/c mice were immunized with IGF-1R expressing NIH-3T3 fibroblast and IGF-1R.Ig fusion protein were used for PEG induced somatic cell fusion. Table 4 summarizes the properties of the anti-IGF-1R murine monoclonal antibodies.

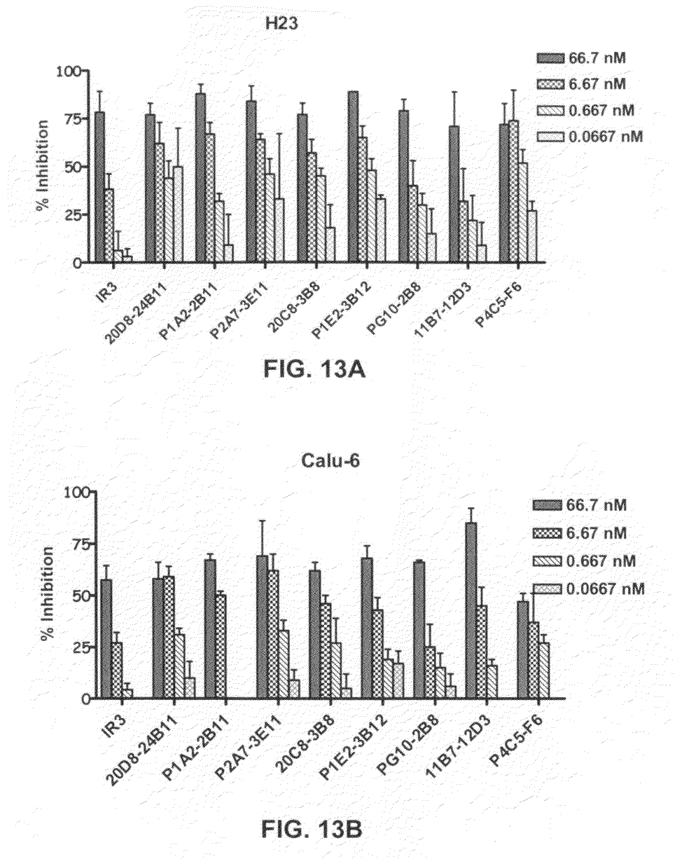

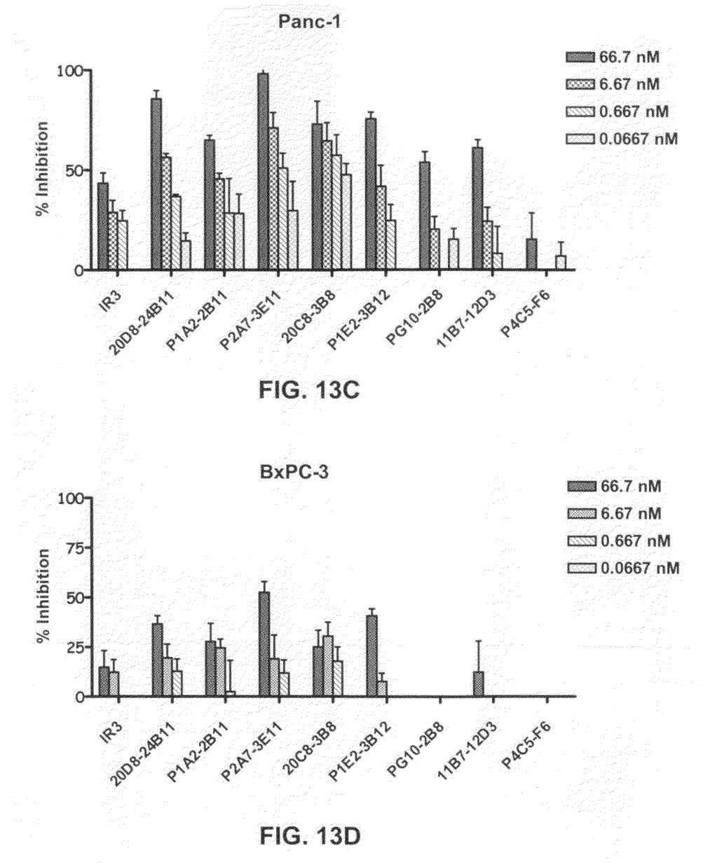

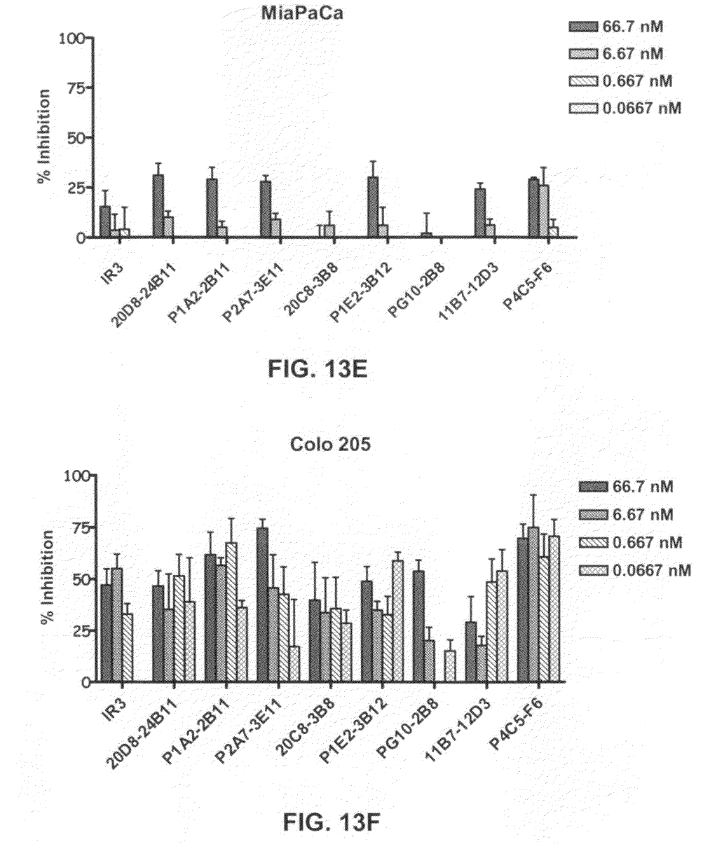

The ability of the selected murine antibodies to inhibit IGF/IGF-1R dependent in vitro growth of several human tumor lines (Lung, H-23, Calu-6; Pancreas, BxPc-3, Panc-1, MiaPaCa and Colon Colo205) was measured by a proliferation assay described in Example 13. FIG. 13 (A)-(F) shows the antibody concentration dependent inhibitory effects of eight of the murine antibodies on tumor cell growth in the presence of IGF-1 at 100 ng/ml.

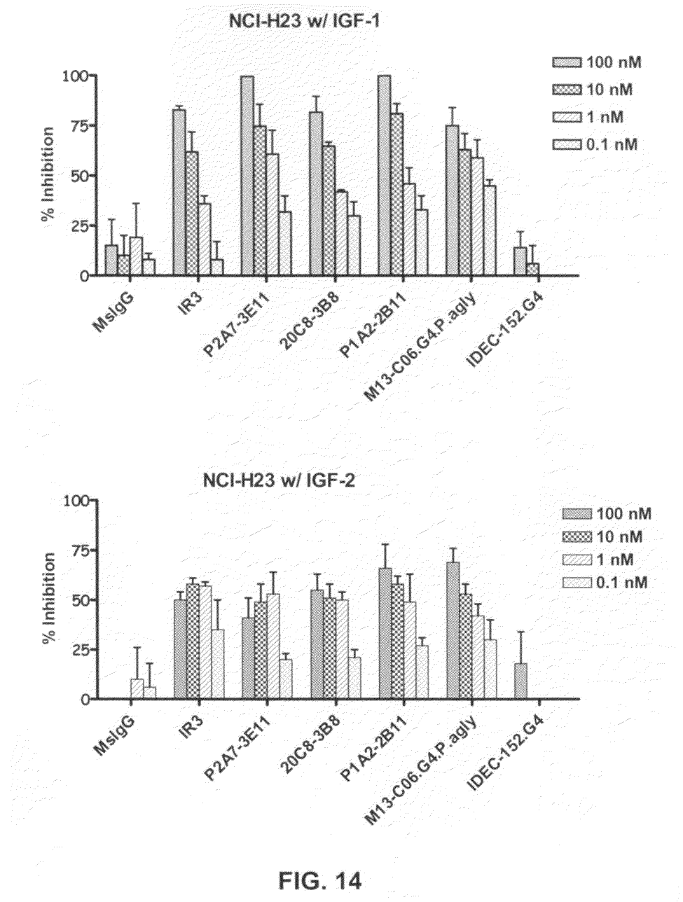

The ability of antibodies to block IGF-1 and IGF-2 driven tumor cell growth was compared using the NCI-H23 lung tumor cell line. FIG. 14 gives an example of the growth inhibitory effects seen with three of the murine MAbs' (P2A7-3E11 (or “P2A7”), 20C8-3E8 (or “20C8”), P1A2-2B11 (or “P1A2”)) and one of the fully human antibody, M13-C06.G4.P.agly. All of the antibodies showed inhibition of IGF-1 and IGF-2 driven tumor growth. A commercially available anti-IGF-1R antibody (IR3) was used as a positive control. The mouse IgG (anti-IDectin, IgG1) and human gamma 4 version of IDEC-152 antibody of irrelevant specificity were used as isotype matched controls for the experiments.

### Example 18

### Cloning of Murine Anti-Human IGF-1R mAbs

### Cloning of Anti-IGF-1R Murine Hybridoma P2A7.3E11 Immunoglobulin Variable Regions

Total cellular RNA from murine hybridoma cells was prepared using a Qiagen RNeasy mini kit following the manufacturer's recommended protocol. cDNAs encoding the variable regions of the heavy and light chains were cloned by RT-PCR from total cellular RNA using the Pharmacia Biotech First Strand cDNA Synthesis kit following the manufacturer's recommended protocol using random hexamers for priming.

The cloning and chimerization of the P2A7.3E11 variable domains will be described in detail as an example (other mAb variable domains were cloned and chimerized by similar methods, but will not be described in detail for the sake of brevity, since standard molecular biology techniques familiar to those skilled in the art of antibody engineering were used). For PCR amplification of the murine immunoglobulin variable domains with intact signal sequences, a cocktail of degenerate forward primers hybridizing to multiple murine immunoglobulin gene family signal sequences and a single back primer specific for 5′ end of the murine constant domain as described in Current Protocols in Immunology (Wiley and Sons, 1999) were used. PCR conditions using Clontech's Advantage Taq polymerase were: initial denaturation for 2 min at 94o, followed by 30 cycles of denature 1 min at 94o, anneal 1 min at 45o, and elongate 1 min at 72o. The P2A7 heavy chain variable domain was amplified with the following primers: 5′ GGG GAT ATC CAC CAT GGR ATG SAG CTG KGT MAT SCT CTT 3′ (M=A/C, K=G/T, R=A/G, and S=C/G) (SEQ ID NO:130) and 5′ AGG TCT AGA AYC TCC ACA CAC AGG RRC CAG TGG ATA GAC 3′ (R=A/G, and Y=C/T). (SEQ ID NO:131) The P2A7 light chain variable domain with its signal sequence was amplified with the following primers: 5′ GGG GAT ATC CAC CAT GGA TTT TCA GGT GCA GAT TTT CAG 3′ (SEQ ID NO:132) and 5′ GCG TCT AGA ACT GGA TGG TGG GAG ATG GA 3′. (SEQ ID NO: 133) The PCR products were gel-purified using a Qiagen Qiaquick gel extraction kit following the manufacturer's recommended protocol. Purified PCR products were subcloned into Invitrogen's pCR2.1TOPO vector using their TOPO cloning kit following the manufacturer's recommended protocol. Inserts from multiple independent subclones were sequenced to guard against PCR errors.

Blast analyses of the variable domain sequences confirmed their immunoglobulin identity. The P2A7 heavy chain variable domain is a member of murine subgroup II(A). The sequence of the P2A7 mature heavy chain variable domain, with its CDRs underlined (with the CDRs, complementarity determining regions, based upon the Kabat designations) is shown below:

```
(SEQ ID NO:38)
    1 QVQLQQSGPE LVKPGASVKM SCKASGNTFT DYVINWVKQR TGQGLEWIGE
  51 IYPGNENTYY NEKFKGKATL TADKSSNTAY MQLSSLTSED SAVYFCARGI
101 YYYGSRTRTM DYWGQGTSVT VSS
```

The P2A7 light chain variable region is a member of murine kappa subgroup IV. The sequence of the P2A7 mature light chain variable domain, with its CDRs underlined, is shown below:

```
(SEQ ID NO:98)
    1 EVVLTQSPTA MAASPGEKIT TTCSASSTLS SNYLHWYQQK PGFSPKLLIY
  51 RTSNLASGVP GRFSGSGSGT SYSLTIGTME AEDVATYYCQ QGSSIPLTFG
101 AGTKLELK
```

Construction and Expression of chP2A7

cDNAs encoding the murine P2A7 variable regions of the heavy and light chains were used to construct vectors for expression of murine-human chimeras (chP2A7) in which the muP2A7 variable regions were linked to human IgG4 and kappa constant regions. For construction of the heavy chain chimera, a 0.47 kb NotI-BsmBI fragment from the P2A7 heavy chain subclone pCN363 and the 1.0 kb BsmBI-NotI fragment from pEAG1995 (a plasmid containing a sequence-confirmed aglycosylated S228P/T299A (Kabat EU nomenclature) variant huIgG4 heavy chain constant domain cDNA with the IgG4 C-terminal lysine residue genetically removed) were subcloned into the phosphatased 6.1 kb NotI-linearized vector backbone of pV90 (a sequence-confirmed pUC-based Biogen Idec proprietary expression vector containing a SV40 early promoter-driven dhfr selectable marker in which heterologous gene expression is controlled by a CMV-IE promoter and a human growth hormone polyadenylation signal). The heavy chain cDNA sequence in the resultant plasmid pEAG2045 was confirmed by DNA sequencing. The sequence of the chimeric P2A7 heavy chain cDNA insert (from the signal sequence's initiator ATG through the terminator TGA) is shown below as SEQ ID NO:134:

```
      1 ATGGAATGGA GCTGTGTCAT GCTCTTCATC CTGTCAGGAA CTGCAGGTGT
    51 CCACTCCCAG GTTCAGCTGC AGCAGTCTGG ACCTGAGCTA GTGAAGCCTG
  101 GGGCTTCAGT GAAGATGTCC TGCAAGGCTT CTGGAAACAC ATTCACTGAC
  151 TATGTTATAA ACTGGGTGAA GCAGAGAACT GGACAGGGCC TTGAGTGGAT
  201 TGGAGAGATT TATCCTGGAA ATGAAAATAC TTATTACAAT GAGAAGTTCA
  251 AGGGCAAGGC CACACTGACT GCAGACAAAT CCTCCAACAC AGCCTACATG
  301 CAGCTCAGTA GCCTGACATC TGAGGACTCT GCGGTCTATT TCTGTGCAAG
  351 AGGGATTTAT TACTACGGTA GTAGGACGAG GACTATGGAC TACTGGGGTC
  401 AAGGAACCTC AGTCACCGTC TCCTCAGCCT CCACCAAGGG CCCATCCGTC
  451 TTCCCCCTGG CGCCCTGCTC CAGATCTACC TCCGAGAGCA CAGCCGCCCT
  501 GGGCTGCCTG GTCAAGGACT ACTTCCCCGA ACCGGTGACG GTGTCGTGGA
  551 ACTCAGGCGC CCTGACCAGC GGCGTGCACA CCTTCCCGGC TGTCCTACAG
  601 TCCTCAGGAC TCTACTCCCT CAGCAGCGTG GTGACCGTGC CCTCCAGCAG
  651 CTTGGGCACG AAGACCTACA CCTGCAACGT AGATCACAAG CCCAGCAACA
  701 CCAAGGTGGA CAAGAGAGTT GAGTCCAAAT ATGGTCCCCC ATGCCCACCG
  751 TGCCCAGCAC CTGAGTTCCT GGGGGGACCA TCAGTCTTCC TGTTCCCCCC
  801 AAAACCCAAG GACACTCTCA TGATCTCCCG GACCCCTGAG GTCACGTGCG
  851 TGGTGGTGGA CGTGAGCCAG GAAGACCCCG AGGTCCAGTT CAACTGGTAC
  901 GTGGATGGCG TGGAGGTGCA TAATGCCAAG ACAAAGCCGC GGGAGGAGCA
  951 GTTCAACAGC GCGTACCGTG TGGTCAGCGT CCTCACCGTC CTGCACCAGG
1001 ACTGGCTGAA CGGCAAGGAG TACAAGTGCA AGGTCTCCAA CAAAGGCCTC
1051 CCGTCCTCCA TCGAGAAAAC CATCTCCAAA GCCAAAGGGC AGCCCCGAGA
1101 GCCACAAGTG TACACCCTGC CCCCATCCCA GGAGGAGATG ACCAAGAACC
1151 AGGTCAGCCT GACCTGCCTG GTCAAAGGCT TCTACCCCAG CGACATCGCC
1201 GTGGAGTGGG AGAGCAATGG GCAGCCGGAG AACAACTACA AGACCACGCC
1251 TCCCGTCCTC GATTCCGACG GCTCCTTCTT CCTCTACAGC AGGCTAACCG
1301 TGGACAAGAG CAGGTGGCAG GAGGGGAATG TCTTCTCATG CTCCGTGATG
1351 CATGAGGCTC TGCACAACCA CTACACACAG AAGAGCCTCT CCCTGTCTCT
1401 GGGTTGA
```

The predicted mature chP2A7 heavy chain protein sequence is shown below as SEQ ID NO:135:

```
    1 QVQLQQSGPE LVKPGASVKM SCKASGNTFT DYVINWVKQR TGQGLEWIGE
  51 IYPGNENTYY NEKFKGKATL TADKSSNTAY MQLSSLTSED SAVYFCARGI
101 YYYGSRTRTM DYWGQGTSVT VSSASTKGPS VFPLAPCSRS TSESTAALGC
151 LVKDYFPEPV TVSWNSGALT SGVHTFPAVL QSSGLYSLSS VVTVPSSSLG
201 TKTYTCNVDH KPSNTKVDKR VESKYGPPCP PCPAPEFLGG PSVFLFPPKP
251 KDTLMTSRTP EVTCVVVDVS QEDPEVQFNW YVDGVEVHNA KTKPREEQFN
301 SAYRVVSVLT VLHQDWLNGK EYKCKVSNKG LPSSTEKTIS KAKGQPREPQ
351 VYTLPPSQEE MTKNQVSLTC LVKGFYPSDT AVEWESNGQP ENNYKTTPPV
401 LDSDGSFFLY SRLTVDKSRW QEGNVFSCSV MHEALHNHYT QKSLSLSLG
```

The murine variable domain is residues 1-122, the human IgG4 heavy chain constant domain is residues 123-459. The Kabat EU-designated S228P hinge substitution (to correct the propensity of IgG4 to form half-antibodies) is residue 231 above, while the T299A substitution in CH2 to genetically remove N-linked glycosylation is residue 302 in the above sequence.

For construction of the light chain chimera, the PCR-amplified P2A7 light chain was subjected to site-directed mutagenesis using a STRATAGENE® Quick-Change mutagenesis kit following the manufacturer's recommended protocol, with the mutagenic primers 5′ CGC CAG TGT GCG GCC GCT GGA ATT CGC CCT TG 3′(SEQ ID NO:136) and its reverse complement, which introduced a unique NotI site 5′ of the heavy chain signal sequence, and 5′ GGA CCA AGC TGG AGC TGA AGC GTA CGG ATG CTG CAC CAA CTG TAT CC 3′ (SEQ ID NO:137) and its reverse complement, which introduced a unique BsiWI site immediately downstream of the light chain variable/kappa constant domain junction. Mutated plasmids were identified by screening for the introduced NotI and BsiWI site changes. The light chain sequence was confirmed by DNA sequencing. The 0.42 kb NotI-BsiWI light chain variable domain fragment produced as described above, and the 0.34 kb BsiWI-NotI fragment from the plasmid pEAG1572, containing a sequence-confirmed humanized anti-LTbR kappa light chain constant domain cDNA were subcloned into the NotI site of the expression vector pEAG1256 (a sequence-confirmed pUC-based expression vector containing a phosphoglycerokinase promoter-driven neo selectable marker in which heterologous gene expression is controlled by a CMV-IE promoter and a human growth hormone polyadenylation signal). The light chain cDNA sequence in the resultant plasmid was confirmed by DNA sequencing. The sequence of the chimeric P2A7 light chain cDNA insert (from the signal sequence's initiator ATG through the terminator TAG) is shown below (SEQ ID NO:138):

```
    1 ATGGATTTTC AGGTGCAGAT TTTCAGCTTG CTGCTAATCA GTGTCACAGT
  51 CATAGTGTCT AATGGAGAAG TTGTGCTCAC CCAGTCTCCA ACCGCCATGG
101 CTGCATCTCC CGGGGAGAAG ATCACTATCA CCTGCAGTGC CAGCTCAACT
151 TTAAGTTCCA ATTACTTGCA TTGGTATCAG CAGAAGCCAG GATTCTCCCC
201 TAAACTCTTG ATTTATAGGA CATCCAATCT GGCCTCTGGA GTCCCAGGTC
251 GCTTCAGTGG CAGTGGGTCT GGGACCTCTT ACTCTCTCAC AATTGGCACC
301 ATGGAGGCTG AAGATGTTGC CACTTACTAC TGCCAGCAGG GTAGTAGTAT
351 ACCGCTCACG TTCGGTGCTG GGACCAAGCT GGAGCTGAAG CGTACGGTGG
401 CTGCACCATC TGTCTTCATC TTCCCGCCAT CTGATGAGCA GTTGAAATCT
451 GGAACTGCCT CTGTTGTGTG CCTGCTGAAT AACTTCTATC CCAGAGAGGC
501 CAAAGTACAG TGGAAGGTGG ATAACGCCCT CCAATCGGGT AACTCCCAGG
551 AGAGTGTCAC AGAGCAGGAC AGCAAGGACA GCACCTACAG CCTCAGCAGC
601 ACCCTGACGC TGAGCAAAGC AGACTACGAG AAACACAAAG TCTACGCCTG
651 CGAAGTCACC CATCAGGGCC TGAGCTCGCC CGTCACAAAG AGCTTCAACA
701 GGGGAGAGTG TTAG
```

The predicted mature chP2A7 light chain protein sequence is shown below (SEQ ID NO:139):

```
    1 EVVLTQSPTA MAASPGEKIT ITCSASSTLS SNYLHWYQQK PGFSPKLLIY
  51 RTSNLASGVP GRFSGSGSGT SYSLTIGTME AEDVATYYCQ QGSSIPLTFG
101 AGTKLELKRT VAAPSVFIFP PSDEQLKSGT ASVVCLLNNF YPREAKVQWK
151 VDNALQSGNS QESVTEQDSK DSTYSLSSTL TLSKADYEKH KVYACEVTHQ
201 GLSSPVTKSF NRGEC
```

The murine variable domain is residues 1-108 above, while the human kappa constant domain is residues 109-215 in the above sequence.

The chP2A7 heavy chain expression vector and the chP2A7 light chain expression vector were co-transfected into 293-EBNA cells and transfected cells were tested for antibody secretion and specificity. Empty vector- and hu5c8-S228P/T299A IgG4 (a molecularly cloned CD40L-specific mAb)-transfected cells served as controls. Western blot analysis (developed with anti-human heavy and light chain antibodies) of conditioned medium indicated that chP2A7-transfected cells synthesized and efficiently secreted heavy and light chains. FACS analysis of IGF-1R-expressing MCF7 human mammary adenocarcinoma cells stained with conditioned medium from transfected cells indicated that the chP2A7 antibody bound and produced staining patterns similar to those of muP2A7, while conditioned medium from mock- and hu5c8-transfected cells failed to stain MCF7 cells (detected with PE-conjugated anti-human heavy and light chain antibodies). Dilution titration indicated that specific staining with the conditioned medium containing chP2A7 mAb demonstrated a dose response. CHO cells were co-transfected with the chP2A7 heavy chain expression vector and the chP2A7 light chain expression vector to generate stable lines expressing chimeric P2A7-aglycosylated huIgG4, kappa mAb.

### Cloning of Anti-IGF-1R Murine Hybridoma 20C8.3B8 Immunoglobulin Variable Regions

Variable domains of other anti-IGF-1R mAbs were cloned and chimerized by standard recombinant DNA techniques similar to those described for the P2A7 mAb.

The predicted mature sequence of the 20C8.3B8 mAb heavy chain variable domain, belonging to murine subgroup I(A), is shown below with its CDRs underlined:

```
(SEQ ID NO:43)
    1 DVQLQESGPD LVKPSQSLSL TCTVTGYSIT SGYSWHWIRQ FPGNKLEWMG
  51 YIHYSGGTNY NPSLKSRISI TRDTSKNQFF LQLNSVTTED TATYYCARSG
101 YGYRSAYYFD YWGQGTTVTV SS
```

The predicted mature sequence of the 20C8 light chain variable domain, belonging to murine kappa subgroup III, is shown below:

```
(SEQ ID NO:103)
    1 DIVLTQSPAS LAVSLGQRAT ISCRASKSVS TSAYSYMHWY QQKPGQPPKL
  51 LIYLASNLES GVPARFSGSG SGTDFTLNIH PVEEEDAATY YCQHSRELPY
101 TFGGGTKLEI K
```

Expression vectors for chimeric 20C8 heavy and light chain cDNAs were constructed as described above. The immunoglobulin cDNA sequence in the plasmids' inserts were confirmed by DNA sequencing. The sequence of the chimeric 20C8 heavy chain cDNA insert (from the signal sequence's initiator ATG through the terminator TGA) is shown below as SEQ ID NO:140:

```
      1 ATGGACTGGA CCTGGAGGGT CTTCTGCTTG CTGGCTGTAG CACCAGGTGC
    51 CCACTCCGAC GTCCAACTGC AGGAGTCTGG ACCTGACCTG GTGAAACCTT
  101 CTCAGTCACT TTCACTCACC TGCACTGTCA CTGGCTACTC CATCACCAGT
  151 GGTTATAGCT GGCACTGGAT CCGGCAGTTT CCAGGAAACA AACTGGAATG
  201 GATGGGCTAC ATACACTACA GTGGTGGCAC TAACTACAAC CCATCTCTCA
  251 AAAGTCGAAT CTCTATCACT CGAGACACAT CCAAGAACCA GTTCTTCCTC
  301 CAGTTGAATT CTGTGACTAC TGAGGACACA GCCACATATT ACTGTGCAAG
  351 ATCGGGGTAC GGCTACAGGA GTGCGTACTA TTTTGACTAC TGGGGCCAAG
  401 GGACCACGGT CACCGTCTCC TCAGCTTCCA CCAAGGGCCC ATCCGTCTTC
  451 CCCCTGGCGC CCTGCTCCAG ATCTACCTCC GAGAGCACAG CCGCCCTGGG
  501 CTGCCTGGTC AAGGACTACT TCCCCGAACC GGTGACGGTG TCGTGGAACT
  551 CAGGCGCCCT GACCAGCGGC GTGCACACCT TCCCGGCTGT CCTACAGTCC
  601 TCAGGACTCT ACTCCCTCAG CAGCGTGGTG ACCGTGCCCT CCAGCAGCTT
  651 GGGCACGAAG ACCTACACCT GCAACGTAGA TCACAAGCCC AGCAACACCA
  701 AGGTGGACAA GAGAGTTGAG TCCAAATATG GTCCCCCATG CCCACCGTGC
  751 CCAGCACCTG AGTTCCTGGG GGGACCATCA GTCTTCCTGT TCCCCCCAAA
  801 ACCCAAGGAC ACTCTCATGA TCTCCCGGAC CCCTGAGGTC ACGTGCGTGG
  851 TGGTGGACGT GAGCCAGGAA GACCCCGAGG TCCAGTTCAA CTGGTACGTG
  901 GATGGCGTGG AGGTGCATAA TGCCAAGACA AAGCCGCGGG AGGAGCAGTT
  951 CAACAGCGCG TACCGTGTGG TCAGCGTCCT CACCGTCCTG CACCAGGACT
1001 GGCTGAACGG CAAGGAGTAC AAGTGCAAGG TCTCCAACAA AGGCCTCCCG
1051 TCCTCCATCG AGAAAACCAT CTCCAAAGCC AAAGGGCAGC CCCGAGAGCC
1101 ACAAGTGTAC ACCCTGCCCC CATCCCAGGA GGAGATGACC AAGAACCAGG
1151 TCAGCCTGAC CTGCCTGGTC AAAGGCTTCT ACCCCAGCGA CATCGCCGTG
1201 GAGTGGGAGA GCAATGGGCA GCCGGAGAAC AACTACAAGA CCACGCCTCC
1251 CGTCCTCGAT TCCGACGGCT CCTTCTTCCT CTACAGCAGG CTAACCGTGG
1301 ACAAGAGCAG GTGGCAGGAG GGGAATGTCT TCTCATGCTC CGTGATGCAT
1351 GAGGCTCTGC ACAACCACTA CACACAGAAG AGCCTCTCCC TGTCTCTGGG
1401 TTGA
```

The predicted mature ch20C8 heavy chain protein sequence is shown below as SEQ ID NO:141:

```
    1 DVQLQESGPD LVKPSQSLSL TCTVTGYSIT SGYSWHWIRQ FPGNKLEWMG
  51 YIHYSGGTNY NPSLKSRISI TRDTSKNQFF LQLNSVTTED TATYYCARSG
101 YGYRSAYYFD YWGQGTTVTV SSASTKGPSV FPLAPCSRST SESTAALGCL
151 VKDYFPEPVT VSWNSGALTS GVHTFPAVLQ SSGLYSLSSV VTVPSSSLGT
201 KTYTCNVDHK PSNTKVDKRV ESKYGPPCPP CPAPEFLGGP SVFLFPPKPK
251 DTLMISRTPE VTCVVVDVSQ EDPEVQFNWY VDGVEVHNAK TKPREEQFNS
301 AYRVVSVLTV LHQDWLNGKE YKCKVSNKGL PSSIEKTISK AKGQPREPQV
351 YTLPPSQEEM TKNQVSLTCL VKGFYPSDIA VEWESNGQPE NNYKTTPPVL
401 DSDGSFFLYS RLTVDKSRWQ EGNVFSCSVM HEALHNHYTQ KSLSLSLG
```

The murine variable domain is residues 1-122, the human IgG4 heavy chain constant domain is residues 123-459.

The sequence of the chimeric 20C8 light chain cDNA insert (from the signal sequence's initiator ATG through the terminator TAG) is shown below as SEQ ID NO:142:

```
    1 ATGGAGACAG ACACACTCCT GTTATGGGTA CTGCTGCTCT GGGTTCCAGG
  51 TTCCACTGGT GACATTGTGC TGACACAGTC TCCTGCTTCC TTAGCTGTAT
101 CTCTGGGGCA GAGGGCCACC ATCTCATGCA GGGCCAGCAA AAGTGTCAGT
151 ACATCTGCCT ATAGTTATAT GCACTGGTAC CAACAGAAAC CAGGACAGCC
201 ACCCAAACTC CTCATCTATC TTGCATCCAA CCTAGAATCT GGGGTCCCTG
251 CCAGGTTCAG TGGCAGTGGG TCTGGGACAG ACTTCACCCT CAACATCCAT
301 CCTGTGGAGG AGGAGGATGC TGCAACCTAT TACTGTCAGC ACAGTAGGGA
351 GCTTCCGTAT ACGTTCGGAG GGGGGACCAA GCTGGAAATC AAACGTACGG
401 TGGCTGCACC ATCTGTCTTC ATCTTCCCGC CATCTGATGA GCAGTTGAAA
451 TCTGGAACTG CCTCTGTTGT GTGCCTGCTG AATAACTTCT ATCCCAGAGA
501 GGCCAAAGTA CAGTGGAAGG TGGATAACGC CCTCCAATCG GGTAACTCCC
551 AGGAGAGTGT CACAGAGCAG GACAGCAAGG ACAGCACCTA CAGCCTCAGC
601 AGCACCCTGA CGCTGAGCAA AGCAGACTAC GAGAAACACA AAGTCTACGC
651 CTGCGAAGTC ACCCATCAGG GCCTGAGCTC GCCCGTCACA AAGAGCTTCA
701 ACAGGGGAGA GTGTTAG
```

The predicted mature ch20C8 light chain protein sequence is shown below as SEQ ID NO:143:

```
    1 DIVLTQSPAS LAVSLGQPAT ISCRASKSVS TSAYSYMHWY QQKPGQPPKL
  51 LIYLASNLES GVPARFSGSG SGTDFTLNIH PVEEEDAATY YCQHSRELPY
101 TFGGGTKLEI KRTVAAPSVF IFPPSDEQLK SGTASVVCLL NNFYPREAKV
151 QWKVDNALQS GNSQESVTEQ DSKDSTYSLS STLTLSKADY EKHKVYACEV
201 THQGLSSPVT KSFNRGEC
```

The murine variable domain is residues 1-111 above, while the human kappa constant domain is residues 112-218 in the above sequence.

The ch20C8 heavy chain expression vector and ch20C8 light chain expression vector were co-transfected into 293-EBNA cells and transfected cells were tested for antibody secretion and specificity. Empty vector- and hu5c8-S228P/T299A IgG4 (a molecularly cloned CD40L-specific mAb)-transfected cells served as controls. Western blot analysis (developed with anti-human heavy and light chain antibodies) of conditioned medium indicated that ch20C8-transfected cells synthesized and efficiently secreted heavy and light chains. FACS analysis of IGF-1R-expressing MCF7 human mammary adenocarcinoma cells stained with conditioned medium from transfected cells indicated that the ch20C8 antibody bound with a titratable dose response, while conditioned medium from mock- and hu5c8-transfected cells failed to stain MCF7 cells (detected with PE-conjugated anti-human heavy and light chain antibodies). CHO cells were co-transfected with the ch20C8 heavy chain expression vector and ch20C8 light chain expression vector to generate stable lines expressing chimeric 20C8-aglycosylated huIgG4, kappa mAb.

### Cloning of Anti-IGF-1R mAb 20D8.24B11 Immunoglobulin Variable Regions

The mAb 20D8.24B11 appears to be a sister clone of 20C8.3B8 (both were derived from fusion 7): sharing a common light chain and having a heavy chain that differs from that of 20C8 by a single residue in FR4. The predicted mature sequence of the 20D8.24B11 mAb heavy chain variable domain, belonging to murine subgroup I(A), is shown below with its CDRs underlined:

```
(SEQ ID NO:53)
    1 DVQLQESGPD LVKPSQSLSL TCTVTGYSIT SGYSWHWIRQ FPGNKLEWMG
  51 YIHYSGGTNY NPSLKSRISI TRDTSKNQFF LQLNSVTTED TATYYCARSG
101 YGYRSAYYFD YWGQGTTLTV SS
```

An alignment of the 20D8 (upper) and 20C8 (lower) heavy chain variable domains, highlighting the single conservative difference corresponding to FR4 Kabat residue 109 (residue 118 below) is shown below:

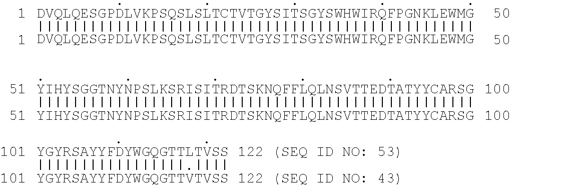

An expression vector for chimeric 20D8 heavy chain cDNA was constructed and the heavy chain cDNA insert in plasmid pCN380 was confirmed by DNA sequencing. The sequence of the chimeric 20D8 heavy chain cDNA insert (from the signal sequence's initiator ATG through the terminator TGA) is shown below as SEQ ID NO:144:

```
      1 ATGGACTGGA CCTGGAGGGT CTTCTGCTTG CTGGCTGTAG CACCAGGTGC
    51 CCACTCCGAC GTCCAACTGC AGGAGTCTGG ACCTGACCTG GTGAAACCTT
  101 CTCAGTCACT TTCACTCACC TGCACTGTCA CTGGCTACTC CATCACCAGT
  151 GGTTATAGCT GGCACTGGAT CCGGCAGTTT CCAGGAAACA AACTGGAATG
  201 GATGGGCTAC ATACACTACA GTGGTGGCAC TAACTACAAC CCATCTCTCA
  251 AAAGTCGAAT CTCTATCACT CGAGACACAT CCAAGAACCA GTTCTTCCTC
  301 CAGTTGAATT CTGTGACTAC TGAGGACACA GCCACATATT ACTGTGCAAG
  351 ATCGGGGTAC GGCTACAGGA GTGCGTACTA TTTTGACTAC TGGGGCCAAG
  401 GGACCACGTT GACAGTCTCC TCAGCTTCCA CCAAGGGCCC ATCCGTCTTC
  451 CCCCTGGCGC CCTGCTCCAG ATCTACCTCC GAGAGCACAG CCGCCCTGGG
  501 CTGCCTGGTC AAGGACTACT TCCCCGAACC GGTGACGGTG TCGTGGAACT
  551 CAGGCGCCCT GACCAGCGGC GTGCACACCT TCCCGGCTGT CCTACAGTCC
  601 TCAGGACTCT ACTCCCTCAG CAGCGTGGTG ACCGTGCCCT CCAGCAGCTT
  651 GGGCACGAAG ACCTACACCT GCAACGTAGA TCACAAGCCC AGCAACACCA
  701 AGGTGGACAA GAGAGTTGAG TCCAAATATG GTCCCCCATG CCCACCGTGC
  751 CCAGCACCTG AGTTCCTGGG GGGACCATCA GTCTTCCTGT TCCCCCCAAA
  801 ACCCAAGGAC ACTCTCATGA TCTCCCGGAC CCCTGAGGTC ACGTGCGTGG
  851 TGGTGGACGT GAGCCAGGAA GACCCCGAGG TCCAGTTCAA CTGGTACGTG
  901 GATGGCGTGG AGGTGCATAA TGCCAAGACA AAGCCGCGGG AGGAGCAGTT
  951 CAACAGCGCG TACCGTGTGG TCAGCGTCCT CACCGTCCTG CACCAGGACT
1001 GGCTGAACGG CAAGGAGTAC AAGTGCAAGG TCTCCAACAA AGGCCTCCCG
1051 TCCTCCATCG AGAAAACCAT CTCCAAAGCC AAAGGGCAGC CCCGAGAGCC
1101 ACAAGTGTAC ACCCTGCCCC CATCCCAGGA GGAGATGACC AAGAACCAGG
1151 TCAGCCTGAC CTGCCTGGTC AAAGGCTTCT ACCCCAGCGA CATCGCCGTG
1201 GAGTGGGAGA GCAATGGGCA GCCGGAGAAC AACTACAAGA CCACGCCTCC
1251 CGTCCTCGAT TCCGACGGCT CCTTCTTCCT CTACAGCAGG CTAACCGTGG
1301 ACAAGAGCAG GTGGCAGGAG GGGAATGTCT TCTCATGCTC CGTGATGCAT
1351 GAGGCTCTGC ACAACCACTA CACACAGAAG AGCCTCTCCC TGTCTCTGGG
1401 TTGA
```

The predicted mature ch20D8 heavy chain protein sequence encoded by the above sequence is shown below as SEQ ID NO: 145:

```
    1 DVQLQESGPD LVKPSQSLSL TCTVTGYSIT SGYSWHWIRQ FPGNKLEWMG
  51 YIHYSGGTNY NPSLKSRISI TRDTSKNQFF LQLNSVTTED TATYYCARSG
101 YGYRSAYYFD YWGQGTTLTV SSASTKGPSV FPLAPCSRST SESTAALGCL
151 VKDYFPEPVT VSWNSGALTS GVHTFPAVLQ SSGLYSLSSV VTVPSSSLGT
201 KTYTCNVDHK PSNTKVDKRV ESKYGPPCPP CPAPEFLGGP SVFLFPPKPK
251 DTLMISRTPE VTCVVVDVSQ EDPEVQFNWY VDGVEVHNAK TKPREEQFNS
301 AYRVVSVLTV LHQDWLNGKE YKCKVSNKGL PSSIEKTISK AKGQPREPQV
351 YTLPPSQEEM TKNQVSLTCL VKGFYPSDIA VEWESNGQPE NNYKTTPPVL
401 DSDGSFFLYS RLTVDKSRWQ EGNVFSCSVM HEALHNHYTQ KSLSLSLG
```

The murine variable domain is residues 1-122, the human S228P/T299A IgG4 heavy chain constant domain is residues 123-458.

The 20D8 light chain variable sequence is identical to that of 20C8: please see the information previously described for 20C8.

### Cloning of Anti-IGF-1R mAb P1G10.2B8 Immunoglobulin Variable Regions

The predicted sequence of the mature P1G10 heavy chain variable domain is shown below as SEQ ID NO:58, with its CDRs underlined:

```
    1 QIQLVQSGPD LKKPGETVKI SCKASGYTFT NHGMNWVKQA PGKDLKWMGW
  51 INTNTGEPTY ADDFKGRFAF SLETSASTAY LQINNLKNED TATYFCASPL
101 YYRNGRYFDV WGAGTTVTVS S
```

P1G10 appears to belong to the murine heavy chain variable domain subgroup II(A), but with only 55% identity to the heavy II(A) consensus sequence.

An expression vector for the chimeric P1G10 heavy chain cDNA was constructed and its cDNA insert was sequence confirmed. The sequence of the chimeric P1G10 heavy chain cDNA insert (from the signal sequence's initiator ATG through the terminator TGA is shown below as SEQ ID NO:146:

```
      1 ATGGGTTGGA TCTGTATCTT TCTATTCTTG GTGGCAGCTG CCCAAAGTGC
    51 CCAAGCACAG ATCCAGTTGG TGCAGTCTGG ACCTGACCTG AAGAAGCCTG
  101 GAGAGACAGT CAAGATCTCC TGCAAGGCTT CTGGGTATAC CTTCACAAAC
  151 CATGGAATGA ACTGGGTGAA GCAGGCTCCA GGAAAGGATT TAAAGTGGAT
  201 GGGCTGGATA AACACCAACA CTGGAGAGCC AACATATGCT GATGACTTCA
  251 AGGGACGGTT TGCCTTCTCT TTGGAAACCT CTGCCAGCAC TGCCTATTTG
  301 CAGATCAACA ACCTCAAAAA TGAGGACACG GCTACATATT TCTGTGCAAG
  351 TCCCCTCTAC TATAGGAACG GGCGATACTT CGATGTCTGG GGCGCAGGGA
  401 CCACGGTCAC CGTCTCCTCA GCTTCCACCA AGGGCCCATC CGTCTTCCCC
  451 CTGGCGCCCT GCTCCAGATC TACCTCCGAG AGCACAGCCG CCCTGGGCTG
  501 CCTGGTCAAG GACTACTTCC CCGAACCGGT GACGGTGTCG TGGAACTCAG
  551 GCGCCCTGAC CAGCGGCGTG CACACCTTCC CGGCTGTCCT ACAGTCCTCA
  601 GGACTCTACT CCCTCAGCAG CGTGGTGACC GTGCCCTCCA GCAGCTTGGG
  651 CACGAAGACC TACACCTGCA ACGTAGATCA CAAGCCCAGC AACACCAAGG
  701 TGGACAAGAG AGTTGAGTCC AAATATGGTC CCCCATGCCC ACCGTGCCCA
  751 GCACCTGAGT TCCTGGGGGG ACCATCAGTC TTCCTGTTCC CCCCAAAACC
  801 CAAGGACACT CTCATGATCT CCCGGACCCC TGAGGTCACG TGCGTGGTGG
  851 TGGACGTGAG CCAGGAAGAC CCCGAGGTCC AGTTCAACTG GTACGTGGAT
  901 GGCGTGGAGG TGCATAATGC CAAGACAAAG CCGCGGGAGG AGCAGTTCAA
  951 CAGCGCGTAC CGTGTGGTCA GCGTCCTCAC CGTCCTGCAC CAGGACTGGC
1001 TGAACGGCAA GGAGTACAAG TGCAAGGTCT CCAACAAAGG CCTCCCGTCC
1051 TCCATCGAGA AAACCATCTC CAAAGCCAAA GGGCAGCCCC GAGAGCCACA
1101 AGTGTACACC CTGCCCCCAT CCCAGGAGGA GATGACCAAG AACCAGGTCA
1151 GCCTGACCTG CCTGGTCAAA GGCTTCTACC CCAGCGACAT CGCCGTGGAG
1201 TGGGAGAGCA ATGGGCAGCC GGAGAACAAC TACAAGACCA CGCCTCCCGT
1251 CCTCGATTCC GACGGCTCCT TCTTCCTCTA CAGCAGGCTA ACCGTGGACA
1301 AGAGCAGGTG GCAGGAGGGG AATGTCTTCT CATGCTCCGT GATGCATGAG
1351 GCTCTGCACA ACCACTACAC ACAGAAGAGC CTCTCCCTGT CTCTGGGTTG
1401 A
```

The predicted mature chP1G10 heavy chain protein sequence encoded the sequence above is shown below as SEQ ID NO:147:

```
    1 QIQLVQSGPD LKKPGETVKI SCKASGYTFT NHGMNWVKQA PGKDLKWMGW
  51 INTNTGEPTY ADDFKGRFAF SLETSASTAY LQINNLKNED TATYFCASPL
101 YYRNGRYFDV WGAGTTVTVS SASTKGPSVF PLAPCSRSTS ESTAALGCLV
151 KDYFPEPVTV SWNSGALTSG VHTFPAVLQS SGLYSLSSVV TVPSSSLGTK
201 TYTCNVDHKP SNTKVDKRVE SKYGPPCPPC PAPEFLGGPS VFLFPPKPKD
251 TLMISRTPEV TCVVVDVSQE DPEVQFNWYV DGVEVHNAKT KPREEQFNSA
301 YRVVSVLTVL HQDWLNGKEY KCKVSNKGLP SSIEKTISKA KGQPREPQVY
351 TLPPSQEEMT KNQVSLTCLV KGFYPSDIAV EWESNGQPEN NYKTTPPVLD
401 SDGSFFLYSR LTVDKSRWQE GNVFSCSVMH EALHNHYTQK SLSLSLG
```

The murine variable domain is residues 1-121, the human S228P/T299A IgG4 heavy chain constant domain is residues 122-457.

The predicted sequence of the mature P1G10 light chain variable domain, belonging to murine kappa subgroup V, is shown below as SEQ ID NO:113, with its CDRs underlined:

```
    1 DIQMTQTTSS LSASLGDRVT ISCRASQDIS NYLNWYQQKP DGSVKLLIYY
  51 TSRLHSGVPS RFSGSGSGTD YSLTISNLEQ EDIATYFCQQ GKTLPWTFGG
101 GTKLEIK
```

An expression vector for the chimeric P1G10 light chain cDNA was constructed and its cDNA insert was sequence confirmed. The sequence of the chimeric P1G10 light chain cDNA insert (from the signal sequence's initiator ATG through the terminator TAG) is shown below as SEQ ID NO:148:

```
    1 ATGAGGTCCC CTGCTCAGTT TCTTGGTCTC CTGTTGCTCT GTTTTCAAGG
  51 TGCCAGATGT GATATCCAGA TGACACAGAC TACATCCTCC CTGTCTGCCT
101 CTCTGGGAGA CAGAGTCACC ATCAGTTGCA GGGCAAGTCA GGACATTAGT
151 AATTATTTAA ATTGGTATCA GCAGAAACCA GATGGATCTG TTAAACTCCT
201 GATCTACTAC ACATCAAGAT TACACTCAGG AGTCCCATCA AGGTTCAGTG
251 GCAGTGGGTC TGGAACAGAT TATTCTCTCA CCATTAGCAA CCTGGAACAA
301 GAAGATATTG CCACTTACTT TTGCCAACAG GGAAAGACGC TTCCGTGGAC
351 GTTCGGTGGA GGCACCAAGC TGGAAATCAA ACGTACGGTG GCTGCACCAT
401 CTGTCTTCAT CTTCCCGCCA TCTGATGAGC AGTTGAAATC TGGAACTGCC
451 TCTGTTGTGT GCCTGCTGAA TAACTTCTAT CCCAGAGAGG CCAAAGTACA
501 GTGGAAGGTG GATAACGCCC TCCAATCGGG TAACTCCCAG GAGAGTGTCA
551 CAGAGCAGGA CAGCAAGGAC AGCACCTACA GCCTCAGCAG CACCCTGACG
601 CTGAGCAAAG CAGACTACGA GAAACACAAA GTCTACGCCT GCGAAGTCAC
651 CCATCAGGGC CTGAGCTCGC CCGTCACAAA GAGCTTCAAC AGGGGAGAGT
701 GTTAG
```

The predicted mature chP1G10 light chain protein sequence encoded by the sequence above is shown below as SEQ ID NO:149:

```
    1 DIQMTQTTSS LSASLGDRVT ISCPASQDIS NYLNWYQQKP DGSVKLLIYY
  51 TSRLHSGVPS RFSGSGSGTD YSLTISNLEQ EDIATYFCQQ GKTLPWTFGG
101 GTKLEIKRTV AAPSVFIFPP SDEQLKSGTA SVVCLLNNFY PREAKVQWKV
151 DNALQSGNSQ ESVTEQDSKD STYSLSSTLT LSKADYEKHK VYACEVTHQG
201 LSSPVTKSFN RGEC
```

The murine variable domain is residues 1-107 above, while the human kappa constant domain is residues 108-214 in the above sequence.

The chP1G10 heavy chain expression vector and chP1G10 light chain expression vector were co-transfected into 293-EBNA cells and transfected cells were tested for antibody secretion and specificity (empty vector- and hu5c8-S228P/T299A IgG4 (a molecularly cloned CD40L-specific mAb)-transfected cells served as controls). Western blot analysis (developed with anti-human heavy and light chain antibodies) of conditioned medium indicated that chP1G10-transfected cells synthesized and efficiently secreted heavy and light chains. FACS analysis of IGF-1R-expressing MCF7 human mammary adenocarcinoma cells stained with conditioned medium from transfected cells indicated that the chP1G10 antibody bound with a titratable dose response, while conditioned medium from mock- and hu5c8-transfected cells failed to stain MCF7 cells (detected with PE-conjugated anti-human heavy and light chain antibodies). CHO cells were co-transfected with the chP1G10 heavy chain expression vector and chP1G10 light chain expression vector to generate stable lines expressing chimeric P1G10-aglycosylated huIgG4, kappa mAb.

Cloning of Anti-IGF-1R mAb P1A2.2B11 Immunoglobulin Variable Regions

The predicted sequence of the mature P1A2 heavy chain variable domain, belonging to murine subgroup II(A) is shown below as SEQ ID NO:48:

```
    1 QIQLVQSGPE LKKPGETVKI SCKASGYTFT NHGMNWVKQA PGKGLKWMGW
  51 NTSTGEPTYA DDFKGRFAFS LETSASTAFL QINNLKNEDT ASYFCASPLY
101 YMYGRYIDVW GAGTAVTVSS
```

The P1A2 heavy chain is 92.6% identical to that of P1G10 (both were derived from fusion 5), with one FR1, one FR2, two CDR2, two FR3, two CDR3, and 1 FR4 differences. The alignment of the P1A2 (upper line) and P1G10 (lower line) heavy chain variable domains is shown below:

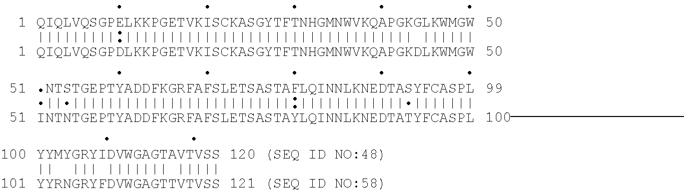

An expression vector for the chimeric P1A2 heavy chain is constructed by the methods described above. The predicted sequence of the chP1A2 heavy chain encoded by that plasmid (SEQ ID NO:150) is:

```
    1 QIQLVQSGPE LKKPGETVKI SCKASGYTFT NHGMNWVKQA PGKGLKWMGW
  51 NTSTGEPTYA DDFKGRFAFS LETSASTAFL QINNLKNEDT ASYFCASPLY
101 YMYGRYIDVW GAGTAVTVSS ASTKGPSVFP LAPCSRSTSE STAALGCLVK
151 DYFPEPVTVS WNSGALTSGV HTFPAVLQSS GLYSLSSVVT VPSSSLGTKT
201 YTCNVDHKPS NTKVDKRVES KYGPPCPPCP APEFLGGPSV FLFPPKPKDT
251 LMISRTPEVT CVVVDVSQED PEVQFNWYVD GVEVHNAKTK PREEQFNSAY
301 RVVSVLTVLH QDWLNGKEYK CKVSNKGLPS SIEKTISKAK GQPREPQVYT
351 LPPSQEEMTK NQVSLTCLVK GFYPSDIAVE WESNGQPENN YKTTPPVLDS
401 DGSFFLYSRL TVDKSRWQEG NVFSCSVMHE ALHNHYTQKS LSLSLG
```

The murine variable domain is residues 1-120, the human S228P/T299A IgG4 heavy chain constant domain is residues 121-456.

The predicted sequence of the mature P1A2 light chain variable domain, belonging to murine kappa subgroup V, is shown below as SEQ ID NO: 108, with its CDRs underlined:

```
    1 DIQMTQTTSS LSASLGDRVT ISCRASQDIS NYLNWYQQKP DGTIKLLIYY
  51 TSRLHSGVPS RFSGSGSGTD YSLTISNLEQ EDFATYFCQQ GKTLPWTFGG
101 GTKLEIK
```

The P1A2 light chain is 97.2% identical to that of P1G10 (both were derived from fusion 5), with two FR2 and one FR3 difference, but sharing identical CDRs. The alignment of the P1A2 (upper line) and P1G10 (lower line) light chain variable domains is shown below:

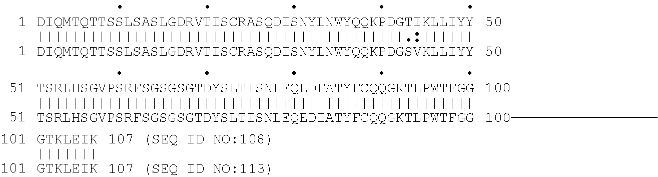

An expression vector for the chimeric P1A2 light chain cDNA was constructed and its cDNA insert was sequence confirmed. The sequence of the chimeric P1A2 light chain cDNA insert (from the signal sequence's initiator ATG through the terminator TAG) is shown below as SEQ ID NO:151:

```
    1 ATGAGGTCCC CTGCTCAGTT TCTTGGAGAC CTGTTGCTCT GTTTTCAAGG
  51 TACCAGATGT GATATCCAGA TGACACAGAC TACATCCTCC CTATCTGCCT
101 CTCTGGGAGA CAGAGTCACC ATCAGTTGCA GGGCAAGTCA GGACATTAGC
151 AATTATTTAA ACTGGTATCA GCAGAAACCA GATGGAACTA TTAAACTCCT
201 GATCTACTAC ACATCAAGAT TACACTCAGG AGTCCCATCA AGGTTCAGTG
251 GCAGTGGGTC TGGAACAGAT TATTCTCTCA CCATTAGCAA CCTGGAACAA
301 GAAGATTTTG CCACTTACTT TTGCCAACAG GGTAAAACGC TTCCGTGGAC
351 GTTCGGTGGA GGCACCAAGC TGGAAATCAA ACGTACGGTG GCTGCACCAT
401 CTGTCTTCAT CTTCCCGCCA TCTGATGAGC AGTTGAAATC TGGAACTGCC
451 TCTGTTGTGT GCCTGCTGAA TAACTTCTAT CCCAGAGAGG CCAAAGTACA
501 GTGGAAGGTG GATAACGCCC TCCAATCGGG TAACTCCCAG GAGAGTGTCA
551 CAGAGCAGGA CAGCAAGGAC AGCACCTACA GCCTCAGCAG CACCCTGACG
601 CTGAGCAAAG CAGACTACGA GAAACACAAA GTCTACGCCT GCGAAGTCAC
651 CCATCAGGGC CTGAGCTCGC CCGTCACAAA GAGCTTCAAC AGGGGAGAGT
701 GTTAG
```

The predicted mature chP1A2 light chain protein sequence encoded by pCN379 is shown below as SEQ ID NO:152:

```
    1 DIQMTQTTSS LSASLGDRVT ISCRASQDIS NYLNWYQQKP DGTIKLLIYY
  51 TSRLHSGVPS RFSGSGSGTD YSLTISNLEQ EDFATYFCQQ GKTLPWTFGG
101 GTKLEIKRTV AAPSVFIFPP SDEQLKSGTA SVVCLLNNFY PREAKVQWKV
151 DNALQSGNSQ ESVTEQDSKD STYSLSSTLT LSKADYEKHK VYACEVTHQG
201 LSSPVTKSFN RGEC
```

The murine variable domain is residues 1-107 above, while the human kappa constant domain is residues 108-214 in the above sequence.

### Cloning of Anti-IGF-1R mAb P1E2.3B12 Immunoglobulin Variable Regions

Cloning of the P1E2 variable domains is carried out by the methods described above. Hence, antibody “P1E2” was developed as a chimeric antibody that contains mouse VH and VL derived from the antibody expressed by the P1E2.3B12 hybridoma cell line (see Table 4), fused to human IgG4Pagly/kappa constant domains.

### Example 19

### IGF-1R Fab Antibodies Bind Soluble IGF-1R with High Affinity

Method: The binding activity of M13-C06, M14-C03, and M14-G11 Fabs to soluble IGF-1R was measured using surface plasmon resonance. Biotinylated PENTA-His Antibody (Qiagen, Inc.) was immobilized onto a Streptavidin coated Sensor Chip. Soluble/Dimeric IGF-1R-His ectodomain (R&D systems, Inc.) was captured on the surface via the PENTA-His antibody. Secondary injections of M13-C06, M14-C03, or M14-G11 Fabs (0.5 nM-1000 nM) were performed. The surfaces were regenerated with three short injections of acetate, pH 4.0.

Results: The M13-C06 Fab bound recombinant IGF-1R with the highest affinity at KD=1.3 nM, whereas M14-G11 Fab bound with a KD=4.0 nM, and M14-C03 Fab bound with a KD=4.9 nM (data not shown).

### Example 20

### Inhibition of IGF-1 and IGF-2 Stimulated Tumor Cell Growth by Fully Human IGF-1R Antibodies

Methods: The effect of antibody on tumor growth in vitro was measured using a CELL TITER-GLO™ assay (Promega Corporation, 2800 Woods Hollow Rd., Madison, Wis. 53711 USA). BxPC3 cells in 10% FBS containing RPMI medium were cultured in Wallac 96-well clear bottom TC-treated plates (8000 cell/well). After 24 hours, culture medium was changed to serum free condition and antibodies at different concentrations (100 nM, 10 nM, 1 nM, and 0.1 nM) were added. Following 30 minute incubation, IGF-1 or IGF-2 was added at 100 ng/ml. The cells were incubated for another 48 hours until lysed to determine the amount of ATP present using the CELL TITER-GLO™ reagent. Inhibition was calculated as [1-(Ab-SFM)/(IGF −SFM)]×100%. An isotype matched antibody, IDEC-151 (human G4), antibody was used as a negative control.

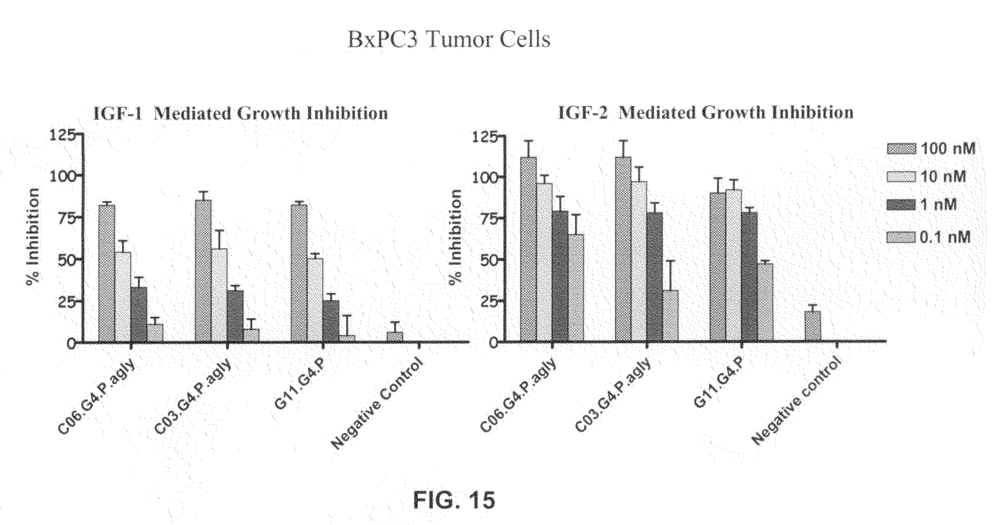

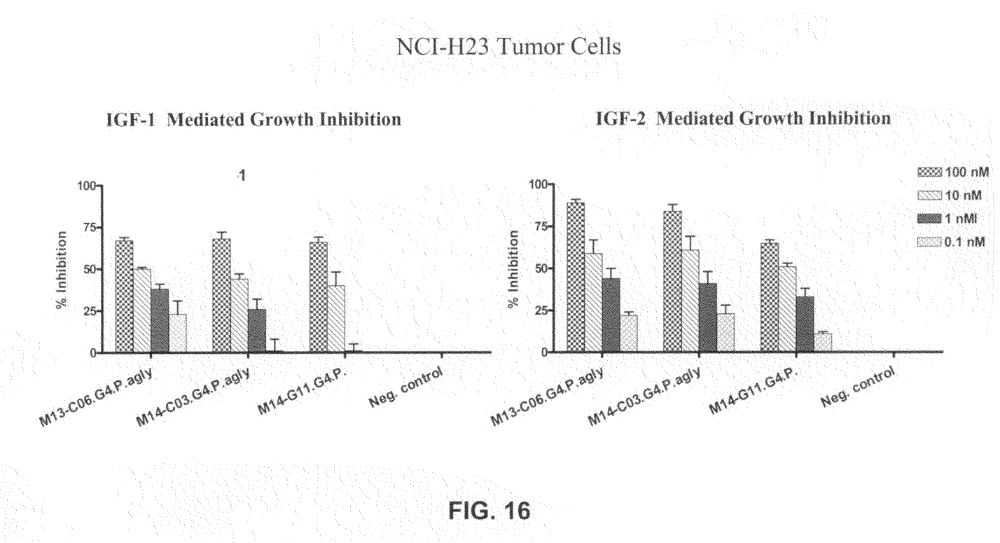

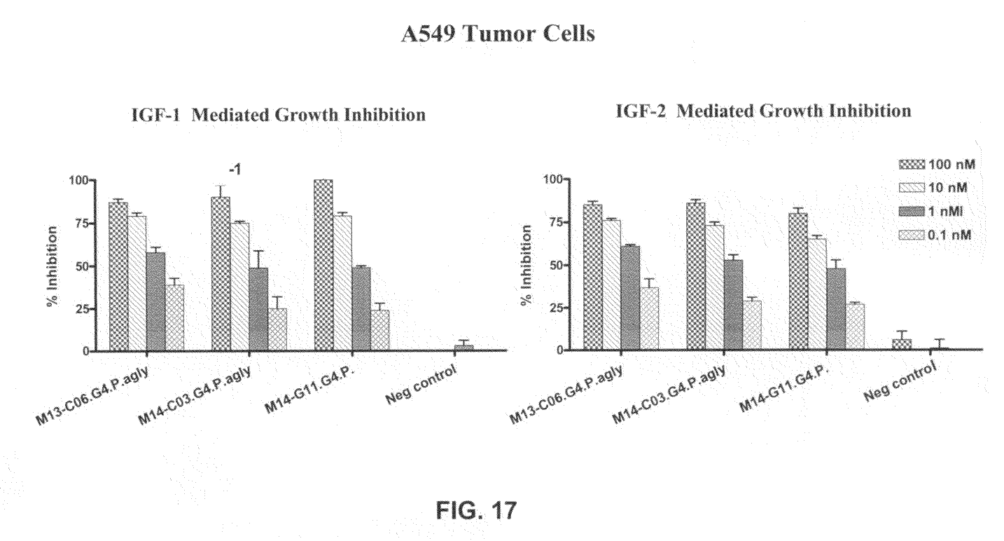

Results: Fully human antibodies M13-C06.G4.P.agly, M14-G11.G4.P and M14-C03.G4.P.agly inhibited BxPC3 (human pancreas adenocarcinoma) cell proliferation driven with recombinant human IGF-1 and IGF-2 (FIG. 15). Similar growth inhibition results were obtained with these antibodies against cell proliferation driven with recombinant human IGF-1 and IGF-2 in human lung cancer cell line NCI-H23 (FIG. 16; M13-C06.G4.P.agly antibody) and human lung cancer cell line A549 (FIG. 17; M13-C06.G4.P.agly antibody). In all three cell lines M14-G11.G4.P.agly showed similar results as M14.G11.G4.P version (data not shown).

### Example 21

### Cell-Cycle Arrest of Tumor Cell Growth In Vitro by Fully Human IGF-1R Antibodies

Methods: The ability of fully human IGF-1R antibodies to arrest cell cycle progression was assessed by FACS analysis; monitoring incorporation of propidium iodide in cultured BxPC3 cells. BxPC3 cells (4×10⁵ cells/well) were plated into 6 well plates. After 24 hours, cells were changed to serum-free media (SFM) for the following 24 hours. Next the IGF-1R antibodies at a final concentration of 133.3 nM (20 micrograms/ml) and IGF-1 at 200 ng/ml was added to the media. After 24 hours, the cells were trypsinized and fixed with ethanol. DNA content was stained with propidium iodide (PI) prior to FACS analysis. An isotype matched antibody, IDEC-151 (human G4), was used as a negative control.

Results: Fully human antibodies M13-C06.G4.P.agly (Table 11), M14-G11.G4.P.agly and M14-C03.G4.P.agly arrested the BxPC3 tumor cells at the G0/G1 phase of the cell cycle.

TABLE 11

| | Non-IGF Treated Cells | | | IGF-1 Treated Cells | | |
|---|---|---|---|---|---|---|
| Antibody | G1/O phase | S phase | G2/M phase | G1/O phase | S phase | G2/M phase |
| (μg/mL) | (% cells) | (% cells) | (% cells) | (% cells) | (% cells) | (% cells) |
| SFM | 70.76 | 24.69 | 7.76 | 37.53 | 55.96 | 11.04 |
| IDEC141 | 69.44 | 23.14 | 9.21 | 36.11 | 57.71 | 11.1 |
| (20) | | | | | | |
| CO3 (20) | 64.71 | 32.94 | 3.68 | 56.95 | 31.42 | 21.75 |
| CO6 (20) | 68.87 | 28.53 | 3.82 | 57.08 | 38.16 | 8.33 |
| G11 (20) | 68.59 | 25.87 | 7.66 | 58.83 | 36.16 | 9.06 |

### Example 22

### In Vivo Inhibition of Tumor Growth in a Pancreatic Cancer Model

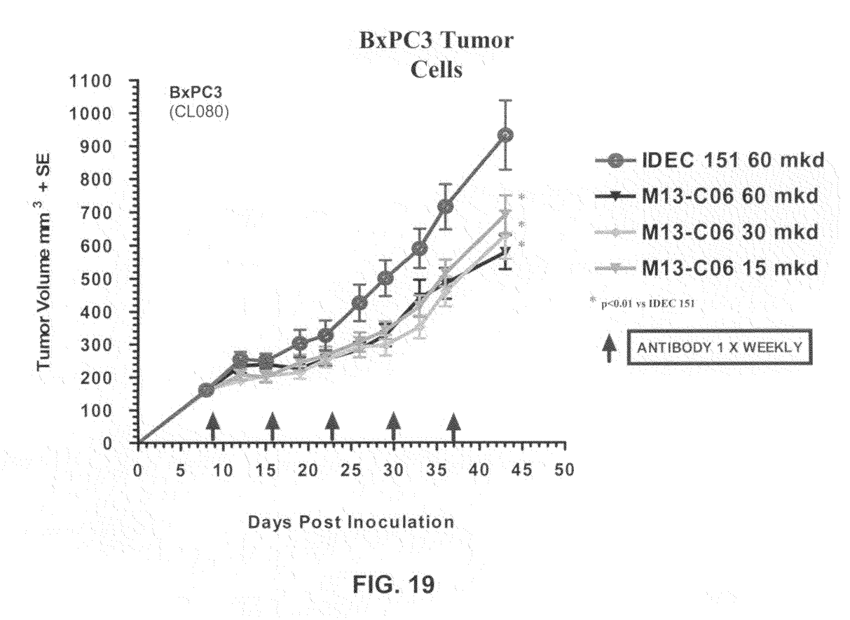

Methods: Single agent in vivo efficacy of M13.C06.G4.P.agly antibody was evaluated in a xenograft pancreatic cancer model system using BxPC3 (pancreatic cancer) cells. CB17 SCID mice were inoculated with 2×10⁶ cells and monitored for tumor growth. Mean tumor volume at the start of the therapy was ˜200 mm³. The M13.C06.G4.P.agly antibody was administered intraperitoneally (i.p.) at 60, 30 and 15 mg/kg administered one time per week for 5 weeks. An isotype matched antibody, IDEC-151 (human G4), was administered as a negative control at 60 mg/kg one time per week for 5 weeks. Tumors were extracted at the indicated intervals post-inoculation (FIG. 19) and total tumor volume was measured.

Results: The fully human M13.C06.G4.P.agly antibody inhibited tumor growth in a dose dependent manner (FIG. 19). The antibody demonstrated statistically significant single agent efficacy at 60, 30 and 15 mg/kg administered weekly for 5 weeks. Moreover, the antibody was efficacious at doses as low as 15 mg/kg administered once a week (FIG. 19).

### Example 23

### In Vivo Inhibition of Tumor Growth in a Lung Cancer Model

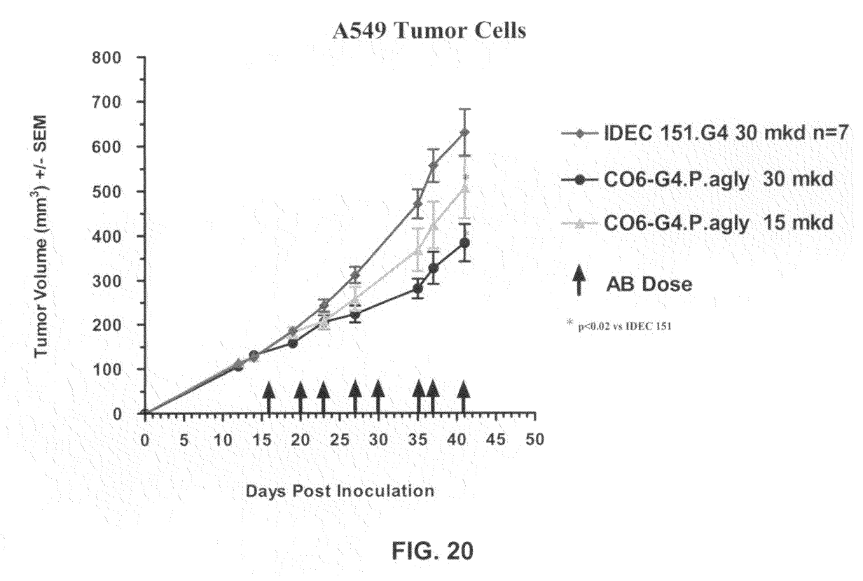

Methods: Single agent in vivo efficacy of M13.C06.G4.P.agly antibody was evaluated in a xenograft lung cancer model system using A549 (lung cancer) cells. CB17 SCID mice were inoculated with 3-5×10⁶ cells and monitored for tumor growth. Mean tumor volume at the start of the therapy was ˜150 mm³. The M13.C06.G4.P.agly antibody was administered intraperitoneally (i.p.) at 30 and 15 mg/kg administered two times per week per week for 4 weeks. An isotype matched antibody, IDEC-151 (human G4), was administered as a negative control at 30 mg/kg. Tumors were extracted at the indicated intervals post-inoculation (FIG. 20) and total tumor volume was measured.

Results: The fully human M13.C06.G4.P.agly antibody inhibited tumor growth in a dose dependent manner (FIG. 20). The antibody demonstrated statistically significant single agent efficacy at 30 and 15 mg/kg doses administered over a 4 week period (FIG. 20). Additional studies performed in this model showed that C06 is efficacious at doses as low as 7.5 mg/kg weekly injections (data not shown).

### Example 24

### In Vivo Inhibition of Tumor Growth Using Combination Therapy

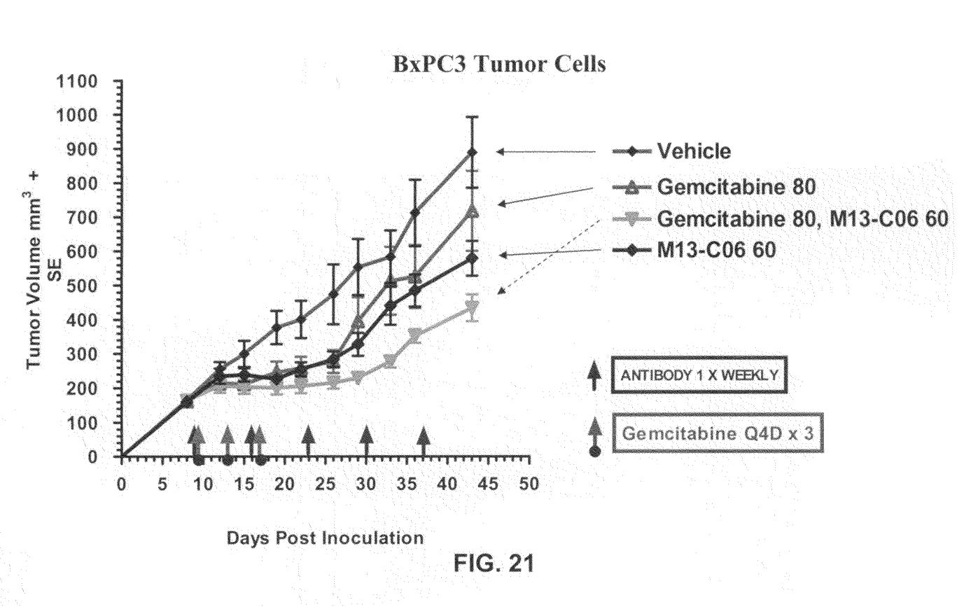

Methods: The efficacy of M13.C06.G4.P.agly antibody in inhibiting tumor growth in combination with gemcitabine (a drug commonly used to treat non-small cell lung cancer, pancreatic, bladder and breast cancer) was tested in a BxPC3 xenograft model. The efficacy of M13.C06.G4.P.agly antibody administered intraperitoneally (i.p.) two times per week at 30 mg/kg for 7 weeks (data not shown) or one time per week at 60 mg/kg for 5 weeks (FIG. 21) was evaluated in combination with gemcitabine administered according to the current standard of care (i.e., 80 mg/kg every 3 days for 4 weeks). Gemcitabine alone, M13.C06.G4.P.agly antibody alone, and sham injections of the delivery vehicle alone were administered as negative controls. Tumor volume at the start of the therapy was approximately 200 mm³.

Results: M13-C06.G4.P.agly antibody and gemcitabine as a single agent (i.e., administered alone) showed similar efficacy. In combination with Gemcitabine, the M13-C06.G4.P.agly antibody at 30 mg/kg on twice a week schedule (data not shown) or 60 mg/kg on a weekly schedule (FIG. 21) showed additive efficacy compared to the single agent treatments. In addition, combination with 15 mg/kg also showed additive efficacy (data not shown).

### Example 25

### Fully Human IGF-1R Antibody Binds to Cynomolgus Macaque Fibroblast Cell Line

Methods: The M13.C06.G4.P.agly antibody binds to a fibroblast cell line established from cynomolgus macaque. The fibroblast cell line was generated from a skin biopsy. Antibody binding was assessed by lifting the fibroblast cells with cell disassociation buffer and incubating with biotinylated M13.C06.G4.P.agly for 45 minutes at 4° C. After washing the cells, streptavidin-PE was added and incubated for additional 30 minutes at 4° C. in the dark. The cells were then washed and 200 ul cold PBS was added followed by fixation with 1% formaldehyde and gentle vortexing. Antibody binding was assessed by FACS analysis.

Results: The M13-C06.G4.P.agly antibody binds to IGF-1R expressed on the cynomolgus fibroblast cell line in a concentration dependent manner (FIG. 22).

### Example 26

### Part I: Summary of Biological Characteristics of Fully Human M13.C06.G4.P.agly Antibody

Biological characteristics assessed for fully human M13.C06.G4.P.agly antibody are presented in Tables 11 and 12. These characteristics were ascertained by methods, experiments, and examples described herein and/or as may be routinely determined via methods and experiments known and performed by those of ordinary skill in the art.

TABLE 11
Biological characteristics of M13.C06.G4.P.agly antibody
(human, non-glycosylated, IgG4)

| Properties Assessed: | Results Obtained: |
|---|---|
| IGF-1R Binding (EC50)* | Solube IGF-1R Protein: 4.22 × 10⁻¹¹M |
| | Tumor cell IGF-1R: 2.2 × 10⁻¹⁰ M |
| | (M13.C06 Fab affinity for IGF-1R = 1.3 nM) |
| Cyno IGF-1R | Cyno IGF-1R/CHO = 4.7 × 10⁻¹⁰ M |
| Rhesus IGF-1R | Rhesus IGF-1R/CHO = 2.7 × 10⁻¹⁰ M |
| Ligand Blocking (IC50 nM) | IGF-1 blocking: 0.979 nM |
| | IGF-2 blocking: 0.525 nM |
| Inhibition of IGF-1 & IGF-2 stimulated | IGF-1 < 0.13 nM |
| phosphorylation of IGF-1R (IC50 nM) | IGF-2 < 0.63 nM |
| Inhibition of IGF-1 & IGF-2 mediated | Positive for IGF-1 and IGF-2 at: |
| phosphorylation of Akt (Thr308, | >1 nM |
| Ser473) and pErk | >1 nM |
| IGF-1R down regulation | >60% down regulation in 1 hour in MCF-7 |
| (internalization) | cells |
| In vitro inhibition of IGF-1 & IGF-2 | Inhibition observed in ~70% cell lines |
| driven tumor cell line growth: | (15 of 21 cell lines) |
| In vivo efficacy of antibody in reducing | Activity in 3 mouse models at doses as low as |
| tumor size: | 7.5 mg/Kg × 1week |

M13. C06. G4.P.agly Antibody Serum Half-Life

A pharmacokinetic (PK) study in non-tumor bearing mice was performed using 3 mg/kg of M13.C06.G4.P.agly antibody (one dose level, intraperitoneal injections) in SCID mice. M13.C06.G4.P.agly antibody in SCID mouse serum was detected using IgG specific ELISA. Goat anti-human IgG (100 ng/well) was immobilized on IMMULON™ plates (Thermo Fisher Scientific Inc., Waltham, Mass., USA). Serums were titrated in triplicate starting at 1:25 with two fold serial dilutions. Binding was determined using Goat anti-human Kappa-HRP. Results of this study indicate a serum-half life of ˜11.5 days in this mouse model system (data not shown).

Serum concentrations of M13.C06.G4.P.agly were assessed after intraperitoneal injections in MCF-7 tumor bearing animals (antibody at 30 ug/kg) and BxPC3 tumor bearing animals (antibody at 15 ug/kg). Binding of M13.C06.G4.P.agly antibody to Goat anti-Human IgG (100 ng/well) immobilized on 96-well (IMMULON2 HB™, Dynax Technologies, Inc., Cat. #3455) was measured via ELISA. Standard curves were titrated starting at 10 ug/ml with 3 fold serial dilutions. Serum was titrated starting at 1:25 dilutions with 2 fold serial dilutions. M13.C06.G4.P.agly antibody was detected using Goat anti-human Kappa-HRP. SOFTMAX PRO software package version 4.3 LS (Molecular Devices Corp.) was used to determine antibody concentrations.

Average serum concentrations were observed as shown below:

| | |
|---|---|
| Bleed Time Points | Average serum |
| (hrs) | concentration (μg/mL) |

MCF-7 Tumor Bearing Mice

| | |
|---|---|
| 0 | 0 |
| 2 | 213 |
| 6 | 253 |
| 12 | 189 |
| 24 | 224 |
| 48 | 137 |

BxPC3 Tumor Bearing Mice

| | |
|---|---|
| 0 | 0 |
| 2 | 102 |
| 6 | 145 |
| 12 | 122 |
| 24 | 115 |
| 48 | 79 |

The pharmacokinetics of M13.C06.G4.P.agly antibody has also been investigated in cynomolgus monkeys after 10 mg/kg and 25 mg/kg dose injections, where the serum half-life was observed to be ˜10 to 12 days (data not shown).

Tables 12 and 13 show the dose dependent inhibition (percent inhibition) of in vitro cell growth observed for various lung, pancreas, and colon tumor cell lines when M13-C06.G4.P.agly antibody is added to cell culture media supplemented with IGF-1 or IGF-2 (Table 12) or supplemented with 10% fetal calf serum (FCS) or fetal bovine serum (FBS) (Table 13).

TABLE 12

| | | IGF-1 in Media | | | | IGF-2 in Media | | | |
|---|---|---|---|---|---|---|---|---|---|
| | | Dose dependent cell growth | | | | | | | |
| | | inhibition with increasing M13- | | | | | | | |
| | | C06.G4.P.agly antibody concentration | | | | | | | |
| | | (% = percent growth inhibition; | | | | | | | |
| Cell | | nM = antibody concentration) | | | | | | | |
| Type: | Cell Line: | 0.1 nM | 1 nM | 10 nM | 100 nM | 0.1 nM | 1 nM | 10 nM | 100 nM |
| Lung | NCI-H23 | 12% | 32% | 61% | 84% | 2% | 32% | 61% | 85% |
| | A549 | 39% | 58% | 79% | 87% | 37% | 61% | 76% | 85% |
| | Calu-6 | 12% | 15% | 19% | 53% | −4% | 16% | 27% | 62% |
| | SK-MES-1 | −30% | −15% | 5% | 46% | ND | ND | ND | ND |
| Pancreas | BXPC3 | 12% | 34% | 54% | 82% | 63% | 79% | 96% | 99% |
| | Panc-1 | 0% | 0% | 18% | 60% | 0% | 12% | 35% | 62% |
| | Capan-1 | 2% | 0% | 20% | 17% | 19% | 12% | 12% | 31% |
| | Capan-2 | 14% | 22% | 36% | 49% | ND | ND | ND | ND |
| Colon | Colo 205 | 15% | 37% | 56% | 76% | 18% | 30% | 45% | ND |
| | SW620 | 10% | 12% | 13% | 27% | ND | ND | ND | ND |

TABLE 13

| | | 10% Serum in Media | | | |
|---|---|---|---|---|---|
| | | Dose dependent cell growth | | | |
| | | inhibition with increasing | | | |
| | | M13-C06.G4.P.agly antibody concentration | | | |
| | | (% = percent growth inhibition; | | | |
| Cell | Cell | nM = antibody concentration) | | | |
| Type: | Line: | 0.2 nM | 2 nM | 20 nM | 200 nM |
| Lung | NCI-H23 | 5% | 12% | 21% | 47% |
| | A549 | 2% | 12% | 22% | 41% |
| | Calu-6 | 0% | 0% | 0% | 9% |
| | SK-MES-1 | 12% | 10% | 6% | 7% |
| Pancreas | BXPC3 | 6% | 3% | 9% | 26% |
| | Panc-1 | 6% | 11% | 12% | 30% |
| | Capan-1 | 0% | 0% | 0% | 0% |
| | Capan-2 | 41% | 45% | 47% | 38% |
| Colon | Colo 205 | 0% | 0% | 11% | 28% |
| | SW620 | 0% | 4% | 6% | 20% |
| | HT-29 | 21% | 21% | 23% | 37% |
| | WiDr | 35% | 45% | 51% | 57% |

### Part II: Antibody Affinity Measurements

Objective:

The objective was to measure the binding affinity of IGF-1R antibodies.

Methods:

### Preparation of M13-C06, M14-C03, and M14-G11 Fabs

M13-C06, M14-C03, and M14-G11 Fab antibodies were prepared by digestion with immobilized papain (Pierce Cat. No. 20341). The papain resin was washed with 20 mM sodium phosphate pH 7.0; 10 mM EDTA; 20 mM Cysteine. Antibodies were mixed with the papain resin in 500 mM EDTA, 100 mM Cysteine pH 7.0 and digested for three hours in a 37° C. water bath followed by mixing on an inverting shaker overnight at room temperature. Completion of each digestion was determined by analytical size exclusion chromatography (SEC). The resin was removed from the digested protein with a sintered glass funnel filter and washed with 20 mM acetate pH 5.0. The flow-through was collected and diluted 10-fold with 20 mM acetate pH 5.0. Fab fragments were purified by S-SEPHAROSE™ cation exchange chromatography using a linear salt gradient. Analytical SEC was performed on the eluted fractions and the desired fractions were pooled and dialyzed into PBS. The Fabs were subsequently alkylated to inhibit the re-formation of hinge disulfides resulting in (Fab)₂ production. Alkylation was carried out by diluting 1 M Tris; 200 mM Iodoacetate pH 8.5 10-fold into the Fab solutions. The mixtures were incubated on an inverting shaker for twenty minutes at room temperature followed by exhaustive dialysis into 1×PBS. Final purification of each Fab was performed using preparative size exclusion chromatography.

### Surface Plasmon Resonance (SPR) Affinity Measurements

All surface plasmon resonance (SPR) experiments were performed on a Biacore 3000 set to 25° C. using HBS-EP (Biacore, Cat. No. BR-1001-88) as the running buffer. A biotin-labeled anti-His Tag antibody (biotin-PENTA-His, Qiagen Cat. No. 34440) was immobilized to saturation on a Biacore SA chip (Cat. No. BR-1000-32) surface by injection at 500 nM in HBS-EP buffer. Recombinant human IGF-1R-10His (R&D Systems, Cat. No. 305-GR-050) was captured on the biotin-PENTA-His surface by injecting 20 μL of 40 nM protein at 2 μL/min. Subsequent to IGF-1R injections, flow rates were increased to 20 μL/min. A second, 130 μL injection of anti-IGF-1R antibody or Fab was performed to investigate interactions with the receptor. Each antibody and Fab was serially diluted from 64 nM to 0.5 nM to obtain concentration dependent kinetic binding curves. Each injection series was regenerated using 3×10 μL injections of 10 mM Acetate, pH 4.0, at 20 μL/min. Each curve was double referenced using (1) data obtained from a streptavidin surface devoid of IGF-1R and (2) data from a primary injection of IGF-1R followed by a secondary injection of HBS-EP buffer. The concentration series for each antibody and Fab was fit to the 1:1 binding model provided within the BiaEvaluation software of the manufacturer.

Results: Three recombinant anti-IGF-1R antibodies, M13-C06, M14-C03, and M14-G11, were tested for binding to IGF-1R using surface plasmon resonance as described above. All three antibodies demonstrated strong binding to the receptor. Concentration dependent binding of each antibody (64 nM serially diluted to 0.5 nM) to immobilized recombinant human IGF-1R was observed (data not shown). The rates at which the antibodies accumulate on the IGF-1R coated surface when applied at various concentrations as well as the rates at which they dissociated during applications of pure buffer were investigated by fitting the data to a 1:1 binding model. Approximate kinetic rate constants and equilibrium dissociation constant were calculated (Table 14).

TABLE 14

| Antibody/Fab | K_{D} (M) | k_{d} (s⁻¹) | kₐ (M⁻¹ s⁻¹) |
|---|---|---|---|
| M13-C06_Ab | 1.3e−10 | 2.5e−4 | 1.8e6 |
| M14-C03_Ab | 3.6e−10 | 2.0e−4 | 5.7e5 |
| M14-G11_Ab | 1.1e−10 | 1.1e−4 | 1.0e6 |

TABLE 15

| Antibody/Fab | K_{D} (M) | k_{d} (s⁻¹) | kₐ (M⁻¹ s⁻¹) |
|---|---|---|---|
| M13-C06_Fab | 1.3e−9 | 1.2e−3 | 8.8e5 |
| M14-C03_Fab | 4.9e−9 | 9.4e−4 | 1.9e5 |
| M14-G11_Fab | 4.0e−9 | 1.2e−3 | 3.0e5 |

To obtain discrete affinities, Fab fragments of each antibody were generated using papain digestion as described above. Due to the presence of a single antigen binding site, the Fabs uniformly demonstrated monophasic binding and dissociation curves when applied to the IGF-1R receptor in an identical fashion as described for the full-length antibodies (data not shown). The affinities of each Fab for IGF-1R are provided in Table 15.

### Example 27

### Part I: M13.C06.G4.P.agly Antibody Has Unique Epitope Binding Characteristics Compared to Other IGF-1R Antibodies

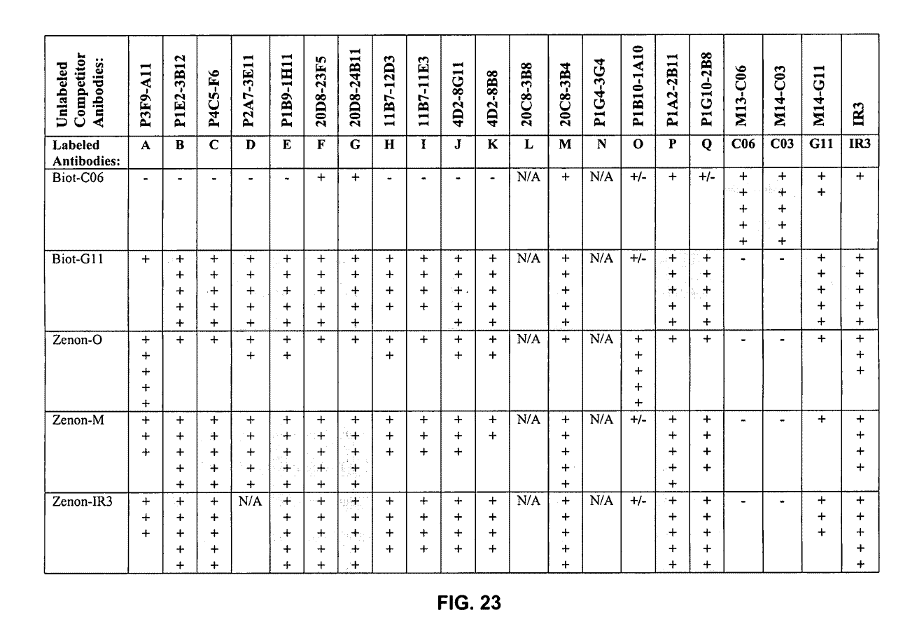

A cross-competition antibody binding study was performed to compare the IGF-1R antibody binding epitopes of M13.C06.G4.P.agly and other IGF-1R antibodies. See, FIG. 23. Unlabeled competitor antibodies were analyzed for their ability to cross-compete with five different labeled antibodies for binding to soluble IGF-1R. The five labeled antibodies used were biotin-labeled M13.C06.G4.P.agly (“Biotin-C06”), biotin labeled M14-G11 (“Biotin-G11”), zenon-labeled P1B10-1A10 (“Zenon-O”), zenon-labeled 20C8-3B4 (“Zenon-M”), or zenon-labeled IR3 antibody (“Zenon-IR3”). See, FIG. 23.

Antibodies were labeled with Biotin using a Biotinylation kit from Pierce Chemical (#21335). Zenon labeling was performed using Zenon mouse IgG labeling kit from Molecular Probes (Z25000).
+++++=antibody binding competition relative to itself (90-100%)
++++=70-90% competition
+++=50-70% competition
++=30-50% competition
+=10-30% competition
+/−=0-10% competition
N/A=results not available.
   The results of this analysis indicate that M13.C06.G4.P.agly and M14.C03.G4.P.agly antibodies bind to the same or a similar region of IGF-1R, which is distinct from all other antibodies tested. In particular, only biotin-labeled M13.C06.G4.P.agly antibody was effectively competed from IGF-1R binding by unlabeled M13.C06.G4.P.agly or by unlabeled M14.C03.G4.P.agly. It is also notable that M13.C06.G4.P.agly does not cross-compete with the well-studied IR3 antibody. Hence, these two antibodies, in particular, bind to different IGF-1R epitopes.

### Part II: M13-C06 Allosterically Decreases the Binding Affinity of IGF-1 for IGF-1R Via Antibody Binding to the N-Terminal Region of the FnIII-1 Domain

Objective: The objective was to elucidate the binding epitope of M13-C06 antibody on IGF-1R and the mechanism behind inhibition of IGF-1/IGF-2 binding to IGF-1R.

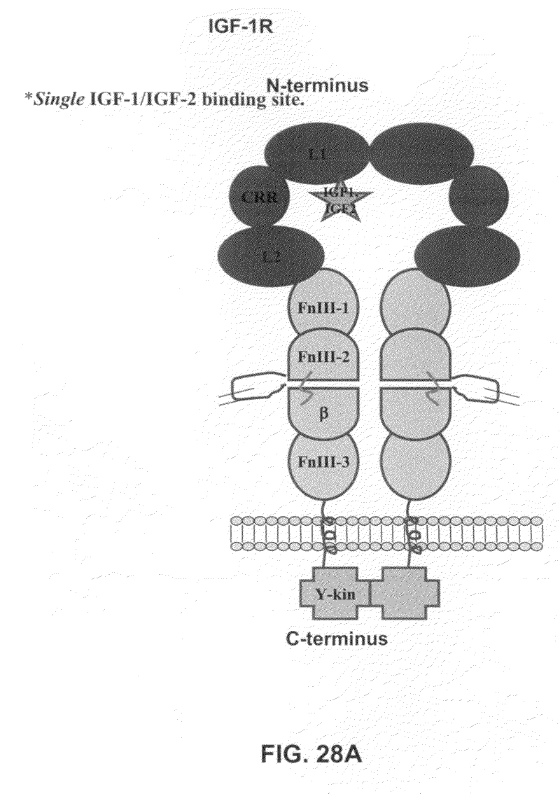

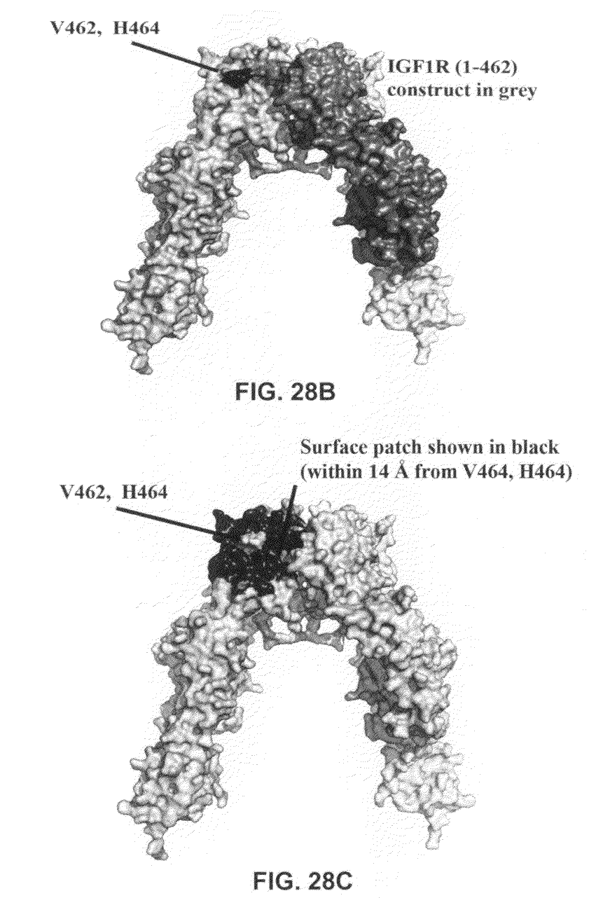

Background: IGF-1R consists of 6 domains (FIG. 28A). It has been published that mutations in the first three domains of IGF-1R, denoted L1 (leucine rich repeat domain 1), CR (cysteine rich repeat domain), and L2, as well as a peptidic loop region in domain 5 (FnIII-2, Fibronectin type III domain 2) have a negative impact on IGF-1 and IGF-2 binding (Whittaker 2001; Sorensen 2004). Here, we demonstrate that M13-C06 antibody does not block IGF-1 and IGF-2 binding to IGF-1R by competitively interacting with the growth factor binding site, but instead binds to FnIII-1 and allosterically inhibits IGF-1/IGF-2 signaling. FnIII-1 is believed to facilitate receptor homodimerization of both IGF-1R and INSR (McKern 2006) and, upon binding ligand, transmit an activating signal through the transmembrane region to the C-terminal tyrosine kinase domains via a quarternary structure change. The data from this example suggests M13-C06 antibody inhibits conformational changes induced by IGF-1/IGF-2 that lead to downstream receptor signaling.

Methods: IGF-1/IGF-1R binding experiments in the presence and absence of M13-C06 antibody. Several constructs were used to investigate antibody/IGF-1 binding to the IGF-1R receptor or insulin receptor: human IGF-1R(1-902)-His₁₀ (denoted hIGF-1R-His₁₀, from R&D systems), human INSR(28-956)-His₁₀ (denoted INSR, from R&D systems), human IGF-1R(1-903)-Fc (denoted hIGF-1R-Fc, generated by Biogen Idec), human IGF-1R(1-462)-Fc (denoted hIGF-1R(1-462)-Fc, generated by Biogen Idec), and murine IGF-1R(1-903)-Fc (denoted mIGF-1R-Fc, generated by Biogen Idec). “His₁₀” denotes a 10-residue histidine tag on the C-terminus of the constructs. “Fc” denotes a C-terminal human IgG1-Fc tag.

Human IGF-1 was purchased from Millipore. The affinity of IGF-1 for hIGF-1R-His₁₀ was determined using surface plasmon resonance (SPR). A biotin-labeled anti-His Tag antibody (biotin-PENTA-His, Qiagen Cat. No. 34440) was immobilized to saturation on a Biacore SA chip (Cat. No. BR-1000-32) surface by injection at 500 nM in HBS-EP buffer. For each sensorgram, hIGF-1R-His₁₀ (described in Example 5 (Part II)) was captured on the biotin-PENTA-His surface by injecting 20 μL of 40 nM protein at 2 μL/min. Subsequent to hIGF-1R-His₁₀ injection, the flow rate was increased to 20 μL/min. A second, 130 μL injection containing IGF-1 was performed to investigate interaction of the growth hormone with its receptor. IGF-1 was serially diluted from 64 nM to 0.125 nM to obtain concentration dependent kinetic binding curves. Each injection series was regenerated using 3×10 μL injections of 10 mM Acetate, pH 4.0, at 20 μL/min. Each curve was double referenced using (1) data obtained from a streptavidin surface devoid of PENTA-His antibody and (2) data from a primary injection of hIGF-1R-His₁₀ followed by a secondary injection of HBS-EP buffer. The concentration series for IGF-1 was fit to the 1:1 binding model provided within the BiaEvaluation software of the manufacturer. Two sets of data were obtained, one in the absence and another in the presence of 400 nM M13-C06 in the running buffer, hIGF-1R-His₁₀ injection buffer, and IGF-1 injection buffer.

Pull-Down and Western Blot Analysis of IGF-1/IGF-1R/M13-C06 Antibody Ternary Complexes

Resuspended Protein A/G beads (300 μl, Pierce Cat. No. 20422) were washed with 1×PBS and mixed with 1.0 mg M13-C06 in a 1.5 ml Eppendorf tube on a rotary shaker for two hours at room temperature. In a separate tube, 12 μg hIGF-1R-His₁₀ (R&D systems) and 460 ng human IGF-1 (Chemicon International Cat. No. GF006) were mixed (1:1 protein:protein ratio) for one hour at room temperature. Protein A/G with bound M13-C06 was washed with PBS and incubated with the hIGF-1R-His₁₀/IGF-1 mixture for 30 minutes at room temperature. Protein A/G with bound protein was washed with PBS followed by elution of bound protein with 300 μL 100 mM glycine, pH 3.0. For the negative control, the addition of 12 μg human IGF-1R(1-902)-His₁₀ was omitted. Eluted proteins were detected by Western Blot with an anti-human IGF-1 antibody (Rabbit anti-Human IGF-1 Biotin, US Biological Cat. No. 17661-01B) and an anti-human IGF-1R antibody (IGF-1Rα 1H7, Santa Cruz Biotechnology Cat. No. sc-461) as primary antibodies followed by HRP-labeled streptavidin (Southern Biotech Cat. No. 7100-05) and HRP-labeled goat anti-mouse IgG (US Biological Cat. No. I1904-40J) as secondary antibodies. To demonstrate the ability of IGF-1/IGF-1R/M13-C06 to form a ternary complex the concentrations of the IGF-1 and IGF-1R used in this experiment were well in excess (>15-fold above) the normal physiological levels of these proteins (particularly IGF-1 in the serum) as well as the measured equilibrium dissociation constant for IGF-1R/IGF-1. See, for example, Hankinson et al., 1998.

Construction of IGF-1R(1-462)-Fc and Comparative Antibody Binding Studies Versus the Full-Length Receptor Ectodomain

Construction of the IGF-1/IGF-2 binding domains, L1-CR-L2 (residues 1-462), of human IGF-1R was published previously (McKern 1997). Utilizing this information, we subcloned human IGF-1R residues 1-462 (along with the N-terminal signal sequence) into the same in-house PV90 vector that was used to produce the full-length human ectodomain (residues 1-903, hIGF-1R-Fc). Expression in CHO was facilitated using methods described previously (Brezinsky 2003). The protein was purified from CHO supernatants by passage over a protein A affinity column as described previously for other Fc-fusion proteins (Demarest 2006). The protein construct is denoted hIGF-1R(1-462)-Fc.

The ability of M13-C06, M14-C03, and M14-G11 antibodies to bind hIGF-1R(1-462)-Fc and the full-length ectodomain construct, hIGF-1R-Fc, was determined by SPR using a Biacore3000. The instrument was set to 25° C. and the running buffer was HBS-EP, pH 7.2 (Biacore, Cat. No. BR-1001-88). The fully human antibodies, M13-C06, M14-C03, and M14-G11, were immobilized to ˜10,000 RU on Biacore CM5 Research Grade Sensor Chip (Cat. No. BR-1000-14) surfaces using the standard NHS/EDC-amine reactive chemistry according to protocols supplied by Biacore. For immobilization, the antibodies were diluted to 40 μg/mL in a 10 mM Acetate pH 4.0 buffer. To investigate the relative kinetics of association and dissociation of hIGF-1R-Fc and hIGF-1R(1-462)-Fc to each of the human antibodies, increasing concentrations of each receptor construct were injected over the sensorchip surfaces. The hIGF-1R-Fc concentration series ranged from 1.0 nM to 100 nM while the hIGF-1R(1-462)-Fc concentration series ranged from 1.0 nM to 2 μM. All antibody surfaces were reliably regenerated with 100 mM Glycine, pH 2.0. Repeated regenerations did not lead to activity losses for any of the antibody surfaces. Flow rates were 20 μl/min.

Epitope Mapping Mutations

The choice of mutants to probe for the epitope of M13-C06 antibody on IGF-1R were based on the observation that the binding affinity of M13-C06 to mouse IGF-1R was significantly reduced or non-detectable in Biacore and FRET binding experiments (Example 5 (Part III)). Mouse and human IGF-1R share 95% primary amino acid sequence identity. Human IGF-1R and human INSR share 57% identity (73% similarity). We identified 33 residues that differ between mouse and human IGF-1R in the ectodomain (Table 16). Twenty of these residues were targeted for mutation because the homologous positions within the INSR ectodomain were exposed to solvent based on the recent INSR crystal structure (pdb code 2DTG, McKern 2006). Accessible surface areas were calculated using StrucTools (http://molbio.info.nih.gov/structbio/basic.html) with a 1.4 Å probe radius. Four additional residues not in the structure of INSR were also chosen for mutagenesis as they resided in the unstructured loop region of the FnIII-2 domain that has been demonstrated to be important for IGF-1/IGF-2 binding (Whittaker 2001; Sorensen 2004). The list of the 24 mutations chosen for the epitope mapping study are shown in Table 17.

TABLE 16
Amino acid differences between human and mouse IGF-
1R. Solvent accessibility of each residue position was determined based
on the publicly available structure of the homologous INSR ectodomain.
Residues shown in bold/italics exposed greater than 30% of their surface
area to solvent and were mutagenized to screen for the IGF-1R epitope of
M13-C06.

| | Human | Mouse | Human | IR | % Solvent |
|---|---|---|---|---|---|
| Residue # | IGF1R | IGF1R | INSR | pdb # | Accessibility |
| [symbol] | [symbol] | [symbol] | [symbol] | [symbol] | [symbol] |
| 125 | V | I | I | 131 | 0 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 214 | N | D | D | 221 | 25.7 |
| 215 | D | N | P | 222 | 20.4 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 257 | L | P | H | 263 | 19.2 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 326 | F | L | I | 335 | 25.5 |
| [symbol] | [symbol] | [symbol] | [symbol] | [symbol] | [symbol] |
| 411 | I | V | T | 421 | 0.5 |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| 471 | S | W | S | 481 | 26.4 |
| [symbol] | [symbol] | [symbol] | [symbol] | [symbol] | [symbol] |
| [symbol] | [symbol] | [symbol] | [symbol] | [symbol] | [symbol] |
| 605 | S | T | S | 615 | N/A |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |

The 24 mutant epitope mapping library was constructed by mutagenizing the wild-type hIGF-1R-Fc PV-90 plasmid using the STRATAGENE™ site-directed mutagenesis kit following the manufacturer's protocols. Incorporation of each mutant (or double mutant in the case of the SD004, SD011, SD014, SD016, and SD019 library members) into the PV-90 vector was confirmed by our in-house DNA sequencing facility. Plasmids were miniprepped and maxiprepped using the Qiagen Miniprep Kit and Qiagen Endotoxin-Free Maxikits, respectively. 200 μg of each mutant plasmid was transiently transfected into 100 mL HEK293 T cells at 2×10⁶ cells/mL using the PolyFect transfection kit (Qiagen) for soluble protein secretion into the media. Cells were cultured in DMEM (Ivrine Scientific), 10% FBS (low IgG bovine serum, Invitrogen—further depleted of bovine IgG by passage over a 20 mL protein A column) at 37° C. in a CO₂ incubator. After 7 days, supernatants containing each IGF-1R-Fc mutant were collected by centrifugation at 1200 rpm and filtration through a 0.2 μm filter. Each mutant was affinity purified by passage of the supernatants over a 1.2 mL protein A Sepharose FF column pre-equilibrated with 1×PBS. The mutants were eluted from the column using 0.1 M glycine, pH 3.0, neutralized with 1 M Tris, pH 8.5, 0.1% Tween-80, and concentrated to ˜300 μL using VivaSpin 6 MWCO 30,000 centrifugal concentration devices (Sartorius, Cat. No. VS0621).

Western Blot Analysis of IGF-1R Mutants

hIGF-1R-Fc mutant samples were run on 4-20% Tris-Glycine gels (Invitrogen Cat. No. EC6028) using Xcell SureLock Mini Cell (Invitrogen Cat. No. E10001) following standard manufacturer protocol. Samples were transferred to nitrocellulose using the iBlot Dry Blotting System (Invitrogen Cat. No. IB1001) and Transfer Stacks (Invitrogen Cat. No. IB3010-01 or 3010-02) following standard manufacturer protocol. Membranes were blocked overnight at 4° C. in 25 ml of PBST; 5 mg/ml non-fat dry milk. After blocking, membranes were washed once with 25 ml PBST for 5 min at room temperature. Membranes were incubated with a primary anti-IGF-1RP antibody (Santa Cruz Biotechnology Cat. No. sc-9038) at 1:100 in 10 ml PBST for 1 hr at room temperature. The membranes were subsequently washed three times in 25 ml PBST for 5 min. For detection, membranes were incubated with a secondary HRP-conjugated Goat anti-Rabbit IgG-Fc antibody (US Biological Cat. No. I1904-40J) at a 1:1000 dilution in 10 ml PBST for 1 hr at room temperature. Membranes were washed three times in 25 ml PBST for 5 min followed by one wash in 25 ml PBST for 20 min. Protein bands were detected using the Amersham ECL Western Blotting Analysis System (GE Healthcare Cat. No. RPN2108) following standard manufacturer protocol.

Biacore Analysis of the IGF-1R-Fc Mutant Library

Both mIGF-1R-Fc and hIGF-1R-Fc bind with high apparent affinity to the M13-C06, M14-C03, and M14-G11 sensorchip surfaces described above due to their highly multivalent nature induced by the incorporation of two separate homodimeric regions (IGF-1R and IgG1-Fc). To distinguish between the actual high affinity binding hIGF-1R-Fc to M13-C06 and the low affinity binding of mIGF-1R-Fc to M13-C06, the receptor-Fc fusions were captured on the M13-C06 sensorchip surface followed by an additional soluble M13-C06 Fab binding event. Receptor-Fc constructs were captured to the M13-C06 chip surface (prepared as described above) by injection of 60 μL of the affinity purified, concentrated material at a 1 μl/min flow rate. After, completion of the receptor-Fc loading step, flow rates were elevated to 5 μl/min. 10 nM, 3 nM, and 1 nM M13-C06 Fab concentrations were injected (50 μL) subsequent to the loading of each receptor-Fc construct. At the end of each sensorgram, the flow rate was elevated to 30 μl/min and the chip surface was regenerated by 2×10 μL injections of 0.1 M glycine, pH 2.

Time-Resolved Fluorescence Resonance Energy Transfer (tr-FRET) Assay for IGF-1R-Fc Mutant Screening

Serial dilutions of mutant receptor, starting at 0.25-0.5 μg (25 μl) were mixed with 0.05 μg IGF1R-His₁₀-Cy5 (12.5 μl) and 0.00375 μg Eu:C06 (12.5 μl) in 384-well microtiter plates (white from Costar). The conjugation levels for IGF1R-His₁₀-Cy5 were 6.8:1 Cy5:IGF1R-His₁₀, and for Eu—C06 were 10.3:1 Eu:C06. The total volume was 50 μl for each sample. Plates were incubated for 1 hr at room temperature on a plate agitator. Fluorescence measurements were carried out on a Wallac Victor² fluorescent plate reader (Perkin Elmer) using the LANCE protocol with the excitation wavelength at 340 nm and emission wavelength at 665 nm. All data were fitted to a one-site binding model from which the corresponding IC₅₀ values were determined.

Results: Inhibition of IGF-1 and/or IGF-2 binding to hIGF-1R-Fc by M13-C06 was demonstrated as previously described in Example 3. Even at saturating conditions, most antibodies do not fully inhibit IGF-1 or IGF-2 binding to hIGF-1R-Fc. Particularly for M13-C06, we hypothesized that inhibition of ligand binding might be non-competitive or allosteric. To test this hypothesis, we determined the affinity of IGF-1 for hIGF-1R-His₁₀ in the presence and absence of 400 nM M13-C06 antibody (˜4000-fold above the affinity of the antibody for hIGF-1R-His₁₀). Using SPR hIGF-1R-His₁₀ was immobilized to chip surfaces using an anti-His Tag antibody followed by injection of increasing concentrations of IGF-1 (up to 64 nM). IGF-1 binding to hIGF-1R-His₁₀ was evident in the absence and presence of 400 nM M13-C06. (Data not shown: Surface plasmon resonance demonstrating binding of IGF-1 to hIGF-1R-His₁₀ in the absence and presence of 400 nM M13-C06. The SPR association phase was between 1400-1800 seconds while the dissociation phase was between 1800-3000 seconds. In the absence of M13-C06, IGF-1 bound to hIGF-1R-His₁₀ with K_{D}=17 nM (kₐ=2.4×10⁻⁵/M*s). In the presence, of 400 nM M13-C06, IGF-1 bound to hIGF-1R-His₁₀ with K_{D}=59 nM (kₐ=7.1×10⁻⁴ μM*s).) The kinetic association rate constant of IGF-1 binding to hIGF-1R-His₁₀ was reduced approximately 3-fold in the presence of M13-C06, suggesting that M13-C06 allosterically reduces the affinity of the ligand for the receptor.

Supporting evidence that M13-C06 does not directly compete with IGF-1 for binding to hIGF-1R-His₁₀ was generated by performing a co-immunoprecipitation of hIGF-1R-His₁₀ and IGF-1 using M13-C06 at concentrations well above the apparent affinities of both IGF-1 and M13-C06 for hIGF-1R-His₁₀. Western blot analysis demonstrated that ˜70-100% of the IGF-1 material mixed with hIGF-1R-His₁₀ was pulled down with M13-C06, thereby demonstrating that co-engagement of M13-C06 and IGF-1 with hIGF-1R-His₁₀ to form the ternary complex is possible (data not shown). These results demonstrate the allosteric nature of M13-C06 inhibition of IGF-1 binding at normal IGF-1 serum concentrations and suggest that the binding site of M13-C06 does not overlap with the IGF-1R ligand-binding pocket.

Next, we investigated whether M13-C06 binds the putative ligand binding domains of IGF-1R (L1-CR-L2). We generated a truncated version of the receptor containing the N-terminal three domains (residues 1-462) fused to an IgG1-Fc and compared its ability to bind M13-C06, M14-C03, and M14-G11 to that of the full-length receptor ectodomain construct, hIGF-1R-Fc, using surface plasmon resonance (SPR). M14-G11 demonstrated equivalent binding to the truncated version of the receptor, while the binding of M13-C06 and M14-C03 was dramatically reduced. (Data not shown: Surface immobilized M13-C06, M14-C03, and M14-G11 antibodies were tested for binding to hIGF-1R(1-903)Fc and truncated hIGF-1R(1-462)-Fc at concentrations ranging from 2 μM, 100 nM, 30 nM, 10 nM, 5 nM and 1 nM. The SPR association phase was between 480-960 seconds while the dissociation phase was between 960-1170 seconds.) Residual binding was apparent for both M13-C06 and M14-C03; however, the data suggests that at least a good portion of the epitopes of these antibodies resides in an IGF-1R region outside the ligand binding domains.

We utilized the fact that murine IGF-1R does not bind M13-C06 antibody to design a library of mouse mutations within hIGF-1R-Fc to assess the location of the M13-C06 binding site on IGF-1R. The various mutations in hIGF-1R tested are shown in Table 17. Western blot analysis was used to confirm expression of each hIGF-1R-Fc mutant and to develop a standard curve to approximate the relative concentration of each mutant protein; using purified hIGF-1R-Fc as a positive control (data not shown).

TABLE 17
Affect of mutations on IGF-1R binding to M13-C06.

| Mutation | | | IC50 values |
|---|---|---|---|
| Number | Individual Mutants | Biacore Relative RUmax | (μg/ml) |
| SDWT | Wild-type | 1.0 | 1.5 |
| mIGF1R | — | 0.0 | >100 |
| SD001 | Y28A | 0.6 | 1.0 |
| SD002 | M156A | 1.2 | 0.3 |
| SD003 | T188F | 1.0 | 0.2 |
| SD004 | S210H_A211Q | 0.8 | ND |
| SD005 | A217T | 0.9 | ND |
| SD006 | A227K | 1.7 | 0.2 |
| SD007 | N237G | 1.3 | <0.1 |
| SD008 | S258F | 1.5 | <0.1 |
| SD009 | E264K | 0.6 | 7.7 |
| SD010 | G271D | 0.8 | 0.1 |
| SD011 | G285S_S286T | 1.8 | <0.1 |
| SD012 | E303G | 0.3 | 0.9 |
| SD013 | D405K | 0.7 | <0.1 |
| SD014 | K412A_A413Q | 0.6 | <0.1 |
| SD015 | H464E | 0.04 | >100 |
| SD016 | D531Q_V532N | 2.0 | 0.1 |
| SD017 | I650S | 2.0 | 0.2 |
| SD018 | E665A | 1.7 | <0.1 |
| SD019 | A739W_I741F | 1.9 | 0.2 |

SD015 is bold-faced as it was the only residue to demonstrate little to no binding to M13-C06 in the two assay formats.
ND = not determined.

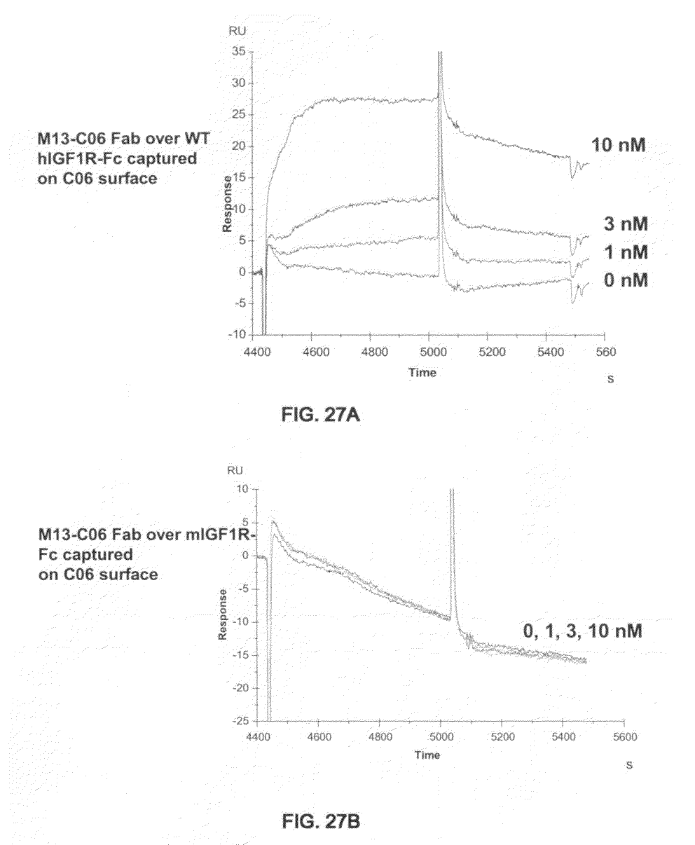

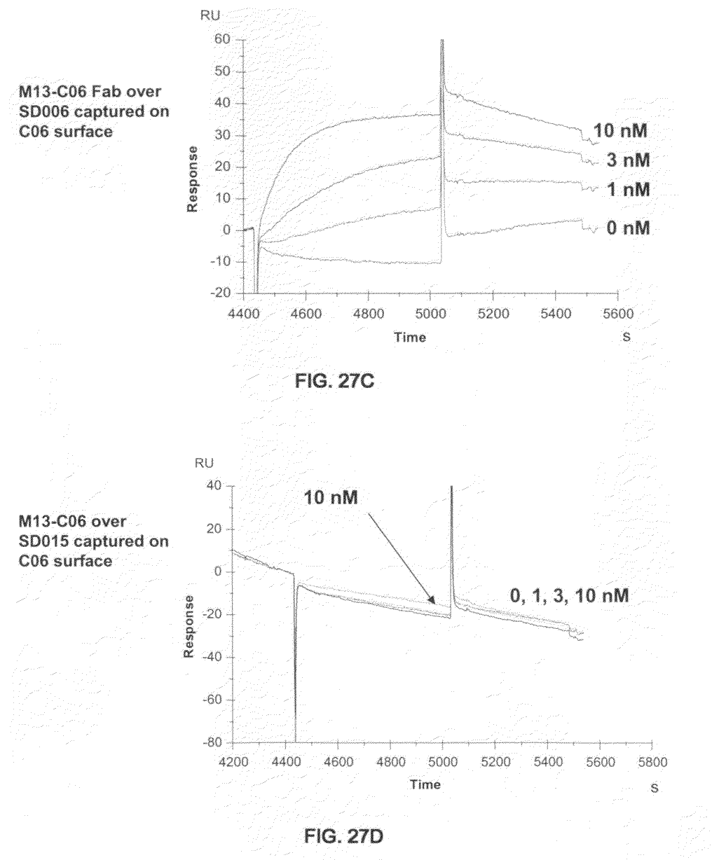

SPR and tr-FRET was used to screen for mutations that inhibit the binding of IGF-1R-Fc to M13-C06. Except for the SD015 mutant, all mutant IGF-1R constructs demonstrated M13-C06 binding activity, or M13-C06 Fab binding activity in the SPR experiments. See: FIG. 27; Table 17; and, data not shown (competitive inhibition analysis was used to establish binding curves for displacement of Eu-M13-C06 bound to Cy5-labeled IGF1R by increasing concentrations of unlabeled hIGF1R-Fc (SDWT), mouse IGF1R-Fc (mIGF1R-Fc) and mutant hIGF1R-Fc constructs).

There was some deviation in the IC₅₀ values determined using tr-FRET and relative binding strengths determined using SPR due to natural variations in expression and quantitation by Western Blot; however, SD015 was the only mutant to demonstrate virtually no binding activity toward M13-C06 in both assays and to parallel the results determined for the mIGF-1R-Fc control. His464 is located 2 amino acids C-terminal in primary amino acid sequence to the C-terminus of the truncated version of hIGF-1R-Fc construct (i.e., hIGF-1R(1-462)-Fc). The residual binding activity of M13-C06 to truncated hIGF-1R(1-462) suggests that the M13-C06 binding epitope minimally encompasses residues Val462-His464. Additional residues are likely involved in the antibody-epitope binding interaction as evidence indicates that M13-C06's epitope is conformationally dependent. Notably, however, residues Val462 and His464 are predicted to reside on the exterior surface of the FnIII-1 domain (FIG. 28).

In an attempt to characterize the extent of the M13-C06 epitope (i.e., what residues peripheral to 462-464 are important for antibody binding and activity), we took a structural modeling approach. Human IGF-1R and human INSR share 57% identity (73% similarity) and a similar tertiary structure. Previous analyses of X-ray crystal structure protein antigen:antibody binding surfaces has suggested an average binding surface of 700 Å² (angstroms-squared) with an approximate radius of 14 Å from the center of the binding epitope (Davies 1996). Using the X-ray crystal structure of the homologous ectodomain of INSR (pdb code 2DTG, (McKern 2006)), we calculated the residues on the surface of the FnIII-1 domain within a 14 Å radius of residues 462-464 by mapping the IGF-1R residues V462 through H464 to INSR residues L472 and K474. The distances cut-off was applied for any atom-to-atom distance within 14 Å, while the average distance was calculated from the Cα to Cα distance of L472 and K474 to each residue within the surface patch. The average distance calculated is listed as 14 Å for residues for which the Cα to Cα distance was greater than 14 Å but in which the side chains are within the 14 Å cut-off. Residues of likely importance for M13-C06 binding and activity are listed in Table 18.

Table 18. Residues within IGF-1R predicted to be important for M13-C06 binding and activity. Residues 462 and 464 are italicized as these represent the predicted center of the IGF-1R binding epitope and experimental data demonstrates the importance of these residues in M13-C06 binding.

TABLE 18

| | | | | Distance | Distance | |
|---|---|---|---|---|---|---|
| IR AA | | | | to 472 (Å) | to 474 (Å) | Average |
| residue # | Surface | IGF1R AA | | (Cα | (Cα | distance |
| (2DTG) | accessibility | residue # | | to Cα) | to Cα) | (Å) |
| S437 | 0.477792 | S | 427 | 13.785 | 14 | 13.8925 |
| E438 | 0.337716 | E | 428 | 14 | 14 | 14 |
| E469 | 0.320544 | E | 459 | 9.95 | 14 | 11.975 |
| N470 | 0.8196 | S | 460 | 6.29 | 12.42 | 9.355 |
| E471 | 0.349164 | D | 461 | 3.79 | 9.57 | 6.68 |
| [symbol] | [symbol] | [symbol] | [symbol] | | 6.25 | 6.25 |
| [symbol] | [symbol] | [symbol] | [symbol] | 6.25 | 14 | 10.125 |
| S476 | 0.477792 | T | 466 | 12.45 | 6.43 | 9.44 |
| Y477 | 0.524048 | S | 467 | 14 | 9.15 | 11.575 |
| I478 | 0.5405 | T | 468 | 14 | 11.03 | 12.515 |
| R479 | 0.362378 | T | 469 | 14 | 14 | 14 |
| R488 | 0.375476 | T | 478 | 13.98 | 8.75 | 11.365 |
| E490 | 0.37206 | H | 480 | 9.18 | 5.84 | 7.51 |
| Y492 | 0.313493 | Y | 482 | 10.45 | 11.24 | 10.845 |
| W493 | 0.87318 | R | 483 | 11.17 | 13.03 | 12.1 |
| P495 | 0.824499 | P | 485 | 14 | 14 | 14 |
| D496 | 1 | D | 486 | 14 | 14 | 14 |
| E509 | 0.520884 | E | 499 | 14 | 14 | 14 |
| Q513 | 0.515108 | K | 503 | 14 | 14 | 14 |
| N514 | 0.68983 | N | 504 | 14 | 14 | 14 |
| V515 | 0.644094 | V | 505 | 14 | 14 | 14 |
| K544 | 0.865258 | N | 529 | 14 | 14 | 14 |
| S545 | 0.699624 | K | 530 | 14 | 14 | 14 |
| Q546 | 1 | D | 531 | 14 | 14 | 14 |
| N547 | 0.87424 | V | 532 | 14 | 14 | 14 |
| H548 | 0.406778 | E | 533 | 14 | 10.89 | 12.445 |
| W551 | 0.523908 | I | 536 | 14 | 14 | 14 |
| R577 | 0.41477 | H | 563 | 14 | 14 | 14 |
| T578 | 0.43254 | I | 564 | 13.19 | 14 | 13.595 |
| Y579 | 0.603591 | R | 565 | 9.54 | 14 | 11.77 |
| K582 | 0.34027 | K | 568 | 5.54 | 8.98 | 7.26 |
| D584 | 0.602475 | E | 570 | 7.01 | 7.4 | 7.205 |
| I585 | 0.340515 | I | 571 | 10.79 | 10 | 10.395 |
| I586 | 0.308085 | L | 572 | 13.04 | 10.49 | 11.765 |
| Y587 | 0.580196 | Y | 573 | 14 | 13.65 | 13.825 |

Published work has shown that antibodies that recognize residues 440-586 can be both inhibitory and agonistic to IGF-1 binding (Soos 1992; Keyhanfar 2007). 440-586 represents all of L2 and FnIII-1 with many potential non-overlapping surfaces accessible to anti-IGF-1R antibodies. Our study is the first study that we are aware of where the inhibitory epitope of IGF-1R has been mapped to a particular residue(s). A recent structure of INSR was co-crystallized with anti-INSR antibody known to inhibit insulin binding to its receptor (Soos 1986; McKern 2006). The homologous residue to His464 of IGF-1R (K474 of INSR) is part of the binding surface of this antibody with INSR. It is possible that M13-C06 shares a similar inhibitory mechanism for inhibiting IGF-1 binding to IGF-1R as the antagonistic anti-INSR antibody.

### Example 28

### M13.C06.G4.P.agly Antibody Effectively Localizes In Vivo to Tumor Cells, Inhibits Ki67 Expression, and Down-Regulates Expression of IGF-1R

M13.C06.G4.P.agly Antibody Effectively Localizes to Tumor Cells In Vivo

Methods: SCID Beige mice were injected with 2×10⁶ MCF-7 cells (in matrigel) in the presence of estrogen (0.36 mg pellet, 90 day release (Innovative Research of America)). Tumors were grown to 300-500 mm³ then mice were injected intraperitoneally with 30 mg/kg of M13.C06.G4.P.agly antibody. Mice were sacrificed and tumors were removed at 2, 6, 12, 24, and 48 hours post injection frozen in OCT and sectioned at 6 μm for immunohistochemical analysis (IHC). A tumor with no antibody injection was excised as a control. Tumors were frozen in OCT and sectioned at 6 μm for IHC. Substrate is Vector VIP, a purple stain. Bound antibody was detected using goat anti-human IgG H+L (Human Elite ABC kit, Vector Labs) on M13.C06.G4.P.agly or IDEC151 (negative control antibody) treated tumors. IGF-1R expression was detected using an α-IGF-1R Mab (clone 24-31, NeoMarkers/Lab Vision) on M13.C06.G4.P.agly or IDEC151 treated tumors. Similar studies were conducted in BxPC3 pancreatic cancer xenograft model.

Results (data not shown): In vivo efficacy experiments using a mouse MCF-7 breast or BxPC3 pancreatic tumor xenograft models revealed that intraperitoneal injection of M13.C06.G4.P.agly was effective at inhibiting tumor cell growth at 30 and 15 mg/kg. A time-course experiment was performed to study the pharmacodynamics of a single 30 mg/kg or 15 mg/kg dose of M13.C06.G4.P.agly in either MCF-7 or BX-Pc3 tumor-bearing mice, respectively. M13.C06.G4.P.agly localized to tumors as early as 6 hours post treatment, with maximum localization at 48 hours as determined by immunohistochemical analysis (1HC). The expression of IGF-1R as determined by Western and IHC analysis showed significant loss of IGF-1R in M13.C06.G4.P.agly treated tumors 6 hours post-treatment, with almost complete loss of IGF-1R at 24 hours. No change was observed in tumors treated with isotype-matched control antibody. Analysis of tumor lysates for signaling pathways revealed transient reduction of phosphorylated Erk and Akt in 2-12 hours.

M13. C06.G4.P.agly Antibody Inhibits Ki67 Expression

Ki67 staining of M13.C06.G4.P.agly treated tumors also showed a reduced number of proliferating cells compared to control antibody treated tumors (data not shown). These data indicate that M13.C06.G4.P.agly effectively localizes to tumors in vivo, and inhibits tumor growth by down-regulation of IGF-1R and inhibition of IGF-1R mediated signaling.

M13.C06.G4.P.agly Down-Regulates and Degrades IGF-1R in Tumors

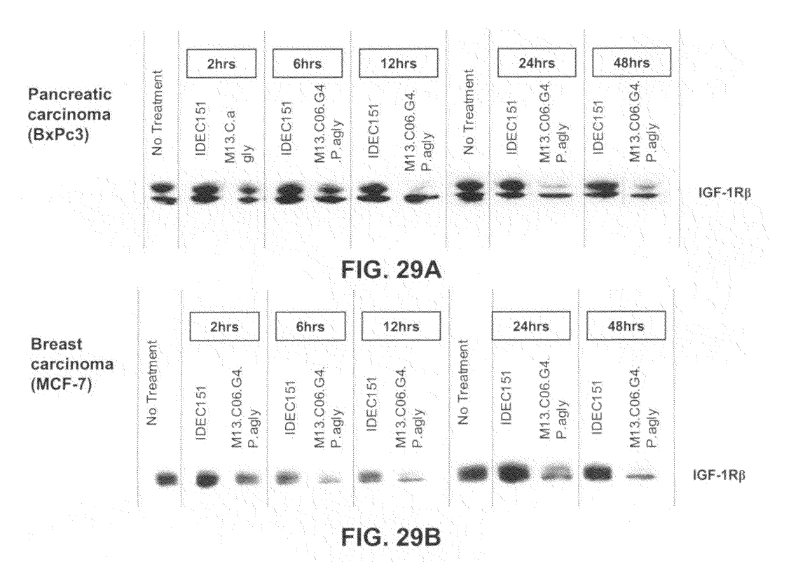

IGF-1R was immunoblotted from lysates of SCID mouse tumors generated with human pancreatic cells (BxPC3; FIG. 29 (A)) and breast cancer cells (MCF-7; FIG. 29(B)). Tumors were excised at designated time points after treatment with M13.C06.G4.P.agly or IDEC-151 negative control antibody. Tumors were snap frozen, pulverized and lysed. Protein concentration of tumor cell lysates were normalized and separated on 4-12% NuPAGE® gel (Invitrogen Inc., SD, CA). The gel was blotted to nitrocellulose filter, probed with polyclonal anti-IGF-1Rβ and detected by enzymatic reaction with anti-rabbit-horse radish peroxidase antibody. Results show that M13.C06.G4.P.agly resulted in down-regulation and degradation of IGF-1R compared to negative control antibody.

### Example 29

### M13.C06.G4.P.agly Antibody Demonstrates In Vivo Anti-Tumor Activity in a Variety of Tumor Model Systems

In addition to the in vivo inhibition of tumor growth demonstrated for M13.C06.G4.P.agly in lung and pancreatic model systems as described in previous examples, the following experiments further demonstrate the diversity of tumor cell models in which M13.C06.G4.P.agly exhibits activity.

Anti-tumor activity of M13.C06.G4.P.agly in tumors generated with MiaPaCa2 pancreatic carcinoma cells.

Female SCID mice were innoculated in the right flank with 2×10⁶ MiaPaCa2 pancreatic carcinoma cells in 50% Matrigel (BD Biosciences)/PBS. Tumors were allowed to reach a volume of 150 mm (L×W2/2) and mice were sorted and dosed intraperitoneally with single agent (antibody alone) and combination treatments (M13.C06.G4.P.agly antibody and gemcitabine). Gemcitabine alone (20 mg/kg, Q4D×3) and in combination with M13.C06.G4.P.agly (30 mg/kg) as well as M13.C06.G4.P.agly alone (at both 15 mg/kg and 30 mg/kg; 1× week×6) inhibited tumor growth.

In addition to gemcitabine, many other combination therapies could also be tested and used in conjunction with antibodies of the present invention. For example, combination therapies of compounds in the following categories, to list a small exemplary sampling, could be utilized with antibodies of the present invention:
EGFR tyrosine kinase inhibitors, for example:
   Tarceva (Erlotinib)
   Iressa (Gefitinib)
EGFR antibodies, for example:
   Erbitux (cetuximab)
   Victibix (panitumumab)
mTOR inhibitors, for example:
   temsirolimus
   rapamycin
and other anti-cancer compounds, for example:
   Gleevec (Imatinib)
   Sutent (Sunitinib)
   Sorafenib (Bay-439006)
   SAHA (HDAC inhibitor)
   HSP90 inhibitors
   M200 (Volociximab).

Anti-tumor activity of M13.C06.G4.P.agly in tumors generated with cells derived from a primary human colon adenocarcinoma.

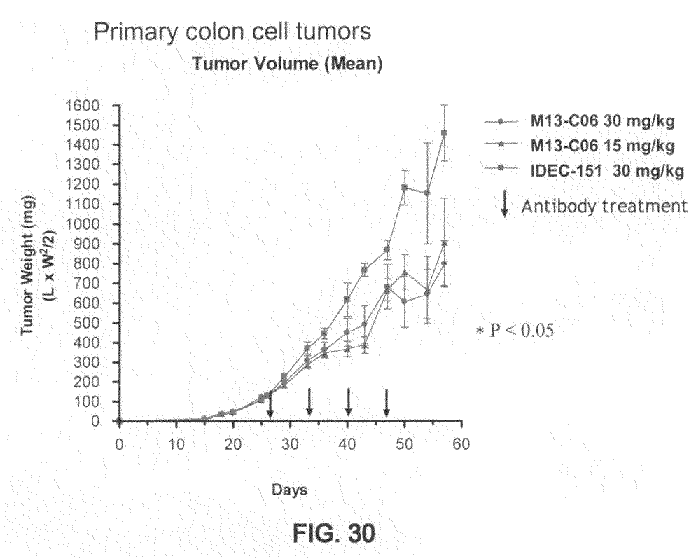

Female SCID mice were innoculated in the right flank with 1 mm³ of colon tumor fragments. The tumor fragment was derived by serial passage (5×) of colon tumor tissue obtained following surgical resection of a tumor from a patient with colon adenocarcinoma. Tumors were allowed to reach a volume of 150 mm³ (L×W2/2) and mice were sorted and dosed with the indicated treatments (n=6) (FIG. 30). Antibodies at 15 mg/kg or 30 mg/kg were dosed intraperitoneally 1× weekly.

Results: M13.C06.G4.P.agly effectively inhibited primary colon tumor (CT3) growth in SCID mice (FIG. 30).

Anti-tumor activity of M13.C06.G4.P.agly in tumors generated with MCF-7 breast carcinoma cells.

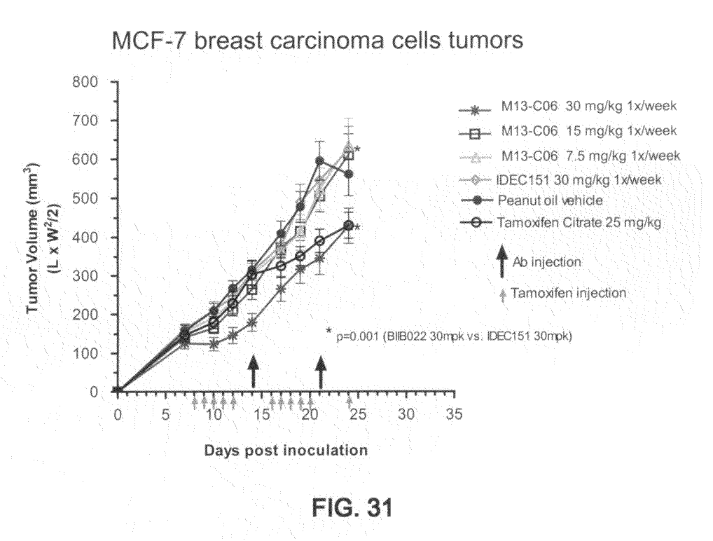

Female SCID Beige mice were innoculated in the right flank with 2×10⁶ MCF-7 cells (estrogen dependent) in 50% Matrigel/PBS. An estradiol pellet was implanted in the left flank 24 hours prior to cell inoculation (0.36 mg pellet estradiol, 90 day release (Innovative Research of America)). Tumors were allowed to reach a volume of 150 mm³ (L×W2/2) and mice were sorted and dosed with the indicated treatments (n=10) (FIG. 31). Antibodies were dosed intraperitoneally ix/week, while Tamoxifen Citrate (Sigma-Aldrich Corp. (St. Louis, Mo., USA)) in peanut oil was dosed sub-cutaneously 5 times a week for each regimen. Statistical analysis was performed using a paired student t test.

Results: M13.C06.G4.P.agly effectively inhibited growth of MCF-7 breast carcinoma tumors (FIG. 31).

Of course, the tumor inhibiting efficacy antibodies of the invention could also be readily tested in numerous other cancer cell types (such as: lung cancer cell lines H-1299, H-460, H-23; colon cancer cell lines Colo205 and HT-29; pancreatic cancer cell lines such as Panc-1; and, prostate cancer cell lines such as PC-3 to name a small exemplary sampling).

### Example 30

### M13.C06.G4.P.agly Antibody does not Exhibit In Vitro ADCC Activity

Methods: Human peripheral blood mononuclear cells were purified from heparinized whole blood by standard Ficoll-Paque separation. The cells were resuspended in GIBCO™ RPMI1640 media containing 10% FBS and 200 U/ml of human IL-2 and incubated overnight at 37° C. The following day, the cells were collected and washed once in culture media and resuspended at 1×10⁷ cells/ml.

Target cells (MCF-7, breast carcinoma cells) were incubated with 100 μCi⁵¹Cr for 1 hour at 37° C. The target cells were washed once to remove the unincorporated ⁵¹Cr, and plated at a volume of 1×10⁴ cells/well. Target cells were incubated with 50 μl of effector cells and 50 μl of antibody. A target to effector ratio of 1:50 was used throughout the experiments. Controls included were incubated with and without antibodies, these include M13.C06.G4.P.agly, Herceptin (positive control) and IDEC-151 (negative control—macaque/human chimeric IgG1 monoclonal antibody specific to CD4). Following a 4-hour incubation at 37° C., the supernatants were collected and counted on a gamma counter (Isodata Gamma Counter, Packard Instruments). The % lysis was determined using the following calculation:

% Lysis=[Sample Release (CPM)−spontaneous release (CPM)]÷[Maximum release (CPM)−spontaneous release (CPM)]×100%

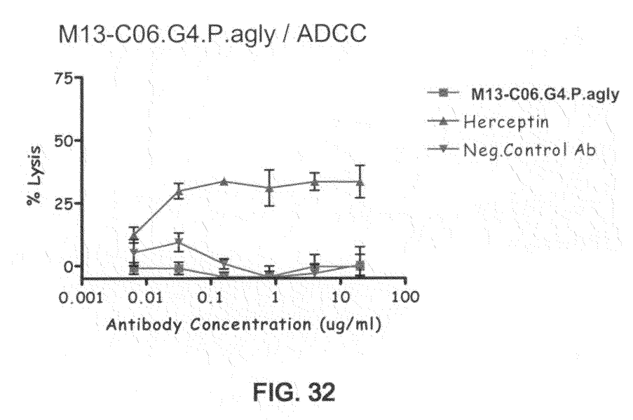

Results: In contrast to the Herceptin antibody positive control, neither M13-C06 or IDEC-151 antibodies exhibited ADCC activity, thereby indicating a lack of effector function for these latter antibodies (FIG. 32).

### Example 31

### Treatment of Human Cancer Using Anti-IGF-1R Antibodies

This example describes methods for treating cancer using antibodies against IGF-1R to target malignant cells, for example, hyperproliferating cells in which IGF-1R expression has been detected.

In certain embodiments, M13.C06.G4.P.agly antibody (or another antibody of the present invention) is purified and formulated with a suitable pharmaceutical vehicle for injection. A human patient with a hyperproliferative disorder is given multiple doses of M13.C06.G4.P.agly antibody (or another antibody of the present invention) by intravenous infusion at about 1 mg/kg body weight to about 100 mg/kg body weight, e.g., once per every two weeks or once a month, for at least six months. Intervals can also be irregular as indicated by measuring prognostic indicators in the patient.

Antibodies can be administered prior to, concurrently with, or after standard radiotherapy regimens as described herein. The patient is monitored to determine whether treatment has resulted in an anti-tumor response, for example, based on tumor regression, reduction in the incidences of new tumors, lower tumor antigen expression, or other means of evaluating disease prognosis.

### Example 32

### Residue Specific Epitope Mapping of Allosteric and Competitive Antibody Inhibitors of IGF-1R

Objective: The objective was to elucidate the binding epitopes of inhibitory anti-IGF-1R antibodies and the mechanism behind IGF-1/IGF-2 blockade.

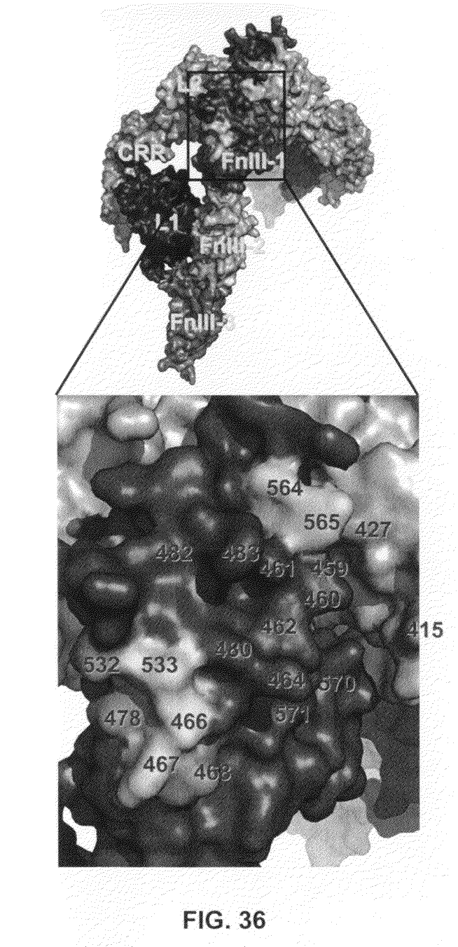

Background: IGF-1R (type 1 insulin-like growth factor receptor) is a receptor tyrosine kinase expressed on many normal cell types (Pollak et al., Nature Reviews Cancer, (2004) 4:505-516). IGF-1R is also involved in tumor growth and survival and has therefore been the target of both antibody and small molecule-based approaches for therapeutic intervention. Inhibitory antibodies have been targeted to the extracellular ligand-binding domain of the receptor. The IGF-1R extracellular region consists of 6 protein domains; an N-terminal Leucine Rich Repeat domain known as L1, a Cysteine Rich Region (CRR), a second Leucine Rich Repeat domain (L2), and three C-terminal Fibronectin Type III domains, denoted FnIII-1, FnIII-2, and FnIII-3 (FIG. 36). Here, we demonstrate that two separate epitopes on the surface of the IGF-1R ectodomain can lead to inhibition of the receptor. We generated novel, residue specific epitope mapping information concerning these two epitopes based on a dataset of 46 individual or double IGF-1R mutations. The first epitope resides in FnIII-1 and leads to allosteric blockade of both IGF-1 and IGF-2 binding. The second epitope is within the CRR domain and near the putative IGF-1/IGF-2 binding site. We discovered that subtle differences in antibody epitope within this region differentiate the ability to allosterically block the binding of a single ligand, IGF-1, from the ability to block both IGF-1 and IGF-2 competitively. Particular residues that must be targeted to achieve competitive blockade of both ligands have been identified here for the first time.

Materials: The anti-IGF-1R antibodies M13-C06, M14-C03, and P1E2 were purified as described above (for example, see Example 10). A commercially available inhibitory IGF-1R antibody (αIR3, (Jacobs et al., 1986)) was purchased from Calbiochem (Cat. No. GR11LSP5). Human IGF-1 with an N-terminal octahistidine tag was produced recombinantly in Pichia and purified using Ni²⁺-NTA agarose. A recombinant soluble human IGF-1R ectodomain construct containing a C-terminal 10-histidine tag, denoted hIGF-1R(1-902)-His₁₀, was purchased from R&D systems (Cat. No. 305-GR-050). Human and mouse IGF-1R(1-903)-IgG1-Fc fusion proteins were constructed and purified using standard protein A chromatography methods.

Methods: Antibody cross-blocking studies. The ability of various antibodies to block M13-C06 or M14-G11 was determined using biotinylated version of both antibodies and hIGF-1R-Fc. Briefly, 50 μL of 2 μg/mL hIGF-1R-Fc in 1×PBS were coated per well of a 96-well clear MaxiSorp plate (Nunc) for 2 hours at room temperature (RT, no shaking). Plates were washed with 1XPBS and blocked overnight at 2-8° C. using a PBS/1% BSA solution. Plates were washed and incubated with a 100 μL mixture of biotinylated M13-C06 or biotinylated M14-G11 (80 ng/mL) and inhibitor antibody for 1 hour at RT. Inhibitor antibodies were serially diluted (5-fold dilutions) from 40 μg/mL to 3 ng/mL. M13-C06 and M14-G11 were biotinylated using EZ-Link Sulfo-NHS-LC-Biotin according to protocol provided by the manufacturer (Pierce Cat. No. 21335). A control was also performed by serial dilution of a non-IGF-1R specific IgG4 isotype control antibody with biotinylated M13-C06 or biotinylated M14-G11. Plates were washed and shaken for 1 hour at RT with 100 μL/well streptavidin-HRP (1:4000 dilution into blocking buffer, Southern Biotech Cat. No. 7100-05). Plates were washed and 100 mL/well SureBlue Reserve TMB Microwell Peroxidase Substrate (KPL, Cat. No. 53-00-01) was added to the wells. Detection of the presence of biotinylated M13-C06 or M14-G11 was performed by reading the absorbance at 650 nm every 5 minutes using a Wallac 1420-041 Multilabel Counter plate reader.

The ability of various antibodies to block murine αIR3 was determined using “Zenon-Fab-HRP” labeled αIR3 and hIGF-1R-Fc. αIR (IgG1) was Zenon®-Fab-HRP labeled as described by the manufacturer (Invitrogen Cat. No. Z25054). Briefly, 50 μL of 2 μg/mL hIGF-1R-Fc in 1×PBS were coated per well of a 96-well clear MaxiSorp plate. (Nunc) for 2 hours at RT (no shaking). Plates were washed with 1×PBS and blocked overnight at 2-8° C. using a PBS/1% BSA solution. Plates were washed and incubated with a 100 μL mixture of Zenon-labeled αIR3 (40 ng/mL) and inhibitor antibody for 1 hour at RT. Inhibitor antibodies were serially diluted (5-fold dilutions) from 40 μg/mL to 3 ng/mL. A control inhibition was performed by serial dilution of a non-IGF-1R specific IgG4 isotype control antibody with Zenon-labeled αIR3. Plates were washed and 100 μL/well SureBlue Reserve TMB Microwell Peroxidase Substrate (KPL, Cat. No. 53-00-01) was added to the wells. Detection of Zenon-labeled αIR3 was performed by reading the absorbance at 650 nm every 5 minutes using a Wallac 1420-041 Multilabel Counter plate reader.

### IGF-1 and IGF-2 Blocking

hIGF-1R-Fc was biotinylated using EZ-Link Sulfo-NHS-LC-Biotin according to the protocol provided by the manufacturer (Pierce Cat. No. 21335). Biotinylated human IGF-1R-Fc at 5 μg/ml was added to the wells of SigmaScreen streptavidin-coated 96-well plates (Sigma, Cat. No. M5432-5EA; Sigma-Aldrich Corp. (St. Louis, Mo., USA)) at 100 μL/well and incubated overnight at 2-8° C. The plates were then washed four times with 200 μL/well PBST. Human IGF-1 His was prepared at 320 nM in PBST, 1.0 mg/ml BSA. Serial dilutions of anti-IGF-1R antibodies M13-C06, M14-C03, M14-G11, P1E2, and αIR3 (Calbiochem, Cat. No. GR11LSP5) were made up in the 320 nM IGF-1 His solution. Dilutions were made from 1.3 μM to 10 pM for M13-C06 and M14-C03, from 5.2 μM to 10 pM for M14-G11, and from 2.6 μM to 10 μM for both P1E2 and αIR3. Human IGF-2 His was prepared at 320 nM in PBST, 1.0 mg/ml BSA. The antibodies were serial diluted (from 1.3 μM to 5 μM for M13-C06 and M14-C03, from 5.2 μM to 5 pM for M14-G11 and αIR3, and from 5.2 μM to 20 μM for P1E2) using a solution of 320 nM IGF-2 His. The dilutions were added to the plates in duplicate at 100 μL/well and the plates were incubated at RT for 1 hour. The plates were then washed four times with 200 μL/well PBST. An HRP-conjugated anti-His Tag antibody (Penta-His HRP Conjugate, QIAGEN, Cat. No. 1014992) was diluted 1:1000 in PBST and added to plates at 100 μL/well, and the plates were incubated at RT for one hour. The plates were then washed four times with 200 μL/well PBST. SureBlue Reserve TMB Microwell Peroxidase Substrate (KPL, Cat. No. 53-00-01) was added to plates at 100 μL/well followed by 1% phosphoric acid at 100 μL/well once the desired reaction was observed. The absorbance of each well was determined at 450 nm, and the results were normalized and plotted against the log of antibody concentration.

### Epitope Mapping Mutations

The 46 mutant epitope mapping library was constructed by mutagenizing the wild-type hIGF-1R-Fc PV-90 plasmid using the STRATAGENE™ site-directed mutagenesis kit following the manufacturer's protocols. Incorporation of each mutant (or double mutant) within the PV-90 vector was confirmed by DNA sequencing. For DNA production, plasmids were transformed into DH5α (Invitrogen, Cat. No. 18258-012), cultured overnight at 37° C., and miniprepped or maxiprepped using the Qiagen Miniprep Kit or Qiagen Endotoxin-Free MaxiPrep Kit, respectively. 200 μg of each mutant plasmid was transiently transfected into 100 mL HEK293 T cells at 2×10⁶ cells/mL using the PolyFect transfection kit (Qiagen) for soluble protein secretion into the media. Cells were cultured in DMEM (Irvine Scientific), 10% FBS (low IgG bovine serum, Invitrogen—further depleted of bovine IgG by passage over a 20 mL protein A column) at 37° C. in a CO₂ incubator. After 7 days, supernatants containing each IGF-1R-Fc mutant were collected by centrifugation at 1200 rpm and filtration through a 0.2 μm filter. Each mutant was affinity purified by passage of its supernatant over a 1.2 mL protein A Sepharose FF column pre-equilibrated with 1×PBS. The mutants were eluted from the column using 0.1 M glycine, pH 3.0, neutralized with 1 M Tris, pH 8.5, 0.1% Tween-80, and concentrated to ˜300 μL using VivaSpin 6 MWCO 30,000 centrifugal concentration devices (Sartorius, Cat. No. VS0621).

### Western Blot Analysis of IGF-1R Mutants

hIGF-1R-Fc mutant samples were run on 4-20% Tris-Glycine gels (Invitrogen Cat. No. EC6028) using the Xcell SureLock Mini Cell (Invitrogen, Cat. No. EI0001) following the standard manufacturer protocol. Samples were transferred to nitrocellulose using the iBlot Dry Blotting System (Invitrogen, Cat. No. IB1001) and Transfer Stacks (Invitrogen, Cat. No. IB3010-01 or 3010-02) following the standard manufacturer protocol. Membranes were blocked overnight at 4° C. in 25 ml of PBST; 5 mg/mL non-fat dry milk. After blocking, membranes were washed once with 25 ml PBST for 5 min at room temperature. Membranes were incubated with a primary anti-IGF-1Rβ antibody (Santa Cruz Biotechnology Cat. No. sc-9038) at 1:100 in 10 mL PBST for 1 hr at room temperature. The membranes were subsequently washed three times in 25 ml PBST for 5 min. For detection, membranes were incubated with a secondary HRP-conjugated Goat anti-Rabbit IgG-Fc antibody (US Biological Cat. No. I1904-40J) at a 1:1000 dilution in 10 mL PBST for 1 hr at room temperature. Membranes were washed three times in 25 mL PBST for 5 min followed by one wash in 25 mL PBST for 20 min. Protein bands were detected using the Amersham ECL Western Blotting Analysis System (GE Healthcare, Cat. No. RPN2108) following the standard manufacturer protocol.

### Surface Plasmon Resonance Analysis of the IGF-1R-Fc Mutant Library

Surface plasmon resonance (SPR) experiments were performed on a Biacore 3000 instrument set to 25° C. Both mIGF-1R-Fc and hIGF-1R-Fc bind with high apparent affinity to research grade CM5 sensorchip surfaces containing immobilized M13-C06, M14-C03, and M14-G11. The antibody sensorchip surfaces were prepared by injecting each antibody (diluted 100 μg/mL in 10 mM Acetate, pH 4.0) over EDC/NHS-activated sensorchip surfaces according to the standard protocol of the manufacturer. The ability of mIGF-1R-Fc to bind the antibody surfaces was the result of high apparent avidity of the protein. Both hIGF-1R-Fc and mIGF-1R-Fc proteins oligomerize due to the incorporation of two separate homodimeric regions (IGF-1R and IgG1-Fc). To distinguish between actual high affinity antibody binding to hIGF-1R-Fc and low affinity antibody binding to mIGF-1R-Fc, the receptor-Fc fusions were captured on the M13-C06 and M14-G11 sensorchip surfaces followed by an additional injection of antibody (αIR3 and P1E2) or antibody Fab (M13-C06, M14-C03, and M14-G11). Receptor-Fc constructs were captured onto antibody surfaces by injection of 60 μL of the affinity-purified, concentrated material at a 1 μl/min over the sensorchip surfaces. After, completion of the receptor-Fc loading step, flow rates were elevated to 5 μl/min. Solutions containing M13-C06 Fab or αIR3 antibody at 10 nM, 3 nM, or 1 nM or M14-C03 Fab, M14-G11 Fab, or P1E2 antibody at 30 nM, 10 nM, or 3 nM were injected (50 μL) subsequent to the loading of each receptor-Fc construct. Dissociation was measured for 7 minutes after the antibody injections were complete. Finally, the flow rate was elevated to 30 μL/min and the chip surfaces were regenerated by 2×10 μL injections of 0.1 M glycine, pH 2.

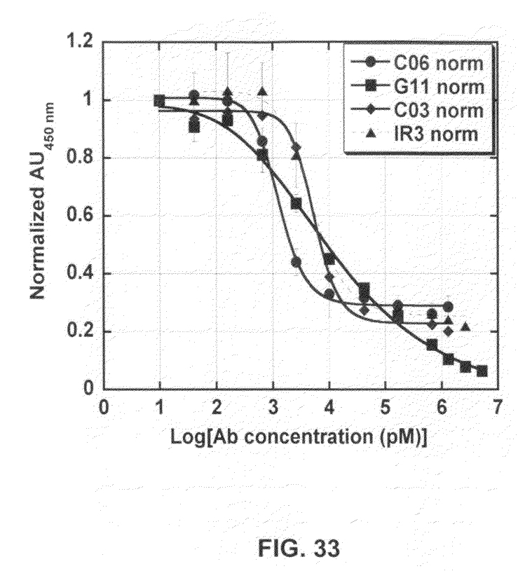

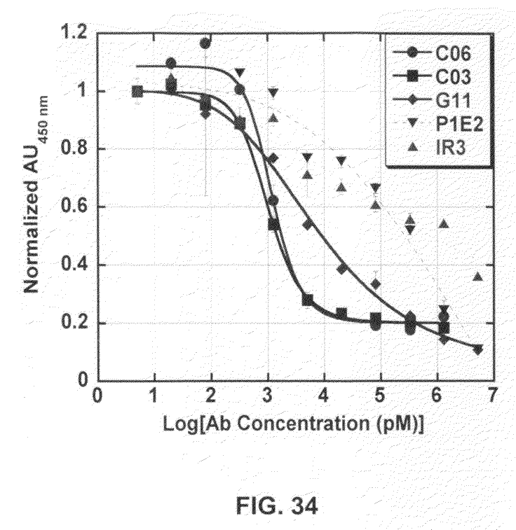

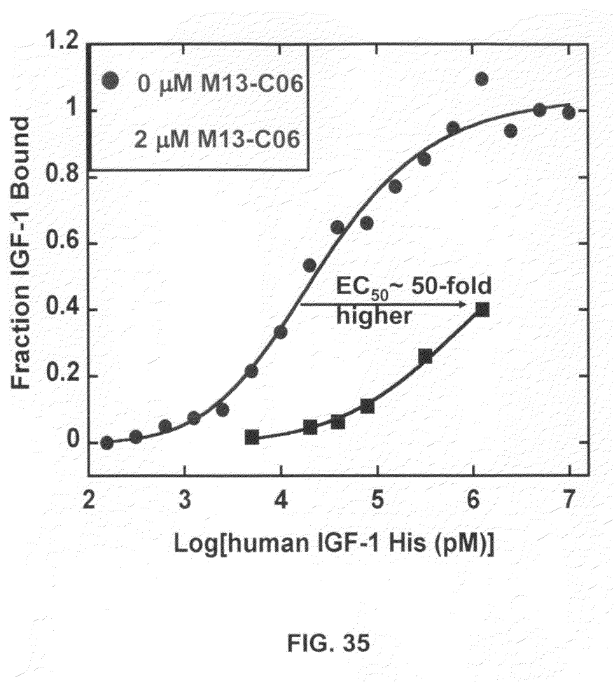

Results: IGF-1 and IGF-2 blocking properties of the anti-IGF-1R antibodies. Five antibodies (M13-C06, M14-C03, M14-G11, P1E2, and αIR3) were tested for their ability to block IGF-1 and IGF-2 from binding IGF-1R in an ELISA-based competition assay. M13-C06 and M14-C03 block both IGF-1 and IGF-2 binding to IGF-1R (FIGS. 33 & 34). Partial IGF-1 or IGF-2 binding could be restored by increasing the concentration of ligand in the assay even in the presence of saturating levels of M13-C06 or M14-C03. Additionally, the midpoint of the inhibition curves of M13-C06 and M14-C03 (IC₅₀) was independent of the concentration of IGF-1 or IGF-2 in the assay. Both results suggest an allosteric mechanism of ligand blockade. Titrating human IGF-1 His in the assay in the presence and absence of saturating levels of M13-C06 allowed us to measure an apparent affinity loss of the ligand for hIGF-1R-Fc. The data suggests that the presence of the M13-C06 antibody leads to an approximately 50-fold loss in affinity of human IGF-1 His for hIGF-1R-Fc (FIG. 35). P1E2 and αIR3 also block IGF-1 allosterically, but have little effect on IGF-2 binding to IGF-1R (FIGS. 33 & 34). These results for αIR3 are consistent with published results (Jacobs 1986). M14-G11 appeared to block both IGF-1 and IGF-2 in a competitive fashion (FIGS. 33 & 34). The IC₅₀ of M14-G11 depended on the IGF-1 concentration used in the assay. Saturating levels of the M14-G11 managed to block 100% of both ligands, albeit at much higher M14-G11 concentrations than the IC₅₀ of the allosteric blockers.

Cross-Blocking Properties of the Anti-IGF-1R Antibodies

The antibodies were all tested for their ability to cross-block one another in an IGF-1R ELISA binding assay (Table 19). M13-C06 and M14-C03 cross-blocked one another in the assay, but had no cross-blocking activity towards P1E2, αIR3 or M14-G11 in the assay. P1E2 and αIR3 were both able to completely cross-block labeled αIR3 and M14-G11 in the assays. M14-G11 demonstrated moderate cross-blocking activity towards αIR3 suggesting that M14-G11's epitope may overlap, but not be identical to the epitope(s) of αIR3 and P1E2.

Preliminary Epitope Mapping—Determination of the Epitope Locations

A preliminary set of 19 mutations was constructed to determine the location of the inhibitory anti-IGF-1R antibody epitopes. Based on the observation that M13-C06, M14-C03, and M14-G11 demonstrated little activity towards mouse IGF-1R, we identified a limited set of mutations within human IGF-1R that should enable our ability to locate the epitopes of the inhibitory anti-IGF-1R antibodies (for example, see Example 27). Mouse and human IGF-1R share 95% primary amino acid sequence identity. Thirty-three (33) residues differ between mouse and human IGF-1R in the ectodomain. Twenty (20) of these residues were targeted for mutation because their homologous positions within the homologous INSR ectodomain structure were exposed to solvent (pdb code 2DTG, (McKern 2006)). Accessible surface areas were calculated using StrucTools (hypertext transfer protocol://molbio.info.nih.gov/structbio/basic.html) with a 1.4 Å probe radius. Four pairs of these mutants were identified where the proposed mutations were next to one another in primary sequence. In these cases, each pair was double mutated within a single construct. Therefore, the 20 residues positions led to 16 initial mutant constructs. Four additional mutations were constructed due to mouse/human IGF-1R amino acid differences within the unstructured loop region of the FnIII-2 domain known to be important for IGF-1/IGF-2 binding (Whittaker 2001; Sorensen 2004). Two of these positions were close in primary sequence and could be combined within a single mutant construct. The final list of the 19 preliminary mutations (SD001-SD019) is provided in Table 20. The residue numbering shown in Table 20 assumes that the 30-residue IGF-1R signal sequence has been cleaved. Each of the constructs were expressed by transient transfection in 100 mL HEK293 cells for 1 week and purified using protein A chromatography. Purified mutant IGF-1R constructs were concentrated and assayed for expression/folding by Western Blot analysis. Expression was 10-30 μg for all the mutant constructs.

M13-C06, M14-C03, M14-G11, P1E2 and αIR3 were assayed for their ability to interact with each of the mutant IGF-1R-Fc fusion constructs using surface plasmon resonance (Biacore). To remove the uncertain concentrations of the IGF-1R-Fc fusion constructs as a variable in the assay, each mutant construct was captured on a research grade CM5 chip containing ˜10,000 RU immobilized M13-C03, M14-C03, and M14-G11 antibody. To enhance our ability to visualize attenuations in antibody binding to the captured mutant IGF-1R constructs, we utilized enzymatically derived M13-C06, M14-C03, and M14-G11 antigen binding fragments (Fabs).

Of these preliminary 19 mutant constructs, only SD015 (E464H) affected the ability of the M13-C06 and M14-C03 Fabs to bind IGF-1R. Mutation of residue 464 to histidine led to complete ablation of the binding reaction for both Fabs. All other mutant IGF-1R constructs bound with comparative equilibrium dissociation constants (K_{D}=1 nM and 5 nM for the M13-C06 and M14-C03 Fabs, respectively). These experiments localize the epitope of the M13-C06 and M14-C03 antibodies to the surface of the FnIII-1 domain. The V_{H} CDR regions of the two antibodies are highly similar (26 of 38 residues are identical) while the CDR regions of the V_{L} domain are unrelated suggesting a strong V_{H} bias towards antigen recognition. Not surprisingly, the two antibodies effectively cross-block one another. Soos and coworkers have shown using IR/IGF-1R chimeras that one or more epitopes within the 2^{nd} leucine rich repeat domain (L2) and 1^{st} fibronectin type III domain (FnIII-1) can lead to receptor inhibition (Soos 1992). This spans residues 333-609; a total of 276 residues. For the first time, we localize this inhibitory epitope directly to a single residue within the FnIII-1 domain, E464.

Of the 19 mutants, only SD008 (S257F) and SD012 (E303G), mutations in cysteine rich repeat (CRR) and L2 domains, respectively, attenuated the ability of the M14-G11 Fab to recognize human IGF-1R (Table 20). In both cases, mutation led to approximately 3-fold losses in affinity based on the measured K_{D}. All other mutant IGF-1R constructs, including SD015, which demonstrated no reactivity towards M13-C06 and M14-C03, bound the M14-G11 Fab with wild-type affinity (K_{D}˜4-6 nM).

αIR3 and P1E2, were also screened against the preliminary mutant library. Both of these antibodies exhibited a similar reduction in their affinity to SD012 compared to wild-type human IGF-1R-Fc; however, only P1E2 exhibited reduced binding to SD008 (Table 20).

Detailed Epitope Mapping: Residue Specific Definition of the M13-C06 and M14-C03 Antibody Epitopes

Based on the results of the preliminary IGF-1R mutant library that localized the M13-C06 and M14-C03 epitope(s) to the FnIII-1 domain of IGF-1R, a second set of mutations were designed to probe the surface of IGF-1R surrounding the original mutation, E464H, that led to ablation of antibody binding. A total of 21 residues were chosen for mutagenesis based on their 3D proximity to E464 (including a different mutation at residue 464 than the original histidine mutation). The 3D structure of the insulin receptor was used to estimate the proximity of residues surrounding 464. 7 pairs of residues were identified for mutation that were adjacent in primary sequence. Mutation of these residue pairs was done simultaneously to yield double mutants. Therefore, the second set of mutations consisted of 14 total constructs listed in Table 20 as SD101-SD114.

Expression, purification, and quality control of the 14 mutant constructs was performed as described for the first set of preliminary mutations (SD001-SD019). All 14 constructs expressed well and appeared folded based on Western Blot analysis except SD114. This construct expressed poorly and did not react in our Biacore experiment with M13-C06, M14-C03, or M14-G11—which recognizes a completely different epitope. Therefore, the data for this mutant construct was disregarded. The other 13 constructs allowed the precise, residue-specific definition of the M13-C06 and M14-C03 epitope. The residue-specific results are listed in Table 20. In summary, the epitopes of M13-C06 and M14-C03 were nearly identical and entirely contained within the FnIII-1 domain. The most crucial (perhaps central) residues were 461 and 462. SD103, which contains mutations at residues 461 and 462, demonstrated no reactivity towards the M13-C06 and M14-C03 Fabs and no reactivity towards the M13-C06 and M14-C03 surfaces. SD103 binding to the M14-G11 surface was no different than for any other FnIII-2 mutant construct indicating that this complete ablation was epitope specific. Other mutations that led to ablation or large decreases in antibody affinity (>100-fold decrease in affinity) for IGF-1R were found at IGF-1R residues 459, 460, 464, 480, 482, 483, 570, and 571. Mutations that led to small decreases in antibody affinity (2.5≧K_{D}≧10 nM) compared to wild-type human IGF-1R were found at residues 466, 467, 564, 565. The positions of these residues were mapped to the surface of the homologous IR structure (FIG. 36, McKern et al., 2006). Only two differential affects were observed for mutant IGF-1R binding to M13-C06 and M14-C03. Mutation at residue 533 strongly affected M14-C03 binding, but only had a weak affect on the binding of M13-C06. Mutation at residue 568 weakly attenuated M14-C03 binding, but had no affect on M13-C06 binding.

Based on the position and surface area coverage of the epitope, it is not surprising that both M13-C06 and M14-C03 were shown to allosterically inhibit IGF-1 and IGF-2 from binding IGF-1R. The epitope is on a receptor face opposite to the known ligand binding surface (Whittaker 2001; Sorensen 2004). Published work has shown that antibodies that recognize residues 440-586 can be both inhibitory and agonistic to IGF-1 binding (Soos 1992; Keynanfar 2007). Within IGF-1R, amino acid residues 440-586 represent all of L2 and FnIII-1 with many potential non-overlapping surfaces accessible to anti-IGF-1R antibodies. Our study is the first study that we are aware of that localizes the inhibitory epitope to a specific area on the receptor at residue specific resolution. A recent structure of the insulin receptor (IR) was co-crystallized with an anti-IR antibody known to inhibit insulin binding to its receptor (McKern 2006). The homologous residue to His464 of IGF-1R (K474 of IR) is part of the binding surface of this antibody with IR. It is possible that M13-C06 shares a similar inhibitory mechanism for inhibiting IGF-1 binding to IGF-1R as the antagonistic anti-IR antibody. Based on Biacore results (for example, see Example 27), M13-C06 appears to inhibit IGF-1 (and likely IGF-2) by reducing the kinetic association rate. The antibody appears to trap the receptor ectodomain in a conformation that makes it difficult for IGF-1 and IGF-2 to access the receptor-binding site.

### Detailed Epitope Mapping—Residue Specific Definition of the M14-G11, P1E2, and αIR3 Antibody Epitopes

Based on the results of the preliminary IGF-1R mutant library that localized the M14-G11, P1E2, and αIR3 epitopes to the CRR and L2 domains of IGF-1R, a third set of mutations were designed that cover the surface of IGF-1R surrounding the original mutations, S257F and E303G, that led to a reduction of antibody affinity towards the receptor. A total of 15 residues were chosen for mutagenesis based on their 3D proximity to S257 and E303 (including a different mutation at residue 257 than the original phenylalanine mutation). The 3D structure of the insulin receptor was used to estimate the proximity of residues surrounding S257 and E303. Two (2) pairs of residues were identified for mutation that were adjacent in primary sequence. Mutation of these residue pairs was done simultaneously to yield double mutants. Therefore, the second set of mutations consisted of 13 total constructs listed in Table 20 as SD201-SD213.

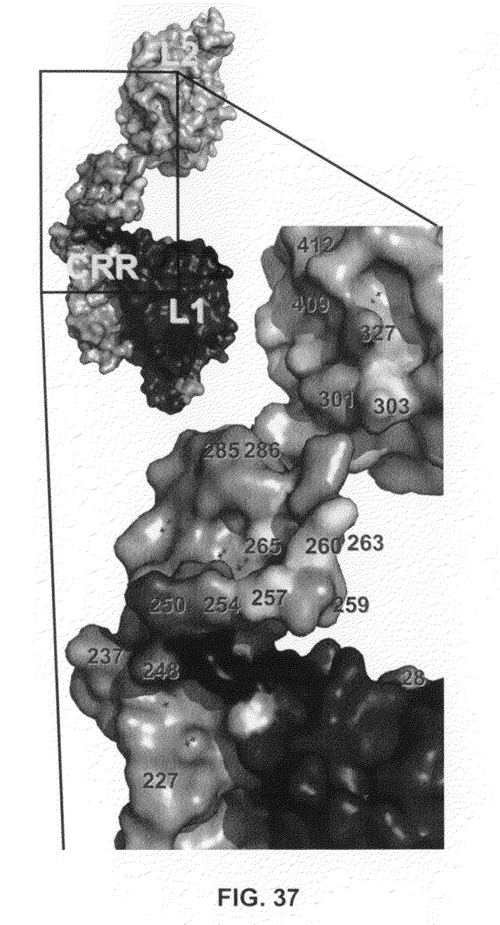

Expression, purification, and quality control of the 13 mutant constructs was performed as described for the first set of preliminary mutations (SD001-SD019). All of these constructs expressed well and appeared folded based on Western Blot analysis except for SD213. Data for SD213 was disregarded due to the ambiguity surrounding the folded state of the receptor. The other 12 mutant constructs led to the precise, residue-specific definition of the M14-G11, P1E2, and αIR3 epitopes. The residue-specific results are listed in Table 20. The epitopes differed between M14-G11, P1E2 and αIR3. This was not surprising, considering M14-G11 was shown to be a competitive inhibitor of both IGF-1 and IGF-2 while P1E2 and αIR3 were shown to allosterically inhibit the binding of IGF-1 only. The epitope of M14-G11 is near the center of the CRR domain on a surface that directly contacts residues that are known to have an effect on ligand binding (Whittaker 2001; Sorensen 2004). Mutations that ablated M14-G11 binding were found at positions 248 and 250. Mutation at residue 254 led to a moderate decrease in antibody affinity towards the receptor (10≧K_{D}≧100-fold above that of wild-type IGF-1R). Many other mutations predominantly in the CRR marginally reduced M14-G11 affinity for the receptor (2.5≧K_{D}≧10-fold above that of wild-type IGF-1R) including residues 257, 259, 260, 263, 265, and 303. The positions of these residues were mapped to the surface of the published structure of the first three ectodomains of IGF-1R (FIG. 37) (Garrett, et al., “Crystal structure of the first three domains of the type-1 insulin-like growth factor receptor,” Nature, (1998) Jul. 23; 394(6691):395-9).

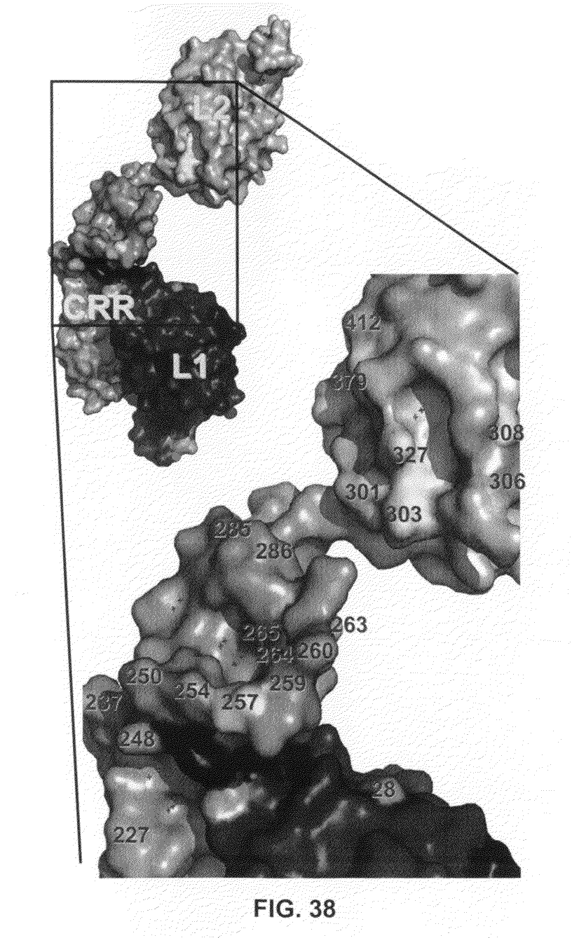

The epitopes of P1E2 and αIR3 were similar to one another, with a few minor differences. The epitopes are primarily within the CRR domain on residues overlapping with those of M14-G11, but residing on a face of the receptor rotated slightly away from the IGF-1/IGF-2 binding pocket. Additionally, residues at the C-terminus of the CRR domain and well into the L2 domain (beyond those that had any effect on M14-G11 binding) were found to marginally reduce the affinity of αIR3 alone, (Table 20). P1E2 binding to IGF-1R was ablated by mutation at residues 254 and 265; moderately reduced (10≧K_{D}≧100-fold above that of wild-type IGF-1R) by mutation at residue 257; and marginally reduced (2.5≧K_{D}≧10-fold above that of wild-type IGF-1R) by mutation at residues 248 and 303. αIR3 binding to IGF-1R was ablated by mutation at residues 248 and 265; moderately reduced (10≧K_{D}≧100-fold above that of wild-type IGF-1R) by mutation at residue 254; and marginally reduced (2.5≧K_{D}≧10-fold above that of wild-type IGF-1R) by mutation at residues 263, 301, 303, 308, 327, and 379. The position of the residues that affect P1E2 and αIR3 binding to IGF-1R (the average affect on the two antibodies) were mapped to the surface of the published structure of the first three ectodomains of IGF-1R (FIG. 38) (Garrett, et al., “Crystal structure of the first three domains of the type-1 insulin-like growth factor receptor,” Nature, (1998) Jul. 23; 394(6691):395-9). αIR3 and P1E2 appear to have the same allosteric/IGF-1 only blocking characteristic of two antibodies described recently in the literature (Keyhanfar 2007). It was shown that residues 241, 242, 251, and 266 affect the ability of these antibodies to bind receptor. Our data is consistent with this report and suggests additional importance for residues 257 and 265.

The major difference between M14-G11 (competitive IGF-1 and IGF-2 blocker) and P1E2/αIR3 epitopes are in the area adjacent to the IGF-1 binding site. The ability to simultaneously recognize residues 248, 250, and 254 may be a defining factor that enables M14-G11 to competitively block both IGF-1 and IGF-2 binding. Both P1E2 and αIR3 are completely unaffected by the D250S mutation, which completely ablates M14-G11 binding to the receptor. The binding of M14-G11 to IGF-1R is also attenuated by mutations on the inner cleft of the CRR domain near the IGF-1 binding site (residues 259 and 260, FIGS. 37 & 38) perhaps explaining how this antibody sterically and competitively blocks ligand from engaging the receptor. Mutations at these positions had no effect on P1E2 or αIR3 binding. P1E2 and αIR3 affinity is attenuated by mutations on a surface slightly outside the IGF-1 binding groove (FIGS. 37 & 38). Therefore, residues that appear to be specifically recognized by M14-G11 that may lead to competitive ligand blockade are D250, E259, and S260.

Residue mutations that attenuate αIR3 and M14-G11 binding to IGF-1R extend from the center of the CRR domain into the L2 domain. It is unlikely that all these residues engage in simultaneous direct interactions with the antibodies based on published results describing average antibody epitope areas (Davies 1996). Recent data has demonstrated that the stability and folding of repeat proteins is different from most globular domains (Kajander 2005). Repeat domains tend to be elongated structures that undergo non-cooperative folding/unfolding reactions similar to helix-coil transitions of isolated α-helices. From a simplistic view, globular domains are generally cooperatively folded and exist in either a single natively folded state or a denatured state. The structures of globular domains are not partially disrupted by single mutations provided the mutation does not lead to the overall unfolding of the domain. In contrast, folded repeat domains may gradually revert to unfolded domains upon mutation. Thus, mutations along the surface of the IGF-1R CRR or L2 domains that affect antibody binding may do so by modifying the overall structure (or order) of the these domain. This mechanism also explains how antibody stabilization of a particular CRR or L2 domain conformation may affect the dynamic binding reaction of the CRR domain with ligand. This would be expected to happen in an allosteric fashion (as observed for P1E2 and αIR3) provided the antibody doesn't also sterically block ligand from binding (as observed for M14-G11).

TABLE 19
Summary results of antibody cross-blocking experiments.

| Antibody | M13-C06 cross- | M14-G11 cross- | αIR3 |
|---|---|---|---|
| Inhibitor | blocking | blocking | cross-blocking |
| M13-C06 | +++++ | − | − |
| M14-C03 | +++++ | − | − |
| M14-G11 | − | +++++ | +++ |
| αIR3 | − | +++++ | +++++ |
| P1E2 | − | +++++ | +++++ |

+++++ = antibody binding competition relative to itself (90-100%)
++++ = 70-90% competition
+++ = 50-70% competition
++ = 30-50% competition
+ = 10-30% competition
+/− = 0-10% competition
N/A = results not available.

TABLE 20
Complete list of IGF-1R mutants and their affect on antibody binding.

| Mutation | | | | | | | | |
|---|---|---|---|---|---|---|---|---|
| IGF-1R | | | | | | | | |
| position | | | IGF- | | | | | |
| (w/out | | IR 3D | 1R | C06 | C03 | G11 | P1E2 | αIR3 |
| signal) | SD# | struct # | Domain | binding^{a} | binding | binding | binding | binding |
| Y28A | SD001 | 32 | L1 | NE | nd | NE | nd | nd |
| M156A | SD002 | 163 | L1 | NE | nd | NE | nd | nd |
| T188F | SD003 | 195 | L1 | NE | nd | NE | nd | nd |
| S210H | SD004 | 218 | CRR | NE | NE | NE | nd | nd |
| A211Q | | | | | | | | |
| A217T | SD005 | 224 | CRR | NE | NE | NE | nd | nd |
| A227K | SD006 | 234 | CRR | NE | NE | NE | NE | NE |
| N237G | SD007 | 244 | CRR | NE | NE | NE | NE | NE |
| S257F | SD008 | 264 | CRR | NE | NE | W | W | NE |
| E264K | SD009 | 275 | CRR | NE | NE | NE | NE | NE |
| G271D | SD010 | 282 | CRR | NE | NE | NE | NE | NE |
| G285S | SD011 | 295 | CRR | NE | NE | NE | NE | NE |
| S286T | | 296 | | | | | | |
| E303G | SD012 | 313 | L2 | NE | NE | W | W | W |
| D405K | SD013 | 415 | L2 | NE | NE | NE | nd | Nd |
| K412A | SD014 | 422 | L2 | NE | NE | NE | NE | NE |
| A413Q | | | | | | | | |
| H464E | SD015 | 474 | FnIII-1 | S | S | NE | nd | nd |
| D531Q | SD016 | 547 | FnIII-1 | NE | NE | NE | nd | nd |
| V532N | | | | | | | | |
| I650S | SD017 | * | FnIII-2 | NE | NE | NE | nd | nd |
| | | | loop | | | | | |
| E665A | SD018 | * | FnIII-2 | NE | NE | NE | nd | nd |
| | | | loop | | | | | |
| E739W | SD019 | * | FnIII-2 | NE | NE | NE | nd | nd |
| L741F | | | loop | | | | | |
| S427L | SD101 | 437 | L2 | NE | NE | nd | nd | nd |
| E459A | SD102 | 469 | FnIII-1 | S | S | nd | nd | nd |
| S460A | | 470 | | | | | | |
| D461A | SD103 | 471 | FnIII-1 | S-most | S-most | nd | nd | nd |
| V462T | | 472 | | critical | critical | | | |
| H464A | SD104 | 474 | FnIII-1 | S | S | nd | nd | nd |
| T466L | SD105 | 476 | FnIII-1 | W | W | nd | nd | nd |
| S467Y | | 477 | | | | | | |
| T468R | SD106 | 478 | FnIII-1 | NE | NE | nd | nd | nd |
| T478R | SD107 | 488 | FnIII-1 | NE | W | nd | nd | nd |
| H480E | SD108 | 490 | FnIII-1 | S | S | nd | nd | nd |
| Y482A | SD109 | 492 | FnIII-1 | S | S | nd | nd | nd |
| R483W | | 493 | | | | | | |
| E533H | SD110 | 548 | FnIII-1 | W | S | nd | nd | nd |
| I564T | SD111 | 578 | FnIII-1 | W | W | nd | nd | nd |
| R565A | | 579 | | | | | | |
| K568A | SD112 | 582 | FnIII-1 | NE | W | nd | nd | nd |
| E570A | SD113 | 584 | FnIII-1 | S | S | nd | nd | nd |
| I571T | | 585 | | | | | | |
| L572D | SD114 | 586 | FnIII-1 | *Fold | *Fold | nd | nd | nd |
| Y573D | | 587 | | Affected | Affected | | | |
| D248A | SD201 | 255 | CRR | nd | nd | S | W | S |
| D250S | SD202 | 257 | CRR | nd | nd | S | NE | NE |
| N254A | SD203 | 261 | CRR | nd | nd | M | S | M |
| S257K | SD204 | 264 | CRR | nd | nd | W | M | NE |
| E259K | SD205 | 270 | CRR | nd | nd | W | NE | NE |
| S260N | | 271 | | | | | | |
| S263R | SD206 | 274 | CRR | nd | nd | W | NE | W |
| G265Y | SD207 | 276 | CRR | nd | nd | W | S | S |
| V301Y | SD208 | 311 | L2 | nd | nd | NE | NE | W |
| K306E | SD209 | 316 | L2 | nd | nd | NE | NE | NE |
| T308E | SD210 | 318 | L2 | nd | nd | NE | NE | W |
| K327N | SD211 | 337 | L2 | nd | nd | NE | NE | W |
| L379R | SD212 | 389 | L2 | nd | nd | NE | NE | W |
| E381K | SD213 | 391 | L2 | nd | nd | *Fold | *Fold | *Fold |
| E382L | | 392 | | | | Affected | Affected | Affected |

^{a}No effect (NE): measured K_{D} within 2.5-fold of WT hIGF-1R-Fc; Weak (W): measured K_{D} between 2.5-10-fold higher than WT; Medium (M): measured K_{D} between 10-100-fold higher than WT; Strong (S): binding to antibody was ablated by mutation; and nd: not determined.
*“Fold affected” implies that the mutant receptor expression was attenuated and the protein behaved in an aberrant fashion presumably because the “folding” of the receptor was “affected.”

### Example 33

### Combined Targeting of Distinct IGF-1R Epitopes with Ligand-Blocking Antibodies Results in Enhanced Inhibition of Tumor Cell Growth

Objective: Investigate the functional effects of combining inhibitory anti-IGF-1R antibodies that bind to non-overlapping epitopes in cell-based tumor growth assays.

Background: Biochemical studies described herein demonstrate that combinations of inhibitory anti-IGF-1R antibodies that bind to non-overlapping epitopes can lead to synergistic improvement in the blocking of the IGF-1 and IGF-2 ligands to the receptor. Such combinations can lead to complete ligand blockade with greater potency (i.e. at lower antibody concentrations).

### Materials and Methods: Cell Growth Inhibition Assay

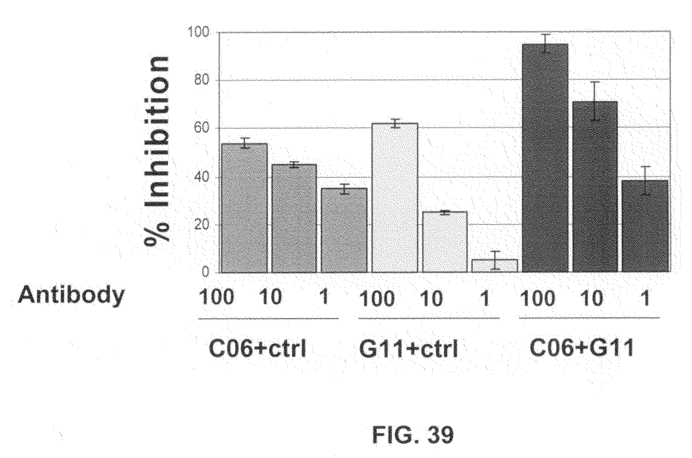

The ability of antibodies to block IGF-1 and IGF-2 driven tumor cell growth was tested using a cell viability assay. BxPC3 (human pancreas adenocarcinoma) and H322 M (human non-small cell lung tumor) (ATCC) tumor lines were purchased from ATCC. Cell lines were grown in complete growth medium containing RPMI-1640 (ATCC) and 10% fetal bovine serum (Irvine Scientific Inc. (Santa Ana, Calif., USA)). Trypsin-EDTA solution (Sigma-Aldrich Corp. (St. Louis, Mo., USA)) was used for removal of adherent cells from culture vessels. Phosphate buffered saline, pH 7.2, was from MediaTech Inc. (Herndon, Va., USA). The 96-well clear bottom plates for luminescent assay was purchased from Wallac Inc. Cells grown to 80% confluent monolayers were trypsinized, washed, resuspended, and plated into 96-well plates in 200 μl of 0.5% growth medium at 8×10³ cells/well for both BxPC3 and H322 M cells. After 24 hours, the culture medium was replaced with 50 μl or 100 μl of serum free medium (SFM), and 50 μl of serially diluted antibodies (at 4× concentrations shown in FIGS. 39-41) were added. Following another 30 minutes of incubation at 37° C., 50 μl of IGF-1 and IGF-2 at 4× concentrations was added was added. All treatments were done in triplicate. The cells were incubated for another 72 hours until lysed to determine the amount of ATP present using the CELL TITER-GLOB Luminescent Cell Viability Assay ((Promega Corporation, 2800 Woods Hollow Rd., Madison, Wis. 53711 USA). The 1:1 mixture of reagent and SFM was added at 200 μl/well. Luminescence was detected and quantitated in Relative Luminescence Units (RLU) on a Wallac (Boston, Mass.) plate reader. Inhibition was calculated as [1-(Ab-SFM RLU)/(IGF-SFM RLU)]×100%. An isotype matched antibody, IDEC-151 (human G4.P antibody), was used as a negative control (“ctr” or “ctrl” in FIGS. 39-41).

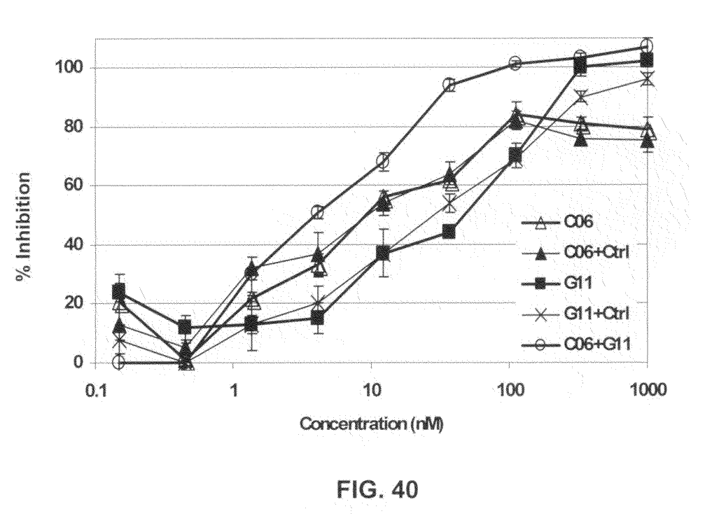

Results: The ability of M13.C06.G4.P.agly (C06) and M14.G11.G4.P.agly (G11) anti-IGF1-R antibodies to inhibit cell growth in vitro was measured indirectly by relative comparisons of cellular ATP as a measure of metabolic activity. Both C06 and G11 inhibited IGF-1 and IGF-2 stimulated growth of BxPC3 pancreatic tumor cell lines under serum-free conditions in a dose dependent manner (FIG. 39). Importantly the cells exposed to equimolar amounts of C06 and G11 antibodies combined resulted in a significantly enhanced inhibition of growth at 10 and 1 nM concentrations compared to that of either antibody alone (FIG. 39). These results were further confirmed in an experiment where a combination of C06 and G11 was tested at wide range of antibody concentrations (1 uM to 0.15 nM). FIG. 40 shows that the combination of equimolar amounts of C06 and G11 antibodies at concentrations between 500 nM and 5 nM resulted in significantly enhanced inhibition of cell growth compared to that observed with either antibody alone at the same corresponding antibody concentrations.

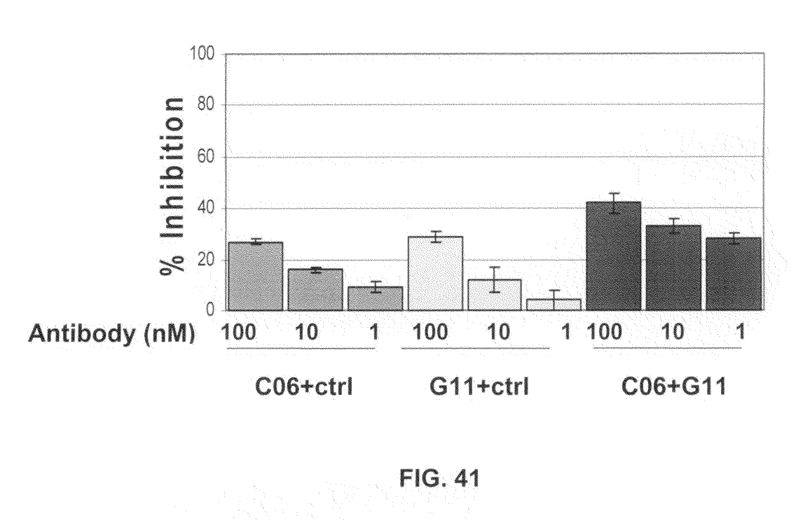

To demonstrate that the inhibition observed with the pancreatic cancer cell line (BxPC3) is also applicable to other tumor types, the combinations of C06 and G11 was evaluated in H322 M cell line of non-small cell lung cancer origin. FIG. 41 shows an example of the effects observed in H322 M grown under standard cell culture conditions in the presence of 10% fetal bovine serum, where a significantly greater inhibition of cell growth resulted from the C06/G11 antibody combination compared to either antibody alone.

### Example 34

### Further Discrimination of Allosteric and Competitive IGF-1 and/or IGF-2 Ligand Binding Inhibition Properties of Anti-(Human)IGF-1R Antibodies

Background: Antibody Fab phage panning using hIGF-1R-Fc (see, Example 1) and a murine immunization strategy using hIGF-1R-Fc (see, Example 17) yielded a spectrum of antibodies with various ligand binding inhibition properties. In this Example further discrimination of the ligand binding properties of inhibitory anti-IGF-1R antibodies (as originally described in Example 32) were categorized into different subgroups. For example, it has been determined that antibodies P1E2, P1A2, and αIR3 allosterically inhibit IGF-1 binding to IGF-1R; that antibody P3F9 allosterically inhibits IGF-2 binding to IGF-1R; and that antibodies M13-C06, M14-C03, and 20C8 allosterically inhibit binding of both IGF-1 and IGF-2 to IGF-1R. Additionally, one antibody, M14-G11, competitively inhibits binding of IGF-1 and IGF-2 to IGF-1R.

TABLE 21
IGF-1R antibody ligand binding inhibition properties as
presently determined are shown below.

| | Inhibition of | Proposed | |
|---|---|---|---|
| | IGF- | mechanism of | |
| | 1 and/or IGF-2 | ligand binding | IGF-1R domain of |
| Antibody | Binding | inhibition | antibody binding |
| M13-C06 | 1 & 2 | Allosteric | FNIII-1 |
| M14-C03 | 1 & 2 | Allosteric | FNIII-1 |
| 20C8 | 1 & 2 | Allosteric | CRR (primarily center) |
| M14-G11 | 1 & 2 | Competitive | CRR (primarily center) |
| P1E2 | 1 only | Allosteric | CRR (center to C- |
| | | | terminus and L2 domain) |
| P1A2 | 1 only | Allosteric | CRR and L2 |
| αIR3 | 1 only | Allosteric | CRR (C-terminus) and L2 |
| P3F9 | 2 only | Allosteric | CRR and L2***** |

Methods: Antibodies were constructed and purified as described in Example 32, except for αIR3 and P3F9. As described in Example 32, the control αIR3 MAb known to block IGF-1 (Jacobs et al., 1986) was purchased commercially. Generation of M13-C06, M14-C03, and M14-G11 is described in Example 1. Generation of P1E2, P1A2, 20C8 is described in Examples 17-19. The P3F9 antibody was derived from the same mouse immunizations with hIGF-1R-Fc via standard hybridoma technologies as described for P1A2, P1E2, and 20C8 antibodies.

Ligand Blocking Properties. The ability of the antibodies to block IGF-1 and IGF-2 was determined using the IGF-1 and IGF-2 blocking ELISA as described in Example 32. IGF-1 and IGF-2 concentrations in the assay were 320 nM and 640 nM, respectively.

Results: The eight antibodies described above (i.e., P1E2, P1A2, αIR3, P3F9, M13-C06, M14-C03, and 20C8, and M14-G11) were assayed using the IGF-1 and IGF-2 blocking ELISAs. The concentrations of IGF-1 and IGF-2 in the assays were kept at 320 nM and 640 nM, respectively (i.e., well above their physiological concentrations at which the ligands are expected to be active biologically) to discriminate allosteric vs. competitive ligand blocking. Allosteric ligand blocking is expected to simply change the affinity of the ligands for the receptor. The high ligand concentrations (i.e., 320 nM IGF-1 and 640 nM IGF-2) are approximately 10-20-fold above the natural affinities (KD) for IGF-1R. Under these conditions, allosteric inhibitors may not completely abrogate binding of IGF-1 or IGF-2 to the receptor, thus leading to incomplete inhibition upon reaching saturating levels of inhibitory antibody. A competitive antibody should be capable of completely inhibiting IGF-1 or IGF-2 binding at saturating levels of antibody. Additionally, a second means of identifying a competitive inhibitor is via the antibody's IC50 value which should depend on the concentration of IGF-1 or IGF-2 in the assay (since the antibody must compete for binding to IGF-1R in direct competition with the quantity of ligand present in solution).

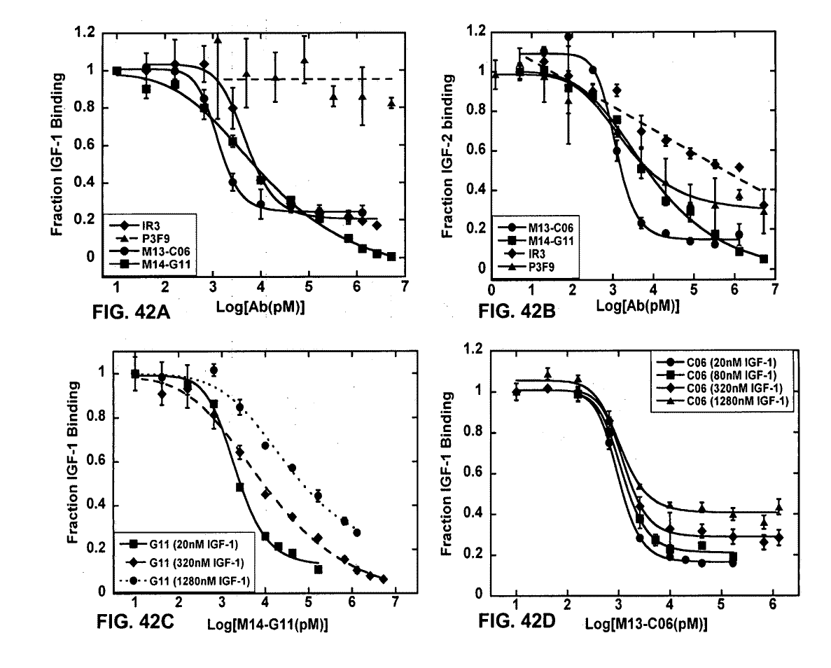

Assay results demonstrate that the antibodies can be broken into four separate ligand binding inhibition categories (Table 22, FIG. 42). P1E2, P1A2, and αIR3 (Jacobs et al., 1986) were shown to inhibit only IGF-1 allosterically. P3F9 was the only antibody that specifically inhibited only IGF-2 allosterically. M13-C06, M14-C03, and 20C8 were shown to inhibit both IGF-1 and IGF-2 allosterically. None of the allosteric inhibitors led to 100% blocking at saturating levels of antibody (Table 22, FIG. 42 A & B). M14-G11 was shown to inhibit IGF-1 and IGF-2 competitively. M14-G11 not only led to complete IGF-1 and IGF-2 inhibition (FIG. 42 A & B), but its IC50 value was highly dependent on the ligand concentrations used in the assay (FIG. 42 C); as opposed to allosteric inhibitors such as M13-C06, wherein the IC50 value was unaffected by the level of IGF-1 or IGF-2 (FIG. 42 D).

TABLE 22
IGF-1 and IGF-2 blocking characteristics of select anti-IGF-1R
antibodies (and αIR3).

| | | | | |
|---|---|---|---|---|
| | | | | % IGF-2 |
| | IC₅₀: | % IGF-1 | | inhibition |
| | 320 nM | inhibition | | upon |
| | IGF-1 | upon antibody | IC₅₀: 640 nM | antibody |
| Antibody | inhibition | saturation | IGF-2 inhibition | saturation |

IGF-1 only inhibitors (allosteric)

| | | | | |
|---|---|---|---|---|
| P1E2 | 13 | 38 | Did not inhibit | ~50 |
| P1A2 | 12 | 23 | Did not inhibit | ~50 |
| αIR3 | 4.9 | 23 | Did not inhibit | ~60 |

IGF-2 only inhibitors (allosteric)

| | | | | |
|---|---|---|---|---|
| P3F9 | Did not | ~80 | 1.7 | 34 |
| | block | | | |

IGF-1 & IGF-2 inhibitors (allosteric)

| | | | | |
|---|---|---|---|---|
| M13-C06 | 1.3 | 25 | 1.9 | 16 |
| M14-C03 | 5.4 | 19 | 1.0 | 21 |
| 20C8 | 2.9 | 24 | 1.0 | 22 |

IGF-1 & IGF-2 inhibitors (competitive)

| | | | | |
|---|---|---|---|---|
| M14-G11 | 13 | 1 | 7.9 | 1 |

FIG. 42 shows discrimination of the allosteric or competitive IGF-1 and IGF-2 ligand inhibition properties of anti-IGF-1R antibodies. A) IGF-1 and B) IGF-2 binding inhibition by antibodies representing the four inhibitory classes: (i) allosteric IGF-1 only inhibitor; (ii) allosteric IGF-2 only inhibitor; (iii) allosteric IGF-1 and IGF-2 inhibitor; and (iv) competitive IGF-1 and IGF-2 inhibitor. C) Shows IGF-1 concentration dependent activity of a representative allosteric IGF-1R inhibitor, M13-C06. D) Shows a representative competitive inhibitor, M14-G11.

### Example 35

### Additional Epitope Mapping of Anti-(Human)IGF-1R Antibodies

Background: Previous examples show data obtained by analyzing the binding properties of antibodies M13-C06, M14-C03, M14-G11, αIR3, and P1E2 against a library of 46 different single or double mutant human IGF-1R constructs. This example presents additional data obtained by analyzing the binding properties of antibodies P1A2, P3F9, and 20C8 against select CRR/L2 IGF-1R mutants. The data demonstrate cross-blocking activity of these antibodies towards M14-G11, whose epitope resides in the CRR/L2 region.

Previously, in Example 34 it was shown that P1A2 inhibits only IGF-1 binding (allosterically); similar to the activity of P1E2 and αIR3. It was also shown that P3F9 inhibits IGF-2 (allosterically) and is unique in this regard. Likewise, it was shown that 20C8 inhibits both IGF-1 and IGF-2 (allosterically); similar to the activity of M13-C06 and M14-C03. However, 20C8 inhibits IGF-1 & IGF-2 binding by specifically binding to an epitope that closely overlaps the epitope bound by M14-G11, and not that of M13-C06 or M14-C03 (which bind to the FnIII-1 domain).

To provide additional antibody epitope and ligand-binding inhibition data, the antibody binding activities of M14-G11, P1E2, αIR3, P1A2, P3F9, and 20C8 were screened against five additional hIGF-1R-Fc mutant constructs. In particular, these 5 additional IGF-1R mutant constructs provided a further enhanced assessment of the CRR/L2 region epitopes in IGF-1R to which these antibodies bind. Hence, these additional five mutants further assist in differentiating the basis of ligand inhibitory activity by antibodies that inhibit receptor binding of IGF-1 only, IGF-2 only, or both IGF-1 and IGF-2.

Methods: Construction of the additional 5 mutations in the CRR domain of human IGF-1R. IGF-1R residues 229 (Y to L), 249 (R to F), 251 (F to L), 255 (I to A), and 290 (Y to L) were mutated within our human IGF-1R-Fc construct based on either a neutral (i.e. not drastic amino acid type change) or a change to the amino acid that exists within INSR. The additional 5 mutations were constructed, expressed, and purified as described in Example 27.

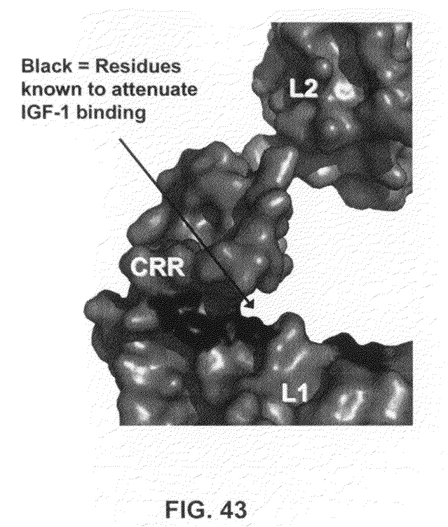

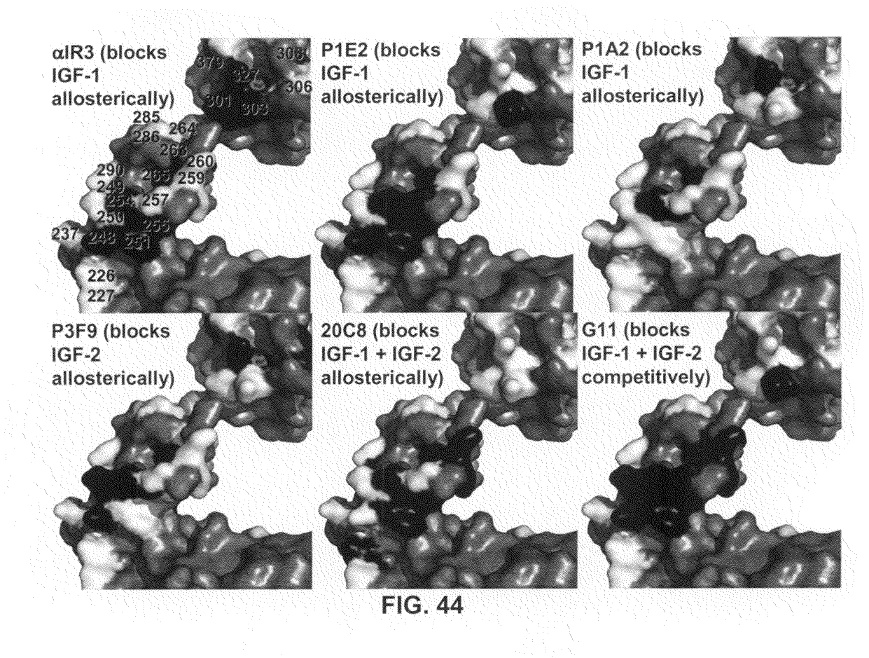

Results: The additional five mutant IGF-1R proteins were screened against M14-G11 and different categories of antibodies (i.e., IGF-1 inhibiting, IGF-2 inhibiting, or IGF-1 and IGF-2 inhibiting) that cross-inhibit binding of M14-G11. Additionally, screening of the P1A2, P3F9, and 20C8 antibodies was also performed against some of the mutants described in Example 27 (thereby providing additional data with respect to epitopes specifically bound by M14-G11, P1E2, and αIR3. Results from the screening are shown in Table 23. The positions of the IGF-1R mutants that were generated (for screening binding by anti-IGF-1R antibodies) are mapped to the surface of a crystal structure of the L1/CRR/L2 domains of IGF-1R (Garrett et al., 1998) in FIG. 43. In FIG. 44, positions whose mutation had an effect on antibody binding are shown in black while those that had no effect on antibody binding are shown in white; see also, Table 23 for additional information on antibody binding effects produced by mutations in IGF-1R. (Note: In FIG. 44 the identity of each mutant position is indicated only in the first panel (demarcating the results with αIR3)).

There are clear differences in the epitopes between the IGF-1 only inhibitors, the IGF-2 only inhibitors, and the IGF-1 and IGF-2 inhibitors. The epitope of the IGF-1 only inhibiting antibodies spans a surface on both the CRR and L1 domains (FIGS. 43 and 44). Mutation at residue 251, which is known to be important for IGF-1 binding (Whittaker et al., 2001), only weakly attenuated the binding affinity of the IGF-1 only inhibitors. The binding of the IGF-2 inhibitor, P3F9, was not affected by mutations in the ligand binding pocket—including residues 251, 259, and 260. The P3F9 antibody instead appeared to bind to an IGF-1R surface on the outer face of the CRR domain. In contrast to the IGF-1 only or IGF-2 only inhibitors, the IGF-1 and IGF-2 inhibitors, 20C8 and M14-G11, both demonstrated attenuated binding to IGF-1R constructs containing mutations at positions 259 and 260, on the inside of the CRR domain facing the ligand binding pocket. These IGF-1 and IGF-2 inhibitors also demonstrated stronger attenuations in binding upon mutation at residue 251 compared to the single ligand inhibitors. Residue 251 is implicated directly in ligand binding (Whittaker et al., 2001; Sorenson et al., 2004). The data indicates that the dual ligand inhibitors 20C8 and M14-G11 bind to areas more deeply embedded within the ligand binding pocket than the single ligand inhibitors. M14-G11 was significantly more affected by mutations in or near the ligand binding pocket than 20C8.

TABLE 23
IGF-1R mutants and their effect on antibody binding.

| Mutation | | | | | | | | | | |
|---|---|---|---|---|---|---|---|---|---|---|
| IGF-1R | | | | | | | | | | |
| position | | | | | | | | | | |
| (w/out | IR 3D | IGF-1R | M13- | M14- | M14- | | | | | |
| signal) | struct# | Domain | C06 | C03 | G11 | P1E2 | αIR3 | P1A2 | P3F9 | 20C8 |
| Epitope | | | | | | | | | | |
| search | | | | | | | | | | |
| (mouse | | | | | | | | | | |
| IGF-1R | | | | | | | | | | |
| mutants) | | | | | | | | | | |
| Y28A | 32 | L1 | NE^{a} | nd | NE | nd | nd | Nd | Nd | Nd |
| M156A | 163 | L1 | NE | Nd | NE | nd | nd | Nd | Nd | Nd |
| T188F | 195 | L1 | NE | Nd | NE | nd | nd | Nd | Nd | Nd |
| S210H | 218 | CRR | NE | NE | NE | nd | nd | Nd | Nd | Nd |
| A211Q | | | | | | | | | | |
| A217T | 224 | CRR | NE | NE | NE | nd | nd | Nd | Nd | Nd |
| A227K | 234 | CRR | NE | NE | NE | NE | NE | Nd | Nd | Nd |
| N237G | 244 | CRR | NE | NE | NE | NE | NE | Nd | Nd | Nd |
| S257F | 264 | CRR | NE | NE | W | W | NE | Nd | Nd | Nd |
| E264K | 275 | CRR | NE | NE | NE | NE | NE | Nd | Nd | Nd |
| G271D | 282 | CRR | NE | NE | NE | NE | NE | Nd | Nd | Nd |
| G285S | 295 | CRR | NE | NE | NE | NE | NE | Nd | Nd | Nd |
| S286T | 296 | | | | | | | | | |
| E303G | 313 | L2 | NE | NE | W | W | W | Nd | Nd | Nd |
| D405K | 415 | L2 | NE | NE | NE | nd | Nd | Nd | Nd | Nd |
| K412A | 422 | L2 | NE | NE | NE | NE | NE | Nd | Nd | Nd |
| A413Q | | | | | | | | | | |
| H464E | 474 | FnIII-1 | S | S | NE | nd | nd | Nd | Nd | Nd |
| D531Q | 547 | FnIII-1 | NE | NE | NE | nd | nd | Nd | Nd | Nd |
| V532N | | | | | | | | | | |
| I650S | * | FnIII-2 | NE | NE | NE | nd | nd | Nd | Nd | Nd |
| | | loop | | | | | | | | |
| E665A | * | FnIII-2 | NE | NE | NE | nd | nd | Nd | Nd | Nd |
| | | loop | | | | | | | | |
| E739W | * | FnIII-2 | NE | NE | NE | nd | nd | Nd | Nd | Nd |
| L741F | | loop | | | | | | | | |
| Epitope | | | | | | | | | | |
| map | | | | | | | | | | |
| FuIII-1 | | | | | | | | | | |
| mutants | | | | | | | | | | |
| S427L | 437 | L2 | NE | NE | nd | nd | nd | Nd | Nd | Nd |
| E459A | 469 | FnIII-1 | S | S | nd | nd | nd | Nd | Nd | Nd |
| S460A | 470 | | | | | | | | | |
| D461A | 471 | FnIII-1 | S | S | nd | nd | nd | Nd | Nd | Nd |
| V462T | 472 | | | | | | | | | |
| H464A | 474 | FnIII-1 | S | S | nd | nd | nd | Nd | Nd | Nd |
| T466L | 476 | FnIII-1 | W | W | nd | nd | nd | Nd | Nd | Nd |
| S467Y | 477 | | | | | | | | | |
| T468R | 478 | FnIII-1 | NE | NE | nd | nd | nd | Nd | Nd | Nd |
| T478R | 488 | FnIII-1 | NE | W | nd | nd | nd | Nd | Nd | Nd |
| H480E | 490 | FnIII-1 | S | S | nd | nd | nd | Nd | Nd | Nd |
| Y482A | 492 | FnIII-1 | S | S | nd | nd | nd | Nd | Nd | Nd |
| R483W | 493 | | | | | | | | | |
| E533H | 548 | FnIII-1 | W | S | nd | nd | nd | Nd | Nd | Nd |
| I564T | 578 | FnIII-1 | W | W | nd | nd | nd | Nd | Nd | Nd |
| R565A | 579 | | | | | | | | | |
| K568A | 582 | FnIII-1 | NE | W | nd | nd | nd | Nd | Nd | Nd |
| E570A | 584 | FnIII-1 | S | S | nd | nd | nd | Nd | Nd | Nd |
| I571T | 585 | | | | | | | | | |
| L572D | 586 | FnIII-1 | DNE | DNE | nd | nd | nd | Nd | nd | nd |
| Y573D | 587 | | | | | | | | | |
| Epitope | | | | | | | | | | |
| map | | | | | | | | | | |
| CRR/L2 | | | | | | | | | | |
| mutants | | | | | | | | | | |
| Y226L | 233 | CRR | Nd | Nd | NE | NE | NE | NE | NE | W* |
| D248A | 255 | CRR | nd | nd | S | W | S | NE | M | NE |
| R249F | 256 | CRR | Nd | Nd | NE | NE | NE | NE | NE | W |
| D250S | 257 | CRR | nd | nd | S | NE | NE | NE | W | NE |
| F251L | 258 | CRR | Nd | Nd | M | W | W | NE | NE | M* |
| N254A | 261 | CRR | nd | nd | M | S | M | M | M | S |
| I255A | 262 | CRR | Nd | Nd | M | W | W | NE | NE | W |
| S257K | 264 | CRR | nd | nd | W | M | NE | NE | NE | NE |
| E259K | 270 | CRR | nd | nd | W | NE | NE | NE | NE | M |
| S260N | 271 | | | | | | | | | |
| S263R | 274 | CRR | nd | nd | W | NE | W | NE | NE | NE |
| G265Y | 276 | CRR | nd | nd | W | S | S | W | W | S |
| Y290L | 300 | CRR | Nd | Nd | NE | NE | NE | NE | NE | NE |
| V301Y | 311 | L2 | nd | nd | NE | NE | W | Nd | Nd | NE |
| K306E | 316 | L2 | nd | nd | NE | NE | NE | NE | W | Nd |
| T308E | 318 | L2 | nd | nd | NE | NE | W | NE | M | Nd |
| K327N | 337 | L2 | nd | nd | NE | NE | W | W | M | Nd |
| L379R | 389 | L2 | nd | nd | NE | NE | W | W | M | Nd |
| E381K | 391 | L2 | nd | Nd | DNE | DNE | DNE | DNE | DNE | DNE |
| E382L | 392 | | | | | | | | | |

^{a}No effect (NE): measured K_{D} within 2.5-fold of WT hIGF-1R-Fc; Weak (W): measured K_{D} between 2.5-10-fold higher than WT; Medium (M): measured K_{D} between 10-100-fold higher than WT; Strong (S): binding to antibody was ablated by mutation; and nd: not determined. “DNE” means did not express or expressed poorly due to intolerance of the protein fold for the mutation. (M*): measured >10-fold increase in affinity upon mutation. (W*): measured >5-fold increase in affinity upon mutation.

### Example 36

### Isothermal Titration Calorimetry (ITC) Experiments Demonstrates a Spectrum of Inhibitory Antibody Binding Properties Ranging from Fully Competitive to Fully Allosteric

Background: Experiments described in this example were performed to further characterize biophysical binding properties of the anti-IGF-1R MAbs. In particular, isothermal titration calorimetry (ITC) was used to study both the binding of IGF-1 and anti-IGF-1R antibodies to IGF-1R. Also studied was the effect that saturating levels of inhibitory antibodies have on the binding of IGF-1 to the receptor. In particular, these experiments were designed to further reveal the allosteric vs. competitive nature of the antibody inhibitors. For these studies, three different classes of antibodies (each of which inhibit both IGF-1 and IGF-2 binding; i.e., M13-C06, 20C8, and M14-G11) were further investigated.

Methods: Isothermal Titration Calorimetry (ITC)— The M13-C06 (75 μM), 20C8 (75 μM), and M14-G11 (67 μM) antibodies, IGF-1 (60 μM, Chemicon International Cat# GF006), and a purified, soluble, recombinant, human IGF-1R ectodomain (sIGF-1R(1-903)) protein (5 μM) were dialyzed against PBS, pH 7.2 in the same container. These protein solutions were adjusted to the concentrations listed above by dialyzing in PBS. ITC experiments were performed on an ITC200 microcalorimeter (MicroCal, LLC). For each reaction, the reaction cell was filled with 5 M hIGF-1R(1-903). To investigate antibody or ligand binding to sIGF-1R(1-903), the injection syringe was filled with antibody or ligand and titrated into the reaction cell using 1.5 or 2.0 μL injections. A four minute equilibration period was used between all injections with an initial delay of 60 seconds. To investigate the effect of the inhibitory antibodies on IGF-1R/IGF-1 binding, antibodies were titrated to a 2:1 saturation of their receptor binding sites, followed by a second set of injections with IGF-1. Numerical integration of the data was performed using the ITC data analysis software supplied by MicroCal (Origin). ΔHA°(T) values were calculated based on the difference between the average heat liberated/absorbed during the binding phase of the injections and the average heat of dilution found once the receptor, IGF-1R, was saturated with antibody or ligand. At 25° C. and lower for the antibodies binding to IGF-1R and at 5° C. for IGF-1 binding to IGF-1R, the binding reaction was too tight to be measured*—as indicated by an absence of multiple titration points as antibody or ligand concentration saturation of the receptor binding sites was achieved.
*Binding being “too tight to measure” by ITC is intended to indicate that the affinity constant (KD) is <500-fold below the concentration at which the measurements were performed. The lower limit of the protein concentrations capable of generating sufficient signal by calorimetry to measure a change in heat was ˜2-4 micromolar protein. Therefore, a KD lower than 5 nM is “too tight” to measure.

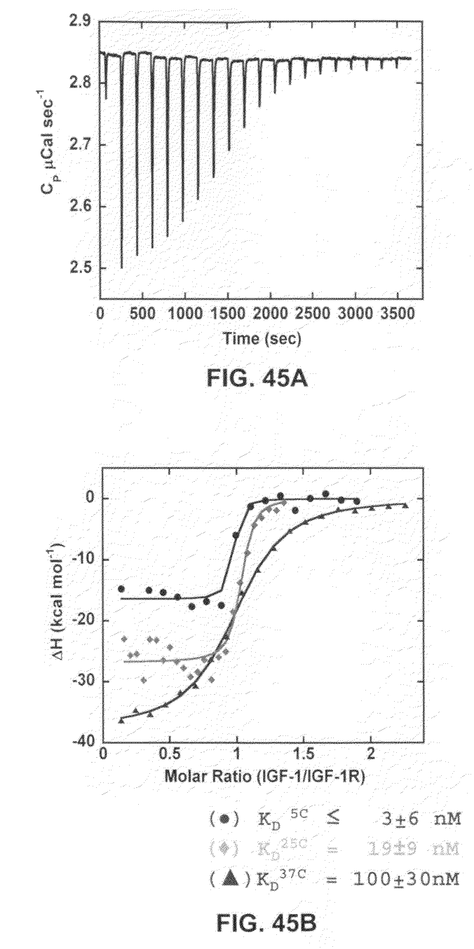

Results: An initial goal was to establish the binding properties of IGF-1 binding to sIGF-1R(1-903) using ITC to serve as a baseline for the interactions of the ligand with the receptor in the presence of antibody. It was determined that the affinity of IGF-1 binding to sIGF-1R(1-903) is highly temperature dependent (Table 24, FIG. 45 A & B). At 5° C., IGF-1 binding to the receptor was too tight to measure the affinity accurately (KD ˜10-9 M). However, at 25° C., the affinity is weakened (KD=19 nM). At 37° C., the affinity weakens further (KD=130 nM). The strong enthalpy of binding that is observed for IGF-1 binding to the receptor suggests there are entropy (i.e., ordering) penalties to ligand binding. One likely reason for the drop in affinity is that the peptidic nature of the ligand becomes increasingly less ordered at higher temperatures. The stoichiometry of IGF-1 binding to the receptor was 1:1 suggesting that a conformational changes likely occurs in the receptor upon ligand binding that occludes the second potential ligand binding pocket from engaging ligand. These results are consistent with published work (Jansson, 1998)

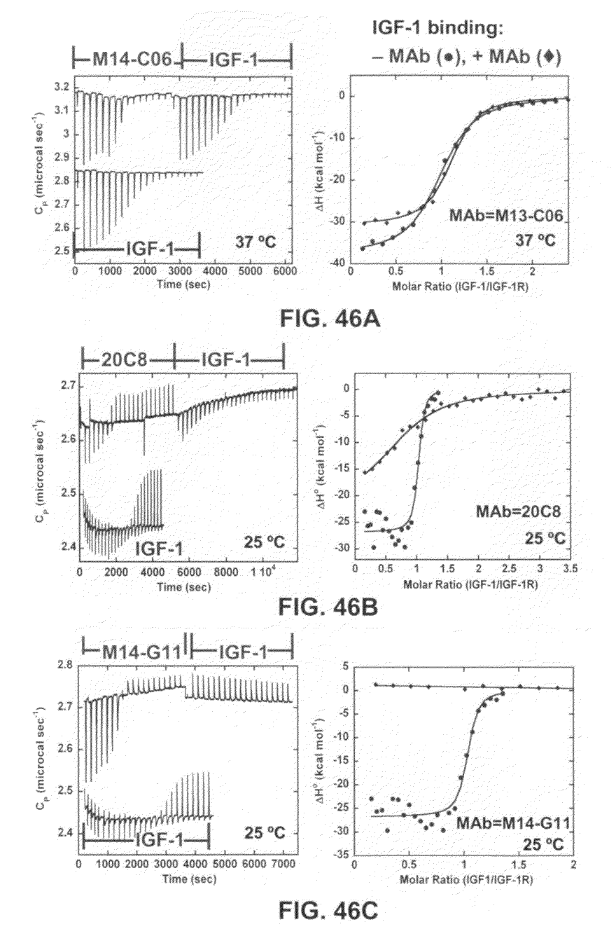

The M13-C06, M14-G11, and 20C8 antibodies were then tested for their ability to bind sIGF-1R(1-903) by ITC. All three antibodies bind tightly to the receptor—too tightly for accurate measurement of their affinities using ITC (Table 24; FIG. 46 A-C). Interestingly, 20C8 and M14-G11 demonstrate vastly different energetics of binding, even though their epitopes are similar to one another and they both inhibit IGF-1 and IGF-2.

Dual titration experiments were performed to investigate the ability of IGF-1 to bind sIGF-1R(1-903) in the presence of saturating levels of inhibitory MAbs (FIG. 46 A-C). The data show a large spectrum of IGF-1 binding inhibition properties—from M13-C06 which appears completely allosteric, to 20C8 which changes the affinity of the ligand for the receptor significantly, but without appear to induce complete binding inhibition, to M14-G11 which appears to completely inhibits IGF-1 binding to the receptor in a competitive fashion (Table 25; FIG. 46 A-C).

TABLE 24
Temperature dependence of IGF-1 and inhibitory antibody binding to
sIGF-1R(1-903) determined by ITC.

| | | −TΔS° | ΔG° | | |
|---|---|---|---|---|---|
| Temperature | ΔH° | (kcal/ | (kcal/ | K_{D} | Stoichiometry |
| (° C.) | (kcal/mol) | mol) | mol) | (nM) | of binding (N) |
| IGF-1 | | | | | |
| 5 | −16.4 ± 0.5 | 5.6 | −10.8 | 3.3 ± 6.0 | 0.93 |
| 25 | −26.8 ± 0.6 | 16.2 | −10.6 | 19 ± 9 | 1.00 |
| 37^{♦} | −33.4 ± 5 | 23.6 | −9.8 | 130 ± 60 | 1.02 |
| M13-C06 | | | | | |
| 5 | −11.3 ± 0.8 | Ø | Ø | Ø | 0.89 |
| 15 | −14.4 ± 0.6 | Ø | Ø | Ø | 0.98 |
| 25^{Δ} | −20.1 ± 2.5 | Ø | Ø | Ø | 0.98 |
| 37 | −29.2 ± 0.4 | Ø | Ø | Ø | 0.97 |
| M14-G11 | | | | | |
| 5 | −6.7 ± 1.0 | Ø | Ø | Ø | 0.90 |
| 25 | −26.6 ± 0.6 | Ø | Ø | Ø | 0.91 |
| 37 | −39.5 ± 1.2 | Ø | Ø | Ø | 0.70 |
| 20C8 | | | | | |
| 25 | −14.1 | Ø | Ø | Ø | 0.84 |

^{♦}Average of three experiments.
^{Δ}Average of two experiments.
Ø = Tight binding resulted in too few points in the transition region to measure K_{D}.

TABLE 25
ITC parameters for the binding of IGF-1 to sIGF-1R(1-903) at 25° C. in the absence and
presence of saturated levels of inhibitory antibodies.

| IGF-1R/Antibody | ΔH° | −TΔS° | ΔG° | | Stoichiometry |
|---|---|---|---|---|---|
| Complex | (kcal/mol) | (kcal/mol) | (kcal/mol) | K_{D} (nM) | of binding (N) |
| IGF-1R | −26.8 ± 0.6 | 16.2 | −10.6 | 19 ± 9 | 1.00 |
| IGF-1R/M13-C06 | −28.4 ± 2.0 | 17.9 | −10.5 | 20 ± 4 | 1.09 |
| IGF-1R/20C8 | −21.7 ± 1.8 | 13.5 | −8.2 | 1000 ± 100 | 0.80 |
| IGF-1R/M14-G11 | Ø | Ø | Ø | Ø | Ø |

Ø = No apparent binding.

### Example 37

### Equilibrium IGF-1 Binding to IGF-1R Using Surface Plasmon Resonance Demonstrates a Spectrum of Inhibitory Antibody Properties Ranging from Fully Competitive to Fully Allosteric

The inhibitory ELISA (Example 34), kinetic Biacore binding experiments (Example 27), and ITC binding experiments (Example 36) that measured IGF-1 binding to IGF-1R in the presence and absence of inhibitory MAbs indicated different molecular mechanisms for inhibiting ligand binding to the receptor. However, while the data in the previous examples indicates that some inhibitors, namely M14-G11 and 20C8, represent classical competitive and allosteric antibodies, respectively, the data for M13-C06 suggests that this allosteric inhibitor leads to decreases in IGF-1 affinity to IGF-1R ranging from 0-50-fold. In this example, the effects of the three dual ligand inhibitors, M14-G11, 20C8, and M13-C06, on the binding of IGF-1 to IGF-1R are examined using a different (orthogonal) technique. In particular, in this example antibodies were analyzed via equilibrium solution phase surface plasmon resonance binding experiments.

Methods: Equilibrium IGF-1 binding to IGF-1R using surface plasmon resonance. All experiments were performed on a Biacore3000 instrument (Biacore). A human IGF-1R-Fc construct was immobilized (˜15000 RUs) to a standard CM5 chip surface using standard amine chemistry protocols provided by the manufacturer. At these high immobilization levels of hIGF-1R-Fc, flowing low concentrations of IGF-1 (<50 nM) led to mass-transfer limited linear binding curves whose initial velocity of binding, Vi (RU/s), depended linearly on the concentration of the IGF-1 solution flowed over the chip surface. Binding constants between soluble sIGF-1R(1-903) protein (described in Example 36) and IGF-1 could be determined by flowing mixtures of sIGF-1R(1-903) and IGF-1 over the sensorchip surface containing sIGF-1R-Fc. The sIGF-1R-Fc sensorchip surface measures the concentration of unbound IGF-1 in solution under each sIGF-1R(1-903)/IGF-1 mixture. The equilibrium dissociation constant, KD, and binding stoichiometry, n, between IGF-1 and sIGF-1R(1-903) are determined by the concentration of unbound IGF-1 using the equation below:

$$Vi = m \cdot [ \begin{matrix}{[ I G F - ]}_{T} - \\ \frac{1}{2} \{ \begin{matrix}( {n [ R ]}_{T} + {[ I G F - 1 ]}_{T} + {K}_{D} ) - \\ \sqrt{\begin{matrix}{( {n [ R ]}_{T} + {[ I G F - 1 ]}_{T} + {K}_{D} )}^{2} - \\ 4 {{n [ R ]}_{T} [ I G F - 1 ]}_{T}\end{matrix}}\end{matrix} \}\end{matrix} ]$$

where Vi=initial rate of binding, m=slope of the IGF-1 concentration-dependent standard curve, [IGF-1]f=unbound IGF-1 concentration=Vi/m, [IGF-1]t=total IGF-1 concentration and [R]t=total sIGF-1R(1-903) ectodomain receptor concentration (Day et. al., 2005).

IGF-1 binding to pre-complexed mixtures of sIGF-1R(1-903) and inhibitory MAbs was performed in an identical fashion. The MAbs and sIGF-1R(1-903) were equimolar (240 nM) in the experiment and size exclusion chromatography was used to insure that there was no excess MAb that would lead to binding to the hIGF-1R-Fc sensorchip surface. Additionally, the sIGF-1R(1-903)/MAb mixtures were passed over the sIGF-1R-Fc sensorchip in the absence of IGF-1, to insure no residual MAb binding to the surface occurred that might complicate the results.

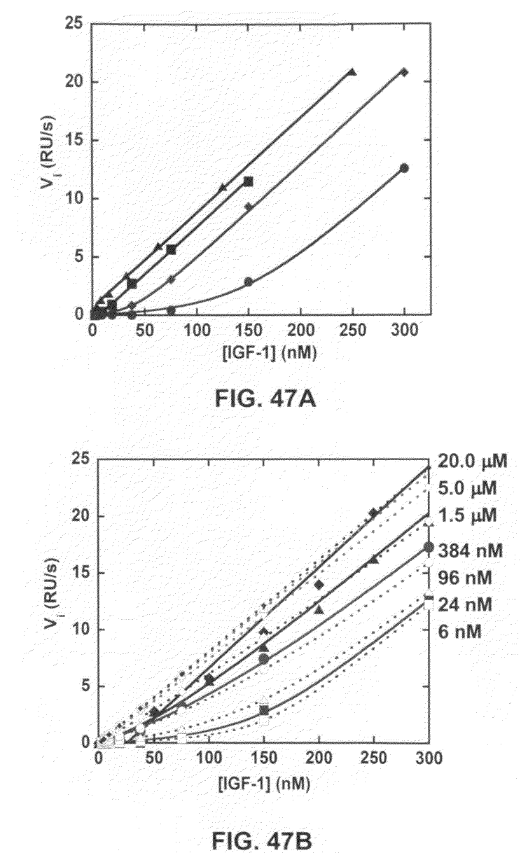

Results: Solution-based surface plasmon resonance experiments are another method for investigating the affinity of IGF-1 for IGF-1R. In this example, a highly active IGF-1R Biacore chip surface was used to capture IGF-1 under mass-transport limiting conditions. The signal arising in the Biacore instrument is linearly proportional to the free IGF-1 concentration in solution that is passed over the sensorchip surface. By adding sIGF-1R(1-903) into solutions with IGF-1, ligand binding to the sensorchip surface is displaced in proportion to (i) the concentration of active sIGF-1R(1-903) and (ii) the affinity of IGF-1 for sIGF-1R(1-903). When the experiments were performed using different starting concentrations of sIGF-1R(1-903), between 24 nM and 240 nM, a binding affinity in the range of ˜6-10 nM was consistently observed for the ligand receptor pair (Table 26, FIG. 47).

Next, the effect of the inhibitory anti-IGF-1R antibodies on the apparent affinity of the ligand for the IGF-1R receptor was analyzed. To do this, 240 nM sIGF-1R(1-903) was pre-incubated at a 1:1 ratio with each inhibitory antibody, and placed in the affinity measurement assay (FIG. 47). M13-C06 appeared to reduce the affinity, between the ligand and the receptor ˜20 fold (Table 26). 20C8 appeared to reduce the affinity between the ligand and the receptor by ˜50-100-fold (Table 26, FIG. 47). Saturating IGF-1R with M14-G11 appeared to completely inhibit IGF-1 binding to the sIGF-1R(1-903) protein leading to a linear IGF-1 binding curve similar to results obtained in the absence of sIGF-1R(1-903) (Table 26, FIG. 47). These experiments demonstrate that both M13-C06 and 20C8 lead to allosteric changes in the receptor that reduce the receptor's ligand binding affinity. In contrast to allosteric inhibition by M13-C06 and 20C8, these results also indicate that M14-G11 is a competitive inhibitor.

TABLE 28
Solution-based IGF-1 binding to sIGF-1R(1-903) in the absence
and presence of inhibitory anti-IGF-1R MAbs.

| [sIGF-1R(1-903)], MAb | KD (nM) | n (IGF-1/sIGF-1R(1-903)) |
|---|---|---|
| 64 nM IGF-1R, No MAb | 5.9 ± 2.5 | 0.66 ± 0.032 |
| 240 nM IGF-1R, No MAb | 10 ± 1.9 | 0.64 ± 0.014 |
| 240 nM IGF-1R, M13-C06 | 150 ± 30 | 0.60 ± 0.1 |
| 240 nM IGF-1R, 20C8 | 550 ± 100 | 0.60 ± 0.1 |
| 240 nM IGF-1R, M14-G11 | Ø | Ø |

Ø = No observed IGF-1 binding to sIGF-1R(1-903).

In addition to the above description, embodiments of the invention are intended to also encompass the following (“E”=Embodiment):
E1. An antibody produced by a cell line selected from the group consisting of:
   a) a Chinese Hamster Ovary (CHO) cell line deposited as American Type Culture Collection (ATCC) deposit number PTA-7444;
   b) a CHO cell line designated deposited as ATCC deposit number PTA-7445;
   c) a CHO cell line deposited as ATCC deposit number PTA-7855;
   d) a hybridoma cell line deposited as ATCC deposit number PTA-7485;
   e) a hybridoma cell line deposited as ATCC deposit number PTA-7732;
   f) a hybridoma cell line deposited as ATCC deposit number PTA-7457;
   g) a hybridoma cell line deposited as ATCC deposit number PTA-7456;
   h) a hybridoma cell line deposited as ATCC deposit number PTA-7730; and,
   i) a hybridoma cell line deposited as ATCC deposit number PTA-7731.
E2. An antibody produced by the cell line of Embodiment 1, wherein said cell line is designated by an ATCC deposit description selected from the group consisting of:
   a) Chinese Hamster Ovary (CHO): C06-40B5; CHO DG44Biogen Idec EA03.14.06;
   b) Chinese Hamster Ovary (CHO): C03-2 CHO DG44Biogen Idec DA 03.14.06;
   c) Chinese hamster ovary cell line: G11 70 8e6 cells 08.09.2006;
   d) Hybridoma 8.P2A7.3D11;
   e) Hybridoma cell line: 7.20C8.3B8;
   f) Hybridoma: 5.P1A2.2B11;
   g) Hybridoma: 7.20D8.24.B11;
   h) Hybridoma Cell Line: 9.P1E2.3B12; and,
   i) Hybridoma Cell Line: 5P1G10.2B8.
E3. The isolated cell line of Embodiments 1 or 2.
E4. An isolated antibody or antigen-binding fragment thereof which specifically binds to a polypeptide domain consisting of the Fibronectin Type-III domain-1 (FNIII-1) of Insulin-like Growth Factor-1 Receptor (IGF-1R).
E5. The antibody or antigen-binding fragment of Embodiment 4, wherein said antibody inhibits binding of Insulin-like Growth Factor-1 (IGF-1) and Insulin-like Growth Factor-2 (IGF-2) to IGF-1R.
E6. The antibody or antigen-binding fragment of Embodiment 5, wherein said inhibition is allosteric.
E7. An isolated antibody or antigen-binding fragment thereof which specifically binds to IGF-1R, wherein the affinity of said antibody binding is reduced by mutation of one or more IGF-1R residues selected from the group consisting of:
   a) Glu-459; b) Ser-460; c) Asp-461; d) Val-462; e) His-464; f) Thr-466; g) Ser-467; h) Thr-478; i) His-480; j) Tyr-482; k) Arg-483; 1) Glu-533; m) Ile-564; n) Arg-565; o) Lys-568; p) Glu-570; and, q) Ile-571.
E8. The antibody or antigen-binding fragment of Embodiment 7, wherein said mutation is a substitution mutation selected from the group consisting of:
   a) Glu-459 to Ala; b) Ser-460 to Ala; c) Asp-461 to Ala; d) Val-462 to Thr; e) His-464 to Ala; f) His-464 to Glu; g) Thr-466 to Leu; h) Ser-467 to Tyr; i) Thr-478 to Arg; j) His-480 to Glu; k) Tyr-482 to Ala; 1) Arg-483 to Trp; m) Glu-533 to His; n) Ile-564 to Thr; o) Arg-565 to Ala; p) Lys-568 to Ala; q) Glu-570 to Ala; and, r) Ile-571 to Thr.
E9. The antibody or antigen-binding fragment of Embodiment 7 or 8, wherein said antibody binding affinity is decreased between about 2.5 to about 10-fold.
E10. The antibody or antigen-binding fragment of Embodiment 7 or 8, wherein said antibody binding affinity is decreased between about 10 to about 100-fold.
E11. The antibody or antigen-binding fragment of Embodiment 7 or 8, wherein said antibody binding affinity is decreased by greater than 100-fold.
E12. The antibody or antigen-binding fragment of Embodiment 7 or 8, wherein said antibody binding affinity is abolished.
E13. The antibody or antigen-binding fragment of any one of Embodiments 7-12, wherein said antibody or antigen-binding fragment inhibits binding of IGF-1 and IGF-2 ligand to IGF-1R.
E14. The antibody or antigen-binding fragment of Embodiment 13, wherein said inhibition is allosteric.
E15. An isolated antibody or antigen-binding fragment thereof which specifically binds to a polypeptide domain consisting of the Cysteine Rich Region (CRR) of IGF-1R.
E16. The antibody or antigen-binding fragment of Embodiment 15, wherein said antibody inhibits binding of IGF-1 and IGF-2 ligand to IGF-1R.
E17. The antibody or antigen-binding fragment of Embodiment 16, wherein said inhibition is competitive.
E18. An isolated antibody or antigen-binding fragment thereof which specifically binds to IGF-1R, wherein the affinity of said antibody binding is reduced by mutation of one or more IGF-1R residues selected from the group consisting of:
   a) Asp-248; b) Asp-250; c) Asn-254; d) Ser-257; e) Glu-259; f) Ser-260; g) Ser-263; h) Gly-265; and, i) Glu-303.
E19. The antibody or antigen-binding fragment of Embodiment 18, wherein said mutation is a substitution mutation selected from the group consisting of:
   a) Asp-248 to Ala; b) Asp-250 to Ser; c) Asn-254 to Ala; d) Ser-257 to Phe; e) Ser-257 to Lys; f) Glu-259 to Lys; g) Ser-260 to Asn; h) Ser-263 to Arg; i) Gly-265 to Tyr; and, j) Glu-303 to Gly.
E20. The antibody or antigen-binding fragment of Embodiment 18 or 19, wherein said antibody binding affinity is decreased between about 2.5 to about 10-fold.
E21. The antibody or antigen-binding fragment of Embodiment 18 or 19, wherein said antibody binding affinity is decreased between about 10 to about 100-fold.
E22. The antibody or antigen-binding fragment of Embodiment 18 or 19, wherein said antibody binding affinity is decreased by greater than 100-fold.
E23. The antibody or antigen-binding fragment of Embodiment 18 or 19, wherein said antibody binding affinity is abolished.
E24. The antibody or antigen-binding fragment of any one of Embodiments 18-23, wherein said antibody or antigen-binding fragment inhibits binding of IGF-1 and IGF-2 ligand to IGF-1R.
E25. The antibody or antigen-binding fragment of Embodiment 24, wherein said inhibition is competitive.
E26. An isolated antibody or antigen-binding fragment thereof which specifically binds to a polypeptide domain consisting of the Cysteine Rich Region (CRR) and second Leucine Rich Repeat domain (L2) of IGF-1R.
E27. The antibody or antigen-binding fragment of Embodiment 26, wherein said antibody inhibits binding of IGF-1 but not IGF-2 ligand to IGF-1R.
E28. The antibody or antigen-binding fragment of Embodiment 27, wherein said inhibition is allosteric.
E29. An isolated antibody or antigen-binding fragment thereof which specifically binds to IGF-1R, wherein the affinity of said antibody binding is reduced by mutation of one or more IGF-1R residues selected from the group consisting of:
   a) Asp-248; b) Asn-254; c) Ser-257; and, d) Gly-265.
E30. The antibody or antigen-binding fragment of Embodiment 29, wherein said mutation is a substitution mutation selected from the group consisting of:
   a) Asp-248 to Ala; b) Asn-254 to Ala; c) Ser-257 to Lys; and, d) Gly-265 to Tyr.
E31. The antibody or antigen-binding fragment of Embodiment 29 or 30, wherein said antibody binding affinity is decreased by about 10 to about 100-fold.
E32. The antibody or antigen-binding fragment of Embodiment 29 or 30, wherein said antibody binding affinity is abolished.
E33. The antibody or antigen-binding fragment of any one of Embodiments 29-32, wherein said antibody inhibits binding of IGF-1 but not IGF-2 ligand to IGF-1R.
E34. The antibody or antigen-binding fragment of Embodiment 33, wherein said inhibition is allosteric.
E35. An isolated antibody or antigen-binding fragment thereof which specifically binds to the same insulin-like growth factor receptor-1 (IGF-1R) epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.
E36. An isolated antibody or antigen-binding fragment thereof which specifically binds to IGF-1R, wherein said antibody or fragment thereof competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8 from binding to IGF-1R.
E37. An isolated antibody or antigen-binding fragment thereof which specifically binds to IGF-1R, wherein said antibody or fragment thereof is comprises an antigen binding domain identical to that of a monoclonal Fab antibody fragment selected from the group consisting of M13-C06, M14-G11, M14-C03, M14-B01, M12-E01, and M12-G04, or a monoclonal antibody produced by a hybridoma selected from the group consisting of P2A7.3E11, 20C8.3B8, P1A2.2B11, 20D8.24B11, P1E2.3B12, and P1G10.2B8.
E38. An isolated antibody or fragment thereof which specifically binds to IGF-1R, wherein the heavy chain variable region (VH) of said antibody or fragment thereof comprises an amino acid sequence at least 90% identical to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.
E39. An isolated antibody or fragment thereof which specifically binds to IGF-1R, wherein the light chain variable region (VL) of said antibody or fragment thereof comprises an amino acid sequence at least 90% identical to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.
E40. An isolated antibody or fragment thereof which specifically binds to IGF-1R, wherein the VH of said antibody or fragment thereof comprises an amino acid sequence identical, except for 20 or fewer conservative amino acid substitutions, to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.
E41. An isolated antibody or fragment thereof which specifically binds to IGF-1R, wherein the VL of said antibody or fragment thereof comprises an amino acid sequence identical, except for 20 or fewer conservative amino acid substitutions, to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.
E42. An isolated antibody or fragment thereof which specifically binds to IGF-1R, wherein the VH of said antibody or fragment thereof comprises an amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.
E43. An isolated antibody or fragment thereof which specifically binds to IGF-1R, wherein the VL of said antibody or fragment thereof comprises an amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.
E44. An isolated antibody or fragment thereof which specifically binds to IGF-1R, wherein the VH and VL of said antibody or fragment thereof comprise, respectively, amino acid sequences at least 90% identical to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO:63 and 118.
E45. An isolated antibody or fragment thereof which specifically binds to IGF-1R, wherein the VH and VL of said antibody or fragment thereof comprise, respectively, amino acid sequences identical, except for 20 or fewer conservative amino acid substitutions each, to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118.
E46. An isolated antibody or fragment thereof which specifically binds to IGF-1R, wherein the VH and VL of said antibody or fragment thereof comprise, respectively, amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118.
E47. An isolated antibody or fragment thereof which specifically binds to IGF-1R, wherein the VH of said antibody or fragment thereof comprises a Kabat heavy chain complementarity determining region-1 (VH-CDR1) amino acid sequence identical, except for two or fewer amino acid substitutions, to a reference VH-CDR1 amino acid sequence selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64.
E48. The antibody or fragment thereof of Embodiment 47, wherein said VH-CDR1 amino acid sequence is selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64.
E49. An isolated antibody or fragment thereof which specifically binds to IGF-1R, wherein the VH of said antibody or fragment thereof comprises a Kabat heavy chain complementarity determining region-2 (VH-CDR2) amino acid sequence identical, except for four or fewer amino acid substitutions, to a reference VH-CDR2 amino acid sequence selected from the group consisting of: SEQ ID NO: 6, SEQ ID NO: 11, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65.
E50. The antibody or fragment thereof of Embodiment 49, wherein said VH-CDR2 amino acid sequence is selected from the group consisting of: SEQ ID NO: 6, SEQ ID NO: 11, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65.
E51. An isolated antibody or fragment thereof which specifically binds to IGF-1R, wherein the VH of said antibody or fragment thereof comprises a Kabat heavy chain complementarity determining region-3 (VH-CDR3) amino acid sequence identical, except for four or fewer amino acid substitutions, to a reference VH-CDR3 amino acid sequence selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66.
E52. The antibody or fragment thereof of Embodiment 51, wherein said VH-CDR3 amino acid sequence is selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66.
E53. An isolated antibody or fragment thereof which specifically binds to IGF-1R, wherein the VL of said antibody or fragment thereof comprises a Kabat light chain complementarity determining region-1 (VL-CDR1) amino acid sequence identical, except for four or fewer amino acid substitutions, to a reference VL-CDR1 amino acid sequence selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119.
E54. The antibody or fragment thereof of Embodiment 53, wherein said VL-CDR1 amino acid sequence is selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119.
E55. An isolated antibody or fragment thereof which specifically binds to IGF-1R, wherein the VL of said antibody or fragment thereof comprises a Kabat light chain complementarity determining region-2 (VL-CDR2) amino acid sequence identical, except for two or fewer amino acid substitutions, to a reference VL-CDR2 amino acid sequence selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 110, SEQ ID NO: 115, and SEQ ID NO: 120.
E56. The antibody or fragment thereof of Embodiment 55, wherein said VL-CDR2 amino acid sequence is selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 10, SEQ ID NO: 115, and SEQ ID NO: 120.
E57. An isolated antibody or fragment thereof which specifically binds to IGF-1R, wherein the VL of said antibody or fragment thereof comprises a Kabat light chain complementarity determining region-3 (VL-CDR3) amino acid sequence identical, except for four or fewer amino acid substitutions, to a reference VL-CDR3 amino acid sequence selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO: 111, SEQ ID NO: 116, and SEQ ID NO: 121.
E58. The antibody or fragment thereof of Embodiment 57, wherein said VL-CDR3 amino acid sequence is selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO: 111, SEQ ID NO: 116, and SEQ ID NO: 121.
E59. An isolated antibody or fragment thereof which specifically binds to IGF-1R, wherein the VH of said antibody or fragment thereof comprises VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66, except for one, two, three, or four amino acid substitutions in at least one of said VH-CDRs.
E60. An isolated antibody or fragment thereof which specifically binds to IGF-1R, wherein the VH of said antibody or fragment thereof comprises VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66.
E61. An isolated antibody or fragment thereof which specifically binds to IGF-1R, wherein the VL of said antibody or fragment thereof comprises VL-CDR1, VL-CDR2, and VL-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121, except for one, two, three, or four amino acid substitutions in at least one of said VL-CDRs.
E62. An isolated antibody or fragment thereof which specifically binds to IGF-1R, wherein the VL of said antibody or fragment thereof comprises VL-CDR1, VL-CDR2, and VL-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121.
E63. The antibody or fragment thereof of any one of Embodiments 35 to 62, wherein the VH framework regions are human, except for five or fewer amino acid substitutions.
E64. The antibody or fragment thereof of any one of Embodiments 35 to 63, wherein the VL framework regions are human, except for five or fewer amino acid substitutions.
E65. The antibody or fragment thereof of any one of Embodiments 35 to 64, which binds to a non-linear conformational epitope.
E66. An isolated antibody or antigen-binding fragment thereof which specifically binds to an IGF-1R epitope comprising amino acid residue valine-462.
E67. An isolated antibody or antigen-binding fragment thereof which specifically binds to an IGF-1R epitope comprising amino acid residue histidine-464.
E68. An isolated antibody or antigen-binding fragment thereof which specifically binds to an IGF-1R epitope comprising amino acid residues valine-462 and histidine-464.
E69. An isolated antibody or antigen-binding fragment thereof which specifically binds to an IGF-1R epitope comprising a solvent accessible surface radius of 1, 2, 3, 4, 5, 6, 7, 8, 9, 10, 11, 12, 13 or 14 angstroms from residue valine-462.
E70. An isolated antibody or antigen-binding fragment thereof which specifically binds to an IGF-1R epitope comprising a solvent accessible surface radius of 1, 2, 3, 4, 5, 6, 7, 8, 9, 10, 11, 12, 13 or 14 angstroms from residue histidine-464.
E71. An isolated antibody or antigen-binding fragment thereof which specifically binds to an IGF-1R epitope comprising a solvent accessible surface radius of 1, 2, 3, 4, 5, 6, 7, 8, 9, 10, 11, 12, 13 or 14 angstroms from residues valine-462 and histidine-464.
E72. An isolated antibody or antigen-binding fragment thereof which specifically binds to an IGF-1R epitope comprising a solvent accessible surface radius of 1, 2, 3, 4, 5, 6, 7, 8, 9, 10, 11, 12, 13 or 14 angstroms from the center of residues valine-462 and histidine-464 of human IGF-1R.
E73. The antibody or fragment thereof of any one of Embodiments 35 to 64, which binds to a linear epitope.
E74. The antibody or fragment thereof of any one of Embodiments 35 to 73, which is a multivalent, and comprises at least two heavy chains and at least two light chains.
E75. The antibody or fragment thereof of any one of Embodiments 35 to 74, which is multispecific.
E76. The antibody or fragment thereof of Embodiment 75, which is bispecific.
E77. The antibody or fragment thereof of any one of Embodiments 35 to 76, which is bispecific.
E78. The antibody or fragment thereof of any one of Embodiments 35 to 77, wherein the heavy and light chain variable domains are fully human.
E79. The antibody or fragment thereof of Embodiment 78, wherein said heavy and light chain variable domains are from a monoclonal Fab antibody fragment selected from the group consisting of M13-C06, M14-G11, M14-C03, M14-B01, M12-E01, and M12-G04.
E80. The antibody or fragment thereof of any one of Embodiments 35 to 77, wherein the heavy and light chain variable domains are murine.
E81. The antibody or fragment thereof of Embodiment 80, wherein said heavy and light chain variable domains are from a monoclonal antibody produced by a hybridoma selected from the group consisting of P2A7.3E11, 20C8.3B8, P1A2.2B11, 20D8.24B11, P1E2.3B12, and P1G10.2B8.
E82. The antibody or fragment thereof of any one of Embodiments 35 to 77 and 80-81, which is humanized.
E83. The antibody or fragment thereof of any one of Embodiments 35 to 77 and 80-81, which is chimeric.
E84. The antibody or fragment thereof of any one of Embodiments 35 to 77 and 80-81, which is primatized.
E85. The antibody or fragment thereof of any one of Embodiments 35 to 79, which is fully human.
E86. The antibody or fragment thereof of any one of Embodiments 35 to 85, which is an Fab fragment.
E87. The antibody or fragment thereof of any one of Embodiments 35 to 85, which is an Fab′ fragment.
E88. The antibody or fragment thereof of any one of Embodiments 35 to 85, which is an F(ab)2 fragment.
E89. The antibody or fragment thereof of any one of Embodiments 35 to 85, which is an Fv fragment.
E90. The antibody or fragment thereof of any one of Embodiments 35 to 85, which is a single chain antibody.
E91. The antibody or fragment thereof of any one of Embodiments 35 to 88 and 90, which comprises a light chain constant regions selected from the group consisting of a human kappa constant region and a human lambda constant region.
E92. The antibody or fragment thereof of any one of Embodiments 35 to 88 and 90, which comprises at a heavy chain constant region or fragment thereof.
E93. The antibody or fragment thereof of Embodiment 92, wherein said heavy chain constant region or fragment thereof is human IgG4.
E94. The antibody or fragment thereof of Embodiment 93, wherein said IgG4 is mutagenized to remove glycosylation sites.
E95. The antibody or fragment thereof of Embodiment 94, wherein said mutations comprise S241P and T318A using the Kabat numbering system.
E96. The antibody or fragment thereof of any one of Embodiments 35 to 95, which specifically binds to an IGF-1R polypeptide or fragment thereof, or an IGF-1R variant polypeptide, with an affinity characterized by a dissociation constant (KD) which is less than the KD for said reference monoclonal antibody.
E97. The antibody or fragment thereof of any one of Embodiments 35 to 96, which specifically binds to an IGF-1R polypeptide or fragment thereof, or an IGF-1R variant polypeptide with an affinity characterized by a dissociation constant (KD) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.
E98. The antibody or fragment thereof of any one of Embodiments 35 to 97, wherein said affinity is characterized by a dissociation constant (KD) in a range of about 1×10⁻¹⁰ to about 5×10⁻⁹ M.
E99. The antibody or fragment thereof of any one of Embodiments 35 to 98, wherein said affinity is characterized by a dissociation constant (KD) selected from the group consisting of:
   (a) about 1.1×10⁻¹⁰ M; (b) about 1.3×10⁻¹⁰ M; (c) about 3.6×10⁻¹⁰ M; (d) about 1.3×10⁻⁹ M; (e) about 4.0×10⁻⁹ M; and, (f) about 4.9×10⁻⁹ M.
E100. The antibody or fragment thereof of any one of Embodiments 35 to 99, wherein said affinity is characterized by a dissociation constant (KD) selected from the group consisting of:
   (a) 1.1×10⁻¹⁰ M; (b) 1.3×10⁻¹⁰ M; (c) 3.6×10⁻¹⁰ M; (d) 1.3×10⁻⁹ M; (e) 4.0×10⁻⁹ M; and, (f) 4.9×10⁻⁹ M.
E101. The antibody or fragment thereof of any one of Embodiments 35 to 100, which preferentially binds to a human IGF-1R polypeptide or fragment thereof, relative to a murine IGF-1R polypeptide or fragment thereof or a non-human primate IGF-1R polypeptide or fragment thereof.
E102. The antibody or fragment thereof of any one of Embodiments 35 to 100, which binds to human IGF-1R polypeptide or fragment thereof, and also binds to a non-human primate IGF-1R polypeptide or fragment thereof.
E103. The antibody or fragment thereof of any one of Embodiments 35 to 102, which binds to IGF-1R expressed on the surface of a cell.
E104. The antibody or fragment thereof of Embodiment 103, wherein said cell is a malignant cell, a neoplastic cell, a tumor cell, or a metastatic cell.
E105. The antibody or fragment thereof of any one of Embodiments 35 to 104, which blocks insulin growth factor from binding to IGF-1R.
E106. The antibody or fragment thereof of Embodiment 105, wherein said insulin growth factor is insulin growth factor-1 (IGF-1).
E107. The antibody or fragment thereof of Embodiment 105, wherein said insulin growth factor is insulin growth factor-2 (IGF-2).
E108. The antibody or fragment thereof of Embodiment 105, which blocks both IGF-1 and IGF-2 from binding to IGF-1R.
E109. The antibody or fragment thereof of any one of Embodiments 35 to 108, which inhibits IGF-1R-mediated cell proliferation.
E110. The antibody or fragment thereof of any one of Embodiments 35 to 109, which inhibits IGF-1 or IGF-2-mediated IGF-1R phosphorylation.
E111. The antibody or fragment thereof of any one of Embodiments 35 to 110, which inhibits tumor cell growth.
E112. The antibody or fragment thereof of any one of Embodiments 35 to 111, which inhibits IGF-1R internalization.
E113. The antibody or fragment thereof of any one of Embodiments 35 to 112, further comprising a heterologous polypeptide fused thereto.
E114. The antibody or fragment thereof of any one of Embodiments 35 to 113, wherein said antibody is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents.
E115. The antibody or fragment thereof of Embodiment 114, wherein said cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents.
E116. The antibody or fragment thereof of Embodiment 114, wherein said detectable label is selected from the group consisting of an enzyme, a fluorescent label, a chemiluminescent label, a bioluminescent label, a radioactive label, or a combination of two or more of any said detectable labels.
E117. A composition comprising the antibody or fragment thereof of any one of Embodiments 35 to 116, and a carrier.
E118. An isolated polynucleotide comprising a nucleic acid which encodes an antibody VH polypeptide, wherein the amino acid sequence of said VH polypeptide is at least 90% identical to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63; and wherein an antibody or antigen binding fragment thereof comprising said VH polypeptide specifically binds to IGF-1R.
E119. The polynucleotide of Embodiment 118, wherein the amino acid sequence of said VH polypeptide is selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63.
E120. The polynucleotide of Embodiment 118 or 119, wherein the nucleotide sequence encoding said VH polypeptide is optimized for increased expression without changing the amino acid sequence of said VH polypeptide.
E121. The polynucleotide of Embodiment 120, wherein said optimization comprises identification and removal of splice donor and splice acceptor sites.
E122. The polynucleotide of Embodiment 120 or 121, wherein said optimization comprises optimization of codon usage for the cells expressing said polynucleotide.
E123. The polynucleotide of any one of Embodiments 118 to 122, wherein said nucleic acid comprises a nucleotide sequence selected from the group consisting of: SEQ ID NO: 3, SEQ ID NO: 8, SEQ ID NO: 13, SEQ ID NO: 18, SEQ ID NO: 19, SEQ ID NO: 24, SEQ ID NO: 25, SEQ ID NO: 30, SEQ ID NO: 31, SEQ ID NO: 36, SEQ ID NO: 37, SEQ ID NO: 42, SEQ ID NO: 47, SEQ ID NO: 52, SEQ ID NO: 57, and SEQ ID NO: 62.
E124. An isolated polynucleotide comprising a nucleic acid which encodes an antibody VL polypeptide, wherein the amino acid sequence of said VL polypeptide is at least 90% identical to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118; and wherein an antibody or antigen binding fragment thereof comprising said VL polypeptide specifically binds to IGF-1R.
E125. The polynucleotide of Embodiment 124, wherein the amino acid sequence of said VL polypeptide is selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118.
E126. The polynucleotide of Embodiment 124 or 125, wherein the nucleotide sequence encoding said VL polypeptide is optimized for increased expression without changing the amino acid sequence of said VL polypeptide.
E127. The polynucleotide of Embodiment 126, wherein said optimization comprises identification and removal of splice donor and splice acceptor sites.
E128. The polynucleotide of Embodiment 126 or 127, wherein said optimization comprises optimization of codon usage for the cells expressing said polynucleotide.
E129. The polynucleotide of any one of Embodiments 124 to 128, wherein said nucleic acid comprises a nucleotide sequence selected from the group consisting of: SEQ ID NO: 67, SEQ ID NO: 72, SEQ ID NO: 77, SEQ ID NO: 82, SEQ ID NO: 87, SEQ ID NO: 92, SEQ ID NO: 97, SEQ ID NO: 102, SEQ ID NO: 107, SEQ ID NO: 112, and SEQ ID NO: 117.
E130. An isolated polynucleotide comprising a nucleic acid which encodes an antibody VH polypeptide, wherein the amino acid sequence of said VH polypeptide is identical, except for 20 or fewer conservative amino acid substitutions, to a reference amino acid sequence selected from the group consisting of: SEQ ID NO: 4, SEQ ID NO: 9, SEQ ID NO: 14, SEQ ID NO: 20, SEQ ID NO: 26, SEQ ID NO: 32, SEQ ID NO: 38, SEQ ID NO: 43, SEQ ID NO: 48, SEQ ID NO: 53, SEQ ID NO: 58, and SEQ ID NO: 63; and wherein an antibody or antigen binding fragment thereof comprising said VH polypeptide specifically binds to IGF-1R.
E131. An isolated polynucleotide comprising a nucleic acid which encodes an antibody VL polypeptide, wherein the amino acid sequence of said VL polypeptide is identical, except for 20 or fewer conservative amino acid substitutions, to a reference amino acid sequence selected from the group consisting of: SEQ ID NO: 68, SEQ ID NO: 73, SEQ ID NO: 78, SEQ ID NO: 83, SEQ ID NO: 88, SEQ ID NO: 93, SEQ ID NO: 98, SEQ ID NO: 103, SEQ ID NO: 108, SEQ ID NO: 113, and SEQ ID NO: 118; and wherein an antibody or antigen binding fragment thereof comprising said VL polypeptide specifically binds to IGF-1R.
E132. An isolated polynucleotide comprising a nucleic acid which encodes a VH-CDR1 amino acid sequence identical, except for two or fewer amino acid substitutions, to a reference VH-CDR1 amino acid sequence selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64; and wherein an antibody or antigen binding fragment thereof comprising said VH-CDR1 specifically binds to IGF-1R.
E133. The polynucleotide or fragment thereof of Embodiment 132, wherein said VH-CDR1 amino acid sequence is selected from the group consisting of: SEQ ID NO: 5, SEQ ID NO: 10, SEQ ID NO: 15, SEQ ID NO: 21, SEQ ID NO: 27, SEQ ID NO: 33, SEQ ID NO: 39, SEQ ID NO: 44, SEQ ID NO: 49, SEQ ID NO: 54, SEQ ID NO: 59, and SEQ ID NO: 64.
E134. An isolated polynucleotide comprising a nucleic acid which encodes a VH-CDR2 amino acid sequence identical, except for four or fewer amino acid substitutions, to a reference VH-CDR2 amino acid sequence selected from the group consisting of: SEQ ID NO: 6, SEQ ID NO: 11, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65; and wherein an antibody or antigen binding fragment thereof comprising said VH-CDR2 specifically binds to IGF-1R.
E135. The polynucleotide or fragment thereof of Embodiment 134, wherein said VH-CDR2 amino acid sequence is selected from the group consisting of: SEQ ID NO: 6, SEQ ID NO: 1, SEQ ID NO: 16, SEQ ID NO: 22, SEQ ID NO: 28, SEQ ID NO: 34, SEQ ID NO: 40, SEQ ID NO: 45, SEQ ID NO: 50, SEQ ID NO: 55, SEQ ID NO: 60, and SEQ ID NO: 65.
E136. An isolated polynucleotide comprising a nucleic acid which encodes a VH-CDR3 amino acid sequence identical, except for four or fewer amino acid substitutions, to a reference VH-CDR3 amino acid sequence selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66; and wherein an antibody or antigen binding fragment thereof comprising said VH-CDR3 specifically binds to IGF-1R.
E137. The polynucleotide or fragment thereof of Embodiment 136, wherein said VH-CDR3 amino acid sequence is selected from the group consisting of: SEQ ID NO: 7, SEQ ID NO: 12, SEQ ID NO: 17, SEQ ID NO: 23, SEQ ID NO: 29, SEQ ID NO: 35, SEQ ID NO: 41, SEQ ID NO: 46, SEQ ID NO: 51, SEQ ID NO: 56, SEQ ID NO: 61, and SEQ ID NO: 66.
E138. An isolated polynucleotide comprising a nucleic acid which encodes a VL-CDR1 amino acid sequence identical, except for four or fewer amino acid substitutions, to a reference VL-CDR1 amino acid sequence selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119; and wherein an antibody or antigen binding fragment thereof comprising said VL-CDR1 specifically binds to IGF-1R.
E139. The polynucleotide or fragment thereof of Embodiment 138, wherein said VL-CDR1 amino acid sequence is selected from the group consisting of: SEQ ID NO: 69, SEQ ID NO: 74, SEQ ID NO: 79, SEQ ID NO: 84, SEQ ID NO: 89, SEQ ID NO: 94, SEQ ID NO: 99, SEQ ID NO: 104, SEQ ID NO: 109, SEQ ID NO: 114, and SEQ ID NO: 119.
E140. An isolated polynucleotide comprising a nucleic acid which encodes a VL-CDR2 amino acid sequence identical, except for two or fewer amino acid substitutions, to a reference VL-CDR2 amino acid sequence selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 110, SEQ ID NO: 115, and SEQ ID NO: 120; and wherein an antibody or antigen binding fragment thereof comprising said VL-CDR2 specifically binds to IGF-1R.
E141. The polynucleotide or fragment thereof of Embodiment 140, wherein said VL-CDR2 amino acid sequence is selected from the group consisting of: SEQ ID NO: 70, SEQ ID NO: 75, SEQ ID NO: 80, SEQ ID NO: 85, SEQ ID NO: 90, SEQ ID NO: 95, SEQ ID NO: 100, SEQ ID NO: 105, SEQ ID NO: 110, SEQ ID NO: 115, and SEQ ID NO: 120.
E142. An isolated polynucleotide comprising a nucleic acid which encodes a VL-CDR3 amino acid sequence identical, except for four or fewer amino acid substitutions, to a reference VL-CDR3 amino acid sequence selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO: 111, SEQ ID NO: 116, and SEQ ID NO: 121; and wherein an antibody or antigen binding fragment thereof comprising said VL-CDR3 specifically binds to IGF-1R.
E143. The polynucleotide or fragment thereof of Embodiment 142, wherein said VL-CDR3 amino acid sequence is selected from the group consisting of: SEQ ID NO: 71, SEQ ID NO: 76, SEQ ID NO: 81, SEQ ID NO: 86, SEQ ID NO: 91, SEQ ID NO: 96, SEQ ID NO: 101, SEQ ID NO: 106, SEQ ID NO: 111, SEQ ID NO: 116, and SEQ ID NO: 121.
E144. An isolated polynucleotide comprising a nucleic acid which encodes an antibody VH polypeptide, wherein said VH polypeptide comprises VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66; and wherein an antibody or antigen binding fragment thereof comprising said VL-CDR3 specifically binds to IGF-1R.
E145. An isolated polynucleotide comprising a nucleic acid which encodes an antibody VL polypeptide, wherein said VL polypeptide comprises VH-CDR1, VH-CDR2, and VH-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121; and wherein an antibody or antigen binding fragment thereof comprising said VL-CDR3 specifically binds to IGF-1R.
E146. The polynucleotide of any one of Embodiments 118 to 111, further comprising a nucleic acid encoding a signal peptide fused to said antibody VH polypeptide.
E147. The polynucleotide of any one of Embodiments 118 to 146, further comprising a nucleic acid encoding a signal peptide fused to said antibody VL polypeptide.
E148. The polynucleotide of any one of Embodiments 118 to 147, further comprising a nucleic acid encoding a heavy chain constant region CH1 domain fused to said VH polypeptide.
E149. The polynucleotide of any one of Embodiments 118 to 148, further comprising a nucleic acid encoding a heavy chain constant region CH2 domain fused to said VH polypeptide.
E150. The polynucleotide of any one of Embodiments 118 to 149, further comprising a nucleic acid encoding a heavy chain constant region CH3 domain fused to said VH polypeptide.
E151. The polynucleotide of any one of Embodiments 118 to 150, further comprising a nucleic acid encoding a heavy chain hinge region fused to said VH polypeptide.
E152. The polynucleotide of any one of Embodiments 148 to 151, wherein said heavy chain constant region is human IgG4.
E153. The polynucleotide of Embodiment 152, wherein said IgG4 is mutagenized to remove glycosylation sites.
E154. The polynucleotide of Embodiment 153, wherein said mutations comprise S241P and T318A using the Kabat numbering system.
E155. The polynucleotide of any one of Embodiments 118 to 154, further comprising a nucleic acid encoding a light chain constant region domain fused to said VL polypeptide.
E156. The polynucleotide of Embodiment 155, wherein said light chain constant region is human kappa.
E157. The polynucleotide of any one of Embodiments 118 to 156, wherein an antibody or antigen-binding fragment thereof comprising a polypeptide encoded by said nucleic acid specifically binds the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.
E158. The polynucleotide of any one of Embodiments 118 to 157, wherein an antibody or antigen-binding fragment thereof comprising a polypeptide encoded by said nucleic acid competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.
E159. The polynucleotide of any one of Embodiments 118 to 158, wherein the framework regions of said VH polypeptide are human, except for five or fewer amino acid substitutions.
E160. The polynucleotide of any one of Embodiments 118 to 159, wherein the framework regions of said VL polypeptide are human, except for five or fewer amino acid substitutions.
E161. The polynucleotide of any one of Embodiments 118 to 160, wherein an antibody or antigen-binding fragment thereof comprising the polypeptide encoded by said nucleic acid binds to a linear epitope.
E162. The polynucleotide of any one of Embodiments 118 to 160, wherein an antibody or antigen-binding fragment thereof comprising the polypeptide encoded by said nucleic acid binds to a non-linear conformational epitope.
E163. The polynucleotide of any one of Embodiments 118 to 162, wherein an antibody or antigen-binding fragment thereof comprising the polypeptide encoded by said nucleic acid is a multivalent, and comprises at least two heavy chains and at least two light chains.
E164. The polynucleotide of any one of Embodiments 118 to 163, wherein an antibody or antigen-binding fragment thereof comprising the polypeptide encoded by said nucleic acid is multispecific.
E165. The polynucleotide of any one of Embodiments 118 to 164, wherein an antibody or antigen-binding fragment thereof comprising the polypeptide encoded by said nucleic acid is bispecific.
E166. The polynucleotide of any one of Embodiments 118 to 165, wherein an antibody or antigen-binding fragment thereof comprising the polypeptide encoded by said nucleic acid comprises heavy and light chain variable domains which are fully human.
E167. The polynucleotide of Embodiment 166, wherein said heavy and light chain variable domains are identical to those of a monoclonal Fab antibody fragment selected from the group consisting of M13-C06, M14-G11, M14-C03, M14-B01, M12-E01, and M12-G04.
E168. The polynucleotide of any one of Embodiments 118 to 165, wherein an antibody or antigen-binding fragment thereof comprising the polypeptide encoded by said nucleic acid comprises heavy and light chain variable domains which are murine.
E169. The polynucleotide of Embodiment 168, wherein said heavy and light chain variable domains are identical to those of a monoclonal antibody produced by a hybridoma selected from the group consisting of P2A7.3E11, 20C8.3B8, P1A2.2B11, 20D8.24B11, P1E2.3B12, and P1G10.2B8.
E170. The polynucleotide of any one of Embodiments 118 to 165 and 168 to 169, wherein an antibody or antigen-binding fragment thereof comprising the polypeptide encoded by said nucleic acid is humanized.
E171. The polynucleotide of any one of Embodiments 118 to 165 and 168 to 169, wherein an antibody or antigen-binding fragment thereof comprising the polypeptide encoded by said nucleic acid is chimeric.
E172. The polynucleotide of any one of Embodiments 118 to 165 and 168 to 169, wherein an antibody or antigen-binding fragment thereof comprising the polypeptide encoded by said nucleic acid is primatized.
E173. The polynucleotide of any one of Embodiments 118 to 167, wherein an antibody or antigen-binding fragment thereof comprising the polypeptide encoded by said nucleic acid is fully human.
E174. The polynucleotide of any one of Embodiments 118 to 173, wherein an antibody or antigen-binding fragment thereof comprising the polypeptide encoded by said nucleic acid is an Fab fragment.
E175. The polynucleotide of any one of Embodiments 118 to 173, wherein an antibody or antigen-binding fragment thereof comprising the polypeptide encoded by said nucleic acid is an Fab′ fragment.
E176. The polynucleotide of any one of Embodiments 118 to 173, wherein an antibody or antigen-binding fragment thereof comprising the polypeptide encoded by said nucleic acid is an F(ab)2 fragment.
E177. The polynucleotide of any one of Embodiments 118 to 173, wherein an antibody or antigen-binding fragment thereof comprising the polypeptide encoded by said nucleic acid is an Fv fragment.
E178. The polynucleotide of any one of Embodiments 118 to 173, wherein an antibody or antigen-binding fragment thereof comprising the polypeptide encoded by said nucleic acid is a single chain antibody.
E179. The polynucleotide of any one of Embodiments 118 to 178, wherein an antibody or antigen-binding fragment thereof comprising the polypeptide encoded by said nucleic acid specifically binds to an IGF-1R polypeptide or fragment thereof, or an IGF-1R variant polypeptide, with an affinity characterized by a dissociation constant (KD) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.
E180. The polynucleotide of any one of Embodiments 118 to 179, wherein an antibody or antigen-binding fragment thereof comprising the polypeptide encoded by said nucleic acid preferentially binds to a human IGF-1R polypeptide or fragment thereof, relative to a murine IGF-1R polypeptide or fragment thereof or a non-human primate IGF-1R polypeptide or fragment thereof.
E181. The polynucleotide of any one of Embodiments 118 to 179, wherein an antibody or antigen-binding fragment thereof comprising the polypeptide encoded by said nucleic acid binds to a human IGF-1R polypeptide or fragment thereof, and also binds to a non-human primate IGF-1R polypeptide or fragment thereof.
E182. The polynucleotide of any one of Embodiments 118 to 181, wherein an antibody or antigen-binding fragment thereof comprising the polypeptide encoded by said nucleic acid binds to IGF-1R expressed on the surface of a cell.
E183. The polynucleotide of Embodiment 182, wherein said cell is a malignant cell, a neoplastic cell, a tumor cell, or a metastatic cell.
E184. The polynucleotide of any one of Embodiments 118 to 183, wherein an antibody or antigen-binding fragment thereof comprising the polypeptide encoded by said nucleic acid blocks insulin growth factor from binding to IGF-1R.
E185. The polynucleotide of Embodiment 184, wherein said insulin growth factor is insulin growth factor-1 (IGF-1).
E186. The polynucleotide of Embodiment 184, wherein said insulin growth factor is insulin growth factor-2 (IGF-2).
E187. The polynucleotide of Embodiment 184, which blocks both IGF-1 and IGF-2 from binding to IGF-1R.
E188. The polynucleotide of any one of Embodiments 118 to 153, wherein an antibody or antigen-binding fragment thereof comprising the polypeptide encoded by said nucleic acid inhibits IGF-1R-mediated cell proliferation.
E189. The polynucleotide of any one of Embodiments 118 to 188, wherein an antibody or antigen-binding fragment thereof comprising the polypeptide encoded by said nucleic acid inhibits IGF-1 or IGF-2-mediated IGF-1R phosphorylation.
E190. The polynucleotide of any one of Embodiments 118 to 189, wherein an antibody or antigen-binding fragment thereof comprising the polypeptide encoded by said nucleic acid inhibits tumor cell growth.
E191. The polynucleotide of any one of Embodiments 118 to 190, wherein an antibody or antigen-binding fragment thereof comprising the polypeptide encoded by said nucleic acid inhibits IGF-1R internalization.
E192. The polynucleotide of any one of Embodiments 118 to 191, further comprising a nucleic acid encoding a heterologous polypeptide.
E193. The polynucleotide of any one of Embodiments 118 to 192, wherein an antibody or antigen-binding fragment thereof comprising the polypeptide encoded by said nucleic acid is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents.
E194. The polynucleotide of Embodiment 193, wherein said cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents.
E195. The polynucleotide of Embodiment 193, wherein said detectable label is selected from the group consisting of an enzyme, a fluorescent label, a chemiluminescent label, a bioluminescent label, a radioactive label, or a combination of two or more of any said detectable labels.
E196. A composition comprising the polynucleotide any one of Embodiments 118 to 195, and a carrier.
E197. A vector comprising the polynucleotide of any one of Embodiments 118 to 196.
E198. The vector of Embodiment 197, wherein said polynucleotide is operably associated with a promoter.
E199. A host cell comprising the vector of Embodiment 197 or Embodiment 198.
E200. A method of producing an antibody or fragment thereof which specifically binds-IGF-1R, comprising culturing the host cell of Embodiment 199, and recovering said antibody, or fragment thereof.
E201. An isolated polypeptide produced by the method of Embodiment 200.
E202. An isolated polypeptide encoded by the polynucleotide of any one of Embodiments 118 to 161.
E203. The isolated polypeptide of Embodiment 202, wherein an antibody or fragment thereof comprising said polypeptide specifically binds to IGF-1R.
E204. An isolated antibody or fragment thereof comprising the polypeptide of Embodiment 168 or 169.
E205. A composition comprising an isolated VH encoding polynucleotide and an isolated VL encoding polynucleotide, wherein said VH encoding polynucleotide and said VL encoding polynucleotide, respectively, comprise nucleic acids encoding amino acid sequences at least 90% identical to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118; and wherein an antibody or fragment thereof encoded by said VH and VL encoding polynucleotides specifically binds IGF-1R.
E206. The composition of Embodiment 205, wherein said VH encoding polynucleotide and said VL encoding polynucleotide, respectively, comprise nucleic acids encoding amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118
E207. A composition comprising an isolated VH encoding polynucleotide and an isolated VL encoding polynucleotide, wherein said VH encoding polynucleotide and said VL encoding polynucleotide, respectively, comprise nucleic acids encoding amino acid sequences identical, except for less than 20 conservative amino acid substitutions, to reference amino acid sequences selected from the group consisting of: SEQ ID NO: 4 and SEQ ID NO: 68; SEQ ID NO: 8 and SEQ ID NO: 73; SEQ ID NO: 14 and SEQ ID NO: 78; SEQ ID NO: 20 and SEQ ID NO: 83; SEQ ID NO: 26 and SEQ ID NO: 88; SEQ ID NO: 32 and SEQ ID NO: 93; SEQ ID NO: 38 and SEQ ID NO: 98; SEQ ID NO: 43 and SEQ ID NO: 103; SEQ ID NO: 48 and SEQ ID NO: 108; SEQ ID NO: 53 and SEQ ID NO: 103; SEQ ID NO: 58 and SEQ ID NO: 113; and SEQ ID NO: 63 and 118; and wherein an antibody or fragment thereof encoded by said VH and VL encoding polynucleotides specifically binds IGF-1R.
E208. A composition comprising an isolated VH encoding polynucleotide and an isolated VL encoding polynucleotide, wherein said VH encoding polynucleotide encodes a VH polypeptide comprising VH-CDR1, VH-CDR2, and VH-CDR3 amino acid sequences selected from the group consisting of: SEQ ID NOs: 5, 6, and 7; SEQ ID NOs: 10, 11, and 12; SEQ ID NOs: 15, 16, and 17; SEQ ID NOs: 21, 22, and 23; SEQ ID NOs: 27, 28, and 29; SEQ ID NOs: 33, 34, and 35; SEQ ID NOs: 39, 40, and 41; SEQ ID NOs: 44, 45, and 46; SEQ ID NOs: 49, 50, and 51; SEQ ID NOs: 54, 55, and 56; SEQ ID NOs: 59, 60, and 61; and SEQ ID NOs: 64, 65, and 66; wherein said VL encoding polynucleotide encodes a VL polypeptide comprising VL-CDR1, VL-CDR2, and VL-CDR3 amino acid sequences selected from the group consisting of: SEQ ID NOs: 69, 70, and 71; SEQ ID NOs: 74, 75, and 76; SEQ ID NOs: 79, 80, and 81; SEQ ID NOs: 84, 85, and 86; SEQ ID NOs: 89, 90, and 91; SEQ ID NOs: 94, 95, and 96; SEQ ID NOs: 99, 100, and 101; SEQ ID NOs: 104, 105, and 106; SEQ ID NOs: 109, 110, and 111; SEQ ID NOs: 114, 115, and 116; and SEQ ID NOs: 119, 120, and 121; and wherein an antibody or fragment thereof encoded by said VH and VL encoding polynucleotides specifically binds IGF-1R.
E209. The composition of any one of Embodiments 205-208, wherein said VH encoding polynucleotide further comprises a nucleic acid encoding a signal peptide fused to said antibody VH polypeptide.
E210. The composition of any one of Embodiments 205-208, wherein said VL encoding polynucleotide further comprises a nucleic acid encoding a signal peptide fused to said antibody VL polypeptide.
E211. The composition of any one of Embodiments 205 to 210, wherein said VH encoding polynucleotide further comprises a nucleic acid encoding a heavy chain constant region CH1 domain fused to said VH polypeptide.
E212. The composition of any one of Embodiments 205 to 211, wherein said VH encoding polynucleotide further comprises a nucleic acid encoding a heavy chain constant region CH2 domain fused to said VH polypeptide.
E213. The composition of any one of Embodiments 205 to 212, wherein said VH encoding polynucleotide further comprises a nucleic acid encoding a heavy chain constant region CH3 domain fused to said VH polypeptide.
E214. The composition of any one of Embodiments 205 to 213, wherein said VH encoding polynucleotide further comprises a nucleic acid encoding a heavy chain hinge region fused to said VH polypeptide.
E215. The composition of any one of Embodiments 205 to 214, wherein said heavy chain constant region is human IgG4.
E216. The composition of Embodiment 215, wherein said IgG4 is mutagenized to remove glycosylation sites.
E217. The composition of Embodiment 216, wherein said mutations comprise S241P and T318A using the Kabat numbering system.
E218. The composition of any one of Embodiments 205 to 217, wherein said VL encoding polynucleotide further comprises a nucleic acid encoding a light chain constant region domain fused to said VL polypeptide.
E219. The composition of Embodiment 174, wherein said light chain constant region is human kappa.
E220. The composition of any one of Embodiments 205 to 219, wherein an antibody or fragment thereof encoded by said VH and VL encoding polynucleotides specifically binds the same IGF-1R epitope as a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8.
E221. The composition of any one of Embodiments 205 to 220, wherein an antibody or fragment thereof encoded by said VH and VL encoding polynucleotides competitively inhibits a reference monoclonal Fab antibody fragment selected from the group consisting of M13-C06, M14-G11, M14-C03, M14-B01, M12-E01, and M12-G04, or a reference monoclonal antibody produced by a hybridoma selected from the group consisting of P2A7.3E11, 20C8.3B8, P1A2.2B11, 20D8.24B11, P1E2.3B12, and P1G10.2B8 from binding to IGF-1R.
E222. The composition of any one of Embodiments 205 to 221, wherein the framework regions of said VH and VL polypeptides are human, except for five or fewer amino acid substitutions.
E223. The composition of any one of Embodiments 205 to 222, wherein an antibody or fragment thereof encoded by said VH and VL encoding polynucleotides binds to a linear epitope.
E224. The composition of any one of Embodiments 205 to 222, wherein an antibody or fragment thereof encoded by said VH and VL encoding polynucleotides binds to a non-linear conformational epitope.
E225. The composition of any one of Embodiments 205 to 222, wherein an antibody or fragment thereof encoded by said VH and VL encoding polynucleotides is multivalent, and comprises at least two heavy chains and at least two light chains.
E226. The composition of any one of Embodiments 205 to 225, wherein an antibody or fragment thereof encoded by said VH and VL encoding polynucleotides is multispecific.
E227. The composition of any one of Embodiments 205 to 226, wherein an antibody or fragment thereof encoded by said VH and VL encoding polynucleotides is bispecific.
E228. The composition of any one of Embodiments 205 to 227, wherein an antibody or fragment thereof encoded by said VH and VL encoding polynucleotides comprises heavy and light chain variable domains which are fully human.
E229. The composition of Embodiment 228, wherein said heavy and light chain variable domains are identical to those of a monoclonal Fab antibody fragment selected from the group consisting of M13-C06, M14-G11, M14-C03, M14-B01, M12-E01, and M12-G04.
E230. The composition of any one of Embodiments 205 to 227, wherein an antibody or fragment thereof encoded by said VH and VL encoding polynucleotides comprises heavy and light chain variable domains which are murine.
E231. The composition of Embodiment 230, wherein said heavy and light chain variable domains are identical to those of a monoclonal antibody produced by a hybridoma selected from the group consisting of P2A7.3E11, 20C8.3B8, P1A2.2B11, 20D8.24B11, P1E2.3B12, and P1G10.2B8.
E232. The composition of any one of Embodiments 205 to 227 and 230 to 231, wherein an antibody or fragment thereof encoded by said VH and VL encoding polynucleotides is humanized.
E233. The composition of any one of Embodiments 205 to 227 and 230 to 231, wherein an antibody or fragment thereof encoded by said VH and VL encoding polynucleotides is chimeric.
E234. The composition of any one of Embodiments 205 to 227 and 230 to 231, wherein an antibody or fragment thereof encoded by said VH and VL encoding polynucleotides is primatized.
E235. The composition of any one of Embodiments 205 to 229, wherein an antibody or fragment thereof encoded by said VH and VL encoding polynucleotides is fully human.
E236. The composition of any one of Embodiments 205 to 235, wherein an antibody or fragment thereof encoded by said VH and VL encoding polynucleotides is an Fab fragment.
E237. The composition of any one of Embodiments 205 to 235, wherein an antibody or fragment thereof encoded by said VH and VL encoding polynucleotides is an Fab′ fragment.
E238. The composition of any one of Embodiments 205 to 235, wherein an antibody or fragment thereof encoded by said VH and VL encoding polynucleotides is an F(ab)₂ fragment.
E239. The composition of any one of Embodiments 205 to 235, wherein an antibody or fragment thereof encoded by said VH and VL encoding polynucleotides is an Fv fragment.
E240. The composition of any one of Embodiments 205 to 235, wherein an antibody or fragment thereof encoded by said VH and VL encoding polynucleotides is a single chain antibody.
E241. The composition of any one of Embodiments 205 to 240, wherein an antibody or fragment thereof encoded by said VH and VL encoding polynucleotides specifically binds to an IGF-1R polypeptide or fragment thereof, or an IGF-1R variant polypeptide, with an affinity characterized by a dissociation constant (KD) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.
E242. The composition of any one of Embodiments 205 to 241, wherein an antibody or fragment thereof encoded by said VH and VL encoding polynucleotides preferentially binds to a human IGF-1R polypeptide or fragment thereof, relative to a murine IGF-1R polypeptide or fragment thereof or a non-human primate IGF-1R polypeptide or fragment thereof.
E243. The composition of any one of Embodiments 205 to 241, wherein an antibody or fragment thereof encoded by said VH and VL encoding polynucleotides binds to a human IGF-1R polypeptide or fragment thereof, and also binds to a non-human primate IGF-1R polypeptide or fragment thereof.
E244. The composition of any one of Embodiments 205 to 243, wherein an antibody or fragment thereof encoded by said VH and VL encoding polynucleotides binds to IGF-1R expressed on the surface of a cell.
E245. The composition of Embodiment 224, wherein said cell is a malignant cell, a neoplastic cell, a tumor cell, or a metastatic cell.
E246. The composition of any one of Embodiments 205 to 245, wherein an antibody or fragment thereof encoded by said VH and VL encoding polynucleotides blocks insulin growth factor from binding to IGF-1R.
E247. The composition of Embodiment 246, wherein said insulin growth factor is insulin growth factor-1 (IGF-1).
E248. The composition of Embodiment 246, wherein said insulin growth factor is insulin growth factor-2 (IGF-2).
E249. The composition of Embodiment 246, which blocks both IGF-1 and IGF-2 from binding to IGF-1R.
E250. The composition of any one of Embodiments 205 to 249, wherein an antibody or fragment thereof encoded by said VH and VL encoding polynucleotides inhibits IGF-1R-mediated cell proliferation.
E251. The composition of any one of Embodiments 205 to 250, wherein an antibody or fragment thereof encoded by said VH and VL encoding polynucleotides inhibits IGF-1 or IGF-2-mediated IGF-1R phosphorylation.
E252. The composition of any one of Embodiments 205 to 251, wherein an antibody or fragment thereof encoded by said VH and VL encoding polynucleotides inhibits tumor cell growth.
E253. The composition of any one of Embodiments 205 to 252, wherein an antibody or fragment thereof encoded by said VH and VL encoding polynucleotides inhibits IGF-1R internalization.
E254. The composition of any one of Embodiments 205 to 253, wherein said VH encoding polynucleotide, said VL encoding polynucleotide, or both said VH and said VL encoding polynucleotides further comprise a nucleic acid encoding a heterologous polypeptide.
E255. The composition of any one of Embodiments 205 to 254, wherein an antibody or fragment thereof encoded by said VH and VL encoding polynucleotides is conjugated to an agent selected from the group consisting of cytotoxic agent, a therapeutic agent, cytostatic agent, a biological toxin, a prodrug, a peptide, a protein, an enzyme, a virus, a lipid, a biological response modifier, pharmaceutical agent, a lymphokine, a heterologous antibody or fragment thereof, a detectable label, polyethylene glycol (PEG), and a combination of two or more of any said agents.
E256. The composition of Embodiment 255, wherein said cytotoxic agent is selected from the group consisting of a radionuclide, a biotoxin, an enzymatically active toxin, a cytostatic or cytotoxic therapeutic agent, a prodrugs, an immunologically active ligand, a biological response modifier, or a combination of two or more of any said cytotoxic agents.
E257. The composition of Embodiment 255, wherein said detectable label is selected from the group consisting of an enzyme, a fluorescent label, a chemiluminescent label, a bioluminescent label, a radioactive label, or a combination of two or more of any said detectable labels.
E258. The composition of any one of Embodiments 205 to 257, wherein said VH encoding polynucleotide is contained on a first vector and said VL encoding polynucleotide is contained on a second vector.
E259. The composition of Embodiment 258, wherein said VH encoding polynucleotide is operably associated with a first promoter and said VL encoding polynucleotide is operably associated with a second promoter.
E260. The composition of Embodiment 259, wherein said first and second promoters are copies of the same promoter.
E261. The composition of Embodiment 259, wherein said first and second promoters non-identical.
E262. The composition of any one of Embodiments 258 to 261, wherein said first vector and said second vector are contained in a single host cell.
E263. The composition of any one of Embodiments 258 to 261, wherein said first vector and said second vector are contained in a separate host cells.
E264. A method of producing an antibody or fragment thereof which specifically binds IGF-1R, comprising culturing the host cell of Embodiment 262, and recovering said antibody, or fragment thereof.
E265. A method of producing an antibody or fragment thereof which specifically binds IGF-1R, comprising co-culturing the separate host cells of Embodiment 263, and recovering said antibody, or fragment thereof.
E266. A method of producing an antibody or fragment thereof which specifically binds IGF-1R, comprising separately culturing the separate host cells of Embodiment 263, combining said VH and VL encoding polypeptides, and recovering said antibody, or fragment thereof.
E267. An antibody or fragment thereof which specifically binds IGF-1R, produced by the method of any one of Embodiments 264 to 266.
E268. The composition of any one of Embodiments 205 to 267, wherein said VH encoding polynucleotide and said VL encoding polynucleotide are on the same vector.
E269. The vector of Embodiment 268.
E270. The vector of Embodiment 269, wherein said VH encoding polynucleotide and said VL encoding polynucleotide are each operably associated with a promoter.
E271. The vector of Embodiment 269, wherein said VH encoding polynucleotide and said VL encoding polynucleotide are fused in frame, are co-transcribed from a single promoter operably associated therewith, and are cotranslated into a single chain antibody or antigen-binding fragment thereof.
E272. The vector of Embodiment 269, wherein said VH encoding polynucleotide and said VL encoding polynucleotide are co-transcribed from a single promoter operably associated therewith, but are separately translated.
E273. The vector of Embodiment 272, further comprising an IRES sequence disposed between said VH encoding polynucleotide and said VL encoding polynucleotide.
E274. The vector of Embodiment 269, wherein said polynucleotide encoding a VH and said polynucleotide encoding a VL are separately transcribed, each being operably associated with a separate promoter.
E275. The vector of Embodiment 274, wherein said separate promoters are copies of the same promoter.
E276. The vector of Embodiment 274, wherein said separate promoters are non-identical.
E277. A host cell comprising the vector of any one of Embodiments 269 to 276.
E278. A method of producing an antibody or fragment thereof which specifically binds IGF-1R, comprising culturing the host cell of Embodiment 277, and recovering said antibody, or fragment thereof.
E279. An antibody or fragment thereof which specifically binds IGF-1R, produced by the method of Embodiment 244.
E280. A method for treating a hyperproliferative disorder in an animal, comprising administering to an animal in need of treatment a composition comprising:
   a) the isolated antibody or fragment thereof of any one of Embodiments 1 to 82, 170, 233 and 245; and
   b) a pharmaceutically acceptable carrier.
E281. The method of Embodiment 280, wherein said hyperproliferative disease or disorder is selected from the group consisting of cancer, a neoplasm, a tumor, a malignancy, or a metastasis thereof.
E282. The method of Embodiment 281, wherein said antibody or fragment thereof specifically binds to IGF-1R expressed on the surface of a malignant cell.
E283. The method of Embodiment 282, wherein binding of said antibody or fragment thereof to said malignant cell results in growth inhibition of said malignant cell.
E284. The method of any one of Embodiments 280 to 283, wherein said antibody or fragment thereof inhibits IGF binding to said malignant cell.
E285. The method of Embodiment 284, wherein said IGF is IGF-1.
E286. The method of Embodiment 284, wherein said IGF is IGF-2.
E287. The method of Embodiment 284, wherein said IGF is IGF-1 and IGF-2.
E288. The method of Embodiment 287, wherein said antibody or fragment thereof inhibits IGF-1 from binding to said malignant cell but does not inhibit IGF-2.
E289. The method of Embodiment 287, wherein said antibody or fragment thereof inhibits IGF-2 from binding to said malignant cell but does not inhibit IGF-1.
E290. The method of any one of Embodiments 280 to 283, wherein said aid antibody or fragment thereof promotes internalization if IGF-1R into said malignant cell.
E291. The method of any one of Embodiments 280 to 283, wherein said aid antibody or fragment thereof inhibits IGF-1R phosphorylation.
E292. The method of any one of Embodiments 280 to 283, wherein said aid antibody or fragment thereof inhibits tumor cell proliferation.
E293. The method of Embodiment 292, wherein tumor cell proliferation is inhibited through the prevention or retardation of metastatic growth.
E294. The method of any one of Embodiments 280 to 283, wherein said aid antibody or fragment thereof inhibits tumor cell migration.
E295. The method of Embodiment 292, wherein tumor cell proliferation is inhibited through the prevention or retardation of tumor spread to adjacent tissues.
E296. The method of Embodiment 281, wherein said hyperproliferative disease or disorder is a neoplasm located in the: prostate, colon, abdomen, bone, breast, digestive system, liver, pancreas, peritoneum, adrenal gland, parathyroid gland, pituitary gland, testicles, ovary, thymus, thyroid, eye, head, neck, central nervous system, peripheral nervous system, lymphatic system, pelvis, skin, soft tissue, spleen, thoracic region, or urogenital tract.
E297. The method of Embodiment 281, wherein said hyperproliferative disease is cancer, said cancer selected from the group consisting of: epithelial squamous cell cancer, melanoma, leukemia, myeloma, stomach cancer, brain cancer, lung cancer, pancreatic cancer, cervical cancer, ovarian cancer, liver cancer, bladder cancer, breast cancer, colon cancer, renal cancer, prostate cancer, testicular cancer, thyroid cancer, and head and neck cancer.
E298. The method of Embodiment 297, wherein said cancer is selected from the group consisting of stomach cancer, renal cancer, brain cancer, bladder cancer, colon cancer, lung cancer, breast cancer, pancreatic cancer, ovarian cancer, and prostate cancer.
E299. The method of any one of Embodiments 280 to 298, wherein said animal is a mammal.
E300. The method of Embodiment 299, wherein said mammal is a human.
E301. A method for treating a hyperproliferative disorder in an animal, comprising administering a combination of two or more antibodies, or fragments thereof, wherein said antibodies, or fragments thereof, specifically bind at least two different IGF-1R epitopes.
E302. The method of Embodiment 301, wherein said two or more antibodies, or fragments thereof, inhibit IGF-1 and/or IGF-2 ligand binding.
E303 The method of Embodiment 301, wherein said two or more antibodies, or fragments thereof, inhibit IGF-1R signal transduction.
E304. The method of any one of Embodiments 301-303, wherein said two or more antibodies, or fragments thereof, inhibit ligand binding both competitively and allosterically.
E305. The method of any one of Embodiments 301-304, wherein said two or more antibodies, or fragments thereof, inhibit tumor cell proliferation or IGF-1R signal transduction, more effectively in combination than any one of said antibodies, or fragments thereof, alone, wherein comparison of antibodies, or fragments thereof, in combination versus alone compares said antibodies, or fragments thereof, at approximately the same final total molar concentrations.
E306. The method of any one of Embodiments 301-304, wherein said two or more antibodies, or fragments thereof, have a synergistic effect on the inhibition of tumor cell proliferation or IGF-1R signal transduction.
E307. The method of any one of Embodiments 301-304, wherein said two or more antibodies, or fragments thereof, inhibit IGF-1 and/or IGF-2 binding to IGF-1R, more effectively in combination than any one of said antibodies, or fragments thereof, alone, wherein comparison of antibodies, or fragments thereof, in combination versus alone compares said antibodies, or fragments thereof, at approximately the same final total molar concentrations.
E308. The method of any one of Embodiments 301-304, wherein said two or more antibodies, or fragments thereof, have a synergistic effect on the inhibition of IGF-1 and/or IGF-2 binding to IGF-1R.
E309. The method of any one of Embodiments 301-308, wherein said animal is a human.
E310. The method of any one of Embodiments 301-309, wherein said hyperproliferative disorder is cancer.
E311. The method of any one of Embodiments 301-310, wherein said hyperproliferative disorder is a tumor.
E312. An isolated polypeptide comprising, or consisting of, the Fibronectin type III domain 1 (FNIII-1) of human IGF-1R.
E313. An isolated polypeptide comprising, or consisting of, the cysteine rich repeat domain (CRR) and the leucine rich repeat domain 2 (L2) of human IGF-1R.
E314. An isolated polypeptide comprising, or consisting of, the cysteine rich repeat domain (CRR) of human IGF-1R.
E315. An isolated polypeptide having 300 amino acid residues or less in length and comprising a sequence, wherein the sequence is selected from the group consisting of:
   a) Glu Ser Asp Val Leu His Phe Thr Ser Thr; b) Thr Thr Ser Lys Asn Arg Ile Ile He Thr; c) Trp His Arg Tyr Arg Pro Pro Asp Tyr Arg; d) Asp Leu Ile Ser Phe Thr Val Tyr Tyr Lys; e) Glu Ala Pro Phe Lys Asn Val Thr Glu Tyr; f) Asp Gly Gln Asp Ala Cys Gly Ser Asn Ser; g) Trp Asn Met Val Asp Val Asp Leu Pro Pro; h) Asn Lys Asp Val Glu Pro Gly Ile Leu Leu; i) His Gly Leu Lys Pro Trp Thr Gln Tyr Ala; j) Val Tyr Val Lys Ala Val Thr Leu Thr Met; k) Val Glu Asn Asp His Ile Arg Gly Ala Lys; and, l) Asp His Ile Arg Gly Ala Lys Ser Glu Ile.
E316. The polypeptide of Embodiment 315, wherein said polypeptide is 200 amino acids or less in length.
E317. The polypeptide of Embodiment 315, wherein said polypeptide is 250 amino acids or less in length.
E318. The polypeptide of Embodiment 315, wherein said polypeptide is 150 amino acids or less in length.
E319. The polypeptide of Embodiment 315, wherein said polypeptide is 100 amino acids or less in length.
E320. The polypeptide of Embodiment 315, wherein said polypeptide is 50 amino acids or less in length.
E321. The polypeptide of Embodiment 315, wherein said polypeptide is 40 amino acids or less in length.
E322. The polypeptide of Embodiment 315, wherein said polypeptide is 30 amino acids or less in length.
E323. The polypeptide of Embodiment 315, wherein said polypeptide is 20 amino acids or less in length.
E324. The polypeptide of Embodiment 315, wherein said polypeptide is 10 amino acids in length.
E325. The polypeptide of any one of Embodiments 315 to 324, wherein said polypeptide is specifically bound by an antibody selected from the group consisting of:
   a) Fab M13-C06; b) Fab M14-C03; c) M13.C06.G4.P.agly; and, d) M14.C03.G4.P.agly.
E326. An epitope of human IGF-1R comprising one or more amino acids selected from the group consisting of:

a) Glu-459; b) Ser-460; c) Asp-461; d) Val-462; e) His-464; f) Thr-466; g) Ser-467; h) Thr-478; i) His480; j) Tyr-482; k) Arg-483; 1) Glu-533; m) Ile-564; n) Arg-565; o) Lys-568; p) Glu-570; and, q) Ile-571,
wherein said amino acids correspond to the sequence of human IGF-1R without the first 30 amino acids of the signal peptide sequence as indicated in Table 20.
E327. An isolated polypeptide having 300 amino acid residues or less in length and comprising a sequence, wherein the sequence is selected from the group consisting of:
   a) Asp Arg Asp Phe Cys Ala Asn Ee Leu Ser; b) Ala Glu Ser Ser Asp Ser Glu Gly Phe Val; c) Ile His Asp Gly Glu Cys Met Gln Glu Cys; d) Pro Ser Gly Phe Ile Arg Asn Gly Ser Gln; e) Ser Met Tyr Cys Ile Pro Cys Glu Gly Pro; and, f) Cys Glu Gly Pro Cys Pro Lys Val Cys Glu.
E328. The polypeptide of Embodiment 327, wherein said polypeptide is 200 amino acids or less in length.
E329. The polypeptide of Embodiment 327, wherein said polypeptide is 250 amino acids or less in length.
E330. The polypeptide of Embodiment 327, wherein said polypeptide is 150 amino acids or less in length.
E331. The polypeptide of Embodiment 327, wherein said polypeptide is 100 amino acids or less in length.
E332. The polypeptide of Embodiment 327, wherein said polypeptide is 50 amino acids or less in length.
E333. The polypeptide of Embodiment 327, wherein said polypeptide is 40 amino acids or less in length.
E334. The polypeptide of Embodiment 327, wherein said polypeptide is 30 amino acids or less in length.
E335. The polypeptide of Embodiment 327, wherein said polypeptide is 20 amino acids or less in length.
E336. The polypeptide of Embodiment 327, wherein said polypeptide is 10 amino acids in length.
E337. The polypeptide of any one of Embodiments 327 to 336, wherein said polypeptide is specifically bound by an antibody selected from the group consisting of:
   a) Fab M14-G11; and,
   b) M14-G11.G4.P.agly.
E338. An epitope of human IGF-1R comprising one or more amino acids selected from the group consisting of:
   a) Asp-248; b) Asp-250; c) Asn-254; d) Ser-257; e) Glu-259; f) Ser-260; g) Ser-263; h) Gly-265; and, i) Glu-303,
wherein said amino acids correspond to the sequence of human IGF-1R without the first 30 amino acids of the signal peptide sequence as indicated in Table 20.
E339. An isolated polypeptide having 300 amino acid residues or less in length and comprising a sequence, wherein the sequence is selected from the group consisting of:
   a) Asp Arg Asp Phe Cys Ala Asn Ile Leu Ser; and, b) Leu Ser Ala Glu Ser Ser Asp Ser Glu Gly.
E340. The polypeptide of Embodiment 339, wherein said polypeptide is 200 amino acids or less in length.
E341. The polypeptide of Embodiment 339, wherein said polypeptide is 250 amino acids or less in length.
E342. The polypeptide of Embodiment 339, wherein said polypeptide is 150 amino acids or less in length.
E343. The polypeptide of Embodiment 339, wherein said polypeptide is 100 amino acids or less in length.
E344. The polypeptide of Embodiment 339, wherein said polypeptide is 50 amino acids or less in length.
E345. The polypeptide of Embodiment 339, wherein said polypeptide is 40 amino acids or less in length.
E346. The polypeptide of Embodiment 339, wherein said polypeptide is 30 amino acids or less in length.
E347. The polypeptide of Embodiment 339, wherein said polypeptide is 20 amino acids or less in length.
E348. The polypeptide of Embodiment 339, wherein said polypeptide is 10 amino acids in length.
E349. The polypeptide of any one of Embodiments 339 to 348, wherein said polypeptide is specifically bound by an antibody selected from the group consisting of:
   a) chimeric antibody P1E2; and,
   b) antibodies expressed by hybridoma cell line P1E2.3B12.
E350. An epitope of human IGF-1R comprising one or more amino acids selected from the group consisting of:
   a) Asp-248; b) Asn-254; c) Ser-257; and, d) Gly-265,
   wherein said amino acids correspond to the sequence of human IGF-1R without the first 30 amino acids of the signal peptide sequence as indicated in Table 20.
E351. An isolated polypeptide comprising amino acids residues Glu-459 to His464 of human IGF-1R, wherein said amino acids correspond to the sequence of human IGF-1R without the first 30 amino acids of the signal peptide sequence as indicated in Table 20.
E352. An isolated polypeptide comprising amino acids residues Ser-460 to Thr-466 of human IGF-1R, wherein said amino acids correspond to the sequence of human IGF-1R without the first 30 amino acids of the signal peptide sequence as indicated in Table 20.
E353. An isolated polypeptide comprising amino acids residues Asp-461 to Thr-466 of human IGF-1R, wherein said amino acids correspond to the sequence of human IGF-1R without the first 30 amino acids of the signal peptide sequence as indicated in Table 20.
E354. An isolated polypeptide comprising amino acids residues Val-462 to Ser-467 of human IGF-1R, wherein said amino acids correspond to the sequence of human IGF-1R without the first 30 amino acids of the signal peptide sequence as indicated in Table 20.
E355. An isolated polypeptide comprising amino acids residues His464 to Thr-478 of human IGF-1R, wherein said amino acids correspond to the sequence of human IGF-1R without the first 30 amino acids of the signal peptide sequence as indicated in Table 20.
E356. An isolated polypeptide comprising amino acids residues Thr-466 to Thr-478 of human IGF-1R, wherein said amino acids correspond to the sequence of human IGF-1R without the first 30 amino acids of the signal peptide sequence as indicated in Table 20.
E357. An isolated polypeptide comprising amino acids residues Ser-467 to Thr-478 of human IGF-1R, wherein said amino acids correspond to the sequence of human IGF-1R without the first 30 amino acids of the signal peptide sequence as indicated in Table 20.
E358. An isolated polypeptide comprising amino acids residues Thr-478 to Tyr-482 of human IGF-1R, wherein said amino acids correspond to the sequence of human IGF-1R without the first 30 amino acids of the signal peptide sequence as indicated in Table 20.
E359. An isolated polypeptide comprising amino acids residues His480 to Glu-533 of human IGF-1R, wherein said amino acids correspond to the sequence of human IGF-1R without the first 30 amino acids of the signal peptide sequence as indicated in Table 20.
E360. An isolated polypeptide comprising amino acids residues Tyr-482 to Glu-533 of human IGF-1R, wherein said amino acids correspond to the sequence of human IGF-1R without the first 30 amino acids of the signal peptide sequence as indicated in Table 20.
E361. An isolated polypeptide comprising amino acids residues Arg-483 to Glu-533 of human IGF-1R, wherein said amino acids correspond to the sequence of human IGF-1R without the first 30 amino acids of the signal peptide sequence as indicated in Table 20.
E362. An isolated polypeptide comprising amino acids residues Glu-533 to Ile-564 of human IGF-1R, wherein said amino acids correspond to the sequence of human IGF-1R without the first 30 amino acids of the signal peptide sequence as indicated in Table 20.
E363. An isolated polypeptide comprising amino acids residues Ile-564 to Glu-570 of human IGF-1R, wherein said amino acids correspond to the sequence of human IGF-1R without the first 30 amino acids of the signal peptide sequence as indicated in Table 20.
E364. An isolated polypeptide comprising amino acids residues Arg-565 to Glu-570 of human IGF-1R, wherein said amino acids correspond to the sequence of human IGF-1R without the first 30 amino acids of the signal peptide sequence as indicated in Table 20.
E365. An isolated polypeptide comprising amino acids residues Arg-565 to Ile-571 of human IGF-1R, wherein said amino acids correspond to the sequence of human IGF-1R without the first 30 amino acids of the signal peptide sequence as indicated in Table 20.
E366. An isolated polypeptide comprising amino acids residues Glu-459 to Arg-483 of human IGF-1R, wherein said amino acids correspond to the sequence of human IGF-1R without the first 30 amino acids of the signal peptide sequence as indicated in Table 20.
E367. An isolated polypeptide comprising amino acids residues Arg-483 to Glu-533 of human IGF-1R, wherein said amino acids correspond to the sequence of human IGF-1R without the first 30 amino acids of the signal peptide sequence as indicated in Table 20.
E368. An isolated polypeptide comprising amino acids residues Glu-533 to Ile-571 of human IGF-1R, wherein said amino acids correspond to the sequence of human IGF-1R without the first 30 amino acids of the signal peptide sequence as indicated in Table 20.
E369. An isolated polypeptide comprising amino acids residues Glu-459 to Ile-571 of human IGF-1R, wherein said amino acids correspond to the sequence of human IGF-1R without the first 30 amino acids of the signal peptide sequence as indicated in Table 20.
E370. An isolated polypeptide comprising amino acids residues Asp-248 to Asn-254 of human IGF-1R, wherein said amino acids correspond to the sequence of human IGF-1R without the first 30 amino acids of the signal peptide sequence as indicated in Table 20.
E371. An isolated polypeptide comprising amino acids residues Asp-250 to Ser-257 of human IGF-1R, wherein said amino acids correspond to the sequence of human IGF-1R without the first 30 amino acids of the signal peptide sequence as indicated in Table 20.
E372. An isolated polypeptide comprising amino acids residues Asn-254 to Glu-259 of human IGF-1R, wherein said amino acids correspond to the sequence of human IGF-1R without the first 30 amino acids of the signal peptide sequence as indicated in Table 20.
E373. An isolated polypeptide comprising amino acids residues Ser-257 to Ser-263 of human IGF-1R, wherein said amino acids correspond to the sequence of human IGF-1R without the first 30 amino acids of the signal peptide sequence as indicated in Table 20.
E374. An isolated polypeptide comprising amino acids residues Glu-259 to Gly-265 of human IGF-1R, wherein said amino acids correspond to the sequence of human IGF-1R without the first 30 amino acids of the signal peptide sequence as indicated in Table 20.
E375. An isolated polypeptide comprising amino acids residues Ser-260 to Gly-265 of human IGF-1R, wherein said amino acids correspond to the sequence of human IGF-1R without the first 30 amino acids of the signal peptide sequence as indicated in Table 20.
E376. An isolated polypeptide comprising amino acids residues Ser-263 to Glu-303 of human IGF-1R, wherein said amino acids correspond to the sequence of human IGF-1R without the first 30 amino acids of the signal peptide sequence as indicated in Table 20.
E377. An isolated polypeptide comprising amino acids residues Ser-265 to Glu-303 of human IGF-1R, wherein said amino acids correspond to the sequence of human IGF-1R without the first 30 amino acids of the signal peptide sequence as indicated in Table 20.
E378. An isolated polypeptide comprising amino acids residues Ser-254 to Gly-265 of human IGF-1R, wherein said amino acids correspond to the sequence of human IGF-1R without the first 30 amino acids of the signal peptide sequence as indicated in Table 20.
E379. An isolated antibody capable of allosterically inhibiting IGF-1 binding to IGF-1R
E380. The antibody of Embodiment 379, wherein said antibody does not inhibit IGF-2 binding to IGF-1R.
E381. The isolated antibody of Embodiment 379 or 380, wherein said antibody is selected from the group consisting of: a) P1E2; and, b) P1A2.
E382. A method of allosterically inhibiting IGF-1 binding to IGF-1R wherein said method comprises:
   a) exposing IGF-1R to an IGF-1R specific antibody; and,
   b) allowing said antibody sufficient time to bind said IGF-1R.
E383. The method of Embodiment 382, wherein said antibody does not inhibit IGF-2 binding to IGF-1R.
E384. The method of Embodiment 382 or 383, wherein said antibody is selected from the group consisting of: a) P1E2; and, b) P1A2.
E385. An isolated antibody capable of allosterically inhibiting IGF-2 binding to IGF-1R
E386. The antibody of Embodiment 385, wherein said antibody does not inhibit IGF-1 binding to IGF-1R.
E387. The isolated antibody of Embodiment 385 or 386, wherein said antibody is P3F9.
E388. A method of allosterically inhibiting IGF-2 binding to IGF-1R wherein said method comprises:
   a) exposing IGF-1R to an IGF-1R specific antibody; and,
   b) allowing said antibody sufficient time to bind said IGF-1R.
E389. The method of Embodiment 388, wherein said antibody does not inhibit IGF-1 binding to IGF-1R.
E390. The method of Embodiment 388 or 389, wherein said antibody is P3F9.
E391. An isolated antibody capable of allosterically inhibiting IGF-1 and IGF-2 binding to IGF-1R
E392. The isolated antibody of Embodiment 391, wherein said antibody is selected from the group consisting of: a) M13-C06; b) M14-C03; and, c) 20C8.
E393. A method of allosterically inhibiting IGF-1 and IGF-2 binding to IGF-1R wherein said method comprises:
   a) exposing IGF-1R to one or more IGF-1R specific antibodies; and,
   b) allowing said one or more antibodies sufficient time to bind said IGF-1R.
E394. The method of Embodiment 393, wherein said one or more antibodies is/are selected from the group consisting of:
   a) P1E2; b) P1A2; c) P3F9; d) M13-C06; e) M14-C03; and, f) 20C8.
E395. An isolated antibody capable of competitively inhibiting IGF-1 and IGF-2 binding to IGF-1R
E396. The isolated antibody of Embodiment 395, wherein said antibody is M14-G11.
E397. A method of competitively inhibiting IGF-1 and IGF-2 binding to IGF-1R wherein said method comprises:
   a) exposing IGF-1R to an IGF-1R specific antibody; and,
   b) allowing said antibody sufficient time to bind said IGF-1R.
E398. The method of Embodiment 397, wherein said antibody is M14-G11.
E399. A method of competitively and allosterically inhibiting IGF-1 and IGF-2 binding to IGF-1R wherein said method comprises:
   a) exposing IGF-1R to IGF-1R specific antibodies; and,
   b) allowing said antibodies sufficient time to bind said IGF-1R.
E400. The method of Embodiment 399, wherein said competitive inhibitor antibody is M14-G11 and wherein said allosteric inhibitor antibody is one or more antibodies selected from the group consisting of:
   a) P1E2; b) P1A2; c) P3F9; d) M13-C06; e) M14-C03; and, f) 20C8.

### REFERENCES

Brezinsky, S. C. G., Chiang, G. G., Szilvasi, A., Mohan, S., Shapiro, R. I., MacLean, A., Sisk, W., and Thill, G. (2003) “A simple method for enriching populations of transfected CHO cells for cells of higher specific productivity.” J. Immunol. Methods, 277: 141-155.
Day, E. S., Cachero, T. G., Qian, F., Sun, Y., Wen, D., Pelletier, M., Hsu, Y.-M., Whitty, A. (2005). “Selectivity of BAFF/BLyS and APRIL for Binding to the TNF Family Receptors BAFFR/BR3 and BCMA” Biochemistry, 44: 1919-1931.
Davies, D. R., and Cohen, G. H. (1996) “Interactions of protein antigens with antibodies.” Proc. Natl. Acad. Sci. USA, 93: 7-12.
Demarest, S. J., Chen, G., Kimmel, B. E., Gustafson, D., Wu, J., Salbato, J., Poland, J., Short, J., Hansen, G. (2006) Protein Engng. Des. Select, 19, 325-336.
Demarest, S. J., Hopp, J., Chung, J., Hathaway, K., Mertsching, E., Cao, X., George, J., Miatkowski, K., LaBarre, M. J., Shields, M., and Kehry, M. R. (2006) “An intermediate pH unfolding transition abrogates the ability of IgE to interact with its high affinity receptor FceRIa.” J. Biol. Chem., 281: 30755-30767.
Garrett, T. P., McKern, N. M., Lou, M., Frenkel, M. J., Bentley, J. D., Lovrecz, G. O., Elleman, T. C., Cosgrove, L. J., Ward, C. W. (1998) “Crystal structure of the first three domains of the type-1 insulin-like growth factor receptor,” Nature, 394 (6691):395-9.
Jansson, M., Hallen, D., Koho, H., Andersson, G, Berghard, L., Heidrich, J., Nyberg, E., Uhlen, M., Kordel, J., Nilsson, B. (1997) “Characterization of ligand binding of a soluble human insulin-like growth factor I receptor variant suggests a ligand-induced conformational change.” J. Biol. Chem., 272: 8189-8197.
Jacobs, S., Cook, S., Svoboda, M. E., Van Wyk, J. J., (1986) “Interaction of the monoclonal antibodies alpha IR-1 and alpha IR-3 with insulin and somatomedin-C receptors,” Endocrinology, 118 (1), pp. 223-226.
Kajander, T., Cortajarena, A. L., Main, E. R., Mochrie, S. G., Regan, L., (2005) “A new folding paradigm for repeat proteins,” J. Am. Chem. Soc., July 27; 127(29):10188-90.
Keynhanfar, M., Booker, G. W., Whittaker, J., Wallace, J. C., and Forbes, B. E. (2007) “Precise mapping of an IGF-1-binding site on IGF-1R.” Biochem. J., 401: 269-277.
McKern, N. M., Lawrence, M. C., Streltsov, V. A., Lou, M.-Z., Adams, T. E., Lovrecz, G. O., Elleman, T. C., Richards, K. M., Bentley, J. D., Pilling, P., Hoyne, P. A., Cartledge, K. A., Pham, T. M., Lewis, J. L., Sankovich, S. E., Stoichevska, V., Da Silva, E., Robinson, C. P., Frenkel, M. J., Sparrow, L. G., Fernley, R. T., Epa, V. C., and Ward, C. W. (2006) “Structure of the insulin receptor ectodomain reveals a folded-over conformation,” Nature 443: 218-221.
McKern, N. M., Lou. M., Frenkel, M. J., Verkuylen, A., Bentley, J. D., Lovrecz, G. O., Ivancic, N., Elleman, T. C., Garrett, T. P. J., Cosgrove, L. J., and Ward, C. W. (1997) “Crystallization of the first three domains of the human insulin-like growth factor-1 receptor,” Protein Sci. 6: 2663-2666.
Soos, M. A., Field, C. E., Lammers, R., Ullrich, A., Zhang, B., Roth, R. A., Andersen, A. S., Kjeldsen, T., Siddle, K. (1992) “A panel of monoclonal antibodies for the type I insulin-like growth factor receptor.” J. Biol. Chem. 267: 12955-12963.
Soos, M. A., Siddle, K., Baron, M. D., Heward, J. M., Luzio, J. P., Bellatin, J., and Lennox, E. S. (1986) “Monoclonal antibodies reacting with multiple epitopes on the human insulin receptor,” Biochem. J. 235: 199-208.
Sorensen, H., Whittaker, L., Hinrichsen, J., Groth, A., and Whittaker, J. (2004) “Mapping of the insulin-like growth factor II binding site of the Type I insulin-like growth factor receptor by alanine scanning mutagenesis,” FEBS Lett. 565: 19-22.
Whittaker, J., Groth, A. V., Mynarcik, D.C., Pluzek, L., Gadsboll, V. L., and Whittaker, L. J. (2001) “Alanine scanning mutagenesis of a type 1 insulin-like growth factor receptor ligand binding site,” J. Biol. Chem. 276: 43980-43986.

What is claimed:

1. A method of inhibiting IGF-1R signal transduction, comprising administering a combination of two or more antibodies, or fragments thereof which specifically bind to IGF-1R; wherein said antibodies, or fragments thereof have an additive, greater than additive, or synergistic effect on inhibition of said IGF-1R signal transduction.

2. The method of claim 1, wherein said signal transduction is inhibited in vivo.

3. The method of claim 2, wherein said signal transduction is inhibited in a human.

4. A method of treating a hyperproliferative disorder in an animal, comprising administering a combination of two or more antibodies, or fragments thereof which specifically bind to IGF-1R; wherein said antibodies, or fragments thereof have an additive, greater than additive, or synergistic effect on inhibition of said hyperproliferative disorder.

5. The method of claim 4, wherein said hyperproliferative disorder is cancer.

6. The method of claim 4, wherein said hyperproliferative disorder is a tumor.

7. The method of 4, wherein said animal is human.

8. The method of claim 1, wherein said two or more antibodies, or fragments thereof specifically bind at least two different IGF-1R epitopes.

9. The method of claim 4, wherein said two or more antibodies, or fragments thereof specifically bind at least two different IGF-1R epitopes.

10. The method of claim 8 wherein said two or more different IGF-1R epitopes are in regions of human IGF-1R selected from the group consisting of:
a) the fibronectin type III domain 1 (FNIII-1);
b) the cysteine rich repeat domain (CRR);
c) the leucine rich repeat domain 1 (L1);
d) the leucine rich repeat domain 1 (L2);
e) the center region of the cysteine rich repeat domain (CRR);
f) the carboxyl-terminal (C-terminal) region of the CRR;
g) the amino-terminal (N-terminal) region of the CRR:
h) the center to C-terminus of CRR and the L2 domain;
i) the CRR and L2 regions;
j) the IGF-1 ligand binding region;
k) the IGF-2 ligand binding region; and,
l) any combination of two or more fully or partially overlapping regions in a) through k).

11. The method of claim 8 wherein said two or more different epitopes are in a region comprising any combination of human IGF-1R amino acids selected from the group consisting of:
a) amino acids 241-266 of SEQ ID NO:158;
b) amino acids 241-379 of SEQ ID NO:158;
c) amino acids 248-265 of SEQ ID NO:158;
d) amino acids 248-303 of SEQ ID NO:158;
e) amino acids 248-379 of SEQ ID NO:158;
f) amino acids 301-308 of SEQ ID NO:158;
g) amino acids 327-379 of SEQ ID NO:158;
h) amino acids 424-464 of SEQ ID NO:158;
i) amino acids 437-587 of SEQ ID NO:158;
j) amino acids 440-586 of SEQ ID NO:158;
k) amino acids 459-571 of SEQ ID NO:158;
l) amino acids 461-464 of SEQ ID NO:158;
l) amino acids 462-464 of SEQ ID NO:158; and,
m) amino acids 466-568 of SEQ ID NO:158.

12. The method of claim 8 wherein said two or more different epitopes comprise a human IGF-1R amino acid selected from the group consisting of:
a) amino acid 226 of SEQ ID NO:158;
b) amino acid 241 of SEQ ID NO:158;
c) amino acid 242 of SEQ ID NO:158;
d) amino acid 248 of SEQ ID NO:158;
e) amino acid 249 of SEQ ID NO:158;
f) amino acid 250 of SEQ ID NO:158;
g) amino acid 251 of SEQ ID NO:158;
h) amino acid 254 of SEQ ID NO:158;
i) amino acid 255 of SEQ ID NO:158;
j) amino acid 257 of SEQ ID NO:158;
k) amino acid 259 of SEQ ID NO:158;
l) amino acid 260 of SEQ ID NO:158;
m) amino acid 263 of SEQ ID NO:158;
n) amino acid 265 of SEQ ID NO:158;
o) amino acid 266 of SEQ ID NO:158;
p) amino acid 301 of SEQ ID NO:158;
q) amino acid 303 of SEQ ID NO:158;
r) amino acid 306 of SEQ ID NO:158;
s) amino acid 308 of SEQ ID NO:158;
t) amino acid 327 of SEQ ID NO:158;
u) amino acid 379 of SEQ ID NO:158;
v) amino acid 459 of SEQ ID NO:158;
w) amino acid 460 of SEQ ID NO:158;
x) amino acid 461 of SEQ ID NO:158;
y) amino acid 462 of SEQ ID NO:158;
z) amino acid 464 of SEQ ID NO:158;
aa) amino acid 466 of SEQ ID NO:158;
bb) amino acid 467 of SEQ ID NO:158;
cc) amino acid 478 of SEQ ID NO:158;
dd) amino acid 480 of SEQ ID NO:158;
ee) amino acid 482 of SEQ ID NO:158;
ff) amino acid 483 of SEQ ID NO:158;
gg) amino acid 533 of SEQ ID NO:158;
hh) amino acid 564 of SEQ ID NO:158;
ii) amino acid 565 of SEQ ID NO:158;
jj) amino acid 568 of SEQ ID NO:158;
kk) amino acid 570 of SEQ ID NO:158; and,
ll) amino acid 571 of SEQ ID NO:158.

13. The method of claim 9 wherein said two or more different IGF-1R epitopes are in regions of human IGF-1R selected from the group consisting of:
a) the fibronectin type III domain 1 (FNIII-1);
b) the cysteine rich repeat domain (CRR);
c) the leucine rich repeat domain 1 (L1);
d) the leucine rich repeat domain 1 (L2);
e) the center region of the cysteine rich repeat domain (CRR);
f) the carboxyl-terminal (C-terminal) region of the CRR;
g) the amino-terminal (N-terminal) region of the CRR:
h) the center to C-terminus of CRR and the L2 domain;
i) the CRR and L2 regions;
j) the IGF-1 ligand binding region;
k) the IGF-2 ligand binding region; and,
l) any combination of two or more fully or partially overlapping regions in a) through k).

14. The method of claim 9 wherein said two or more different epitopes are in a region comprising any combination of human IGF-1R amino acids selected from the group consisting of:
a) amino acids 241-266 of SEQ ID NO:158;
b) amino acids 241-379 of SEQ ID NO:158;
c) amino acids 248-265 of SEQ ID NO:158;
d) amino acids 248-303 of SEQ ID NO:158;
e) amino acids 248-379 of SEQ ID NO:158;
f) amino acids 301-308 of SEQ ID NO:158;
g) amino acids 327-379 of SEQ ID NO:158;
h) amino acids 424-464 of SEQ ID NO:158;
i) amino acids 437-587 of SEQ ID NO:158;
j) amino acids 440-586 of SEQ ID NO:158;
k) amino acids 459-571 of SEQ ID NO:158;
l) amino acids 461-464 of SEQ ID NO:158;
1) amino acids 462-464 of SEQ ID NO:158; and,
m) amino acids 466-568 of SEQ ID NO:158.

15. The method of claim 9 wherein said two or more different epitopes comprise a human IGF-1R amino acid selected from the group consisting of:
a) amino acid 226 of SEQ ID NO:158;
b) amino acid 241 of SEQ ID NO:158;
c) amino acid 242 of SEQ ID NO:158;
d) amino acid 248 of SEQ ID NO:158;
e) amino acid 249 of SEQ ID NO:158;
f) amino acid 250 of SEQ ID NO:158;
g) amino acid 251 of SEQ ID NO:158;
h) amino acid 254 of SEQ ID NO:158;
i) amino acid 255 of SEQ ID NO:158;
j) amino acid 257 of SEQ ID NO:158;
k) amino acid 259 of SEQ ID NO:158;
l) amino acid 260 of SEQ ID NO:158;
m) amino acid 263 of SEQ ID NO:158;
n) amino acid 265 of SEQ ID NO:158;
o) amino acid 266 of SEQ ID NO:158;
p) amino acid 301 of SEQ ID NO:158;
q) amino acid 303 of SEQ ID NO:158;
r) amino acid 306 of SEQ ID NO:158;
s) amino acid 308 of SEQ ID NO:158;
t) amino acid 327 of SEQ ID NO:158;
u) amino acid 379 of SEQ ID NO:158;
v) amino acid 459 of SEQ ID NO:158;
w) amino acid 460 of SEQ ID NO:158;
x) amino acid 461 of SEQ ID NO:158;
y) amino acid 462 of SEQ ID NO:158;
z) amino acid 464 of SEQ ID NO:158;
aa) amino acid 466 of SEQ ID NO:158;
bb) amino acid 467 of SEQ ID NO:158;
cc) amino acid 478 of SEQ ID NO:158;
dd) amino acid 480 of SEQ ID NO:158;
ee) amino acid 482 of SEQ ID NO:158;
ff) amino acid 483 of SEQ ID NO:158;
gg) amino acid 533 of SEQ ID NO:158;
hh) amino acid 564 of SEQ ID NO:158;
ii) amino acid 565 of SEQ ID NO:158;
jj) amino acid 568 of SEQ ID NO:158;
kk) amino acid 570 of SEQ ID NO:158; and,
ll) amino acid 571 of SEQ ID NO:158.

16. The method of claim 1, wherein said two or more antibodies, or fragments thereof inhibit IGF-1 ligand binding.

17. The method of claim 4, wherein said two or more antibodies, or fragments thereof inhibit IGF-1 ligand binding.

18. The method of claim 1, wherein said two or more antibodies, or fragments thereof inhibit IGF-2 ligand binding.

19. The method of claim 4, wherein said two or more antibodies, or fragments thereof inhibit IGF-2 ligand binding.

20. The method of claim 1, wherein said two or more antibodies, or fragments thereof inhibit IGF-1 and IGF-2 ligand binding.

21. The method of claim 4, wherein said two or more antibodies, or fragments thereof inhibit IGF-1 and IGF-2 ligand binding.

22. The method of claim 1, wherein said two or more antibodies, or fragments thereof allosterically inhibit ligand binding.

23. The method of claim 4, wherein said two or more antibodies, or fragments thereof allosterically inhibit ligand binding.

24. The method of claim 1, wherein said two or more antibodies, or fragments thereof competitively inhibit ligand binding.

25. The method of claim 4, wherein said two or more antibodies, or fragments thereof competitively inhibit ligand binding.

26. The method of claim 1, wherein said two or more antibodies, or fragments thereof allosterically and competitively inhibit ligand binding.

27. The method of claim 4, wherein said two or more antibodies, or fragments thereof allosterically and competitively inhibit ligand binding.

28. A method of inhibiting IGF-1R signal transduction, comprising administering a combination of two or more antibodies, or fragments thereof which specifically bind to IGF-1R; wherein said administration inhibits signal transduction more effectively than any one of said antibodies, or fragments thereof administered alone, at approximately the same final total molar concentration.

29. The method of claim 28, wherein said signal transduction is inhibited in vivo.

30. The method of claim 29, wherein said signal transduction is inhibited in a human.

31. A method of treating a hyperproliferative disorder in an animal, comprising administering a combination of two or more antibodies, or fragments thereof which specifically bind IGF-1R, wherein said administration treats said hyperproliferative disorder more effectively than any one of said antibodies, or fragments thereof administered alone, at approximately the same final total molar concentration.

32. The method of claim 31, wherein said hyperproliferative disorder is cancer.

33. The method of claim 31, wherein said hyperproliferative disorder is a tumor.

34. The method of 31, wherein said animal is human.

35. The method of claim 31 wherein said administration of two or more antibodies, or fragments thereof inhibits IGF-1 binding to IGF-1R more effectively than any one of said antibodies, or fragments thereof administered alone, at approximately the same final total molar concentration.

36. The method of claim 31 wherein said administration of two or more antibodies, or fragments thereof, inhibits IGF-2 binding to IGF-1R more effectively than any one of said antibodies, or fragments thereof administered alone, at approximately the same final total molar concentration.

37. The method of claim 31 wherein said administration of two or more antibodies, or fragments thereof inhibit IGF-1 and IGF-2 binding to IGF-1R more effectively than any one of said antibodies, or fragments thereof administered alone, at approximately the same final total molar concentration.

38. The method of claim 31, wherein said combination of two or more antibodies, or fragments thereof specifically bind IGF-1R epitopes in regions of human IGF-1R selected from the group consisting of:
a) the fibronectin type III domain 1 (FNIII-1);
b) the cysteine rich repeat domain (CRR);
c) the leucine rich repeat domain 1 (L1);
d) the leucine rich repeat domain 1 (L2);
e) the center region of the cysteine rich repeat domain (CRR);
f) the carboxyl-terminal (C-terminal) region of the CRR;
g) the amino-terminal (N-terminal) region of the CRR:
h) the center to C-terminus of CRR and the L2 domain;
i) the CRR and L2 regions;
j) the IGF-1 ligand binding region;
k) the IGF-2 ligand binding region; and,
l) any combination of two or more fully or partially overlapping regions in a) through k).

39. The method of claim 31, wherein said combination of two or more antibodies, or fragments thereof specifically bind IGF-1R epitopes in a region comprising any combination of human IGF-1R amino acids selected from the group consisting of:
a) amino acids 241-266 of SEQ ID NO:158;
b) amino acids 241-379 of SEQ ID NO:158;
c) amino acids 248-265 of SEQ ID NO:158;
d) amino acids 248-303 of SEQ ID NO:158;
e) amino acids 248-379 of SEQ ID NO:158;
f) amino acids 301-308 of SEQ ID NO:158;
g) amino acids 327-379 of SEQ ID NO:158;
h) amino acids 424-464 of SEQ ID NO:158;
i) amino acids 437-587 of SEQ ID NO:158;
j) amino acids 440-586 of SEQ ID NO:158;
k) amino acids 459-571 of SEQ ID NO:158;
l) amino acids 461-464 of SEQ ID NO:158;
l) amino acids 462-464 of SEQ ID NO:158; and,
m) amino acids 466-568 of SEQ ID NO:158.

40. The method of claim 31, wherein said combination of two or more antibodies, or fragments thereof specifically bind two or more different epitopes comprising a human IGF-1R amino acid selected from the group consisting of:
a) amino acid 226 of SEQ ID NO:158;
b) amino acid 241 of SEQ ID NO:158;
c) amino acid 242 of SEQ ID NO:158;
d) amino acid 248 of SEQ ID NO:158;
e) amino acid 249 of SEQ ID NO:158;
f) amino acid 250 of SEQ ID NO:158;
g) amino acid 251 of SEQ ID NO:158;
h) amino acid 254 of SEQ ID NO:158;
i) amino acid 255 of SEQ ID NO:158;
j) amino acid 257 of SEQ ID NO:158;
k) amino acid 259 of SEQ ID NO:158;
l) amino acid 260 of SEQ ID NO:158;
m) amino acid 263 of SEQ ID NO:158;
n) amino acid 265 of SEQ ID NO:158;
o) amino acid 266 of SEQ ID NO:158;
p) amino acid 301 of SEQ ID NO:158;
q) amino acid 303 of SEQ ID NO:158;
r) amino acid 306 of SEQ ID NO:158;
s) amino acid 308 of SEQ ID NO:158;
t) amino acid 327 of SEQ ID NO:158;
u) amino acid 379 of SEQ ID NO:158;
v) amino acid 459 of SEQ ID NO:158;
w) amino acid 460 of SEQ ID NO:158;
x) amino acid 461 of SEQ ID NO:158;
y) amino acid 462 of SEQ ID NO:158;
z) amino acid 464 of SEQ ID NO:158;
aa) amino acid 466 of SEQ ID NO:158;
bb) amino acid 467 of SEQ ID NO:158;
cc) amino acid 478 of SEQ ID NO:158;
dd) amino acid 480 of SEQ ID NO:158;
ee) amino acid 482 of SEQ ID NO:158;
ff) amino acid 483 of SEQ ID NO:158;
gg) amino acid 533 of SEQ ID NO:158;
hh) amino acid 564 of SEQ ID NO:158;
ii) amino acid 565 of SEQ ID NO:158;
jj) amino acid 568 of SEQ ID NO:158;
kk) amino acid 570 of SEQ ID NO:158; and,
ll) amino acid 571 of SEQ ID NO:158.

41. A method of allosterically inhibiting IGF-1 binding to IGF-1R wherein said method comprises:
a) exposing IGF-1R to an IGF-1R specific antibody, or fragment thereof; and,
b) allowing said antibody, or fragment thereof sufficient time to bind said IGF-1R.

42. The method of claim 41, wherein said antibody, or fragment thereof does not inhibit IGF-2 binding to IGF-1R.

43. The method of claim 42, wherein said antibody is selected from the group consisting of:
a) P1E2; and,
b) P1A2.

44. A method of allosterically inhibiting IGF-2 binding to IGF-1R wherein said method comprises:
a) exposing IGF-1R to an IGF-1R specific antibody, or fragment thereof, and,
b) allowing said antibody, or fragment thereof sufficient time to bind said IGF-1R.

45. The method of claim 44, wherein said antibody, or fragment thereof does not inhibit IGF-1 binding to IGF-1R.

46. The method of claim 45, wherein said antibody is P3F9.

47. A method of allosterically inhibiting IGF-1 and IGF-2 binding to IGF-1R wherein said method comprises:
a) exposing IGF-1R to one or more IGF-1R specific antibodies, or fragments thereof; and,
b) allowing said one or more antibodies, or fragment thereof sufficient time to bind said IGF-1R.

48. A method of allosterically inhibiting IGF-1 or IGF-2 binding to IGF-1R wherein said method comprises:
a) exposing IGF-1R to one or more IGF-1R specific antibodies, or fragments thereof; and,
b) allowing said one or more antibodies, or fragments thereof sufficient time to bind said IGF-1R.

49. The method of claim 47, wherein said one or more antibodies is selected from the group consisting of:
a) P1E2;
b) P1A2;
c) P3F9;
d) M13-C06;
e) M14-C03;
f) 20C8; and,
g) a combination of two or more of said antibodies.

50. The method of claim 48, wherein said one or more antibodies is selected from the group consisting of:
a) P1E2;
b) P1A2;
c) P3F9;
d) M13-C06;
e) M14-C03;
f) 20C8; and,
g) a combination of two or more of said antibodies.

51. A method of competitively inhibiting IGF-1 and IGF-2 binding to IGF-1R wherein said method comprises:
a) exposing IGF-1R to an IGF-1R specific antibody, or fragment thereof; and,
b) allowing said antibody, or fragment thereof sufficient time to bind said IGF-1R.

52. A method of competitively inhibiting IGF-1 or IGF-2 binding to IGF-1R wherein said method comprises:
a) exposing IGF-1R to an IGF-1R specific antibody, or fragment thereof; and,
b) allowing said antibody, or fragment thereof sufficient time to bind said IGF-1R.

53. The method of claim 51, wherein said antibody is M14-G11.

54. The method of claim 52, wherein said antibody is M14-G11.

55. A method of competitively and allosterically inhibiting IGF-1 and IGF-2 binding to IGF-1R wherein said method comprises:
a) exposing IGF-1R to IGF-1R specific antibodies, or fragments thereof; and,
b) allowing said antibodies, or fragments thereof sufficient time to bind said IGF-1R.

56. A method of competitively and allosterically inhibiting IGF-1 or IGF-2 binding to IGF-1R wherein said method comprises:
a) exposing IGF-1R to IGF-1R specific antibodies, or fragments thereof; and,
b) allowing said antibodies, or fragments thereof sufficient time to bind said IGF-1R.

57. The method of claim 55, wherein said competitive inhibitor antibody is M14-G11 and wherein said allosteric inhibitor antibody is one or more antibodies selected from the group consisting of:
a) P1E2;
b) P1A2;
c) P3F9;
d) M13-C06;
e) M14-C03;
f) 20C8; and,
g) a combination of two or more of said antibodies.

58. The method of claim 56, wherein said competitive inhibitor antibody is M14-G11 and wherein said allosteric inhibitor antibody is one or more antibodies selected from the group consisting of:
a) P1E2;
b) P1A2;
c) P3F9;
d) M13-C06;
e) M14-C03;
f) 20C8; and,
g) a combination of two or more of said antibodies.


SEQUENCE LISTING (158 sequences)

SEQ ID NO: 1 - DNA, 4989 bp, Homo sapiens
ttttttttttttttgagaaagggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggaggcaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctgatccttggatcctgaatctgtgcaaacagtaacgtgtgcgcacgcgcagcggggtggggggggagagagagttttaacaatccattcacaagcctcctgtacctcagtggatcttcagttctgcccttgctgcccgcgggagacagcttctctgcagtaaaacacatttgggatgttccttttttcaatatgcaagcagctttttattccctgcccaaacccttaactgacatgggcctttaagaaccttaatgacaacacttaatagcaacagagcacttgagaaccagtctcctcactctgtccctgtccttccctgttctccctttctctctcctctctgcttcataacggaaaaataattgccacaagtccagctgggaagccctttttatcagtttgaggaagtggctgtccctgtggccccatccaaccactgtacacacccgcctgacaccgtgggtcattacaaaaaaacacgtggagatggaaatttttacctttatctttcacctttctagggacatgaaatttacaaagggccatcgttcatccaaggctgttaccattttaacgctgcctaattttgccaaaatcctgaactttctccctcatcggcccggcgctgattcctcgtgtccggaggcatgggtgagcatggcagctggttgctccatttgagagacacgctggcgacacactccgtccatccgactgcccctgctgtgctgctcaaggccacaggcacacaggtctcattgcttctgactagattattatttgggggaactggacacaataggtctttctctcagtgaaggtggggagaagctgaaccggc

SEQ ID NO: 2 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 3 - DNA, 393 bp, Artificial Sequence
  Antibody variable heavy chain sequence
gaagttcaattgttagagtctggtggcggtcttgttcagcctggtggttctttacgtctttcttgcgctgcttccggattcactttctctccttactctatgctttgggttcgccaagctcctggtaaaggtttggagtgggtttcttctatcggttcttctggtggctctactcgttatgctgactccgttaaaggtcgcttcactatctctagagacaactctaagaatactctctacttgcagatgaacagcttaagggctgaggacaccgccatgtattactgtgcacgggtacgggggatccttcattacgatattttgattggtagaaatctctactactactacatggacgtctggggcaaagggaccacggtcaccgtctcaagc

SEQ ID NO: 4 - protein, 131 aa, Artificial Sequence
  Antibody variable heavy chain sequence
EVQLLESGGGLVQPGGSLRLSCAASGFTFSPYSMLWVRQAPGKGLEWVSSIGSSGGSTRYADSVKGRFTISRDNSKNTLYLQMNSLRAEDTAMYYCARVRGILHYDILIGRNLYYYYMDVWGKGTTVTVSS

SEQ ID NO: 5 - protein, 5 aa, Artificial Sequence
  Antibody variable heavy chain sequence
PYSML

SEQ ID NO: 6 - protein, 17 aa, Artificial Sequence
  Antibody variable heavy chain sequence
SIGSSGGSTRYADSVKG

SEQ ID NO: 7 - protein, 22 aa, Artificial Sequence
  Antibody variable heavy chain sequence
VRGILHYDILIGRNLYYYYMDV

SEQ ID NO: 8 - DNA, 390 bp, Artificial Sequence
  Antibody variable heavy chain sequence
gaagttcaattgttagagtctggtggcggtcttgttcagcctggtggttctttacgtctttcttgcgctgcttccggattcactttctctaagtacactatgcattgggttcgccaagctcctggtaaaggtttggagtgggtttcttctatcgtttcttctggtggctggactgattatgctgactccgttaaaggtcgcttcactatctctagagacaactctaagaatactctctacttgcagatgaacagcttaagggctgaggacacggccgtgtattactgtgcgagagatcggagtatagcagcagctggtaccggttggtctgtgagttttgtggactggttcgacccctggggccagggaaccctggtcaccgtctcaagc

SEQ ID NO: 9 - protein, 130 aa, Artificial Sequence
  Antibody variable heavy chain sequence
EVQLLESGGGLVQPGGSLRLSCAASGFTFSKYTMHWVRQAPGKGLEWVSSIVSSGGWTDYADSVKGRFTISRDNSKNTLYLQMNSLRAEDTAVYYCARDRSIAAAGTGWSVSFVDWFDPWGQGTLVTVSS

SEQ ID NO: 10 - protein, 5 aa, Artificial Sequence
  Antibody variable heavy chain sequence
KYTMH

SEQ ID NO: 11 - protein, 17 aa, Artificial Sequence
  Antibody variable heavy chain sequence
SIVSSGGWTDYADSVKG

SEQ ID NO: 12 - protein, 21 aa, Artificial Sequence
  Antibody variable heavy chain sequence
DRSIAAAGTGWSVSFVDWFDP

SEQ ID NO: 13 - DNA, 360 bp, Artificial Sequence
  Antibody variable heavy chain sequence
gaagttcaattgttagagtctggtggcggtcttgttcagcctggtggttctttacgtctttcttgcgctgcttccggattcactttctctatttaccgtatgcagtgggttcgccaagctcctggtaaaggtttggagtgggtttctggtatctctccttctggtggcactacttggtatgctgactccgttaaaggtcgcttcactatctctagagacaactctaagaatactctctacttgcagatgaacagcttaagggctgaggacacggccgtgtattactgtgcgagatggagcgggggttcgggctatgcttttgatatctggggccaagggacaatggtcaccgtctcaagc

SEQ ID NO: 14 - protein, 120 aa, Artificial Sequence
  Antibody variable heavy chain sequence
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 15 - protein, 5 aa, Artificial Sequence
  Antibody variable heavy chain sequence
IYRMQ

SEQ ID NO: 16 - protein, 17 aa, Artificial Sequence
  Antibody variable heavy chain sequence
GISPSGGTTWYADSVKG

SEQ ID NO: 17 - protein, 11 aa, Artificial Sequence
  Antibody variable heavy chain sequence
WSGGSGYAFDI

SEQ ID NO: 18 - DNA, 360 bp, Artificial Sequence
  Antibody variable heavy chain sequence
gaggtccagctgttggagtccggcggtggcctggtgcagcctggggggtccctgagactctcctgcgcagctagcggcttcaccttcagcatttaccgtatgcagtgggtgcgccaggctcctggaaaggggctggagtgggtttccggtatctctccctctggtggcacgacgtggtatgctgactccgtgaagggccggttcacaatctccagagacaattccaagaacactctgtacctgcaaatgaacagcctgagagctgaggatactgcagtgtactactgcgccagatggtccgggggctccggatacgccttcgacatctggggacagggaaccatggtcaccgtctcaagc

SEQ ID NO: 19 - DNA, 363 bp, Artificial Sequence
  Antibody variable heavy chain sequence
gaagttcaattgttagagtctggtggcggtcttgttcagcctggtggttctttacgtctttcttgcgctgcttccggattcactttctctaattaccatatggcttgggttcgccaagctcctggtaaaggtttggagtgggtttctgttatctctcctactggtggccgtactacttatgctgactccgttaaaggtcgcttcactatctctagagacaactctaagaatactctctacttgcagatgaacagcttaagggctgaggacacagccacatattactgtgcgagagcggggtacagctatggttatggctactttgactactggggccagggaaccctggtcaccgtctcaagc

SEQ ID NO: 20 - protein, 121 aa, Artificial Sequence
  Antibody variable heavy chain sequence
EVQLLESGGGLVQPGGSLRLSCAASGFTFSNYHMAWVRQAPGKGLEWVSVISPTGGRTTYADSVKGRFTISRDNSKNTLYLQMNSLRAEDTATYYCARAGYSYGYGYFDYWGQGTLVTVSS

SEQ ID NO: 21 - protein, 5 aa, Artificial Sequence
  Antibody variable heavy chain sequence
NYHMA

SEQ ID NO: 22 - protein, 17 aa, Artificial Sequence
  Antibody variable heavy chain sequence
VISPTGGRTTYADSVKG

SEQ ID NO: 23 - protein, 12 aa, Artificial Sequence
  Antibody variable heavy chain sequence
AGYSYGYGYFDY

SEQ ID NO: 24 - DNA, 363 bp, Artificial Sequence
  Antibody variable heavy chain sequence
gaggtccagctgttggagtccggcggtggcctggtgcagcctggggggtccctgagactctcctgcgcagctagcggcttcaccttcagcaattaccacatggcctgggtgcgccaggctcctggaaaggggctggagtgggtttccgtgatctctcctaccggtggcaggaccacttacgctgactccgtgaagggccggttcacaatctccagagacaattccaagaacactctgtacctgcaaatgaacagcctgagagctgaggatactgcaacatactactgcgccagagccgggtactcctacggctacggatacttcgactactggggacagggaaccctggtcaccgtctcaagc

SEQ ID NO: 25 - DNA, 357 bp, Artificial Sequence
  Antibody variable heavy chain sequence
gaagttcaattgttagagtctggtggcggtcttgttcagcctggtggttctttacgtctttcttgcgctgcttccggattcactttctctaagtacatgatgtcttgggttcgccaagctcctggtaaaggtttggagtgggtttcttatatctctccttctggtggccttacttggtatgctgactccgttaaaggtcgcttcactatctctagagacaactctaagaatactctctacttgcagatgaacagcttaagggctgaggacacggccgtgtattactgtgcgagagatggagctagaggctacggtatggacgtctggggccaagggaccacggtcaccgtctcaagc

SEQ ID NO: 26 - protein, 119 aa, Artificial Sequence
  Antibody variable heavy chain sequence
EVQLLESGGGLVQPGGSLRLSCAASGFTFSKYMMSWVRQAPGKGLEWVSYISPSGGLTWYADSVKGRFTISRDNSKNTLYLQMNSLRAEDTAVYYCARDGARGYGMDVWGQGTTVTVSS

SEQ ID NO: 27 - protein, 5 aa, Artificial Sequence
  Antibody variable heavy chain sequence
KYMMS

SEQ ID NO: 28 - protein, 17 aa, Artificial Sequence
  Antibody variable heavy chain sequence
YISPSGGLTWYADSVKG

SEQ ID NO: 29 - protein, 10 aa, Artificial Sequence
  Antibody variable heavy chain sequence
DGARGYGMDV

SEQ ID NO: 30 - DNA, 357 bp, Artificial Sequence
  Antibody variable heavy chain sequence
gaggtccagctgttggagtccggcggtggcctggtgcagcctggggggtccctgagactctcctgcgcagctagcggcttcaccttcagcaagtacatgatgtcttgggtgcgccaggctcctggaaaggggctggagtgggtttcctatatctctccctctggtggcctgacgtggtatgctgactccgtgaagggccggttcacaatctccagagacaattccaagaacactctgtacctgcaaatgaacagcctgagagctgaggatactgcagtgtactactgcgccagagatggggctagaggatacggaatggacgtctggggacagggaaccaccgtcaccgtctcaagc

SEQ ID NO: 31 - DNA, 372 bp, Artificial Sequence
  Antibody variable heavy chain sequence
gaagttcaattgttagagtctggtggcggtcttgttcagcctggtggttctttacgtctttcttgcgctgcttccggattcactttctctaattaccctatgtattgggttcgccaagctcctggtaaaggtttggagtgggtttctcgtatctcttcttctggtggccgtactgtttatgctgactccgttaaaggtcgcttcactatctctagagacaactctaagaatactctctacttgcagatgaacagcttaagggctgaggacacggccgtgtattactgtgcgagagatcgatggtccagatctgcagctgaatatgggttgggtggctactggggccagggaaccctggtcaccgtctcaagc

SEQ ID NO: 32 - protein, 124 aa, Artificial Sequence
  Antibody variable heavy chain sequence
EVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSS

SEQ ID NO: 33 - protein, 5 aa, Artificial Sequence
  Antibody variable heavy chain sequence
NYPMY

SEQ ID NO: 34 - protein, 17 aa, Artificial Sequence
  Antibody variable heavy chain sequence
RISSSGGRTVYADSVKG

SEQ ID NO: 35 - protein, 15 aa, Artificial Sequence
  Antibody variable heavy chain sequence
DRWSRSAAEYGLGGY

SEQ ID NO: 36 - DNA, 372 bp, Artificial Sequence
  Antibody variable heavy chain sequence
gaggtccagctgttggagtccggcggtggcctggtgcagcctggggggtccctgagactctcctgcgcagctagcggcttcaccttcagcaattaccccatgtactgggtgcgccaggctcctggaaaggggctggagtgggtttccaggatctctagcagcggtggcaggaccgtgtacgctgactccgtgaagggccggttcacaatctccagagacaattccaagaacactctgtacctgcaaatgaacagcctgagagctgaggatactgcagtgtactactgcgccagagataggtggtccagatctgcagccgagtacggactggggggctactggggacagggaaccctggtcaccgtctcaagc

SEQ ID NO: 37 - DNA, 369 bp, Artificial Sequence
  Antibody variable heavy chain sequence
caggttcagctgcagcagtctggacctgagctagtgaagcctggggcttcagtgaagatgtcctgcaaggcttctggaaacacattcactgactatgttataaactgggtgaagcagagaactggacagggccttgagtggattggagagatttatcctggaaatgaaaatacttattacaatgagaagttcaagggcaaggccacactgactgcagacaaatcctccaacacagcctacatgcagctcagtagcctgacatctgaggactctgcggtctatttctgtgcaagagggatttattactacggtagtaggacgaggactatggactactggggtcaaggaacctcagtcaccgtctcctca

SEQ ID NO: 38 - protein, 123 aa, Artificial Sequence
  Antibody variable heavy chain sequence
QVQLQQSGPELVKPGASVKMSCKASGNTFTDYVINWVKQRTGQGLEWIGEIYPGNENTYYNEKFKGKATLTADKSSNTAYMQLSSLTSEDSAVYFCARGIYYYGSRTRTMDYWGQGTSVTVSS

SEQ ID NO: 39 - protein, 5 aa, Artificial Sequence
  Antibody variable heavy chain sequence
DYVIN

SEQ ID NO: 40 - protein, 16 aa, Artificial Sequence
  Antibody variable heavy chain sequence
IYPGNENTYYNEKFKG

SEQ ID NO: 41 - protein, 14 aa, Artificial Sequence
  Antibody variable heavy chain sequence
GIYYYGSRTRTMDY

SEQ ID NO: 42 - DNA, 366 bp, Artificial Sequence
  Antibody variable heavy chain sequence
gacgtccaactgcaggagtctggacctgacctggtgaaaccttctcagtcactttcactcacctgcactgtcactggctactccatcaccagtggttatagctggcactggatccggcagtttccaggaaacaaactggaatggatgggctacatacactacagtggtggcactaactacaacccatctctcaaaagtcgaatctctatcactcgagacacatccaagaaccagttcttcctccagttgaattctgtgactactgaggacacagccacatattactgtgcaagatcggggtacggctacaggagtgcgtactattttgactactggggccaagggaccacggtcaccgtctcctca

SEQ ID NO: 43 - protein, 122 aa, Artificial Sequence
  Antibody variable heavy chain sequence
DVQLQESGPDLVKPSQSLSLTCTVTGYSITSGYSWHWIRQFPGNKLEWMGYIHYSGGTNYNPSLKSRISITRDTSKNQFFLQLNSVTTEDTATYYCARSGYGYRSAYYFDYWGQGTTVTVSS

SEQ ID NO: 44 - protein, 6 aa, Artificial Sequence
  Antibody variable heavy chain sequence
SGYSWH

SEQ ID NO: 45 - protein, 16 aa, Artificial Sequence
  Antibody variable heavy chain sequence
YIHYSGGTNYNPSLKS

SEQ ID NO: 46 - protein, 13 aa, Artificial Sequence
  Antibody variable heavy chain sequence
SGYGYRSAYYFDY

SEQ ID NO: 47 - DNA, 363 bp, Artificial Sequence
  Antibody variable heavy chain sequence
caaatacagttggttcagagcggacctgagctgaagaagcctggagagacagtcaagatctcctgcaaggcttctgggtataccttcacaaaccatggaatgaactgggtgaagcaggctccaggaaagggtttaaagtggatgggctggataaacacctccactggagagccaacatatgctgatgacttcaagggacgttttgccttctctttggaaacctctgccagcactgcctttttgcagatcaacaacctcaaaaatgaggacacggcttcatatttctgtgcaagtcccctctactatatgtacgggcggtatatcgatgtctggggcgcagggaccgcggtcaccgtctcctca

SEQ ID NO: 48 - protein, 120 aa, Artificial Sequence
  Antibody variable heavy chain sequence
QIQLVQSGPELKKPGETVKISCKASGYTFTNHGMNWVKQAPGKGLKWMGWNTSTGEPTYADDFKGRFAFSLETSASTAFLQINNLKNEDTASYFCASPLYYMYGRYIDVWGAGTAVTVSS

SEQ ID NO: 49 - protein, 5 aa, Artificial Sequence
  Antibody variable heavy chain sequence
NHGMN

SEQ ID NO: 50 - protein, 15 aa, Artificial Sequence
  Antibody variable heavy chain sequence
NTSTGEPTYADDFKG

SEQ ID NO: 51 - protein, 12 aa, Artificial Sequence
  Antibody variable heavy chain sequence
PLYYMYGRYIDV

SEQ ID NO: 52 - DNA, 315 bp, Artificial Sequence
  Antibody variable heavy chain sequence
acgtccaactgcaggagtctggacctgacctggtgaaaccttctcagtcactttcactcacctgcactgtcactggctactccatcaccagtggttatagctggcactggatccggcagtttccaggaaacaaactggaatggatgggctacatacactacagtggtggcactaactacaacccatctctcaaaagtcgaatctctatcactcgagacacatccaagaaccagttcttcctccagttgaattctgtgactactgaggacacagccacatattactgtgcaagatcggggtacggctacaggagtg

SEQ ID NO: 53 - protein, 122 aa, Artificial Sequence
  Antibody variable heavy chain sequence
DVQLQESGPDLVKPSQSLSLTCTVTGYSITSGYSWHWIRQFPGNKLEWMGYIHYSGGTNYNPSLKSRISITRDTSKNQFFLQLNSVTTEDTATYYCARSGYGYRSAYYFDYWGQGTTLTVSS

SEQ ID NO: 54 - protein, 6 aa, Artificial Sequence
  Antibody variable heavy chain sequence
SGYSWH

SEQ ID NO: 55 - protein, 16 aa, Artificial Sequence
  Antibody variable heavy chain sequence
YIHYSGGTNYNPSLKS

SEQ ID NO: 56 - protein, 13 aa, Artificial Sequence
  Antibody variable heavy chain sequence
SGYGYRSAYYFDY

SEQ ID NO: 57 - DNA, 360 bp, Artificial Sequence
  Antibody variable heavy chain sequence
cagatccagttggtgcagtctggacctgacctgaagaagcctggagagacagtcaagatctcctgcaaggcttctgggtataccttcacaaaccatggaatgaactgggtgaagcaggctccaggaaaggatttaaagtggatgggctggataaacaccaacactggagagccaacatatgctgatgacttcaagggacggtttgccttctctttggaaacctctgccagcactgcctatttgcagatcaacaacctcaaaaatgaggacacggctacatatttctgtgcaagtcccctctactataggaacgggcgatacttcgatgtctggggcgcagggaccacggtcaccgtctcc

SEQ ID NO: 58 - protein, 121 aa, Artificial Sequence
  Antibody variable heavy chain sequence
QIQLVQSGPDLKKPGETVKISCKASGYTFTNHGMNWVKQAPGKDLKWMGWINTNTGEPTYADDFKGRFAFSLETSASTAYLQINNLKNEDTATYFCASPLYYRNGRYFDVWGAGTTVTVSS

SEQ ID NO: 59 - protein, 5 aa, Artificial Sequence
  Antibody variable heavy chain sequence
NHGMN

SEQ ID NO: 60 - protein, 17 aa, Artificial Sequence
  Antibody variable heavy chain sequence
WINTNTGEPTYADDFKG

SEQ ID NO: 61 - protein, 12 aa, Artificial Sequence
  Antibody variable heavy chain sequence
PLYYRNGRYFDV

SEQ ID NO: 62 - DNA, 354 bp, Artificial Sequence
  Antibody variable heavy chain sequence
caggtccaactgcagcagcctggggctgaactggtgaagcctggggcttcagtgaagctgtcctgtaaggcttctggctacaccttcaccagctactggatgcactgggtgaagcagaggcctggacaaggccttgagtggattggagagattaatcctacctacggtcgtagtaattacaatgagaagttcaagagtaaggccacactgactgtagacaaatcctccagcacagcctacatgcaactcagcagcctgacatctgaggactctgcggtctattactgtgcaagattagtacgcctacggtacttcgatgtctggggcgcagggaccacggtcaccgtctcctca

SEQ ID NO: 63 - protein, 118 aa, Artificial Sequence
  Antibody variable heavy chain sequence
QVQLQQPGAELVKPGASVKLSCKASGYTFTSYWMHWVKQRPGQGLEWIGEINPTYGRSNYNEKFKSKATLTVDKSSSTAYMQLSSLTSEDSAVYYCARLVRLRYFDVWGAGTTVTVSS

SEQ ID NO: 64 - protein, 5 aa, Artificial Sequence
  Antibody variable heavy chain sequence
SYWMH

SEQ ID NO: 65 - protein, 17 aa, Artificial Sequence
  Antibody variable heavy chain sequence
EINPTYGRSNYNEKFKS

SEQ ID NO: 66 - protein, 9 aa, Artificial Sequence
  Antibody variable heavy chain sequence
LVRLRYFDV

SEQ ID NO: 67 - DNA, 330 bp, Artificial Sequence
  Antibody variable light chain sequence
cagtacgaattgactcagccgccctcggtgtctgaggccccccggcagagggtcaccatctcctgttctggaagcagctccaacatcggaaataatgctataaactggtaccagcaactcccaggaaagcctcccaaactcctcatctattatgatgatctgttgccctcaggggtctctgaccgattctctggctccaagtctggcacctcaggctccctggccatcagtgggctgcagtctgaggatgaggctgattattactgtgcagcatgggatgacaacctgaatggtgtgattttcggcggagggaccaagctgaccgtccta

SEQ ID NO: 68 - protein, 110 aa, Artificial Sequence
  Antibody variable light chain sequence
QYELTQPPSVSEAPRQRVTISCSGSSSNIGNNAINWYQQLPGKPPKLLIYYDDLLPSGVSDRFSGSKSGTSGSLAISGLQSEDEADYYCAAWDDNLNGVIFGGGTKLTVL

SEQ ID NO: 69 - protein, 13 aa, Artificial Sequence
  Antibody variable light chain sequence
SGSSSNIGNNAIN

SEQ ID NO: 70 - protein, 7 aa, Artificial Sequence
  Antibody variable light chain sequence
YDDLLPS

SEQ ID NO: 71 - protein, 11 aa, Artificial Sequence
  Antibody variable light chain sequence
AAWDDNLNGVI

SEQ ID NO: 72 - DNA, 324 bp, Artificial Sequence
  Antibody variable light chain sequence
gacatccagatgacccagtctccactctccctgtctgcatctgtaggagacagagtcaccatcacttgccgggcaagtcagagcattaacggctacttaaattggtatcagcagaaaccagggaaagcccctaacctcctgatctacgctacatccagtttgcaaagtggggtcccatcaaggttcagtggcagtggatctgggacagatttcactctcaccatcagcagtctgcaacctgaagattttgcaacttactactgtcaacagagttacagtacccccccgtacacttttggccaggggaccaagctggagatcaaa

SEQ ID NO: 73 - protein, 108 aa, Artificial Sequence
  Antibody variable light chain sequence
DIQMTQSPLSLSASVGDRVTITCRASQSINGYLNWYQQKPGKAPNLLIYATSSLQSGVPSRFSGSGSGTDFTLTISSLQPEDFATYYCQQSYSTPPYTFGQGTKLEIK

SEQ ID NO: 74 - protein, 11 aa, Artificial Sequence
  Antibody variable light chain sequence
RASQSINGYLN

SEQ ID NO: 75 - protein, 7 aa, Artificial Sequence
  Antibody variable light chain sequence
ATSSLQS

SEQ ID NO: 76 - protein, 10 aa, Artificial Sequence
  Antibody variable light chain sequence
QQSYSTPPYT

SEQ ID NO: 77 - DNA, 321 bp, Artificial Sequence
  Antibody variable light chain sequence
gacatccagatgacccagtctccactctccctgtctgcatctgtaggagacagagtcaccatcacttgccaggcgagtcgggacattagaaactatttaaattggtatcaacaaaaaccagggaaagccccgaagctcctgatctacgatgcatccagtttgcaaacaggggtcccatcaaggttcggtggcagtggatctgggacagactttagtttcaccatcggcagcctgcagcctgaagatattgcaacatattactgtcaacagtttgatagtctccctcacacttttggccaggggaccaaactggagatcaaa

SEQ ID NO: 78 - protein, 107 aa, Artificial Sequence
  Antibody variable light chain sequence
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 79 - protein, 11 aa, Artificial Sequence
  Antibody variable light chain sequence
QASRDIRNYLN

SEQ ID NO: 80 - protein, 7 aa, Artificial Sequence
  Antibody variable light chain sequence
DASSLQT

SEQ ID NO: 81 - protein, 9 aa, Artificial Sequence
  Antibody variable light chain sequence
QQFDSLPHT

SEQ ID NO: 82 - DNA, 324 bp, Artificial Sequence
  Antibody variable light chain sequence
gacatccagatgacccagtttccagccaccctgtctgtgtctccaggggaaagagccaccctctcctgcagggccagtcagagtgttatgaggaacttagcctggtaccagcagaaacctggccagcctcccaggctcctcatctatggtgcatccaaaagggccactggcatcccagccaggttcagtggcagtgggtctgggacagccttcactctcaccatcagcaacctagagcctgaagattttgcagtttattactgtcaccaacgtagcacctggcctctggggactttcggccctgggaccaaactggaggccaaa

SEQ ID NO: 83 - protein, 108 aa, Artificial Sequence
  Antibody variable light chain sequence
DIQMTQFPATLSVSPGERATLSCRASQSVMRNLAWYQQKPGQPPRLLIYGASKRATGIPARFSGSGSGTAFTLTISNLEPEDFAVYYCHQRSTWPLGTFGPGTKLEAK

SEQ ID NO: 84 - protein, 11 aa, Artificial Sequence
  Antibody variable light chain sequence
RASQSVMRNLA

SEQ ID NO: 85 - protein, 7 aa, Artificial Sequence
  Antibody variable light chain sequence
GASKRAT

SEQ ID NO: 86 - protein, 10 aa, Artificial Sequence
  Antibody variable light chain sequence
HQRSTWPLGT

SEQ ID NO: 87 - DNA, 327 bp, Artificial Sequence
  Antibody variable light chain sequence
gacatccagatgacccagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggaggtcactttcggccctgggaccaaagtggatatcaaa

SEQ ID NO: 88 - protein, 109 aa, Artificial Sequence
  Antibody variable light chain sequence
DIQMTQSPATLSLSPGERATLSCRASQSVSSYLAWYQQKPGQAPRLLIYDASNRATGIPARFSGSGSGTDFTLTISSLEPEDFAVYYCQQRSNWPPEVTFGPGTKVDIK

SEQ ID NO: 89 - protein, 11 aa, Artificial Sequence
  Antibody variable light chain sequence
RASQSVSSYLA

SEQ ID NO: 90 - protein, 7 aa, Artificial Sequence
  Antibody variable light chain sequence
DASNRAT

SEQ ID NO: 91 - protein, 11 aa, Artificial Sequence
  Antibody variable light chain sequence
QQRSNWPPEVT

SEQ ID NO: 92 - DNA, 336 bp, Artificial Sequence
  Antibody variable light chain
gacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttacttggcatctacccgggaatccggggtccctgaccgattcagtggcagcgggtctgggacagatttcactctcaccatcagcagcctgcaggctgaagatgtggcagtttattactgtcagcaatattatagtacttggacgttcggccaagggaccaaggtggaaatcaaa

SEQ ID NO: 93 - protein, 112 aa, Artificial Sequence
  Antibody variable light chain sequence
DIQMTQSPDSLAVSLGERATINCKSSQSVLYSSNNKNYLAWYQQKPGQPPKLLIYLASTRESGVPDRFSGSGSGTDFTLTISSLQAEDVAVYYCQQYYSTWTFGQGTKVEIK

SEQ ID NO: 94 - protein, 17 aa, Artificial Sequence
  Antibody variable light chain sequence
KSSQSVLYSSNNKNYLA

SEQ ID NO: 95 - protein, 7 aa, Artificial Sequence
  Antibody variable light chain sequence
LASTRES

SEQ ID NO: 96 - protein, 8 aa, Artificial Sequence
  Antibody variable light chain sequence
QQYYSTWT

SEQ ID NO: 97 - DNA, 324 bp, Artificial Sequence
  Antibody variable light chain sequence
gaagttgtgctcacccagtctccaaccgccatggctgcatctcccggggagaagatcactatcacctgcagtgccagctcaactttaagttccaattacttgcattggtatcagcagaagccaggattctcccctaaactcttgatttataggacatccaatctggcctctggagtcccaggtcgcttcagtggcagtgggtctgggacctcttactctctcacaattggcaccatggaggctgaagatgttgccacttactactgccagcagggtagtagtataccgctcacgttcggtgctgggaccaagctggagctgaag

SEQ ID NO: 98 - protein, 108 aa, Artificial Sequence
  Antibody variable light chain sequence
EVVLTQSPTAMAASPGEKITITCSASSTLSSNYLHWYQQKPGFSPKLLIYRTSNLASGVPGRFSGSGSGTSYSLTIGTMEAEDVATYYCQQGSSIPLTFGAGTKLELK

SEQ ID NO: 99 - protein, 12 aa, Artificial Sequence
  Antibody variable light chain sequence
SASSTLSSNYLH

SEQ ID NO: 100 - protein, 7 aa, Artificial Sequence
  Antibody variable light chain sequence
RTSNLAS

SEQ ID NO: 101 - protein, 9 aa, Artificial Sequence
  Antibody variable light chain sequence
QQGSSIPLT

SEQ ID NO: 102 - DNA, 330 bp, Artificial Sequence
  Antibody variable light chain sequence
gacattgtgctgacacagtctcctgcttccttagctgtatctctggggcagagggccaccatctcatgcagggccagcaaaagtgtcagtacatctgcctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtatacgttcggaggggggaccaagctggaaatc

SEQ ID NO: 103 - protein, 111 aa, Artificial Sequence
  Antibody variable light chain sequence
DIVLTQSPASLAVSLGQRATISCRASKSVSTSAYSYMHWYQQKPGQPPKLLIYLASNLESGVPARFSGSGSGTDFTLNIHPVEEEDAATYYCQHSRELPYTFGGGTKLEIK

SEQ ID NO: 104 - protein, 15 aa, Artificial Sequence
  Antibody variable light chain sequence
RASKSVSTSAYSYMH

SEQ ID NO: 105 - protein, 7 aa, Artificial Sequence
  Antibody variable light chain sequence
LASNLES

SEQ ID NO: 106 - protein, 9 aa, Artificial Sequence
  Antibody variable light chain sequence
QHSRELPYT

SEQ ID NO: 107 - DNA, 321 bp, Artificial Sequence
  Antibody variable light chain sequence
gatatccagatgacacagactacatcctccctatctgcctctctgggagacagagtcaccatcagttgcagggcaagtcaggacattagcaattatttaaactggtatcagcagaaaccagatggaactattaaactcctgatctactacacatcaagattacactcaggagtcccatcaaggttcagtggcagtgggtctggaacagattattctctcaccattagcaacctggaacaagaagattttgccacttacttttgccaacagggtaaaacgcttccgtggacgttcggtggaggcaccaagctggaaatcaaa

SEQ ID NO: 108 - protein, 107 aa, Artificial Sequence
  Antibody variable light chain sequence
DIQMTQTTSSLSASLGDRVTISCRASQDISNYLNWYQQKPDGTIKLLIYYTSRLHSGVPSRFSGSGSGTDYSLTISNLEQEDFATYFCQQGKTLPWTFGGGTKLEIK

SEQ ID NO: 109 - protein, 11 aa, Artificial Sequence
  Antibody variable light chain sequence
RASQDISNYLN

SEQ ID NO: 110 - protein, 6 aa, Artificial Sequence
  Antibody variable light chain sequence
TSRLHS

SEQ ID NO: 111 - protein, 9 aa, Artificial Sequence
  Antibody variable light chain sequence
QQGKTLPWT

SEQ ID NO: 112 - DNA, 321 bp, Artificial Sequence
  Antibody variable light chain sequence
gatatccagatgacacagactacatcctccctgtctgcctctctgggagacagagtcaccatcagttgcagggcaagtcaggacattagtaattatttaaattggtatcagcagaaaccagatggatctgttaaactcctgatctactacacatcaagattacactcaggagtcccatcaaggttcagtggcagtgggtctggaacagattattctctcaccattagcaacctggaacaagaagatattgccacttacttttgccaacagggaaagacgcttccgtggacgttcggtggaggcaccaagctggaaatcaaa

SEQ ID NO: 113 - protein, 107 aa, Artificial Sequence
  Antibody variable light chain sequence
DIQMTQTTSSLSASLGDRVTISCRASQDISNYLNWYQQKPDGSVKLLIYYTSRLHSGVPSRFSGSGSGTDYSLTISNLEQEDIATYFCQQGKTLPWTFGGGTKLEIK

SEQ ID NO: 114 - protein, 11 aa, Artificial Sequence
  Antibody variable light chain sequence
RASQDISNYLN

SEQ ID NO: 115 - protein, 5 aa, Artificial Sequence
  Antibody variable light chain sequence
TSRLH

SEQ ID NO: 116 - protein, 9 aa, Artificial Sequence
  Antibody variable light chain sequence
QQGKTLPWT

SEQ ID NO: 117 - DNA, 336 bp, Artificial Sequence
  Antibody variable light chain sequence
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccgtacacgttcggaggggggaccaagctggaaatcaaa

SEQ ID NO: 118 - protein, 112 aa, Artificial Sequence
  Antibody variable light chain sequence
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 119 - protein, 16 aa, Artificial Sequence
  Antibody variable light chain sequence
RSSKSLLHSNGITYLY

SEQ ID NO: 120 - protein, 7 aa, Artificial Sequence
  Antibody variable light chain sequence
QMSNLAS

SEQ ID NO: 121 - protein, 9 aa, Artificial Sequence
  Antibody variable light chain sequence
AQNLELPYT

SEQ ID NO: 122 - protein, 19 aa, Artificial Sequence
  Antibody heavy chain signal sequence
MGWSLILLFLVAVATRVLS

SEQ ID NO: 123 - DNA, 106 bp, Artificial Sequence
  Oligonucleotide PCR primer
cgaacaggcccagctggccaccatggacatgagggtccccgctcagctcctggggctccttctgctctggctcccaggtgccagatgtgacatccagatgacccag

SEQ ID NO: 124 - DNA, 37 bp, Artificial Sequence
  Oligonucleotide PCR primer
tcgcacggcgcgcctcaacactctcccctgttgaagc

SEQ ID NO: 125 - DNA, 83 bp, Artificial Sequence
  Oligonucleotide PCR primer
cggccaccatgggttggagcctcatcttgctcttccttgtcgctgttgctacgcgtgtcctgtccgaagttcaattgttagag

SEQ ID NO: 126 - DNA, 47 bp, Artificial Sequence
  Oligonucleotide PCR primer
gggatcggccagctgggccccttcgttgaggcgcttgagacggtgac

SEQ ID NO: 127 - protein, 19 aa, Artificial Sequence
  Antibody heavy chain signal peptide
MGWSCIILFLVATATGAHS

SEQ ID NO: 128 - protein, 22 aa, Artificial Sequence
  Antibody light chain signal peptide
MDMRVPAQLLGLLLLWLRGARC

SEQ ID NO: 129 - protein, 20 aa, Artificial Sequence
  Antibody light chain signal peptide
METDTLLLWVLLLWVPGSTG

SEQ ID NO: 130 - DNA, 39 bp, Artificial Sequence
  Oligonucleotide PCR primer
ggggatatccaccatggratgsagctgkgtmatsctctt

SEQ ID NO: 131 - DNA, 39 bp, Artificial Sequence
  Oligonucleotide PCR primer
aggtctagaayctccacacacaggrrccagtggatagac

SEQ ID NO: 132 - DNA, 39 bp, Artificial Sequence
  Oligonucleotide PCR primer
ggggatatccaccatggattttcaggtgcagattttcag

SEQ ID NO: 133 - DNA, 29 bp, Artificial Sequence
  Oligonucleotide PCR primer
gcgtctagaactggatggtgggagatgga

SEQ ID NO: 134 - DNA, 1407 bp, Artificial Sequence
  cDNA encoding antibody chimeric heavy chain
atggaatggagctgtgtcatgctcttcatcctgtcaggaactgcaggtgtccactcccaggttcagctgcagcagtctggacctgagctagtgaagcctggggcttcagtgaagatgtcctgcaaggcttctggaaacacattcactgactatgttataaactgggtgaagcagagaactggacagggccttgagtggattggagagatttatcctggaaatgaaaatacttattacaatgagaagttcaagggcaaggccacactgactgcagacaaatcctccaacacagcctacatgcagctcagtagcctgacatctgaggactctgcggtctatttctgtgcaagagggatttattactacggtagtaggacgaggactatggactactggggtcaaggaacctcagtcaccgtctcctcagcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcgcgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtcctcgattccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 135 - protein, 449 aa, Artificial Sequence
  Antibody chimeric heavy chain
QVQLQQSGPELVKPGASVKMSCKASGNTFTDYVINWVKQRTGQGLEWIGEIYPGNENTYYNEKFKGKATLTADKSSNTAYMQLSSLTSEDSAVYFCARGIYYYGSRTRTMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 136 - DNA, 32 bp, Artificial Sequence
  Oligonucleotide PCR primer
cgccagtgtgcggccgctggaattcgcccttg

SEQ ID NO: 137 - DNA, 47 bp, Artificial Sequence
  Oligonucleotide PCR primer
ggaccaagctggagctgaagcgtacggatgctgcaccaactgtatcc

SEQ ID NO: 138 - DNA, 714 bp, Artificial Sequence
  cDNA encoding chimeric antibody light chain
atggattttcaggtgcagattttcagcttgctgctaatcagtgtcacagtcatagtgtctaatggagaagttgtgctcacccagtctccaaccgccatggctgcatctcccggggagaagatcactatcacctgcagtgccagctcaactttaagttccaattacttgcattggtatcagcagaagccaggattctcccctaaactcttgatttataggacatccaatctggcctctggagtcccaggtcgcttcagtggcagtgggtctgggacctcttactctctcacaattggcaccatggaggctgaagatgttgccacttactactgccagcagggtagtagtataccgctcacgttcggtgctgggaccaagctggagctgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 139 - protein, 215 aa, Artificial Sequence
  Chimeric antibody light chain
EVVLTQSPTAMAASPGEKITITCSASSTLSSNYLHWYQQKPGFSPKLLIYRTSNLASGVPGRFSGSGSGTSYSLTIGTMEAEDVATYYCQQGSSIPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - DNA, 1404 bp, Artificial Sequence
  cDNA encoding chimeric antibody heavy chain
atggactggacctggagggtcttctgcttgctggctgtagcaccaggtgcccactccgacgtccaactgcaggagtctggacctgacctggtgaaaccttctcagtcactttcactcacctgcactgtcactggctactccatcaccagtggttatagctggcactggatccggcagtttccaggaaacaaactggaatggatgggctacatacactacagtggtggcactaactacaacccatctctcaaaagtcgaatctctatcactcgagacacatccaagaaccagttcttcctccagttgaattctgtgactactgaggacacagccacatattactgtgcaagatcggggtacggctacaggagtgcgtactattttgactactggggccaagggaccacggtcaccgtctcctcagcttccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcgcgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtcctcgattccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 141 - protein, 448 aa, Artificial Sequence
  Chimeric antibody heavy chain
DVQLQESGPDLVKPSQSLSLTCTVTGYSITSGYSWHWIRQFPGNKLEWMGYIHYSGGTNYNPSLKSRISITRDTSKNQFFLQLNSVTTEDTATYYCARSGYGYRSAYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 142 - DNA, 717 bp, Artificial Sequence
  cDNA encoding chimeric antibody light chain
atggagacagacacactcctgttatgggtactgctgctctgggttccaggttccactggtgacattgtgctgacacagtctcctgcttccttagctgtatctctggggcagagggccaccatctcatgcagggccagcaaaagtgtcagtacatctgcctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtatacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 143 - protein, 218 aa, Artificial Sequence
  Chimeric antibody light chain
DIVLTQSPASLAVSLGQRATISCRASKSVSTSAYSYMHWYQQKPGQPPKLLIYLASNLESGVPARFSGSGSGTDFTLNIHPVEEEDAATYYCQHSRELPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - DNA, 1404 bp, Artificial Sequence
  cDNA encoding chimeric antibody heavy chain
atggactggacctggagggtcttctgcttgctggctgtagcaccaggtgcccactccgacgtccaactgcaggagtctggacctgacctggtgaaaccttctcagtcactttcactcacctgcactgtcactggctactccatcaccagtggttatagctggcactggatccggcagtttccaggaaacaaactggaatggatgggctacatacactacagtggtggcactaactacaacccatctctcaaaagtcgaatctctatcactcgagacacatccaagaaccagttcttcctccagttgaattctgtgactactgaggacacagccacatattactgtgcaagatcggggtacggctacaggagtgcgtactattttgactactggggccaagggaccacgttgacagtctcctcagcttccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcgcgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtcctcgattccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 145 - protein, 448 aa, Artificial Sequence
  Chimeric antibody heavy chain
DVQLQESGPDLVKPSQSLSLTCTVTGYSITSGYSWHWIRQFPGNKLEWMGYIHYSGGTNYNPSLKSRISITRDTSKNQFFLQLNSVTTEDTATYYCARSGYGYRSAYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 146 - DNA, 1401 bp, Artificial Sequence
  cDNA encoding chimeric antibody heavy chain
atgggttggatctgtatctttctattcttggtggcagctgcccaaagtgcccaagcacagatccagttggtgcagtctggacctgacctgaagaagcctggagagacagtcaagatctcctgcaaggcttctgggtataccttcacaaaccatggaatgaactgggtgaagcaggctccaggaaaggatttaaagtggatgggctggataaacaccaacactggagagccaacatatgctgatgacttcaagggacggtttgccttctctttggaaacctctgccagcactgcctatttgcagatcaacaacctcaaaaatgaggacacggctacatatttctgtgcaagtcccctctactataggaacgggcgatacttcgatgtctggggcgcagggaccacggtcaccgtctcctcagcttccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcgcgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtcctcgattccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 147 - protein, 447 aa, Artificial Sequence
  Chimeric antibody heavy chain
QIQLVQSGPDLKKPGETVKISCKASGYTFTNHGMNWVKQAPGKDLKWMGWINTNTGEPTYADDFKGRFAFSLETSASTAYLQINNLKNEDTATYFCASPLYYRNGRYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 148 - DNA, 705 bp, Artificial Sequence
  cDNA encoding chimeric antibody light chain
atgaggtcccctgctcagtttcttggtctcctgttgctctgttttcaaggtgccagatgtgatatccagatgacacagactacatcctccctgtctgcctctctgggagacagagtcaccatcagttgcagggcaagtcaggacattagtaattatttaaattggtatcagcagaaaccagatggatctgttaaactcctgatctactacacatcaagattacactcaggagtcccatcaaggttcagtggcagtgggtctggaacagattattctctcaccattagcaacctggaacaagaagatattgccacttacttttgccaacagggaaagacgcttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 149 - protein, 214 aa, Artificial Sequence
  Chimeric antibody light chain
DIQMTQTTSSLSASLGDRVTISCRASQDISNYLNWYQQKPDGSVKLLIYYTSRLHSGVPSRFSGSGSGTDYSLTISNLEQEDIATYFCQQGKTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 446 aa, Artificial Sequence
  Chimeric antibody heavy chain
QIQLVQSGPELKKPGETVKISCKASGYTFTNHGMNWVKQAPGKGLKWMGWNTSTGEPTYADDFKGRFAFSLETSASTAFLQINNLKNEDTASYFCASPLYYMYGRYIDVWGAGTAVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 151 - DNA, 705 bp, Artificial Sequence
  cDNA encoding chimeric antibody light chain
atgaggtcccctgctcagtttcttggagacctgttgctctgttttcaaggtaccagatgtgatatccagatgacacagactacatcctccctatctgcctctctgggagacagagtcaccatcagttgcagggcaagtcaggacattagcaattatttaaactggtatcagcagaaaccagatggaactattaaactcctgatctactacacatcaagattacactcaggagtcccatcaaggttcagtggcagtgggtctggaacagattattctctcaccattagcaacctggaacaagaagattttgccacttacttttgccaacagggtaaaacgcttccgtggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 152 - protein, 214 aa, Artificial Sequence
  Chimeric antibody light chain
DIQMTQTTSSLSASLGDRVTISCRASQDISNYLNWYQQKPDGTIKLLIYYTSRLHSGVPSRFSGSGSGTDYSLTISNLEQEDFATYFCQQGKTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - DNA, 324 bp, Artificial Sequence
  cDNA encoding antibody light chain constant domain
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 154 - protein, 107 aa, Artificial Sequence [sequence omitted]

SEQ ID NO: 155 - DNA, 981 bp, Artificial Sequence
  cDNA encoding antibody aglycosylated heavy chain constant domain
gcctcaacgaaggggcccagcgtgttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcgcgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtcctcgattccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 156 - protein, 326 aa, Artificial Sequence
  Antibody aglycosylated heavy chain constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 157 - protein, 22 aa, Artificial Sequence
  Immunoglobulin light chain signal peptide
MDMRVPAQLLGLLLLWLPGARC

SEQ ID NO: 158 - protein, 1337 aa, Homo sapiens
  IGF-1R minus 30 amino acid signal peptide
EICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC